(19) United States
(12) Patent Application Publication
(10) Pub. No.: US 2024/0374631 A1
(43) Pub. Date: Nov. 14, 2024

(54) A COMPOSITION FOR IMPROVING GUT MICROBIOTA, BEHAVIOURAL PATTERN, ALPHA-SYNUCLEIN LEVELS, SLEEP PATTERN AND/OR SERUM MELATONIN

(71) Applicants: GN Corporation Co Ltd, Yamanashi (JP); Sophy Inc., Kochi (JP)

(72) Inventors: TAKASHI ONAKA, Okayama (JP); Samuel JK ABRAHAM, Yamanashi (JP)

(21) Appl. No.: 18/554,442

(22) PCT Filed: Apr. 7, 2022
(86) PCT No.: PCT/JP2022/017237

§ 371 Date: Oct. 6, 2023

(30) Foreign Application Priority Data
Apr. 8, 2021 (JP) 2021-065938
Apr. 8, 2021 (JP) 2021-076579
Oct. 26, 2021 (JP) 2021-174365

(51) Int. Cl. A61K 31/716; A61K 36/062; A61P 1/00; A61P 25/28

(52) U.S. Cl. CPC A61K 31/716; A61K 36/062; A61P 1/00; A61P 25/28

(57) ABSTRACT

Compositions for improving gut microbiota, improving behavioural pattern and alpha-synuclein levels, and improving sleep pattern and serum melatonin, especially in children with autism, are provided. Methods are also included for control of gut enterobacteria in children with autism, reconstitution of gut microbiota, and control of enterobacteria in autism and neurodegenerative diseases.

### CROSS-REFERENCE TO RELATED APPLICATIONS

This application is a national phase application filed under 35 USC 371 of International Application No. PCT/JP2022/017237, filed Apr. 7, 2022, and claims the benefit of the filing dates of Japanese Application No. 2021-65938, entitled “A COMPOSITION FOR IMPROVING SLEEP PATTERN AND SERUM MELATONIN”, filed Apr. 8, 2021; Japanese Application No. 2021-76579, entitled “A COMPOSITION FOR IMPROVING BEHAVIOURAL PATTERN AND ALPHA-SYNUCLEIN LEVELS”, filed Apr. 28, 2021; and Japanese Application No. 2021-174365, entitled “A COMPOSITION FOR IMPROVING GUT MICROBIOTA”, filed Oct. 26, 2021; the contents of each of which are incorporated herein by reference in their entirety.

### BACKGROUND

### Field of the Invention

The present invention relates to a composition for improving gut microbiota; control of gut enterobacteria in children with autism; reconstitution of gut microbiota and control of enterobacteria in autism and neurodegenerative diseases; a composition for improving behavioural pattern and alpha-synuclein levels, especially in autism; a composition for improving sleep pattern and serum melatonin, especially in children with autism.

### Background Art

### Gut Microbiota

Gut dysbiosis is one of the major pathologies in children with autism spectrum disorder (ASD). Evaluation of gut microbiota of the subjects in the present randomized pilot clinical study was undertaken and compared with an aim of gaining a mechanistic insight.

### Behavioral Pattern and Alph-Synuclein Levels

Autism spectrum disorders (ASDs) are a group of developmental disabilities that can cause significant impairment in social, emotional, and communication skills (cdc.gov). Several causes and underlying mechanisms have been postulated for the pathogenesis of ASD, including genetic, environmental, immune dysregulation, neuroinflammation, and oxidative stress. Neuronal synaptic imbalance and mutation in synaptic proteins and receptors have also been reported to be associated with ASD (Al-Mazeedi et al., 2020). Synucleins are small soluble proteins that are present in presynaptic terminals, and they regulate synaptic plasticity and neurotransmitter release. Synucleins are important in the context of brains and neurons (Al-Mazeedi et al., 2020; Vargas et al., 2017).

Alpha-synuclein has already been reported to be associated with several neurodegenerative disorders, collectively called synucleinopathies such as Alzheimer's disease (AD), Parkinson's disease (PD), dementia with Lewy bodies (DLBs), and multiple system atrophy (Al-Mazeedi et al., 2020). Several studies have recently reported a strong association between lower levels of α-synuclein and ASD. At present, there is no definitive cure for ASD (Al-Mazeedi et al., 2020; Kadak et al., 2015; Sriwimol et al., 2018). Interventions involve speech and behavioural therapies to improve the symptoms. According to the research, the microbiota-gut-brain axis is significant because dysbiosis has been observed in gut-related diseases and other generalized disorders, especially of the nervous system, such as AD, multiple sclerosis, PD, and ASD (Srikantha et al., 2019). Therefore, nutritional supplements are considered potential interventions in alleviating gastrointestinal and behavioural symptoms in ASD (Karhu et al., 2020).

Beta-glucans, especially yeast-derived ones, have shown a considerable positive outcome as food supplements in modulating gut microbiota (Karhu et al., 2020; Xu et al., 2020). Nichi Glucan is a black yeast-derived AFO-202 (also referred to as FO-68 [(accession number) FERM BP-19327]) beta-glucan that has been in consumption for the past two decades (Ikewaki et al., 2007) and has been shown to have potential as a nutritional supplement to balance metabolic levels of glucose, lipids, and immunomodulators (Dedeepiya et al., 2012; Ganesh et al., 2014; Ikewaki et al., 2021). The present study was undertaken to investigate the effects of Nichi Glucan as a food supplement in children with ASD, especially with relevance to the childhood autism rating scale (CARS) score and alpha-synuclein levels.

### Sleep and Melatonin

Inability to have a good quality of sleep is one of the major problems faced by people with autism spectrum disorders (ASD). This is attributed to lower melatonin levels and therefore melatonin supplementation is one of the treatments opted, reported with varying outcome. Beta Glucans having been earlier reported in animal studies to improve melatonin levels, we herein report the outcome of our pilot clinical study in which a Black yeast Aureobasidium pullulans derived Beta 1,3-1,6 Glucan food supplement was administered in children with ASD.

### CITATION LIST

### Non Patent Literature

[NPL 1] Shi H, Yu Y, Lin D, et al. β-glucan attenuates cognitive impairment via the gut-brain axis in diet-induced obese mice. Microbiome. 2020; 8:143.
[NPL 2] Sriwimol W, Limprasert P. Significant changes in plasma alpha-synuclein and beta-synuclein levels in male children with autism spectrum disorder. Biomed Res Int 2018; 2018:1-7.doi:10.1155/2018/4503871
[NPL 3] Dutta S D, Patel D K, Ganguly K, Lim K T. Effects of GABA/β-glucan supplements on melatonin and serotonin content extracted from natural resources. PLoS One. 2021 Mar. 5; 16(3):e0247890. doi: 10.1371/journal.pone.0247890. PMID: 33667254; PMCID: PMC7935273.
[NPL 4] Miller A L, Bessho S, Grando K, Tükel Ç. Microbiome or Infections: Amyloid-Containing Biofilms as a Trigger for Complex Human Diseases. Front Immunol. 2021 Feb. 26; 12:638867.
[NPL 5] Grimaldi R, Gibson G R, Vulevic J, Giallourou N, Castro-Mejia J L, Hansen L H, Leigh Gibson E, Nielsen D S, Costabile A. A prebiotic intervention study in children with autism spectrum disorders (ASDs). Microbiome. 2018 Aug. 2; 6(1):133.

### SUMMARY OF THE INVENTION

The present invention relates to the following:
1. A composition for improving gut microbiota, comprising a beta-glucan produced by Aureobasidium pullulans AFO-202 (FERM BP-19327).
2. The composition according to 1, wherein the improvement of gut microbiota comprises a decrease of Akkermansia muciniphila with an increase of beneficial bacteria including Roseburia in a gut.
3. The composition according to 1 or 2, wherein the composition is for prophylactic, ameliorating and/or curative treatment of autism spectrum disorders (ASD), multiple sclerosis (MS), Alzheimer's disease (AD), Parkinson's disease (PD) and/or epilepsy.
4. The composition according to 1 or 2 for improving behavioural pattern and alpha-synuclein levels.
5. The composition according to 1 or 2, wherein the composition is for improving sleep pattern and serum melatonin.
6. The composition according to 5, wherein the improvement is in a child with autism spectrum disorder.
7. The composition according to any one of 1 to 6, wherein the composition is a pharmaceutical composition.
8. The composition according to any one of 1 to 6, wherein the composition is a food composition.

The present invention also relates to the following:
[1A] A composition for improving gut microbiota, comprising a beta-glucan produced by Aureobasidium pullulans AFO-202 (FERM BP-19327).
[2A] The composition according to [1A], wherein the composition is a pharmaceutical composition.
[3A] The composition according to [1A], wherein the composition is a food composition.
[4A] The composition according to any one of [1A]-[3A], wherein the composition is for prophylactic, ameliorating and/or curative treatment of autism spectrum disorders (ASD), multiple sclerosis (MS), Alzheimer's disease (AD) and/or Parkinson's disease (PD).
[1B] A composition for improving behavioural pattern and alpha-synuclein levels, comprising a beta-glucan produced by Aureobasidium pullulans AFO-202 (FERM BP-19327).
[2B] The composition according to [1B], wherein the composition is a pharmaceutical composition.
[3B] The composition according to [1B], wherein the composition is a food composition.
[1C] A composition for improving sleep pattern and serum melatonin, comprising a beta-glucan produced by Aureobasidium pullulans AFO-202 (FERM BP-19327).
[2C] The composition according to [1C], wherein the composition is a pharmaceutical composition.
[3C] The composition according to [1C], wherein the composition is a food composition.

### Effects of the Invention

Gut microbiota is improved and/or well-balanced by the present invention. Such a control in gut microbiota leads to an improvement of behavioural pattern, alpha-synuclein levels, sleep pattern and/or serum melatonin levels.

The present invention is effective in balancing the gut microbiota, especially, by a decrease in Bacteroides, Curli producing Enterobacteria Escherichia coli, Akkermansia muciniphila CAG:154, Blautia spp., Coprobacillus sp., and Clostridium bolteae with increase in Roseburia, Faecalibacterium prausnitzii and Prevotella copri. The other beta glucans don't produce such an effective and balanced modulation of gut bacteria with effects on diverse species (Shen R L, et al. 2012, Turunen K, et al. 2011, Zhen W, et al. 2021). While the enterobacteria may be decreased, a decrease in Akkermansia muciniphila has not been concomitantly reported. Further the concomitant increase in beneficial bacteria such as Roseburia, Faecalibacterium prausnitzii and Prevotella copri is an added advantage.

The present invention is effective for prophylactic, ameliorating and/or curative treatment of neurological disorders such as autism spectrum disorders (ASD), multiple sclerosis (MS), Alzheimer's disease (AD), Parkinson's disease (PD) and/or epilepsy. The present invention is effective for improving sleep pattern and serum melatonin, especially in children with autism spectrum disorder.

### BRIEF DESCRIPTION OF THE DRAWINGS

FIG. 1. Index of the most abundant taxa across species levels.

FIGS. 2A and 2B. Differences pre and post-intervention in the read count of selected bacteria between A. Enterobacteria; and, B. Firmicutes.

FIGS. 3A-3C. Most abundant taxa at the phyla (A), genus (B), and species (C) levels in the different groups of the study.

FIGS. 4A-4D. Principal component analysis (PCA) of: A, B. all the ten samples (five groups—pre- and post-intervention)—A, C. Score plot; B, D. Loading plot; Study groups (1) Control/Vehicle, 2—Baseline, 12—post-intervention, (2) AFO-202, 4—Baseline, 14—post-intervention, and (3) Telmisartan, 10—Baseline, 20—post-intervention.

FIGS. 5A-5C. Differential abundance analysis, log 2 fold change results for each group before and after intervention: A. Control, B. AFO-202, and C. Telmisartan.

FIGS. 6A-6F. Score plots of principal component analysis (PCA) and compounds with a VIP value of 1 or higher in the OPLS-DA of the different groups: A. Control; B. AFO-202; C. Telmisartan.

FIGS. 7A-7E. Peak height of the detected compounds after normalization. A. Succinic acid, B. Phosphoric acid, C. Fructose, D. Isoleucine, and E. Leucine (**significant;*not significant; p-value significance <0.05).

FIG. 8. Euclidean distance hierarchical clustering analysis demonstrating the different intensity levels of characteristic metabolites in AFO-202.

FIGS. 9A and 9B. Euclidean distance hierarchical clustering analysis demonstrating the different intensity levels of characteristic metabolites in A. Control; and, B. Telmisartan.

FIG. 10 shows decrease in abundance of Enterobacteria in AFO-202 compared to Control, post-intervention.

FIG. 11 shows decrease in abundance of Bacteroides in AFO-202 compared to Control, post-intervention.

FIG. 12 shows increase in abundance of Prevotella in AFO-202 compared to Control, post-intervention.

FIG. 13 shows decrease in Lactobacillus in AFO-202 and Control.

FIG. 14 shows CONSORT flow diagram of the trial.

FIG. 15. Data before and after adapter trimming.

FIG. 16. Average Read quality before and after adapter trimming.

FIG. 17. Average GC % before and after adapter trimming.

FIG. 18. Percentage of alignment to Bacteria, Fungi, Virus, Archae Bacteria and Human genomes.

FIG. 19. Post intervention control vs post intervention treatment SEED Average values.

FIG. 20. Distribution of Phyla: Phylum Proteobacteria was the most abundant followed by Phylum Firmicutes.

FIGS. 21A and 21B. Genus Abundance of major genera identified (A) Group (Gr.) 1 at baseline versus post-intervention and (B) Group (Gr.) 2 at baseline versus post-intervention.

FIGS. 22A-22D. FIG. 22A shows decrease in abundance of Enterobacteria in Group 2 compared to Group (Gr.) 1, post-intervention. FIG. 22B shows decrease in abundance of Bacteroides in Group (Gr.) 2, which had increased in Group 1 post-intervention. FIG. 22C shows increase in Prevotella in Group 1 and Group 2 post-intervention. FIG. 22D shows decrease in Lactobacillus in Group 1 and Group 2 post-intervention.

FIGS. 23A and 23B. Species Abundance of major species analysed; (A) Group (Gr.) 1 at baseline versus post-intervention and (B) Group (Gr.) 2 at baseline versus post-intervention.

FIGS. 24A and 24B. Distribution of Genera and Distribution of Species.

FIGS. 25A-25D. (A) Decrease in abundance of E. coli was significant in Group (Gr.) 2 compared to Group (Gr.) 1, post-intervention; (B) Significant increase in abundance of Faecalibacterium prausnitzii in Group 2 compared to Group 1 post-intervention; (C) Increase in Akkermansia muciniphila in Group 1 but decrease in Group 2 post-intervention; and, (D) Increase in Clostridium bolteae CAG:59 in Group 1 but decrease in Group 2 post-intervention.

FIGS. 26A-26C. A. The score on the childhood autism rating scale (CARS) in Gr. 1 (control), which was very mild and showed no improvement after the study, whereas the score decreased in all children by an average of 3 points for B. Gr. 2 (Nichi Glucan); and, C. Comparison between Gr. 1 and Gr. 2 showed a significant decrease in the CARS score, indicating improvement in autism's signs and symptoms in Gr. 2 (Nichi Glucan) compared to Gr. 1 (control) post-intervention (p-value=0.034517).

FIGS. 27A-27C. A. Plasma alpha-synuclein levels in Gr. 1 (control) pre- and post-intervention; B. Plasma alpha-synuclein in Gr. 2 (Nichi Glucan) pre- and post-intervention; and, C. Plasma alpha-synuclein levels in Nichi Glucan group Gr. 2 showed a significant increase compared to Gr. 1 (p-value=0.091701).

FIG. 28. The total sleep pattern score of the Children's Sleep Habits Questionnaire-Abbreviated (CSHQ-A) showing significant improvement by decrease in score in Nichi Glucan (Gr.2) compared to Gr.1 (Control) after the intervention.

FIGS. 29A-29C. Serum melatonin levels in Gr. 1 (Control group) pre- and post-intervention; serum melatonin levels in Gr. 2 (Nichi Glucan group) pre- and post-intervention; and fold increase in Nichi Glucan group Gr. 2 greater than in Gr.1

FIG. 30 explains, stepwise, the pathogenesis as well as the way beta glucan tackles each stage of the disease process: (A) & (B) Enterobacteria secretion of curli that causes misfolding of α-synuclein (αSyn); its aggregation in enteric neuronal cells is tackled by (1) control of enterobacteria, (2) scavenging of the accumulated amyloids by activated natural killer cells, and (3) reconstitution of beneficial microbiome; (C) The prion like propagation may not occur because the accumulation of curli proteins and amyloids is controlled at the level of production and aggregation (1) as well as clearing of already accumulated deposits (3); and, (D) Deposition of Lewy bodies, amyloid fibrils, and misfolded αSyn are tackled by (4) microglial-based scavenging.

FIG. 31. Increase in abundance of Roseburia hominis after AFO-202, whose increase may be attributed to the increase in melatonin.

FIG. 32. Increase in abundance of Roseburia inulinivorans after AFO-202, whose increase may be attributed to the increase in melatonin.

FIG. 33. Increase in abundance of Roseburia intestinalis after AFO-202, whose increase may be attributed to the increase in melatonin.

FIG. 34. Increase in abundance of Roseburia faecis after AFO-202, whose increase may be attributed to the increase in melatonin.

FIG. 35. Post Intervention Control vs Post Intervention Treatment Seed Average of the different functional properties and metabolic pathways beneficially modulated in AFO-202 (Treatment group).

FIGS. 36A and 36B. Expression of α-synuclein on the neuroblastoma cell line (SHSY-5Y Tet-On: SCC291) α-synuclein polyclonal antibody (Proteintech Co. 10842-1-AP), and expression of α-synuclein on the neuroblastoma cell line (SHSY-5Y Tet-On: SCC291) α-synuclein polyclonal antibody in a wider field of magnification (Proteintech Co. 10842-1-AP).

FIG. 37. Illustration of how AFO-202 prevents and treats neurological diseases by controlling production and propagation of Abnormal Alpha-Synuclein.

FIG. 38. Illustration of how AFO-202 prevents and treats neurological diseases by controlling production and propagation of Abnormal Alpha-Synuclein at the gut level by controlling the production of Alpha-Synuclein and scavenging of aggregates by Natural Killer (NK) cells and in the brain by microglia.

### DETAILED DESCRIPTION OF INVENTION

The present invention relates to a composition for improving gut microbiota. The present invention also relates to an effective control of gut enterobacteria producing Alpha synuclein and Curli amyloids after consumption of Aureobasidium pullulans derived beta 1,3-1,6 glucans in children with autism spectrum disorder in a clinical pilot study. The present invention also relates to a beneficial reconstitution of gut microbiota and control of alpha-synuclein and curli-amyloids-producing enterobacteria, by beta 1,3-1,6 glucans in a clinical pilot study of autism and potentials in neurodegenerative diseases. The present invention also relates to a gut microbiota reconstitution by Beta Glucans in autism.

Further, the present invention relates to a composition for improving behavioural pattern and alpha-synuclein levels, especially in autism spectrum disorder. The present invention also relates to an improvement of behavioural pattern and alpha-synuclein levels in autism spectrum disorder after consumption of a beta-glucan food supplement in a randomized, parallel-group pilot clinical study.

Moreover, the present invention relates to a composition for improving sleep pattern and serum melatonin, especially in children with autism spectrum disorder. The present invention also relates to an improvement of sleep pattern and serum melatonin in children with autism spectrum disorder after consumption of Beta 1,3-1,6 Glucan in a pilot clinical study.

The glucan contained in the composition of the present invention can be a glucan derived from Aureobasidium pullulans strain FO-68 (Also referred to herein as “strain AFO 202”), and preferably β-1,3-1,6 glucan derived from FO-68 (Also referred to herein simply as “glucan”, “AFO 202 glucan” or “AF 202 beta glucan”). “Aureobasidium pullulans strain FO-68” has been deposited at the Patent Biological Depository Center, National Institute of Advanced Industrial Science and Technology, under the deposit number FERMP-19327.

While the domestic deposition was made on Apr. 23, 2003, Aureobasidium pullulans strain FO-68 has then been transferred to international deposition at the International Patent Organism Depositary, National Institute of Technology and Evaluation (Room. 120, 2-5-8, Kazusa Kamatari, Kisarazu-shi, Chiba, 292-0818 Japan) on Apr. 21, 2021 with the accession number: FERM BP-19327.

Aureobasidium pullulans strain FO-68 is also called as Aureobasidium strain FERM P-18099.

### Scientific Properties of FO-68

This fungus produces high-molecular polysaccharide with high viscosity. This substance agglutinates easily with ethanol, making it possible to collect simply. This polysaccharide is of [beta] type, and is acidic polysaccharide having a main chain of 1,3 bond and branches from 3- and 6-positions. It contains carboxylic acids such as malic acid as organic acids and phosphoric acid. Moreover, it agglutinates easily with aluminum ions etc. This substance is also effective for the promotion of growth as a feed and the effluent treatment. It is effective as a food additive and functional food.

FO-68 forms blackish brown colonies on potato-dextrose-agar slant culturing for 7 days at 25 C. The fringe of colonies shows filamentous growth and becomes gradually light blackish brown. The cells are filamentous, and sometimes arthrospores, yeast-like budding conidiospores, oval yeast-like single cells, and, in some time, thick-walled spore cells are formed. The growth temperature is 25 C., and it decomposes hexoses such as glucose, fructose and galactose, sucrose, and starch. The medium becomes conspicuously viscous. Based on FO-68's mycological properties, it is a kind of Aureobasidium pullulans in the black fungus family of deuteromycetes.

### Mycological Features of the Isolated Fungus

A colony of FO-68 has a smooth surface at first and grows into a grayish white, mucous and glossy oil drop-like (fat-like), yeast-like material. The filamentous fungus body grows radially from the fringe thereof, leading to crinkled, filamentous and just dendritic growth. This filamentous fungus body grows well not only on the surface of medium, but also in the medium. In a short time, light dark brown specks appear here and there on the surface of colony, which become black specks gradually, and overall surface becomes dark black eventually. On this filamentous fungus body, a lot of light brown, elliptic or oval conidiospores are produced laterally. This conidiospore falls easily in pieces. While the surface of oil drop-like colony puts on the conidiospores here and there.

As a method for culturing FO-68 and a method for producing β-1,3-1,6 glucan using FO-68, known methods can be used, for example, see JP 2004-329077A.

In some embodiment, the present invention relates to a composition comprising a beta-glucan produced by Aureobasidium pullulans AFO-202 (FERM BP-19327) for improving gut microbiota, behavioural pattern, alpha-synuclein levels, sleep pattern and/or serum melatonin. In another aspect, the present invention also relates to use of Aureobasidium pullulans AFO-202 (FERM BP-19327) for improving gut microbiota, behavioural pattern, alpha-synuclein levels, sleep pattern and/or serum melatonin, and particularly relates to a method of improving gut microbiota, behavioural pattern, alpha-synuclein levels, sleep pattern and/or serum melatonin by administering Aureobasidium pullulans AFO-202 (FERM BP-19327) to a subject.

In the composition used in the present invention, a culture of FO-68 may be used as it is without purification, or glucan isolated from the culture or further purified as necessary may be used. In addition, for example, the culture product of the present invention was crushed into a concentrate, a paste, a spray-dried product, a freeze-dried product, a vacuum-dried product, a drum-dried product, a liquid product dispersed in a medium, a diluted product, and a dried product.

The composition of the present invention exerts its function when ingested by mammals including humans. The term “ingestion” as used herein is not limited to any administration route as long as it can enter the human body, and is realized by all known administration methods such as oral administration, tube administration, and enteral administration. Typically, oral ingestion and enteral ingestion via the digestive tract are preferable.

The dose of the present invention can be appropriately set in consideration of various factors such as administration route, age, body weight, and symptoms. The dose of the composition of the present invention is not particularly limited, but the amount of glucan is preferably 0.05 mg/kg/day or more, more preferably 0.5 mg/kg/day or more, particularly preferably 1.0 mg/kg/day. However, when ingested over a long period of time, the amount may be smaller than the preferable amount described above. In addition, the glucan used in the present invention has a sufficient dietary experience, and there is no problem in terms of safety. Therefore, an amount far exceeding the above amount (for example, 10 mg/kg/day or more) is possible.

The composition of the present invention can be used as a food or drink. The composition of the present invention, as a special-purpose food such as a food for specified health use and a nutritionally functional food, by administering to animals such as humans, can improve gut microbiota, behavioural pattern, alpha-synuclein levels, sleep pattern and/or serum melatonin.

When the composition of the present invention is used as food or drink, the type of food or drink is not particularly limited. Further, the shape of the food or drink is not particularly limited, and may be any shape of food or drink that is usually used. For example, it may be in any form such as solid form (including powder and granule form), paste form, liquid form and suspension form, and is not limited to these forms.

When used as a pharmaceutical, a dosage form that can be orally administered is preferable because the composition of the present invention reaches the intestine. Examples of preferable dosage forms of the drug according to the present invention include tablets, coated tablets, capsules, granules, powders, solutions, syrups, troches and the like. These various preparations are prepared according to a conventional method by using glucan, which is the active ingredient, an excipient, a binder, a disintegrating agent, a lubricant, a coloring agent, a flavoring agent, a solubilizing agent, a suspending agent, a coating agent, etc. It can be formulated by admixing the auxiliaries usually used in the technical field of pharmaceutical formulation.

In some embodiment, the present invention can be used in combination with other food, drink, drugs and any other substances in order to enhance the efficacy of the present invention.

Beneficial Regulation of Gut Microbiota Yielding an Advantageous Fecal Metabolome Profile by Administration of AFO-202 Biological Response Modifier Glucan in an Stam Animal Model of Non-Alcoholic Steatohepatitis, Paving Way for Effective Utilization in Human Health and Disease.

### Introduction:

With approximately 100 trillion micro-organisms existing in the human gastrointestinal tract, the microbiome is now considered as a virtual organ of the body. The microbiome encodes over three million genes producing thousands of metabolites compared to 23,000 genes of the human genome and hence replaces many of the functions of the host influencing the host's fitness, phenotype, and health. Gut microbiota influences several aspects of human health including immune, metabolic and neurobehavioural traits [D1]. In regard to functions, the gut microbiota ferments non-digestible substrates like dietary fibres and endogenous intestinal mucus. The fermentation supports the growth of specialist microbes that produce short chain fatty acids (SCFAs) and gases. Major SCFAs produced are acetate, propionate, and butyrate.

Butryate is needed to maintain colon's cells, it helps in apoptosis of colon cancer cells, activation of intestinal gluconeogenesis, having beneficial effects on glucose and energy homeostasis and maintenance of oxygen balance in the gut, preventing gut microbiota dysbiosis. Propionate is transported to the liver, where it regulates gluconeogenesis and acetate is an essential metabolite for the growth of other bacteria, as well as playing a role in central appetite regulation [D1].

Gut dysbiosis or the altered state of the microbiota community has been associated with several diseases including but not limited to diabetes, metabolic disorders, obesity, cancers, rheumatoid arthritis, neurological disorders such as Parkinson's disease, Alzheimer's′, multiple sclerosis and autism spectrum disorders (ASD) [D2,3]. The fecal metabolome represents the functional readout of the gut microbial activity and can be considered to be an intermediate phenotype mediating host-microbiome interaction. An average 67.7% (±18.8%) of the fecal metabolome's variance represents the gut microbial composition.

Fecal metabolic profiling thus is a novel tool to explore links among microbiome composition, host phenotypes and disease states [D4]. Probiotics and pre-biotic nutritional supplements represent the major strategy other than fecal microbiota transplantation to restore the dsybiotic gut to a healthy state. Beta glucans are one of the most promising nutritional supplements with established efficacy in metabolic diseases, diabetes, cancer, cardiovascular diseases and neurological diseases. Beta glucans produced from two strains AFO-202 and N-163 of a black yeast Aureobasidium pullulans derived beta glucan has been reported with beneficial effects in diabetes [D5], dyslipidemia [D6], ASD [D7,8], Duchenne muscular dystrophy (DMD) [D9], Non-alcoholic steatohepatitis (NASH) [D10] and infectious diseases including COVID-19 [D11,12].

In a previous study, the AFO-202 beta 1,3-1,6 glucan was able to balance the gut microbiome in children with ASD [D13]. In the study on STAM™ murine model of NASH, the AFO-202 beta glucan significantly decreased inflammation-associated hepatic cell ballooning and steatosis while the N-163 beta glucan decreased fibrosis and inflammation. The combination of AFO-202 with N-163 significantly decreased the Non-alcoholic fatty liver disease (NAFLD) Activity Score (NAS) [D10]. The present study was undertaken as an extension of this NASH study to study the fecal microbiome and metabolome profile before and after administration of AFO-202 beta glucan.

### Methods:

### Mice

The study is reported in accordance with Animal Research: Reporting of In Vivo Experiments (ARRIVE) guidelines. C57BL/6J mice were obtained from Japan SLC, Inc. (Japan). All animals used in this study were cared for under the following guidelines: Act on Welfare and Management of Animals (Ministry of the Environment, Japan, Act No. 105 of Oct. 1, 1973), standards relating to the care and management of laboratory animals and relief of pain (Notice No. 88 of the Ministry of the Environment, Japan, Apr. 28, 2006) and the guidelines for proper conduct of animal experiments (Science Council of Japan, Jun. 1, 2006). Protocol approvals were obtained from SMC Laboratories, Japan's IACUC (Study reference no: SP_SLMN128-2107-6_1). Mice were maintained in a specific pathogen-free (SPF) facility under controlled conditions of temperature (23±3° C.), humidity (50±20%), lighting (12-hour artificial light and dark cycles; light from 8:00 to 20:00) and air exchange.

The STAM model of NASH was induced as previously described [D10]. Mice were given a single subcutaneous injection of 200 μg streptozotocin (STZ, Sigma-Aldrich, USA) solution 2 days after birth and fed with a high-fat diet (HFD, 57 kcal % fat, Cat #HFD32, CLEA Japan, Inc., Japan) from 4-9 weeks of age. All mice develop liver steatosis and diabetes and at 3 weeks mice had established steatohepatitis, histologically.

Studygroups: There were five study groups, described below. Eight mice were included in each study group.

### Group 1: Vehicle

Eight NASH mice were orally administered vehicle [RO water] in a volume of 5 mL/kg once daily from 6 to 9 weeks of age.

### Group 2: AFO-202 Beta Glucan

Eight NASH mice were orally administered vehicle supplemented with AFO-202 Beta Glucan at a dose of 1 mg/kg in a volume of 5 mL/kg once daily from 6 to 9 weeks of age.

### Group 3: Telmisartan

Eight NASH mice were orally administered vehicle supplemented with Telmisartan at a dose of 10 mg/kg once daily from 6 to 9 weeks of age.

TABLE 1
Study design and treatment schedule

| | No. | Test | Dose | Volume | | |
|---|---|---|---|---|---|---|
| Group | mice | substance | (mg/kg) | (mL/kg) | Regimen | Sacrifice |
| 1 | 8 | Vehicle | — | 5 | PO, QD, | 9 wks |
| | | | | | 6-9 wks | |
| 2 | 8 | AFO-202 | 1 | 5 | PO, QD, | 9 wks |
| | | Beta | | | 6-9 wks | |
| | | Glucan | | | | |
| 3 | 8 | Telmisartan | 10 | 5 | PO, QD, | 9 wks |
| | | | | | 6-9 wks | |

### Test Substances

AFO-202 Beta Glucan was provided by GN Corporation Co Ltd., Japan. Telmisartan (Micardis(R)) was purchased from Boehringer Ingelheim GmbH (Germany).

### Randomization

NASH model mice were randomized into 3 groups of 8 mice at 6 weeks of age based on their body weight the day before the start of treatment. The randomization was performed by body weight-stratified random sampling using Excel software. NASH model mice were stratified by their body weight to get SD and the difference in the mean weights among groups as small as possible.

### Animal Monitoring and Sacrifice

The viability, clinical signs (lethargy, twitching, labored breathing) and behavior were monitored daily. Body weight was recorded daily before the treatment. Mice were observed for significant clinical signs of toxicity, moribundity and mortality before administration and after administration. The animals were sacrificed at 9 weeks of age by exsanguination through direct cardiac puncture under isoflurane anesthesia (Pfizer Inc.).

### Collection of Fecal Pellets Samples:

Frequency: Fecal samples were collected at the 6 weeks of age (before administration) and 9 weeks of age (before sacrifice).

Procedure: At 6 weeks of age, fecal samples were collected from each mouse by clean catch method. Handle animals with clean gloves sterilized with 70% ethanol. A sterile petri dish was placed on the bench. Gently massage the abdomen, position the bottom of mouse over a fresh petri dish, and collect 1-2 fecal pellets. At sacrifice, fecal samples were aseptically collected from cecum. The tubes with feces were placed on ice immediately. These tubes were snap frozen in liquid nitrogen and stored at −80° C. for shipping.

### Microbiome Analysis:

In this analysis, the 16S rRNA sequence data acquired by the next-generation sequencer from the fecal RNA was used to perform community analysis using the QIIME2 program for microbial community analysis. The raw read data in FASTQ format output from the next-generation sequencer was trimmed to remove adapter sequences and low QV regions that may be included in the data. Cutadapt was used to remove adapter sequences from DNA sequencing reads. Trimmomatic was used as read trimming tool for Illumina NGS data. The adapter sequence was trimmed using the adapter trimming program “cutadapt” if the Trimming of the region at the end of the read sequence overlapped the corresponding sequence by at least one base (mismatch tolerance: 20%). When reads containing N were present in at least one of Read1 and Read2, both Read1 and Read2 were removed.

### Illumina Adapter Sequence Information

### Read1 3′ End Side

CTGTCTTCTATACACATCTCCGAGCCCACGAGAC

### Read2 3′ End Side

CTGTCTTCTATACACATCTGACGCTGCCGACGA

Trimming of low QV regions was performed on the read data after processing using the QV trimming program “Trimmomatic” under the following conditions.

### <QV Trimming Conditions>

The window of 20 bases is slid from the 5′ side, and the area where the average QV is less than 20 is trimmed.

After trimming, only the reads with more than 50 bases remaining in both Read1 and Read2 were taken as output.

### Population Analysis

The microbial community analysis based on 16S rRNA sequence was performed on the sequence data trimmed in the previous section using the microbial community analysis program “QIIME2”. The annotation program “sklearn” included in QIIME2 is used to annotate the ASV (OTU) sequences.

Using “sklearn”, an annotation program included in QIIME2, the ASV (OTU) sequences obtained were annotated with taxonomy information [Kingdom (kingdom)/Phylum (phylum)/Class (class)/Order (order)/Family (family)/Genus/Species] based on the 16S rDNA database.

The data set of 16S rDNA database “greengenes” provided on the QIIME2 Resources site was used for the analysis. The ASVs (OTUs) obtained above were aggregated and graphed based on the taxonomy information a and the read counts of each specimen. Based on the composition of bacterial flora for each specimen compiled above, various index values for α-diversity were calculated.

### Metabolome Analysis:

After lyophilization of the fecal sample, about 10 mg of the sample was separated, extracted by the Bligh-Dyer method, and the resulting aqueous layer1 was collected and lyophilized. The residue was derivatized using 2-methoxyamine hydrochloride and MSTFA, and submitted to gas chromatography-mass spectrometry (GC-MS) as an analytical sample. 2-Isopropylmalic acid was used as an internal standard. In addition, an operational blank test was also conducted.

Analytical equipment used was GCMS-TQ8030 (Shimadzu Corporation); Column BPX-5 (film thickness 0.25 μm, length 30 m, inner diameter 0.25 mm, Shimadzu GC).

### Peak Detection and Analysis:

MS-DIAL ver.4.7 (http://prime.psc.riken.jp/compms/index.html) was used to analyze and prepare the peak list (peak height) under the conditions given in Table 2. In doing so, peaks that were detected in the QC samples and whose C.V. was less than 20% and whose intensity was more than twice that of the operating blank were treated as detected peaks.

TABLE 2
Abundance of all the bacteria analysed at baseline and post-intervention

| | | | | F18S_3 | | | F18S_13- | |
|---|---|---|---|---|---|---|---|---|
| | | | F18S_1 | AFO- | F18S_9- | F18S_11- | AFO- | F18S_19- |
| | | | Control- | 202 - | Telmisartan- | Control- | 202- | Telmisartan- |
| Family | Genus | Species | pre | pre | pre | post | post | post |
| [symbol] Erysipelotrichaceae | [symbol] Allobaculum | [symbol] — | 12083 | 15713 | 6375 | 8007 | 6542 | 622 |
| [symbol] Turicibacteraceae | [symbol] Turicibacter | [symbol] — | 12538 | 6159 | 9408 | 3346 | 2749 | 786 |
| [symbol] Desulfovibrionaceae | [symbol] — | [symbol] — | 306 | 1398 | 3882 | 8016 | 10096 | 6012 |
| [symbol] Clostridiaceae | [symbol] Other | [symbol] Other | 7455 | 3689 | 7143 | 3479 | 2806 | 723 |
| [symbol] Lactobacillaceae | [symbol] Lactobacillus | [symbol] — | 8 [symbol] | 3433 | 416 | 11554 | 5804 | 254 |
| [symbol] Streptococcaceae | [symbol] Lactococeus | [symbol] — | 5038 | 2755 | 2239 | 1081 | 2146 | 1948 |
| [symbol] Bacteroidaceae | [symbol] Bacteroides | [symbol] — | 1091 | 1 [symbol] 07 | 1596 | 1562 | 2831 | 8412 |
| [symbol] [Paraprevotellaceae] | [symbol] [Prevotella] | [symbol] — | 962 | 1777 | 1674 | 540 | 1865 | 7582 |
| [symbol] — | [symbol] — | [symbol] — | 17 | 680 | 1552 | 526 [symbol] | 5964 | 6029 |
| [symbol] Deferribacteraceae | [symbol] Mucispirillum | [symbol] schaedleri | 36 | 1486 | 4795 | 2087 | 1644 | 1921 |
| [symbol] Bacteroidaceae | [symbol] Bacteroides | [symbol] acidifaciens | 322 | 781 | 983 | 2633 | 2161 | 2571 |
| [symbol] — | [symbol] — | [symbol] — | 497 | 760 | 853 | 1386 | 1478 | 2764 |
| [symbol] Verrucomicrobiaceae | [symbol] Akkermansia | [symbol] muciniphila | 21 [symbol] 1 | 1963 | 2305 | 0 | 746 | 320 |
| [symbol] Rikenellaceae | [symbol] — | [symbol] — | 475 | 849 | 696 | 616 | 769 | 117 [symbol] |
| [symbol] Enterobacteriaceae | [symbol] Other | [symbol] Other | 0 | 5 | 17 | 100 | 0 | 40 |
| [symbol] Lactobacillaceae | [symbol] Lactobacillus | [symbol] — | 1729 | 1119 | 883 | 594 | 459 | 513 |
| [symbol] S24-7 | [symbol] — | [symbol] — | 592 | 953 | 623 | 494 | 796 | 411 |
| [symbol] Lachnospiraceae | [symbol] — | [symbol] — | 149 | 914 | 752 | 826 | 1194 | 1545 |
| [symbol] Peptostreptocpccaceae | [symbol] — | [symbol] — | 1705 | 7 | 1121 | 1344 | 6 [symbol] 1 | 181 |
| [symbol] Lachnospiraceae | [symbol] [Ruminococcus] | [symbol] gnavus | 1015 | 1118 | 2041 | 582 | 398 | 898 |
| [symbol] Lachnospiraceae | [symbol] Coprococeus | [symbol] — | 90 | 955 | 444 | 708 | 1191 | 1468 |
| [symbol] Bifidobacteriaceae | [symbol] Bifidobacterium | [symbol] pseudolongum | 711 | 1236 | 1317 | 180 | 324 | 111 |
| [symbol] Enterobacteriaceae | [symbol] Proteus | [symbol] — | 14 | 42 | 0 | 1432 | 520 | 1153 |
| [symbol] Lactobacillaceae | [symbol] Lactobacillus | [symbol] Other | 1456 | 362 | 64 | 2494 | 844 | 111 |
| [symbol] S24-7 | [symbol] — | [symbol] — | 442 | 804 | 740 | 819 | 555 | 329 |
| [symbol] Porphyromonadaceae | [symbol] Parabacteroides | [symbol] distasonis | 111 | 194 | 320 | 699 | 700 | 1437 |
| [symbol] Clostridiaceae | [symbol] — | [symbol] — | 389 | 384 | 1242 | 387 | 572 | 79 |
| [symbol] — | [symbol] — | [symbol] — | 41 | 289 | 420 | 998 | 1118 | 536 |
| [symbol] Desulfovibrionaceae | [symbol] Desulfovibrio | [symbol] — | 0 | 8 | 65 | 734 | 739 | 503 |
| [symbol] S24-7 | [symbol] — | [symbol] — | 331 | 629 | 708 | 155 | 495 | 271 |
| [symbol] Coriobacteriaceae | [symbol] Adlercreutzia | [symbol] — | 1261 | 411 | 625 | 399 | 280 | 135 |
| [symbol] S24-7 | [symbol] — | [symbol] — | 216 | 457 | 480 | 471 | 163 | 527 |
| [symbol] Erysipelotrichaceae | [symbol] Clostridium | [symbol] cocleatum | 0 | 95 | 319 | 519 | 1185 | 696 |
| [symbol] Ruminococcaceae | [symbol] Ruminococcus | [symbol] — | 532 | 1331 | 1164 | 50 | 257 | 266 |
| [symbol] — | [symbol] — | [symbol] — | 250 | 302 | 319 | 522 | 564 | 995 |
| [symbol] Lachnospiraceae | [symbol] Other | [symbol] Other | 35 | 267 | 481 | 611 | 832 | 436 |
| [symbol] Lachnospiraceae | [symbol] [Ruminococcus] | [symbol] gnavus | 238 | 1211 | 311 | 0 | 25 | 87 |
| [symbol] Turicibacteraceae | [symbol] Turicibacter | [symbol] — | 822 | 472 | 586 | 269 | 206 | 0 |
| [symbol] Lachnospiraceae | [symbol] — | [symbol] — | 119 | 46 | 611 | 119 | 119 | 253 |
| [symbol] Bacteroidaceae | [symbol] Bacteroides | [symbol] — | 18 | 73 | 460 | 180 | 283 | 2042 |
| [symbol] Ruminococcaceae | [symbol] Oscillospira | [symbol] — | 82 | 400 | 293 | 267 | 804 | 621 |
| [symbol] Clostridiaceae | [symbol] Other | [symbol] Other | 923 | 362 | [symbol] 17 | 314 | 132 | 70 |
| [symbol] Lachnospiraceae | [symbol] Roseburia | [symbol] — | 88 | 466 | 488 | 408 | 878 | 927 |
| [symbol] Lachnospiraceae | [symbol] — | [symbol] — | 66 | 386 | 338 | 461 | 658 | 698 |
| [symbol] S24-7 | [symbol] — | [symbol] — | 389 | 291 | 305 | 98 | 129 | 104 |
| [symbol] Lachnospiraceae | [symbol] [Ruminococcus] | [symbol] gnavus | 44 | 522 | 123 | 466 | 0 | 126 |
| [symbol] — | [symbol] — | [symbol] — | 224 | 692 | 418 | 104 | 49 | 236 |
| [symbol] — | [symbol] — | [symbol] — | 82 | 358 | 357 | 323 | 299 | 267 |
| [symbol] Lachnospiraceae | [symbol] Coprococcus | [symbol] — | 38 | 349 | 126 | 297 | 454 | 562 |
| [symbol] S24-7 | [symbol] — | [symbol] — | 146 | 230 | 322 | 199 | 549 | 144 |
| [symbol] S24-7 | [symbol] — | [symbol] — | 153 | 3 [symbol] 8 | 237 | 340 | 131 | 410 |
| [symbol] — | [symbol] — | [symbol] — | 7 | 199 | 116 | 283 | 2 [symbol] 5 | 590 |
| [symbol] Lachnospiraceae | [symbol] Other | [symbol] Other | 263 | 65 | 562 | 169 | 100 | 245 |
| [symbol] Lachnospiraceae | [symbol] [Ruminococcus] | [symbol] gnavus | 386 | 162 | 375 | 62 | 433 | 686 |
| [symbol] Erysipelotrichaceae | [symbol] Allobaculum | [symbol] — | 512 | 415 | 286 | 501 | 301 | 0 |
| [symbol] Desulfovibrionaceae | [symbol] Desulfovibrio | [symbol] C21_c20 | 47 | 333 | 604 | 98 | 294 | 43 |
| [symbol] S24-7 | [symbol] — | [symbol] — | 212 | 366 | 383 | 120 | 271 | 152 |
| [symbol] — | [symbol] — | [symbol] — | 53 | 228 | 524 | 177 | 178 | 345 |
| [symbol] Streptococcaceae | [symbol] Lactococcus | [symbol] garvieae | 167 | 0 | 11 | 0 | 0 | 8 |
| [symbol] — | [symbol] — | [symbol] — | 0 | 227 | 0 | 0 | 0 | 0 |
| [symbol] Enterobacteriaceae | [symbol] Proteus | [symbol] — | 0 | 0 | 0 | 507 | 184 | 386 |
| [symbol] Lachnospiraceae | [symbol] — | [symbol] — | 76 | 760 | 44 | 103 | 0 | 786 |
| [symbol] S24-7 | [symbol] — | [symbol] — | 162 | 376 | 399 | 120 | 103 | 74 |
| [symbol] Lachnospiraceae | [symbol] [Ruminococcus] | [symbol] gnavus | 38 | 471 | 278 | 31 | 179 | 331 |
| [symbol] — | [symbol] — | [symbol] — | 273 | 690 | 371 | 0 | 38 | 44 |
| [symbol] Clostridiaceae | [symbol] Clostridium | [symbol] Other | 145 | 179 | 707 | 196 | 284 | 0 |
| [symbol] Lachnospiraceae | [symbol] — | [symbol] — | 37 | 867 | 0 | 179 | 356 | 15 |
| [symbol] Clostridiaceae | [symbol] Other | [symbol] Other | 542 | 252 | 215 | 233 | 97 | 49 |
| [symbol] — | [symbol] — | [symbol] — | 403 | 25 | 313 | 21 | 0 | 54 |
| [symbol] Clostridiaceae | [symbol] Other | [symbol] Other | 7 [symbol] 6 | 307 | 255 | 281 | 127 | 5 [symbol] |
| [symbol] S24-7 | [symbol] — | [symbol] — | 163 | 408 | 196 | 20 [symbol] | 199 | 101 |
| [symbol] Lachnospiraceae | [symbol] [Ruminococcus] | [symbol] gnavus | 301 | 295 | 601 | 151 | 118 | 260 |
| [symbol] Bacteroidaceae | [symbol] Bacteroides | [symbol] — | 11 | 74 | 170 | 20 | 66 | 1596 |
| [symbol] S24-7 | [symbol] — | [symbol] — | 165 | 318 | 327 | 67 | 220 | 125 |
| [symbol] — | [symbol] — | [symbol] — | 264 | 754 | 323 | 0 | 39 | 3 [symbol] |
| [symbol] Lachnospiraceae | [symbol] [Ruminococcus] | [symbol] gnavus | 310 | 363 | 120 | 177 | 58 | 281 |
| [symbol] Ruminococcaceae | [symbol] Oscillospira | [symbol] — | 69 | 329 | 284 | 164 | 288 | 393 |
| [symbol] Ruminococcaceae | [symbol] Anaerotruncus | [symbol] — | 38 | 189 | 638 | 112 | 113 | 12 [symbol] |
| [symbol] Alcaligenaceae | [symbol] Sutterella | [symbol] — | 257 | 217 | 191 | 399 | 107 | 63 |
| [symbol] S24-7 | [symbol] — | [symbol] — | 169 | 368 | 394 | 83 | 85 | 54 |
| [symbol] F16 | [symbol] — | [symbol] — | [symbol] 10 | 0 | 329 | 117 | 0 | 22 [symbol] |
| [symbol] — | [symbol] — | [symbol] — | 305 | 37 | 320 | 0 | 0 | 0 |
| [symbol] Other | [symbol] Other | [symbol] Other | 456 | 0 | 276 | 360 | 170 | 0 |
| [symbol] Lachnospiraceae | [symbol] [Ruminococcus] | [symbol] gnavus | 146 | 175 | 168 | 173 | 91 | 325 |
| [symbol] S24-7 | [symbol] — | [symbol] — | 206 | 230 | 476 | 0 | 0 | 153 |
| [symbol] Rikenellaceae | [symbol] — | [symbol] — | 0 | 162 | 259 | 135 | 264 | 134 |
| [symbol] Lachnospiraceae | [symbol] [Ruminococcus] | [symbol] gnavus | 656 | 0 | 62 | 492 | 0 | 30 |
| [symbol] Lachnospiraceae | [symbol] — | [symbol] — | 0 | 118 | 250 | 126 | 244 | 359 |
| [symbol] Clostridiaceae | [symbol] Other | [symbol] Other | 0 | 164 | 693 | 119 | 235 | 39 |
| [symbol] Lachnospiraceae | [symbol] — | [symbol] — | 46 | 111 | 78 | 160 | 205 | 239 |
| [symbol] Ruminococcaceae | [symbol] Oscillospira | [symbol] — | 117 | 97 | 160 | 249 | 220 | 138 |
| [symbol] Enterococcaceae | [symbol] Enterococcus | [symbol] — | 45 | 42 | 32 | 822 | 61 | 0 |
| [symbol] Rikenellaceae | [symbol] — | [symbol] — | 39 | 147 | 239 | 119 | 230 | 96 |
| [symbol] S24-7 | [symbol] — | [symbol] — | 188 | 153 | 208 | 40 | 59 | 79 |
| [symbol] Lactobacillaceae | [symbol] Lactobacillus | [symbol] reuteri | 517 | 157 | 0 | 428 | 340 | 0 |
| [symbol] Bacteroidaceae | [symbol] Bacteroides | [symbol] acidifaciens | 0 | 90 | 161 | 124 | 202 | 127 |
| [symbol] Ruminococcaceae | [symbol] Oscillospira | [symbol] — | 75 | 162 | 202 | 51 | 2 [symbol] 4 | 451 |
| [symbol] Enterobacteriaceae | [symbol] Escherichia | [symbol] coli | 0 | 0 | 0 | 0 | 0 | 0 |
| [symbol] Ruminococcaceae | [symbol] Oscillospira | [symbol] — | 16 | 262 | 152 | 135 | 137 | 280 |
| [symbol] — | [symbol] — | [symbol] — | 117 | 2 [symbol] 6 | 228 | 29 | 17 | 254 |
| [symbol] S24-7 | [symbol] — | [symbol] — | 84 | 173 | 157 | 2 [symbol] 0 | 62 | 92 |
| [symbol] — | [symbol] — | [symbol] — | 1 [symbol] 9 | 0 | 988 | 0 | 0 | 0 |
| [symbol] Other | [symbol] Other | [symbol] Other | 11 | 149 | 463 | 230 | 27 | 251 |
| [symbol] Lachnospiraceae | [symbol] — | [symbol] — | 51 | 67 | 46 | 287 | 224 | 163 |
| [symbol] Lachnospiraceae | [symbol] [Ruminococcus] | [symbol] gnavus | 88 | 185 | 148 | 190 | 272 | 63 |
| [symbol] Lachnospiraceae | [symbol] — | [symbol] — | 28 | 54 | 390 | 17 | 62 | 117 |
| [symbol] Enterobacteriaceae | [symbol] Klebsiella | [symbol] Other | 0 | 0 | 0 | 225 | 0 | 33 |
| [symbol] Lachnospiraceae | [symbol] — | [symbol] — | 11 | 105 | 76 | 51 | 99 | 187 |
| [symbol] Enterobacteriaceae | [symbol] Klebsiella | [symbol] — | 0 | 0 | 0 | 210 | 0 | [symbol] 7 |
| [symbol] — | [symbol] — | [symbol] — | 11 | 491 | 0 | 32 | 61 | 261 |
| [symbol] Lactobacillaceae | [symbol] Lactobacillus | [symbol] — | 167 | 26 | 4 | 857 | 183 | 0 |
| [symbol] Other | [symbol] Other | [symbol] Other | 27 | 23 [symbol] | 57 | 102 | 117 | 248 |
| [symbol] — | [symbol] — | [symbol] — | 0 | 114 | 209 | 176 | 243 | 0 |
| [symbol] Erysipclotrichaceae | [symbol] Allobaculum | [symbol] — | 0 | 0 | 203 | 0 | 0 | 59 |
| [symbol] — | [symbol] — | [symbol] — | 72 | 267 | 131 | 0 | 0 | 84 |
| [symbol] Lachnospiraceae | [symbol] — | [symbol] — | 34 | 188 | 207 | 15 | 165 | 271 |
| [symbol] — | [symbol] — | [symbol] — | 4 | [symbol] 4 | 362 | 263 | 8 | 173 |
| [symbol] Other | [symbol] Other | [symbol] Other | 18 | 70 | 58 | 149 | 183 | 269 |
| [symbol] Lactobacillaceae | [symbol] Lactobacillus | [symbol] reuteri | 339 | 55 | 0 | 470 | 93 | 0 |
| [symbol] Ruminococcaceae | [symbol] Oscillospira | [symbol] — | 40 | 88 | 158 | 77 | 244 | 273 |
| [symbol] Lachnospiraceae | [symbol] — | [symbol] — | 27 | 93 | 50 | 122 | 193 | 139 |
| [symbol] S24-7 | [symbol] — | [symbol] — | 90 | 0 | 1 [symbol] 8 | 0 | 103 | 125 |
| [symbol] S24-7 | [symbol] — | [symbol] — | 91 | 97 | 64 | 55 | 8 [symbol] | 111 |
| [symbol] Lachnospiraceae | [symbol] — | [symbol] — | 22 | 69 | 315 | 31 | 45 | 119 |
| [symbol] S24-7 | [symbol] — | [symbol] — | 40 | 75 | 104 | 60 | 198 | [symbol] 8 |
| [symbol] S24-7 | [symbol] — | [symbol] — | 123 | 161 | 60 | 36 | 39 | 74 |
| [symbol] Lachnospiraceae | [symbol] — | [symbol] — | 22 | 16 [symbol] | 19 | 229 | 192 | 98 |
| [symbol] — | [symbol] — | [symbol] — | 22 | 18 | 28 | 40 | 109 | 376 |
| [symbol] Coriobacteriaceae | [symbol] Adlercreutzia | [symbol] — | 121 | 196 | 110 | 63 | 33 | 30 |
| [symbol] — | [symbol] — | [symbol] — | 43 | 83 | 200 | 53 | 95 | 118 |
| [symbol] Clostidiaceae | [symbol] Other | [symbol] Other | 100 | 0 | 0 | 0 | 0 | 0 |
| [symbol] Turicibacteraceae | [symbol] Turicibacter | [symbol] — | 521 | 0 | 125 | 0 | 0 | 0 |
| [symbol] Ruminococcaceae | [symbol] Ruminococcus | [symbol] — | 49 | 105 | 193 | 0 | 81 | 143 |
| [symbol] Ruminococcaceae | [symbol] Oscillospira | [symbol] — | 13 | 10 [symbol] | 215 | 36 | 75 | 119 |
| [symbol] Dehalobacteriaceae | [symbol] Dehalobacterium | [symbol] — | 20 | 38 | 43 | 100 | 256 | 268 |
| [symbol] Staphylococcaceae | [symbol] Staphylococcus | [symbol] Other | 177 | 72 | 110 | 88 | 81 | 34 |
| [symbol] Rikenellaceae | [symbol] — | [symbol] — | 13 | 62 | 61 | 139 | 140 | 97 |
| [symbol] Lachnospiraceae | [symbol] — | [symbol] — | 155 | 109 | 87 | 63 | 114 | 98 |
| [symbol] — | [symbol] — | [symbol] — | 53 | 188 | 60 | 0 | 0 | 68 |
| [symbol] Staphylococcaceae | [symbol] Staphylococcus | [symbol] sciuri | 184 | 66 | 34 | 139 | 63 | 41 |
| [symbol] — | [symbol] — | [symbol] — | 356 | 0 | 103 | 25 | 0 | 0 |
| [symbol] Ruminococcaceae | [symbol] Ruminococcus | [symbol] — | 72 | 98 | 157 | 16 | 119 | 124 |
| [symbol] Coriobacteriaceae | [symbol] Adlercreutzia | [symbol] — | 176 | 96 | 101 | 34 | 46 | 0 |
| [symbol] Ruminococcaceae | [symbol] Oscillospira | [symbol] — | 34 | 67 | 61 | 103 | 101 | 120 |
| [symbol] Lachnospiraceae | [symbol] Clostridium | [symbol] Other | 0 | 32 | 85 | 15 | 15 | 40 |
| [symbol] Lachnospiraceae | [symbol] Coprococcus | [symbol] — | 56 | 92 | 151 | 69 | 61 | 85 |
| [symbol] Desulfovibrionaceae | [symbol] — | [symbol] — | 4 | 41 | 50 | 90 | 18 [symbol] | 148 |
| [symbol] — | [symbol] — | [symbol] — | 32 | 162 | 94 | 28 | 23 | 103 |
| [symbol] Lachnospiraceae | [symbol] — | [symbol] — | 22 | 2 [symbol] 5 | 0 | 53 | 0 | 272 |
| [symbol] Dehalobacteriaceae | [symbol] Dehalobacterium | [symbol] — | 0 | 34 | 41 | 9 [symbol] | 203 | 239 |
| [symbol] Coriobacteriaceae | [symbol] Adlercreutzia | [symbol] — | 108 | 127 | 90 | 52 | 63 | 0 |
| [symbol] Lachnospiraceae | [symbol] — | [symbol] — | 14 | 39 | 37 | [symbol] 3 | 108 | 119 |
| [symbol] Lachnospiraceae | [symbol] — | [symbol] — | 87 | 193 | 168 | 48 | 17 | 14 |
| [symbol] Ruminococcaceae | [symbol] Oscillospira | [symbol] — | 0 | 0 | 0 | 0 | 101 | 4 [symbol] 3 |
| [symbol] S24-7 | [symbol] — | [symbol] — | 56 | 158 | [symbol] 5 | 33 | 47 | 51 |
| [symbol] Streptococcaceae | [symbol] Streptococcus | [symbol] — | 0 | 0 | 0 | 112 | 106 | 44 |
| [symbol] Lachnospiraceae | [symbol] [Ruminococcus] | [symbol] gnavus | 0 | 107 | 83 | 126 | 165 | 37 |
| [symbol] Lachnospiraceae | [symbol] — | [symbol] — | 0 | 144 | 102 | 0 | 102 | 173 |
| [symbol] Lachnospiraceae | [symbol] — | [symbol] — | 50 | 158 | 231 | 11 | 17 | 10 |
| [symbol] Coriobacteriaceae | [symbol] Adlercreutzia | [symbol] — | 142 | [symbol] 9 | 74 | 26 | 19 | 8 |
| [symbol] Lachnospiraceae | [symbol] — | [symbol] — | 220 | 7 | 2 [symbol] | 55 | 52 | 57 |
| [symbol] Erysipelotrichaceae | [symbol] Clostridium | [symbol] cocleatum | 0 | 0 | 0 | 106 | 222 | 119 |
| [symbol] S24-7 | [symbol] — | [symbol] — | 15 | 36 | 53 | 93 | 160 | [symbol] 0 |
| [symbol] Lachnospiraceae | [symbol] — | [symbol] — | 7 | 40 | 27 | 64 | 61 | 81 |
| [symbol] Bacteroidaceae | [symbol] Bacteroides | [symbol] — | 0 | 0 | 55 | 0 | 0 | 468 |
| [symbol] Ruminococcaceae | [symbol] Oscillospira | [symbol] — | 0 | 46 | 59 | 87 | 97 | 53 |
| [symbol] Ruminococcaceae | [symbol] — | [symbol] — | 21 | 41 | 41 | 81 | 139 | 72 |
| [symbol] Lachnospiraceae | [symbol] Other | [symbol] Other | 42 | 11 | 52 | 53 | 86 | 90 |
| [symbol] Lachnospiraceae | [symbol] Dorea | [symbol] — | 23 | 46 | 28 | 50 | 63 | 154 |
| [symbol] Lachnospiraceae | [symbol] — | [symbol] — | 0 | 0 | 0 | 70 | 78 | 94 |
| [symbol] S24-7 | [symbol] — | [symbol] — | 34 | 130 | 168 | 0 | 29 | 0 |
| [symbol] Lachnospiraceae | [symbol] — | [symbol] — | 0 | 87 | 92 | 0 | 96 | 138 |
| [symbol] Lactobacillaceae | [symbol] Lactobaccilus | [symbol] Other | 107 | 148 | 0 | 83 | 189 | 0 |
| [symbol] — | [symbol] — | [symbol] — | 0 | 39 | 34 | 29 | 73 | 48 |
| [symbol] Ruminococcaceae | [symbol] Oscillospira | [symbol] — | 24 | 95 | 99 | 0 | 52 | 101 |
| [symbol] S24-7 | [symbol] — | [symbol] — | 0 | 43 | 55 | 64 | 52 | [symbol] 9 |
| [symbol] S24-7 | [symbol] — | [symbol] — | 55 | 52 | 67 | 0 | 53 | 71 |
| [symbol] [Mogibacteriaceae] | [symbol] — | [symbol] — | 55 | 76 | 36 | 44 | 53 | 28 |
| [symbol] Peptococcaceae | [symbol] — | [symbol] — | 18 | 80 | 43 | 36 | 152 | 42 |
| [symbol] — | [symbol] — | [symbol] — | 42 | 42 | 13 | 0 | 0 | 40 |
| [symbol] — | [symbol] — | [symbol] — | 0 | 36 | 0 | 83 | 0 | 213 |
| [symbol] S24-7 | [symbol] — | [symbol] — | 31 | 99 | 76 | 33 | 36 | 19 |
| [symbol] Ruminococcaceae | [symbol] Oscillospira | [symbol] — | 17 | 25 | [symbol] 5 | 26 | 88 | 135 |
| [symbol] Ruminococcaceae | [symbol] Oscillospira | [symbol] — | 15 | 110 | 97 | 4 | 14 | 53 |
| [symbol] Lachnospiraceae | [symbol] [Ruminococcus] | [symbol] gnavus | 43 | 135 | 33 | 0 | 0 | 87 |
| [symbol] Ruminococcaceae | [symbol] Oscillospira | [symbol] — | 15 | 35 | 57 | 33 | 100 | 42 |
| [symbol] Lactobacillaceae | [symbol] Lactobaccilus | [symbol] reuteri | 80 | 103 | 0 | 76 | 178 | 0 |
| [symbol] Coriobacteriaceae | [symbol] Adlercreutzia | [symbol] — | 112 | 73 | 58 | 0 | 0 | 0 |
| [symbol] Lachnospiraceae | [symbol] — | [symbol] — | 12 | 0 | 0 | 36 | 0 | 0 |
| [symbol] S24-7 | [symbol] — | [symbol] — | 0 | 0 | 64 | 48 | 113 | 49 |
| [symbol] Turicibacteraceae | [symbol] Turicibacter | [symbol] — | 0 | 0 | 0 | 134 | 0 | 0 |
| [symbol] S24-7 | [symbol] — | [symbol] — | 0 | 0 | 45 | 128 | 53 | 41 |
| [symbol] Rikenellaceae | [symbol] — | [symbol] — | 11 | 14 | 25 | 84 | 84 | 86 |
| [symbol] Enterobacteriaceae | [symbol] Other | [symbol] Other | 0 | 0 | 0 | 99 | 0 | 0 |
| [symbol] Rikenellaceae | [symbol] Other | [symbol] Other | 5 | 40 | 47 | 37 | 43 | 53 |
| [symbol] Porphy [symbol] ceae | [symbol] Parabacteroides | [symbol] — | 0 | 2 [symbol] | 16 | 63 | 72 | 79 |
| [symbol] Lactobacillaceae | [symbol] Lactobaccilus | [symbol] Other | 87 | 86 | 0 | 81 | 146 | 0 |
| [symbol] — | [symbol] — | [symbol] — | 0 | 47 | 60 | 0 | 38 | 0 |
| [symbol] S24-7 | [symbol] — | [symbol] — | 13 | 53 | 27 | 22 | 73 | 45 |
| [symbol] S24-7 | [symbol] — | [symbol] — | 15 | 30 | 35 | 0 | 83 | 109 |
| [symbol] — | [symbol] — | [symbol] — | 8 | 50 | 85 | 43 | 14 | 19 |
| [symbol] Enterobacteriaceae | [symbol] Klebsiella | [symbol] Other | 0 | 0 | 0 | 82 | 0 | 0 |
| [symbol] — | [symbol] — | [symbol] — | 32 | 73 | 63 | 0 | 5 | 3 |
| [symbol] Lachnospiraceae | [symbol] Other | [symbol] Other | 8 | 6 [symbol] | 48 | 24 | 67 | 137 |
| [symbol] Ruminococcaceae | [symbol] Oscillospira | [symbol] — | 0 | 0 | 0 | 102 | 134 | 0 |
| [symbol] Ruminococcaceae | [symbol] Oscillospira | [symbol] — | 0 | 67 | 71 | 0 | 30 | 65 |
| [symbol] Bacteroidaceae | [symbol] Bacteroides | [symbol] acidifaciens | 0 | 0 | 27 | 31 | 35 | 35 |
| [symbol] Erysipelotrichaceae | [symbol] — | [symbol] — | 85 | 29 | 51 | 7 | 14 | 0 |
| [symbol] Lachnospiraceae | [symbol] Dorea | [symbol] — | 18 | 0 | 98 | 6 | 15 | 74 |
| [symbol] Enterobacteriaceae | [symbol] Enterobacter | [symbol] Other | 125 | 0 | 0 | 30 | [symbol] | 41 |
| [symbol] Ruminococcaceae | [symbol] Oscillospira | [symbol] — | 0 | 0 | 42 | 0 | 82 | 45 |
| [symbol] Ruminococcaceae | [symbol] Other | [symbol] Other | 1 [symbol] | [symbol] 3 | 61 | [symbol] 0 | 44 | 12 |
| [symbol] Ruminococcaceae | [symbol] Oscillospira | [symbol] — | 0 | 0 | 0 | 0 | 68 | 184 |
| [symbol] Rikenellaceae | [symbol] — | [symbol] — | 0 | 0 | 31 | 61 | 55 | 43 |
| [symbol] — | [symbol] — | [symbol] — | 0 | 63 | [symbol] 9 | 12 | 45 | 79 |
| [symbol] Lachnospiraceae | [symbol] Other | [symbol] Other | 0 | 0 | 98 | 0 | 0 | 273 |
| [symbol] Other | [symbol] Other | [symbol] Other | 0 | 0 | 18 | 39 | 65 | 87 |
| [symbol] Ruminococcaceae | [symbol] Oscillospira | [symbol] — | 0 | 47 | 79 | 0 | 37 | 72 |
| [symbol] S24-7 | [symbol] — | [symbol] — | 0 | 23 | 38 | 28 | 78 | 85 |
| [symbol] Lachnospiraceae | [symbol] — | [symbol] — | 6 | 15 | 3 | 111 | 127 | 0 |
| [symbol] Streptococcaceae | [symbol] Staphylococcus | [symbol] — | 53 | 0 | 0 | 168 | 30 | 27 |
| [symbol] — | [symbol] — | [symbol] — | 0 | 77 | 96 | 0 | 0 | 0 |
| [symbol] — | [symbol] — | [symbol] — | 0 | 0 | 87 | 60 | 0 | 0 |
| [symbol] — | [symbol] — | [symbol] — | 0 | 0 | 0 | 0 | 219 | 101 |
| [symbol] Other | [symbol] Other | [symbol] Other | 0 | [symbol] 0 | 10 | 15 | 29 | 71 |
| [symbol] Aerococcaceae | [symbol] Aerococcus | [symbol] — | 7 | 0 | 0 | 22 | 85 | 187 |
| [symbol] Ruminococcaceae | [symbol] — | [symbol] — | 0 | 0 | 0 | 0 | 0 | 0 |
| [symbol] — | [symbol] — | [symbol] — | 0 | 0 | 61 | 0 | 111 | 108 |
| [symbol] Streptococcaceae | [symbol] Staphylococcus | [symbol] — | 40 | 18 | 0 | 120 | 0 | 3 [symbol] |
| [symbol] Ruminococcaceae | [symbol] Oscillospira | [symbol] — | 19 | 31 | 0 | 14 | 71 | 26 |
| [symbol] — | [symbol] — | [symbol] — | 11 | 36 | 0 | 7 | 11 | 51 |
| [symbol] Ruminococcaceae | [symbol] Ruminococcus | [symbol] — | 6 | 15 | 0 | 13 | 110 | 43 |
| [symbol] — | [symbol] — | [symbol] — | 0 | 0 | 6 | 0 | 0 | 266 |
| [symbol] Other | [symbol] Other | [symbol] Other | 0 | 19 | 0 | 0 | 64 | 48 |
| [symbol] Ruminococcaceae | [symbol] Oscillospira | [symbol] — | 12 | 86 | 30 | 0 | 63 | 0 |
| [symbol] Lachnospiraceae | [symbol] [Ruminococcus] | [symbol] gnavus | 51 | 0 | 27 | 18 | 26 | 95 |
| [symbol] Lachnospiraceae | [symbol] — | [symbol] — | 0 | 0 | 0 | 65 | 48 | 0 |
| [symbol] Other | [symbol] Other | [symbol] Other | 0 | 0 | 0 | 0 | 81 | 45 |
| [symbol] Ruminococcaceae | [symbol] Oscillospira | [symbol] — | 11 | 25 | 37 | 23 | 16 | 23 |
| [symbol] — | [symbol] — | [symbol] — | 0 | 0 | 69 | 0 | 99 | 90 |
| [symbol] Other | [symbol] Other | [symbol] Other | 38 | 0 | 0 | 46 | 67 | 11 |
| [symbol] Other | [symbol] Other | [symbol] Other | 0 | 0 | 0 | 0 | 56 | 53 |
| [symbol] Other | [symbol] Other | [symbol] Other | 0 | 40 | 68 | 0 | 0 | 57 |
| [symbol] Lachnospiraceae | [symbol] Clostridium | [symbol] Other | 0 | 0 | 0 | 18 | 0 | 101 |
| [symbol] Lachnospiraceae | [symbol] — | [symbol] — | 0 | 0 | 0 | 16 | 0 | 23 |
| [symbol] Other | [symbol] Other | [symbol] Other | 0 | 42 | 13 | 18 | 62 | 0 |
| [symbol] Other | [symbol] Other | [symbol] Other | 9 | 0 | 23 | 40 | 0 | 51 |
| [symbol] Ruminococcaceae | [symbol] Oscillospira | [symbol] — | 0 | 20 | 25 | 0 | 62 | 38 |
| [symbol] Desulfovibrionaceae | [symbol] — | [symbol] — | 0 | 0 | 23 | 0 | 54 | 42 |
| [symbol] Dehalobacteriaceae | [symbol] Dehalobacterium | [symbol] — | 11 | 31 | 24 | 0 | 0 | 0 |
| [symbol] Porphy [symbol] | [symbol] Parabacteroides | [symbol] — | 0 | 9 | 3 | 29 | 19 | 115 |
| [symbol] Ruminococcaceae | [symbol] Ruminococcus | [symbol] — | 0 | 21 | 44 | 15 | 38 | 22 |
| [symbol] Lachnospiraceae | [symbol] Coprococcus | [symbol] — | 10 | 39 | 23 | 6 | 12 | 46 |
| [symbol] Ruminococcaceae | [symbol] Oscillospira | [symbol] — | 0 | 8 | 0 | 0 | 0 | 77 |
| [symbol] Other | [symbol] Other | [symbol] Other | 0 | 0 | 0 | 0 | 70 | 38 |
| [symbol] Lachnospiraceae | [symbol] Coprococcus | [symbol] — | 0 | 16 | 34 | 25 | 0 | 49 |
| [symbol] Ruminococcaceae | [symbol] Oscillospira | [symbol] — | 0 | 0 | 87 | 0 | 67 | 0 |
| [symbol] Christensenellaceae | [symbol] — | [symbol] — | 49 | 0 | 37 | 8 | 15 | 9 |
| [symbol] S24-7 | [symbol] — | [symbol] — | 0 | 11 | 44 | 8 | 19 | 8 |
| [symbol] Ruminococcaceae | [symbol] Oscillospira | [symbol] — | 0 | 0 | 95 | 0 | 54 | 0 |
| [symbol] Lachnospiraceae | [symbol] Coprococcus | [symbol] — | 0 | 0 | 11 | 19 | 54 | 29 |
| [symbol] Lachnospiraceae | [symbol] Other | [symbol] Other | 0 | 0 | 0 | 0 | 0 | 0 |
| [symbol] Enterococcaceae | [symbol] Va [symbol] coccus | [symbol] — | 11 | 0 | 0 | 0 | 14 | 20 |
| [symbol] Erysipelotrichaceae | [symbol] Clostridium | [symbol] cocleatum | 0 | 0 | 16 | 28 | 61 | 34 |
| [symbol] — | [symbol] — | [symbol] — | 9 | 25 | 92 | 0 | 0 | 0 |
| [symbol] Desulfovibrionaceae | [symbol] Bilophila | [symbol] — | 0 | 8 | 11 | 13 | 46 | 17 |
| [symbol] — | [symbol] — | [symbol] — | 0 | 22 | 107 | 0 | 0 | 0 |
| [symbol] Corynebacteriaceae | [symbol] Corynebacterium | [symbol] stationis | 72 | 0 | 0 | 18 | 23 | 0 |
| [symbol] Lachnospiraceae | [symbol] Other | [symbol] Other | 0 | 43 | 0 | 41 | 0 | 34 |
| [symbol] Lachnospiraceae | [symbol] Other | [symbol] Other | 0 | 0 | 0 | 27 | 26 | 82 |
| [symbol] Lachnospiraceae | [symbol] — | [symbol] — | 22 | 9 | 14 | 7 | 0 | 10 |
| [symbol] Lachnospiraceae | [symbol] — | [symbol] — | 0 | 6 | 31 | 26 | 0 | 0 |
| [symbol] — | [symbol] — | [symbol] — | 0 | 22 | 108 | 0 | 0 | 0 |
| [symbol] — | [symbol] — | [symbol] — | 0 | 0 | 0 | 0 | 67 | 84 |
| [symbol] — | [symbol] — | [symbol] — | 0 | 30 | 0 | 0 | 0 | 0 |
| [symbol] Ruminococcaceae | [symbol] — | [symbol] — | 5 | 52 | 27 | 0 | 0 | 0 |
| [symbol] — | [symbol] — | [symbol] — | 0 | 24 | 96 | 0 | 0 | 0 |
| [symbol] Lachnospiraceae | [symbol] — | [symbol] — | 0 | 54 | 90 | 0 | 0 | 0 |
| [symbol] Dehalobacteriaceae | [symbol] Dehalobacterium | [symbol] — | 3 | 8 | 3 | 19 | 23 | 43 |
| [symbol] Other | [symbol] Other | [symbol] Other | 0 | 71 | 0 | 0 | 0 | 68 |
| [symbol] Streptococcaceae | [symbol] Streptococcus | [symbol] luteciae | 25 | 19 | 12 | 0 | 15 | 0 |
| [symbol] — | [symbol] — | [symbol] — | 0 | 55 | 0 | 0 | 82 | 0 |
| [symbol] S24-7 | [symbol] — | [symbol] — | 9 | 0 | 0 | 38 | 0 | 36 |
| [symbol] Ruminococcaceae | [symbol] A [symbol] otruncus | [symbol] — | 0 | 7 | 6 | 13 | 23 | 11 |
| [symbol] Ruminococcaceae | [symbol] Ruminococcus | [symbol] — | 0 | 63 | 10 | 0 | 0 | 0 |
| [symbol] Lachnospiraceae | [symbol] Clostridium | [symbol] Other | 0 | 0 | 26 | 0 | 0 | 0 |
| [symbol] — | [symbol] — | [symbol] — | 0 | 31 | 10 | 0 | 0 | 10 |
| [symbol] Ruminococcaceae | [symbol] Oscillospira | [symbol] — | 0 | 13 | 11 | 6 | 18 | 22 |
| [symbol] Lachnospiraceae | [symbol] Coprococcus | [symbol] — | 0 | 13 | 20 | 19 | 25 | 30 |
| [symbol] — | [symbol] — | [symbol] — | 20 | 39 | 12 | 12 | 0 | 0 |
| [symbol] S24-7 | [symbol] — | [symbol] — | 0 | 0 | 0 | 0 | 113 | 0 |
| [symbol] A [symbol] ceae | [symbol] | [symbol] — | 21 | 25 | 16 | 0 | 2 | 0 |
| [symbol] Lachnospiraceae | [symbol] — | [symbol] — | 0 | 0 | 0 | 0 | 0 | 60 |
| [symbol] Ruminococcaceae | [symbol] Oscillospira | [symbol] — | 0 | 0 | 54 | 0 | 0 | 30 |
| [symbol] S24-7 | [symbol] — | [symbol] — | 0 | 0 | 47 | 0 | 6 | 0 |
| [symbol] — | [symbol] — | [symbol] — | 0 | 0 | 0 | 0 | 0 | 107 |
| [symbol] Ruminococcaceae | [symbol] Oscillospira | [symbol] — | 0 | 0 | 35 | 0 | 27 | 9 |
| [symbol] Bacteroidaceae | [symbol] Bacteroides | [symbol] acidifaciens | 0 | 0 | 0 | 105 | 0 | 0 |
| [symbol] Bacteroidaceae | [symbol] Bacteroides | [symbol] acidifaciens | 0 | 6 | 0 | 3 | 32 | 11 |
| [symbol] Lachnospiraceae | [symbol] Dorea | [symbol] — | 11 | 21 | 25 | 3 | 0 | 8 |
| [symbol] Ruminococcaceae | [symbol] Other | [symbol] Other | 4 | 14 | 10 | 0 | 8 | 11 |
| [symbol] Lachnospiraceae | [symbol] — | [symbol] — | 0 | 80 | 7 | 0 | 0 | 13 |
| [symbol] Lachnospiraceae | [symbol] — | [symbol] — | 11 | 0 | 20 | 0 | 0 | 0 |
| [symbol] S24-7 | [symbol] — | [symbol] — | 20 | 0 | 1 | 0 | 0 | 1 |
| [symbol] Lachnospiraceae | [symbol] Other | [symbol] Other | 0 | 19 | 14 | 0 | 0 | 0 |
| [symbol] — | [symbol] — | [symbol] — | 4 | 42 | 0 | 0 | 0 | 0 |
| [symbol] Ruminococcaceae | [symbol] Oscillospira | [symbol] — | 0 | 13 | 0 | 9 | 15 | 9 |
| [symbol] Ruminococcaceae | [symbol] Oscillospira | [symbol] — | 0 | 0 | 42 | 0 | 0 | 0 |
| [symbol] Lachnospiraceae | [symbol] — | [symbol] — | 0 | 0 | 14 | 13 | 20 | 19 |
| [symbol] Erysipelotrichaceae | [symbol] — | [symbol] — | 38 | 0 | 0 | 0 | 0 | 0 |
| [symbol] S24-7 | [symbol] — | [symbol] — | 0 | 0 | 6 [symbol] | 0 | 0 | 0 |
| [symbol] Lachnospiraceae | [symbol] — | [symbol] — | 0 | 11 | 13 | 0 | 17 | 9 |
| [symbol] Ruminococcaceae | [symbol] Oscillospira | [symbol] — | 15 | 76 | 0 | 0 | 0 | 0 |
| [symbol] Lachnospiraceae | [symbol] — | [symbol] — | 0 | 21 | 22 | 0 | 0 | 14 |
| [symbol] Other | [symbol] Other | [symbol] Other | 0 | 0 | 49 | 0 | 0 | 0 |
| [symbol] F16 | [symbol] — | [symbol] — | 0 | 66 | 0 | 0 | 18 | 0 |
| [symbol] Christensenellaceae | [symbol] — | [symbol] — | 6 | 17 | 19 | 0 | 5 | 5 |
| [symbol] Lachnospiraceae | [symbol] Other | [symbol] Other | 0 | 0 | 0 | 0 | 0 | 0 |
| [symbol] Ruminococcaceae | [symbol] Oscillospira | [symbol] — | 0 | 9 | 12 | 0 | 0 | 0 |
| [symbol] — | [symbol] — | [symbol] — | 0 | 0 | 80 | 0 | 0 | 0 |
| [symbol] Ruminococcaceae | [symbol] Other | [symbol] Other | 8 | 12 | 14 | 4 | 0 | 0 |
| [symbol] — | [symbol] — | [symbol] — | 0 | 19 | 0 | 0 | 0 | 0 |
| [symbol] Lachnospiraceae | [symbol] Other | [symbol] Other | 15 | 0 | 0 | 0 | 18 | 0 |
| [symbol] Other | [symbol] Other | [symbol] Other | 0 | 0 | 0 | 0 | 0 | 0 |
| [symbol] Enterococcaceae | [symbol] Vagococcus | [symbol] — | 29 | 0 | 0 | 0 | 0 | 26 |
| [symbol] Ruminococcaceae | [symbol] Ruminococcus | [symbol] — | 14 | 9 | 23 | 0 | 0 | 0 |
| [symbol] Erysipelotrichaceae | [symbol] Coprabacillus | [symbol] — | 11 | 2 [symbol] | 11 | 0 | 0 | 0 |
| [symbol] Other | [symbol] Other | [symbol] Other | 0 | 22 | 0 | 0 | 0 | 0 |
| [symbol] Ruminococcaceae | [symbol] Oscillospira | [symbol] — | 0 | 9 | 11 | 0 | 0 | 9 |
| [symbol] Ruminococcaceae | [symbol] Oscillospira | [symbol] — | 0 | 38 | 0 | 0 | 0 | 0 |
| [symbol] — | [symbol] — | [symbol] — | 0 | 14 | 0 | 0 | 0 | 0 |
| [symbol] Lachnospiraceae | [symbol] — | [symbol] — | 0 | [symbol] | 0 | 15 | 14 | 0 |
| [symbol] Ruminococcaceae | [symbol] Oscillospira | [symbol] — | 0 | 0 | 0 | 0 | 0 | 31 |
| [symbol] Lachnospiraceae | [symbol] Coprococcus | [symbol] — | 0 | 0 | 0 | 0 | 0 | 0 |
| [symbol] Ruminococcaceae | [symbol] Other | [symbol] Other | 0 | 0 | 8 | 10 | 9 | 5 |
| [symbol] — | [symbol] — | [symbol] — | 0 | 0 | 0 | 0 | 0 | 0 |
| [symbol] Erysipelotrichaceae | [symbol] — | [symbol] — | 0 | 9 | 6 | 0 | 14 | 2 |
| [symbol] Rikenellaceae | [symbol] — | [symbol] — | 0 | 7 | 0 | 8 | 4 | 11 |
| [symbol] — | [symbol] — | [symbol] — | 0 | 0 | 0 | 0 | 0 | 0 |
| [symbol] — | [symbol] — | [symbol] — | 0 | 0 | 0 | 0 | 0 | 40 |
| [symbol] — | [symbol] — | [symbol] — | 15 | 11 | 8 | 0 | 0 | 0 |
| [symbol] Lachnospiraceae | [symbol] — | [symbol] — | 0 | 0 | 0 | 0 | 0 | 0 |
| [symbol] — | [symbol] — | [symbol] — | 0 | 10 | 15 | 0 | 0 | 0 |
| [symbol] Lachnospiraceae | [symbol] Clostridium | [symbol] Other | 0 | 0 | 0 | 0 | 0 | 28 |
| [symbol] Erysipelotrichaceae | [symbol] — | [symbol] — | 7 | 18 | 9 | 0 | 9 | 0 |
| [symbol] Streptococcaceae | [symbol] Streptococcus | [symbol] minor | 0 | 9 | 9 | 0 | 0 | 0 |
| [symbol] Ruminococcaceae | [symbol] Oscillospira | [symbol] — | 0 | 0 | 0 | 0 | 0 | 0 |
| [symbol] — | [symbol] — | [symbol] — | 0 | 0 | 27 | 0 | 8 | 0 |
| [symbol] Lachnospiraceae | [symbol] Other | [symbol] Other | 13 | 7 | 7 | 0 | 0 | 0 |
| [symbol] Other | [symbol] Other | [symbol] Other | 0 | 0 | 36 | 0 | 0 | 0 |
| [symbol] — | [symbol] — | [symbol] — | 0 | 0 | 0 | 0 | 0 | 0 |
| [symbol] — | [symbol] — | [symbol] — | 0 | 0 | 0 | 0 | 0 | 0 |
| [symbol] — | [symbol] — | [symbol] — | 14 | 0 | 0 | 0 | 0 | 0 |
| [symbol] — | [symbol] — | [symbol] — | 0 | 13 | 8 | 0 | 0 | 0 |
| [symbol] Lachnospiraceae | [symbol] — | [symbol] — | 0 | 0 | 7 | 11 | 0 | 12 |
| [symbol] Streptococcaceae | [symbol] Streptococcus | [symbol] — | 0 | 0 | 0 | 0 | 0 | 0 |
| [symbol] — | [symbol] — | [symbol] — | 0 | 0 | 0 | 0 | 0 | 0 |
| [symbol] S24-7 | [symbol] — | [symbol] — | 0 | 0 | 0 | 0 | 0 | 0 |
| [symbol] — | [symbol] — | [symbol] — | 0 | 0 | 42 | 0 | 0 | 0 |
| [symbol] Other | [symbol] Other | [symbol] Other | 0 | 0 | 0 | 0 | 0 | 42 |
| [symbol] [Mogibacteriaceae] | [symbol] — | [symbol] — | 7 | 4 | 0 | 0 | 12 | 0 |
| [symbol] Enterobacteriaceae | [symbol] Enterobacter | [symbol] Other | 13 | 0 | 0 | 0 | 0 | 0 |
| [symbol] Eubacteriaceae | [symbol] A [symbol] tis | [symbol] — | 0 | 13 | 5 | 2 | 0 | 0 |
| [symbol] Erysipelotrichaceae | [symbol] — | [symbol] — | 0 | 0 | 0 | 0 | 0 | 19 |
| [symbol] Aerococcaceae | [symbol] Aerococcus | [symbol] — | 0 | 0 | 0 | 0 | 0 | 39 |
| [symbol] Ruminococcaceae | [symbol] Oscillospira | [symbol] — | 0 | 21 | 0 | 0 | 0 | 0 |
| [symbol] Ruminococcaceae | [symbol] Oscillospira | [symbol] — | 0 | 0 | 0 | 0 | 10 | 12 |
| [symbol] Other | [symbol] Other | [symbol] Other | 0 | 16 | 5 | 0 | 0 | 0 |
| [symbol] — | [symbol] — | [symbol] — | 0 | 0 | 5 | 9 | 10 | 8 |
| [symbol] Lachnospiraceae | [symbol] Coprococcus | [symbol] — | 0 | 17 | 0 | 0 | 0 | 0 |
| [symbol] Other | [symbol] Other | [symbol] Other | 0 | 0 | 35 | 0 | 0 | 0 |
| [symbol] — | [symbol] — | [symbol] — | 0 | 33 | 0 | 0 | 0 | 0 |
| [symbol] Lachnospiraceae | [symbol] Other | [symbol] Other | 0 | 12 | 11 | 0 | 0 | 0 |
| [symbol] Ruminococcaceae | [symbol] Oscillospira | [symbol] — | 0 | 8 | 0 | 0 | 14 | 0 |
| [symbol] S24-7 | [symbol] — | [symbol] — | 0 | 0 | 0 | 32 | 0 | 0 |
| [symbol] Ruminococcaceae | [symbol] Ruminococcus | [symbol] — | 0 | 15 | 0 | 0 | 0 | 0 |
| [symbol] Ruminococcaceae | [symbol] Oscillospira | [symbol] — | 0 | 0 | 31 | 0 | 0 | 0 |
| [symbol] — | [symbol] — | [symbol] — | 0 | 30 | 0 | 0 | 0 | 0 |
| [symbol] — | [symbol] — | [symbol] — | 0 | 14 | 0 | 0 | 0 | 0 |
| [symbol] — | [symbol] — | [symbol] — | 0 | 0 | 0 | 0 | 0 | 0 |
| [symbol] — | [symbol] — | [symbol] — | 0 | 0 | 16 | 0 | 8 | 0 |
| [symbol] Ruminococcaceae | [symbol] Ruminococcus | [symbol] — | 0 | 0 | 0 | 12 | 8 | 0 |
| [symbol] Lachnospiraceae | [symbol] — | [symbol] — | 6 | 7 | 0 | 0 | 0 | 0 |
| [symbol] Ruminococcaceae | [symbol] Oscillospira | [symbol] — | 0 | 14 | 0 | 0 | 0 | 0 |
| [symbol] — | [symbol] — | [symbol] — | 0 | 0 | 14 | 0 | 0 | 0 |
| [symbol] — | [symbol] — | [symbol] — | 0 | 0 | 20 | 0 | 0 | 0 |
| [symbol] Corynebacteriaceae | [symbol] Corynebacterium | [symbol] stationis | 26 | 0 | 0 | 0 | 0 | 0 |
| [symbol] Ruminococcaceae | [symbol] Other | [symbol] Other | 0 | 4 | 5 | 0 | 0 | 0 |
| [symbol] S24-7 | [symbol] — | [symbol] — | 0 | 0 | 0 | 0 | 0 | 13 |
| [symbol] Ruminococcaceae | [symbol] Oscillospira | [symbol] — | 0 | 0 | 0 | 0 | 0 | 26 |
| [symbol] Ruminococcaceae | [symbol] Oscillospira | [symbol] — | 0 | 0 | 0 | 0 | 0 | 0 |
| [symbol] Ruminococcaceae | [symbol] Ruminococcus | [symbol] — | 13 | 11 | 0 | 0 | 0 | 0 |
| [symbol] [Mogibacteriaceae] | [symbol] — | [symbol] — | 11 | 0 | 0 | 0 | 4 | 0 |
| [symbol] Erysipelotrichaceae | [symbol] Coprobacillus | [symbol] — | 0 | 0 | 0 | 0 | 0 | 0 |
| [symbol] Erysipelotrichaceae | [symbol] Clostridium | [symbol] cocleatum | 0 | 0 | 0 | 2 | 0 | 1 |
| [symbol] S24-7 | [symbol] — | [symbol] — | 0 | 0 | 0 | 0 | 0 | 0 |
| [symbol] Coriobacteriaceae | [symbol] — | [symbol] — | 0 | 7 | 0 | 0 | 0 | 0 |
| [symbol] Coriobacteriaceae | [symbol] Adlercreutzia | [symbol] — | 4 | 0 | 0 | 0 | 0 | 0 |
| [symbol] Leucon [symbol] ceae | [symbol] Weissella | [symbol] parame [symbol] enteroides | 2 | 0 | 0 | 0 | 3 | 12 |
| [symbol] Enterococcaceae | [symbol] Vagococcus | [symbol] — | 0 | 0 | 0 | 0 | 0 | 0 |
| [symbol] Ruminococcaceae | [symbol] Buty [symbol] coccus | [symbol] pullic [symbol] um | 0 | 0 | 6 | 0 | 0 | 0 |
| [symbol] [Mogibacteriaceae] | [symbol] — | [symbol] — | 0 | 0 | 0 | 0 | 12 | 6 |
| [symbol] — | [symbol] — | [symbol] — | 0 | 0 | 8 | 0 | 0 | 0 |
| [symbol] — | [symbol] — | [symbol] — | 0 | 0 | 20 | 0 | 0 | 0 |
| [symbol] — | [symbol] — | [symbol] — | 0 | 0 | 20 | 0 | 0 | 0 |
| [symbol] S24-7 | [symbol] — | [symbol] — | 0 | 0 | 0 | 0 | 9 | 11 |
| [symbol] Ruminococcaceae | [symbol] Oscillospira | [symbol] — | 0 | 9 | 0 | 0 | 0 | 0 |
| [symbol] Planococcaceae | [symbol] Sporosarcina | [symbol] — | 0 | 0 | 0 | 8 | 0 | 0 |
| [symbol] Lachnospiraceae | [symbol] Bl [symbol] | [symbol] producta | 0 | 7 | 0 | 0 | 6 | 0 |
| [symbol] Erysipelotrichaceae | [symbol] Other | [symbol] Other | 0 | 0 | 7 | 0 | 0 | 0 |
| [symbol] — | [symbol] — | [symbol] — | 0 | 0 | 0 | 0 | 0 | 0 |
| [symbol] Ruminococcaceae | [symbol] Ruminococcus | [symbol] — | 0 | 15 | 0 | 0 | 0 | 0 |
| [symbol] — | [symbol] — | [symbol] — | 0 | 0 | 0 | 0 | 0 | 0 |
| [symbol] — | [symbol] — | [symbol] — | 0 | 0 | 0 | 0 | 0 | 0 |
| [symbol] — | [symbol] — | [symbol] — | 0 | 0 | 0 | 15 | 0 | 0 |
| [symbol] Coriobacteriaceae | [symbol] Adlercreutzia | [symbol] — | 10 | 0 | 0 | 4 | 0 | 0 |
| [symbol] Other | [symbol] Other | [symbol] Other | 0 | 0 | 0 | 0 | 0 | 0 |
| [symbol] Ruminococcaceae | [symbol] Other | [symbol] Other | 0 | 0 | 0 | [symbol] | 9 | 0 |
| [symbol] Erysipelotrichaceae | [symbol] — | [symbol] — | 0 | 0 | 0 | 0 | 14 | 0 |
| [symbol] Deferribacteraceae | [symbol] Mucispirillum | [symbol] schaedleri | 0 | 0 | 0 | 0 | 0 | 0 |
| [symbol] Methylobacteriaceae | [symbol] Methylobacterium | [symbol] Other | 0 | 0 | 0 | 0 | 0 | 0 |
| [symbol] Methanobacteriaceae | [symbol] Methanobrevibacter | [symbol] — | 0 | 0 | 0 | 0 | 0 | 0 |
| [symbol] — | [symbol] — | [symbol] — | 0 | [symbol] | 0 | 2 | 0 | 0 |
| [symbol] Lachnospiraceae | [symbol] Other | [symbol] Other | 0 | 0 | 12 | 0 | 0 | 0 |
| [symbol] Lachnospiraceae | [symbol] Dorea | [symbol] — | 11 | 0 | 0 | 0 | 0 | 0 |
| [symbol] Lachnospiraceae | [symbol] Dorea | [symbol] — | 0 | 4 | 7 | 0 | 0 | 0 |
| [symbol] Lachnospiraceae | [symbol] — | [symbol] — | 0 | 0 | 11 | 0 | 0 | 0 |
| [symbol] Erysipelotrichaceae | [symbol] — | [symbol] — | 0 | 0 | 0 | 11 | 0 | 0 |
| [symbol] — | [symbol] — | [symbol] — | 0 | 0 | 0 | 11 | 0 | 0 |
| [symbol] Other | [symbol] Other | [symbol] Other | 0 | 0 | 0 | 0 | 0 | 0 |
| [symbol] Ruminococcaceae | [symbol] Ruminococcus | [symbol] — | 0 | 0 | 0 | 0 | 0 | 11 |
| [symbol] Lachnospiraceae | [symbol] Other | [symbol] Other | 0 | 10 | 0 | 0 | 0 | 0 |
| [symbol] Erysipelotrichaceae | [symbol] Clostridum | [symbol] cocleatum | 0 | 0 | 0 | 0 | 0 | 0 |
| [symbol] Clostridiaceae | [symbol] — | [symbol] — | 0 | 0 | 10 | 0 | 0 | 0 |
| [symbol] S24-7 | [symbol] — | [symbol] — | 0 | 0 | 0 | 0 | 6 | 0 |
| [symbol] Ruminococcaceae | [symbol] Oscillospira | [symbol] — | 0 | 0 | 0 | 0 | 0 | 10 |
| [symbol] — | [symbol] — | [symbol] — | 9 | 0 | 0 | 0 | 0 | 0 |
| [symbol] — | [symbol] — | [symbol] — | 0 | 0 | 0 | 0 | 0 | 0 |
| [symbol] Erysipelotrichaceae | [symbol] — | [symbol] — | 0 | 0 | 0 | 0 | 0 | 9 |
| [symbol] Streptococcaceae | [symbol] — | [symbol] — | 0 | 0 | 0 | 0 | 0 | 0 |
| [symbol] Erysipelotrichaceae | [symbol] — | [symbol] — | 0 | 0 | 0 | 0 | 0 | 0 |
| [symbol] Turicibacteraceae | [symbol] Turicibacter | [symbol] — | 7 | 0 | 0 | 0 | 0 | 0 |
| [symbol] S24-7 | [symbol] — | [symbol] — | 0 | 7 | 0 | 0 | 0 | 0 |
| [symbol] Ruminococcaceae | [symbol] Oscillospira | [symbol] — | 0 | 7 | 0 | 0 | 0 | 0 |
| [symbol] Lachnospiraceae | [symbol] — | [symbol] — | 0 | 7 | 0 | 0 | 0 | 0 |
| [symbol] — | [symbol] — | [symbol] — | 0 | 0 | 0 | 3 | 0 | 4 |
| [symbol] Erysipelotrichaceae | [symbol] — | [symbol] — | 0 | 0 | 0 | 0 | 0 | 0 |
| [symbol] Ruminococcaceae | [symbol] Ruminococcus | [symbol] — | 0 | 0 | 0 | 0 | 0 | 7 |
| [symbol] Moraxellaceae | [symbol] Psychrobacter | [symbol] Other | 3 | 0 | 0 | 0 | 0 | 0 |
| [symbol] Paenibacillaceae | [symbol] Other | [symbol] Other | 0 | 6 | 0 | 0 | 0 | 0 |
| [symbol] Lachnospiraceae | [symbol] Coprococcus | [symbol] — | 0 | 6 | 0 | 0 | 0 | 0 |
| [symbol] Bacillaceae | [symbol] Bacillus | [symbol] — | 0 | 0 | 0 | 0 | 0 | 0 |
| [symbol] Enterococcaceae | [symbol] Vagococcus | [symbol] — | 0 | 0 | 0 | 0 | 0 | 0 |
| [symbol] — | [symbol] — | [symbol] — | 0 | 0 | 0 | 0 | 0 | 0 |
| [symbol] Lachnospiraceae | [symbol] — | [symbol] — | 0 | 0 | 0 | 0 | 0 | 0 |
| [symbol] — | [symbol] — | [symbol] — | 0 | 0 | 0 | 0 | 0 | 6 |
| [symbol] — | [symbol] — | [symbol] — | 5 | 0 | 0 | 0 | 0 | 0 |
| [symbol] — | [symbol] — | [symbol] — | 0 | 0 | 5 | 0 | 0 | 0 |
| [symbol] Ruminococcaceae | [symbol] Oscillospira | [symbol] — | 0 | 4 | 0 | 0 | 0 | 0 |
| [symbol] — | [symbol] — | [symbol] — | 0 | 0 | 0 | 0 | 0 | 0 |
| [symbol] Erysipelotrichaceae | [symbol] — | [symbol] — | 0 | 0 | 4 | 0 | 0 | 0 |
| [symbol] Other | [symbol] Other | [symbol] Other | 0 | 0 | 0 | 0 | 0 | 4 |
| [symbol] Clostridiaceae | [symbol] Clostridium | [symbol] Other | 3 | 0 | 0 | 0 | 0 | 0 |
| [symbol] — | [symbol] — | [symbol] — | 0 | 0 | 0 | 0 | 0 | 0 |
| [symbol] Ruminococcaceae | [symbol] Buty [symbol] coccus | [symbol] pulli [symbol] um | 0 | 0 | 0 | 0 | 0 | 0 |
| [symbol] Ruminococcaceae | [symbol] — | [symbol] — | 0 | 0 | 3 | 0 | 0 | 0 |
| [symbol] Deferribacteraceae | [symbol] Mucispirillum | [symbol] schaedleri | 0 | 0 | 0 | 0 | 3 | 0 |
| [symbol] Turicibacteraceae | [symbol] Turicibacter | [symbol] — | 0 | 0 | 0 | 0 | 0 | 0 |
| [symbol] — | [symbol] — | [symbol] — | 0 | 0 | 0 | 0 | 0 | 3 |
| [symbol] Verrucomicrobiaceae | [symbol] Akkermansia | [symbol] phil | 2 | 0 | 0 | 0 | 0 | 0 |
| [symbol] [Mogibacteriaceae] | [symbol] — | [symbol] — | 0 | 2 | 0 | 0 | 0 | 0 |
| [symbol] Alcaligenaceae | [symbol] S [symbol] ella | [symbol] — | 0 | 0 | 0 | 0 | 0 | 0 |
| [symbol] Lachnospiraceae | [symbol] Dorea | [symbol] longicateria | 0 | 0 | 2 | 0 | 0 | 0 |
| [symbol] Ruminococcaceae | [symbol] — | [symbol] — | 0 | 0 | 0 | 0 | 2 | 0 |
| [symbol] Lachnospiraceae | [symbol] — | [symbol] — | 0 | 0 | 0 | 0 | 0 | 0 |
| [symbol] — | [symbol] — | [symbol] — | 0 | 0 | 0 | 0 | 0 | 2 |

[symbol]

indicates data missing or illegible when filed

### Differential Abundance Analysis, PCA and PCA, OPLS-DA and Clustering Analysis:

Principal component analysis (PCA) and orthogonal partial least squares-discriminant analysis (OPLS-DA) were performed to visualize the metabolic differences among the experimental groups. For principal component analysis, SIMCA-P+ ver.17 (Umetrics) was used. The normalized peak heights of the sample-derived peaks were used to perform principal component analysis using all samples and five points (F18S-12, F18S-14, F18S-16, F18S-18, and F18S-20). Transform was set to none, and Scaling was set to Pareto scaling. Differential metabolites were selected according to the statistically significant variable importance in the projection (VIP) values obtained from the OPLS-DA model. Hierarchical Cluster Analysis (HCA) and heat maps were performed using R (https://www.r-project.org/).

### Statistical Analysis:

Statistical data were analysed using Microsoft Excel statistics package analysis software. Graphs were prepared using Origin Lab's Originb 2021 software. For normally distributed variables, t-test or ANOVA with Tukey HSD was used and P values<0.05 were considered significant. For OPLS-DA, values from two-tailed Student's t-tests were applied on the normalized peak areas; metabolites with VIP values>1 and P values<0.05 were included. The Euclidean distance and Ward's method were used to analyze the heat map. The mean and variance were normalized so that the mean is 0 and the variance is 1.

### Results:

There were no significant differences in mean body weights at any day during the treatment period between the control group and the other treatment groups. There were no significant differences in mean body weights on the day of sacrifice between the treatment groups.

### Gut Microbiome Analysis:

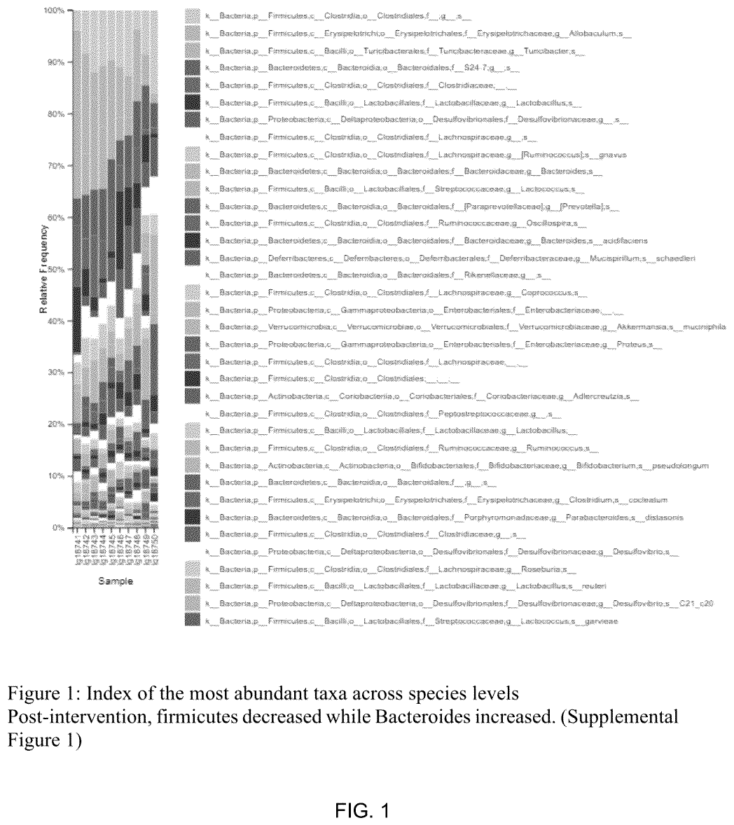

With regard to the taxonomic profiling, firmicutes represented the most abundant phyla followed by Bacteroidetes (FIG. 1)

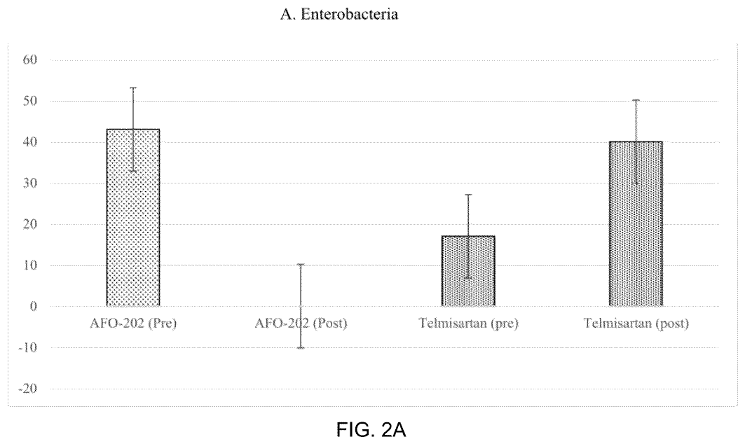

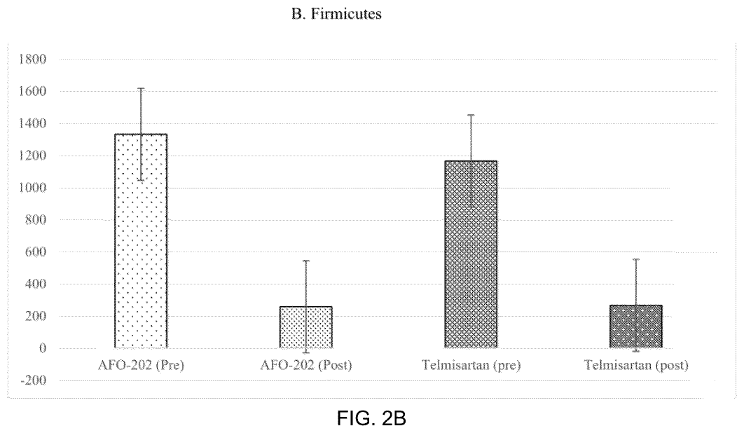

When individual taxa were analysed in each of the beta glucan groups comparing it to Telmisartan (standard), decrease in enterobacteria was highest in AFO-202 group (FIG. 2A). Decrease in Decrease in Firmicutes was highest in AFO-202 group (FIG. 2B).

### Fecal Metabolome Analysis:

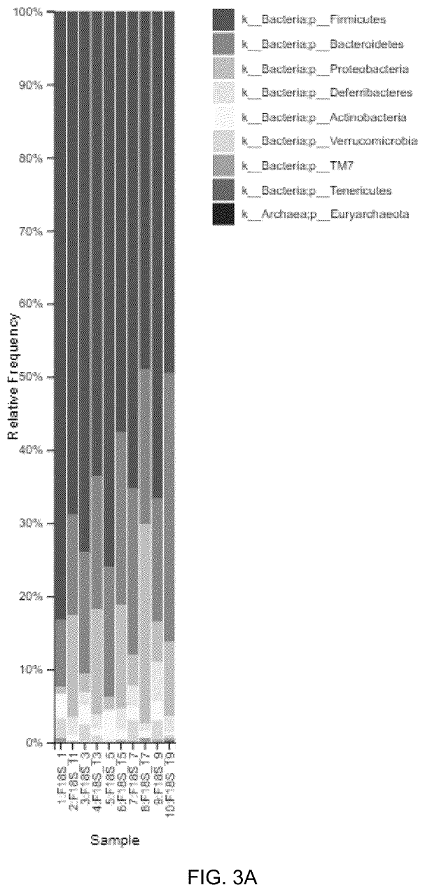

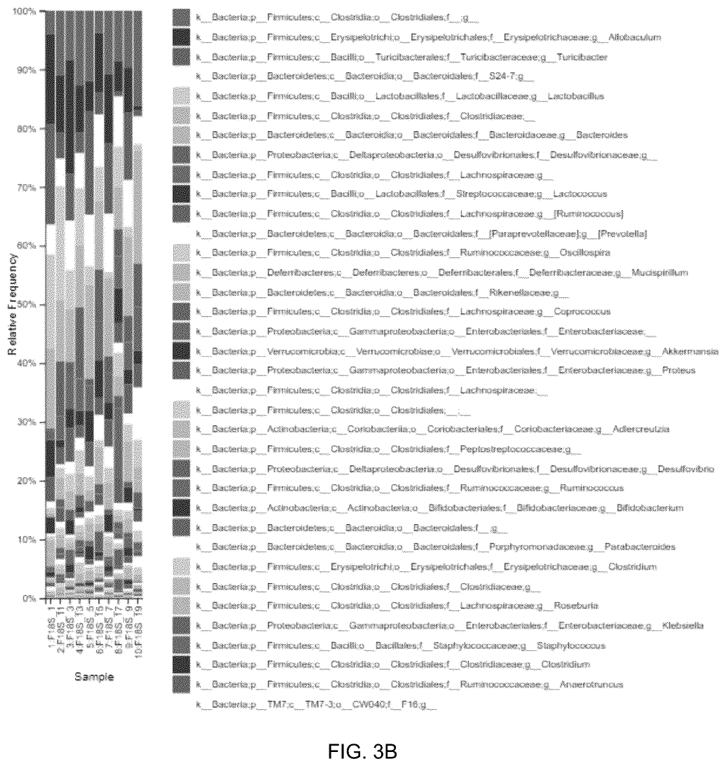

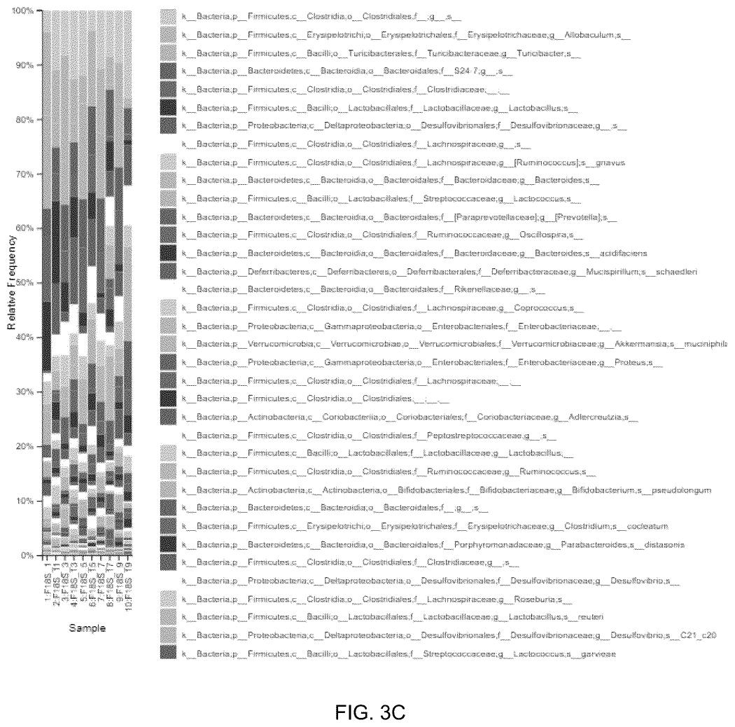

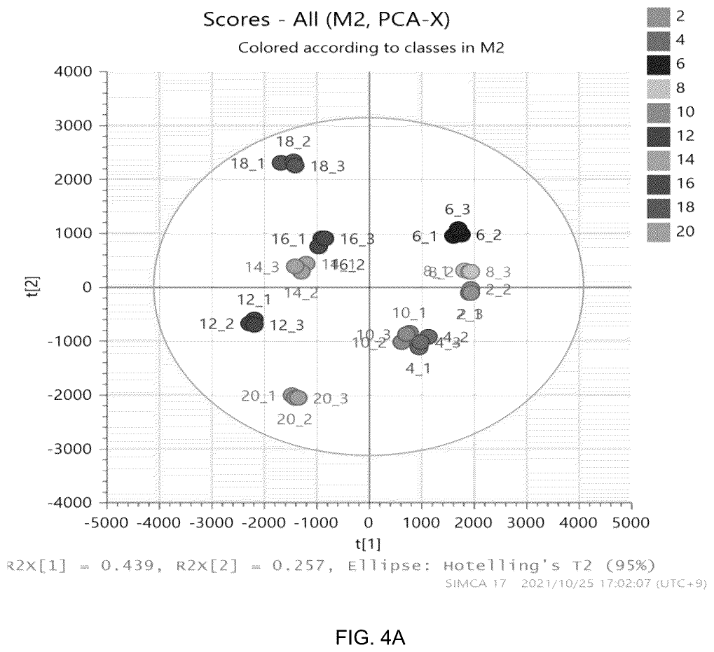

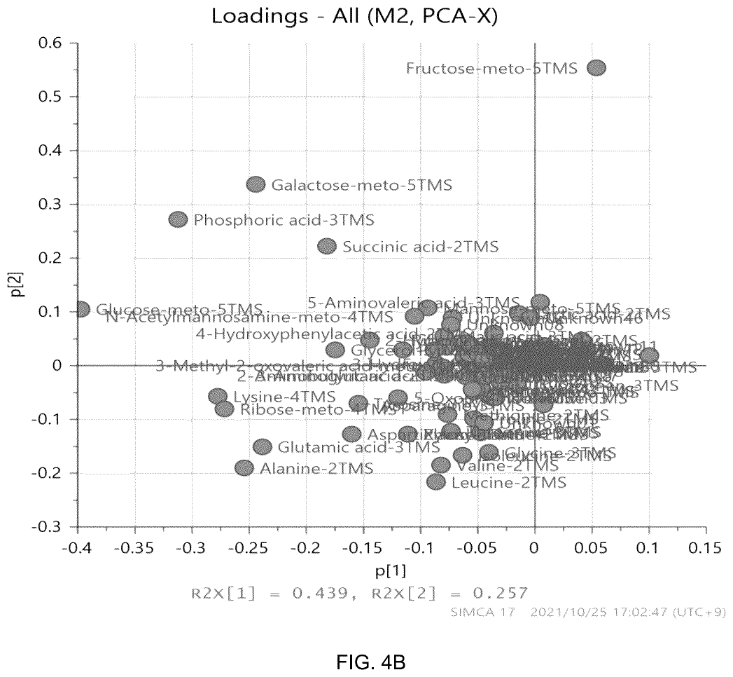

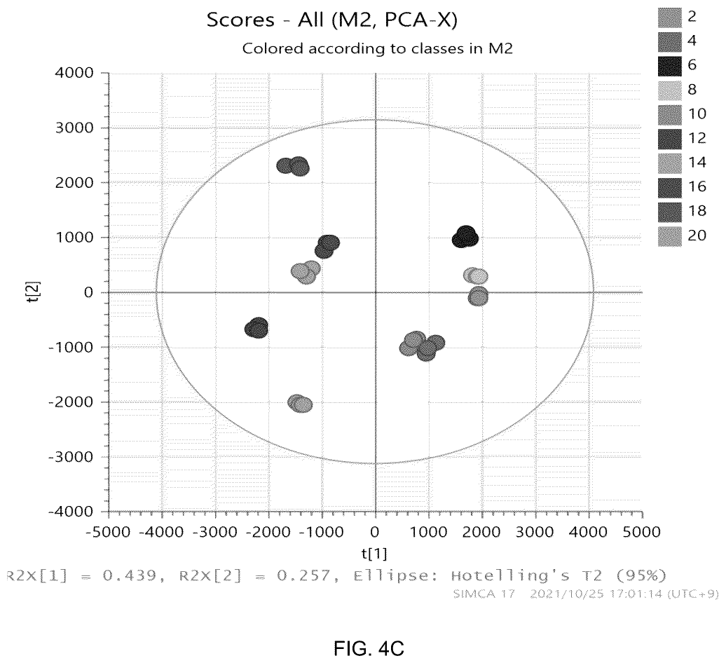

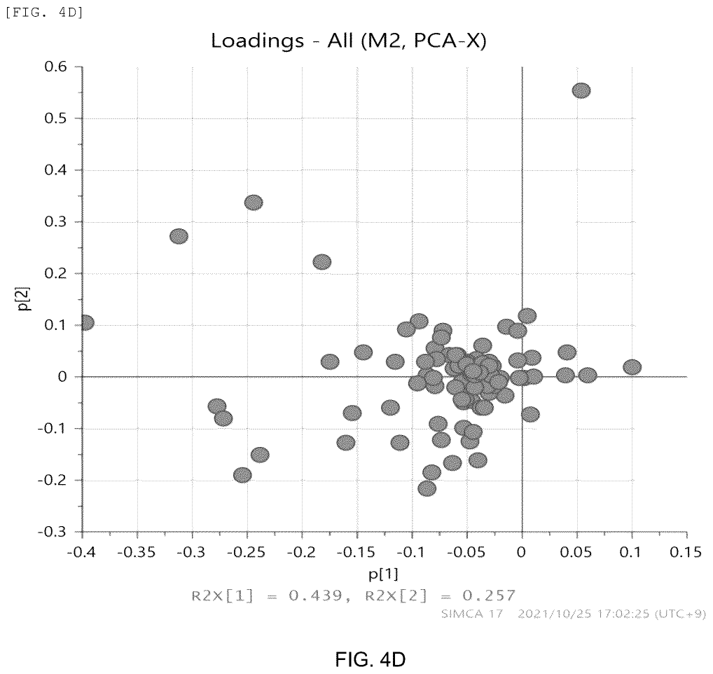

The resulting score plot of the principal component analysis using the normalized peak heights of the 10 samples (Pre- and post intervention of five groups), is shown in FIG. 3. The contribution of the first principal component was 55%, and that of the second principal component was 20%. Principal component analysis of post-intervention samples using the peak heights after normalization and the obtained score plot is shown in FIGS. 4A and 4C and the loading plot in FIGS. 4B and 4D. The contribution of the first principal component was 49% and that of the second principal component was 34%.

Peak height of all the detected metabolite compounds after normalization is available in Table 3. The values which showed decrease post-intervention are highlighted in bold in the different groups. The number of peaks detected in the QC samples was 108, of which 53 peaks were qualitatively determined and 55 peaks were unknown.

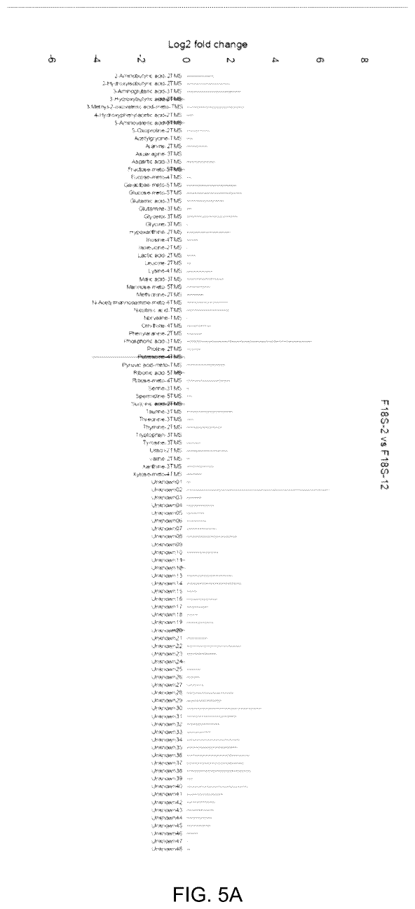

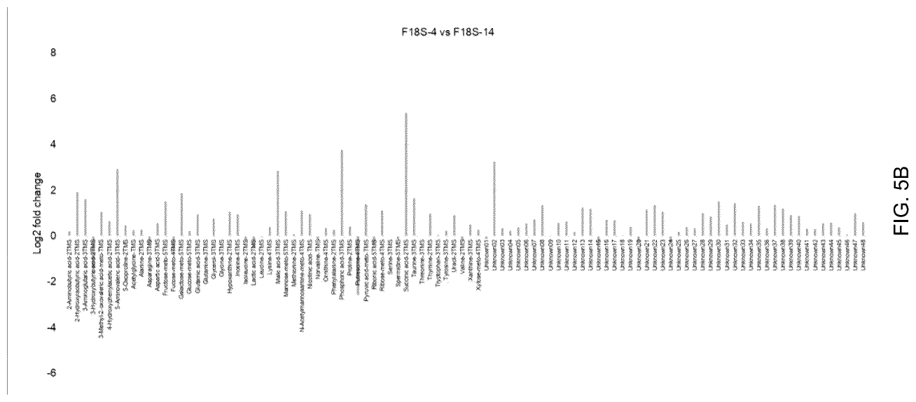

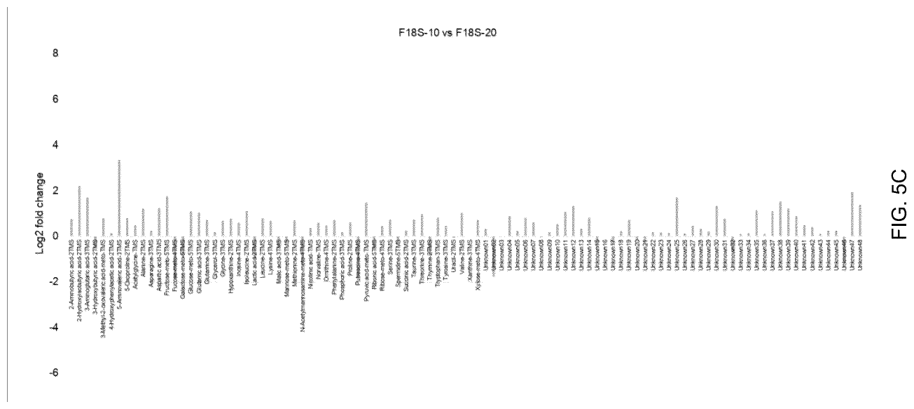

Differential abundance analysis, log 2 fold change results are shown in FIG. 5.

TABLE 3
Peak height of all the detected metabolite compounds after normalization

| | Name | Gr. 1 Control | | | Gr. 2-AFO-202 | | | Telmisartan | | |
|---|---|---|---|---|---|---|---|---|---|---|
| S. | of the | | | Differ- | | | Differ- | | | Differ- |
| No | Compound | Pre | Post | ence | Pre | Post | ence | Pre | Post | ence |
| 1 | 2-Aminobutyric | 16544.74 | 40209.97 | 23665.23 | 23704.19 | 27627.27 | 3 [symbol] 23.08 | 2 [symbol] 46.06 | 43700.57 | 17954.52 |
| | acid-2TMS | | | | | | | | | |
| 2 | 2-Hydroxyisobutyric | 36888.72 | 145407.17 | 108518.45 | 38444.81 | 146808.33 | 108363.52 | [symbol] 2.36 | 170732.7 [symbol] | 133880.36 |
| | acid-2TMS | | | | | | | | | |
| 3 | 3-Aminoglutaric | 2064.63 | 11599.10 | 9534.47 | 2674.96 | 8297.81 | 5622.84 | 3808.51 | 12436.04 | 8627.53 |
| | acid-3TMS | | | | | | | | | |
| 4 | 3-Hydroxybutyric | 78693.42 | 32663.28 | −46030.14 | 49425.90 | 15869.18 | −33556.73 | 15499.52 | 10891.34 | −4608.17 |
| | acid-2TMS | | | | | | | | | |
| 5 | 3-Methyl- | 2494.33 | 15765.26 | 13270.93 | 5771.05 | 12154.53 | 6383.48 | 7152.49 | 12393.37 | 5240.87 |
| | 2-oxo [symbol] leric | | | | | | | | | |
| | acid- [symbol] -TMS | | | | | | | | | |
| 6 | 4-Hydroxyphenyl [symbol] | 71212.24 | 90389.17 | 1 [symbol] 176.93 | 38406.22 | 61406.93 | 23000.73 | 36067.25 | [symbol] 4 | 3815.29 |
| | acid-2TMS | | | | | | | | | |
| 7 | 5-Aminovaleric | 359981.32 | 177455.86 | −182525.46 | 28606.18 | 220398.9 [symbol] | 191792 [symbol] | 28517.91 | 2 [symbol] 2458.93 | 26394.02 |
| | acid-3TMS | | | | | | | | | |
| 8 | 5-Oxoproline- | 86202.33 | 183221.07 | 97018.7 [symbol] | 101052.25 | 142117.70 | 41065.45 | 113169.35 | [symbol] .94 | [symbol] .59 |
| | 2TMS | | | | | | | | | |
| 9 | Acetylglycine- | 622 [symbol] .84 | [symbol] .14 | 16600.30 | 62217.0 [symbol] | 75225.0 [symbol] | 13007.93 | [symbol] .14 | [symbol] .2 | 2 [symbol] |
| | TMS | | | | | | | | | |
| 10 | Alanine-2TMS | 462486.71 | 9 [symbol] 2281.83 | 469795.13 | 575514.07 | 709299.17 | 133785.10 | 544032. [symbol] | 1268525.27 | 724492. [symbol] 0 |
| 11 | Asparag [symbol] - | 50264.14 | 47387.02 | −2877.12 | 52065.54 | 39742.05 | −12323.49 | [symbol] .79 | 68357. [symbol] | 10864.48 |
| | 3TMS | | | | | | | | | |
| 12 | Aspartic | 122316.67 | 309390.39 | 187073.73 | 168305.85 | 250489.61 | 82183.75 | 183416.60 | 4 [symbol] .75 | 2 [symbol] 17.15 |
| | acid-3TMS | | | | | | | | | |
| 13 | Fructose- [symbol] - | [symbol] | 999935.86 | −726073.07 | 323320.36 | 936708.26 | 613387.90 | 325202.45 | 111093 [symbol] .22 | 7857 [symbol] .77 |
| | 5TMS | | | | | | | | | |
| 14 | [symbol] | 36824.30 | 44185.98 | 7361.68 | 57488.33 | 33927.51 | −23560. [symbol] 3 | [symbol] .59 | 24027.13 | −65 [symbol] 28.4 [symbol] |
| | 4TMS | | | | | | | | | |
| 15 | Galactose- [symbol] - | 92513.44 | 457335.67 | [symbol] 822.23 | 177745.54 | 661315.76 | 48 [symbol] .22 | 359608.12 | [symbol] .00 | −170827.12 |
| | 5TMS | | | | | | | | | |
| 16 | Glucose- [symbol] - | 299911.54 | 17403 [symbol] .37 | 1440487.83 | 842456.89 | 996893.55 | 154436.66 | 400546.16 | [symbol] 55701.34 | [symbol] |
| | 5TMS | | | | | | | | | |
| 17 | Glutamic | 197256.75 | 658386.79 | 461130.05 | 264245.78 | 515323.80 | 251078.03 | [symbol] 61041.65 | 74375 [symbol] .06 | 382709.41 |
| | acid-3TMS | | | | | | | | | |
| 18 | Glutamine- | 60900.94 | 73851.25 | 12950.31 | 45289.87 | 46071.10 | 781.23 | 4 [symbol] 99.14 | 7 [symbol] 644.26 | 30745.13 |
| | 3TMS | | | | | | | | | |
| 19 | Glycerol- | 100045.16 | 5 [symbol] 2115.32 | 412070.17 | 193048.44 | 332266.23 | 139217.82 | 283873.86 | 324467.91 | 405 [symbol] |
| | 3TMS | | | | | | | | | |
| 20 | Glycine- | 17 [symbol] 941.04 | 19 [symbol] 667.24 | 14726.20 | 182268.66 | 182802.22 | 533.56 | 179124. [symbol] 5 | 288456.14 | 109331.29 |
| | 3TMS | | | | | | | | | |
| 21 | Hypoxanthine- | 1820 [symbol] .77 | 73610.39 | 554 [symbol] 0.63 | 25970.42 | 54650.62 | 28680.21 | 33144.25 | 5 [symbol] .97 | 23680.72 |
| | 2TMS | | | | | | | | | |
| 22 | I [symbol] - | 20341.44 | 29963.22 | 9621.79 | 27134.0 [symbol] | 52940.46 | 25806.38 | 24548.88 | 37226.82 | 12677.9 [symbol] |
| | 4TMS | | | | | | | | | |
| 23 | Isoleucine- | 240751.6 [symbol] | 255 [symbol] .87 | 14928.19 | 213617.13 | 18 [symbol] .81 | −28538.33 | 175687.57 | 381173.16 | 205 [symbol] |
| | 2TMS | | | | | | | | | |
| 24 | Lactic acid- | 126966.77 | 174366.10 | 43399.33 | 161320.99 | 112769.78 | −48551.21 | 71969.33 | 36363.66 | −35605.88 |
| | 2TMS | | | | | | | | | |
| 25 | Leucine- | 405177.22 | 48496 [symbol] | 79792.64 | 381285.89 | 366743.37 | −14542.52 | 364222. [symbol] 0 | 631954.96 | 267732.46 |
| | 2TMS | | | | | | | | | |
| 26 | Lysine- | 453167.29 | 1042910.27 | 589742. [symbol] | 578069.33 | 771280.17 | 193210.94 | 592034.86 | 952876.14 | 360841.28 |
| | 4TMS | | | | | | | | | |
| 27 | Malic acid- | 15431.92 | 50833.82 | 35401.90 | 18557.13 | 136072.04 | 117514.91 | 54899.38 | 38544.73 | −16354.66 |
| | 3TMS | | | | | | | | | |
| 28 | Mannose- [symbol] - | 46604.74 | 101647.05 | 55042.30 | 58090.57 | 125118.62 | 67028.06 | 79951.11 | 65364.10 | −14587.00 |
| | 3TMS | | | | | | | | | |
| 29 | Methionine- | 72809.85 | 128516. [symbol] 4 | 55706. [symbol] | 84612.94 | 90620. [symbol] | 6007.93 | 103825.98 | 168797.44 | 6 [symbol] 1.46 |
| | 2TMS | | | | | | | | | |
| 30 | N-Acetyl [symbol] - | 51349.02 | 194543.7 [symbol] | 143194.68 | 95958.86 | 210636.96 | 114678.10 | 207515.87 | 90678.54 | −116837.33 |
| | 4TMS | | | | | | | | | |
| 31 | Nicot [symbol] | 8270. [symbol] | 31967.10 | 23696.54 | 12163.20 | 23936.48 | 11773.28 | 1835 [symbol] .32 | 23826.50 | 5467.18 |
| | acid-TMS | | | | | | | | | |
| 32 | Norvaline- | [symbol] 4923.32 | 100191.34 | 5268.03 | 82912.31 | 73583.61 | −9328.70 | 72621.50 | 110365.67 | 37744.18 |
| | TMS | | | | | | | | | |
| 33 | [symbol] thine- | 45698.47 | 101711.56 | 56013.10 | 40654.29 | 52324.86 | 11670.57 | 44496.80 | 62226.84 | 17730.04 |
| | 4TMS | | | | | | | | | |
| 34 | Phenylalamine- | 173673.02 | 290028.18 | 116355.17 | 186230.03 | 228284.46 | 42048.43 | 208441.78 | 342996.16 | 134 [symbol] 4.38 |
| | 2TMS | | | | | | | | | |
| 35 | Phosphoric | 13703.35 | 692470.05 | 678766. [symbol] | [symbol] 467.68 | 765279.47 | 709811.79 | 107073.44 | 122301.92 | 15228.48 |
| | acid-3TMS | | | | | | | | | |
| 36 | Proline- | 57195.44 | 92021.21 | 34825.77 | 77037.72 | 103324.97 | 26287.25 | [symbol] 6.02 | 151136.53 | 52750.51 |
| | 2TMS | | | | | | | | | |
| 37 | P [symbol] - | 90940.03 | 4806.17 | −86133.86 | 30410.15 | 5030.93 | −25379.22 | 21749. [symbol] | 6122. [symbol] | −15626.91 |
| | 4TMS | | | | | | | | | |
| 38 | Pyruvic | 31557.87 | 108630.32 | 77072.65 | 40255.26 | 105996.98 | 65741.72 | 41703.69 | 116493.60 | 74789.91 |
| | acid- [symbol] -TMS | | | | | | | | | |
| 39 | Ribo [symbol] | 90115.51 | 64726.74 | −25388.76 | 85151.16 | 66560.71 | −18390.46 | 95701.95 | 64707.65 | −30994.30 |
| | acid-5TMS | | | | | | | | | |
| 40 | Rib [symbol] - | 199285.35 | 806414.91 | 607129.56 | 321848.24 | 706173.47 | 384325.23 | 510635.39 | 6996 [symbol] 0.70 | 189015.32 |
| | 4TMS | | | | | | | | | |
| 41 | #Sz,899;- | 134100.10 | 150072.29 | 15972. [symbol] 9 | 134521.62 | 135009.56 | 487.94 | 124526.34 | 206603. [symbol] 4 | 820 [symbol] .40 |
| | 3TMS | | | | | | | | | |
| 42 | Spe [symbol] - | 11571. [symbol] 9 | 14335.92 | 2 [symbol] 64.13 | 8257.93 | 7462.86 | −795.07 | 9612. [symbol] 4 | 8122.10 | −1490.65 |
| | 3TMS | | | | | | | | | |
| 43 | Succinic | 200720.47 | 75253.68 | −125466.79 | 32921.32 | 139 [symbol] 291.07 | 1365369.7 [symbol] | 263097.91 | [symbol] 13904.61 | 50806.69 |
| | acid-2TMS | | | | | | | | | |
| 44 | Taurine- | 15049.13 | 66033.11 | 50983.98 | 14685.18 | 46705.91 | 32020.73 | 14857.44 | 24726.77 | 9869.33 |
| | 3TMS | | | | | | | | | |
| 45 | T [symbol] nine- | 137361.71 | 175385.78 | 38024.08 | 137475.15 | 135608.93 | −1866.22 | 122185.79 | 240869.77 | 118683.98 |
| | 3TMS | | | | | | | | | |
| 46 | Thy [symbol] - | 5319.57 | 16561.64 | 11242.07 | 6636.79 | 13272.69 | 6635.90 | 12893.64 | 7133.20 | −5760.44 |
| | 2TMS | | | | | | | | | |
| 47 | Tryptophan- | 21044.89 | 2089 [symbol] .79 | −149.10 | 17365.35 | 18254.73 | 889.38 | 16169.30 | 28678.00 | 12 [symbol] 08.70 |
| | 3TMS | | | | | | | | | |
| 48 | Tyrosine- | 273469.68 | 438287.99 | 164818.32 | 293828.48 | [symbol] 51487.07 | 576 [symbol] 8.59 | 309190.46 | 42582 [symbol] .94 | 116635.49 |
| | 3TMS | | | | | | | | | |
| 49 | Uracil- | 25078.62 | 92837.69 | 67759.07 | 44546.07 | 84845.85 | 40299.79 | 74843.38 | 71567.16 | −3276.22 |
| | 2TMS | | | | | | | | | |
| 50 | Valine- | 353165.15 | 405846.51 | 52681.36 | 290698.34 | 255449.25 | −35249. [symbol] 8 | 254032.97 | 521841.20 | 267808.23 |
| | 2TMS | | | | | | | | | |
| 51 | Xanthine- | 53777.98 | 130586.54 | 76808.56 | 79540.73 | 113793.80 | 34253.08 | 101684.60 | 103194.40 | 1 [symbol] 09.80 |
| | 3TMS | | | | | | | | | |
| 52 | Xylose- [symbol] - | 174559.36 | 290187.08 | 115627.72 | 186404.46 | 228374.90 | 41970.44 | 208 [symbol] 74.29 | 343176.94 | 134602.65 |
| | 4TMS | | | | | | | | | |

[symbol]

indicates data missing or illegible when filed

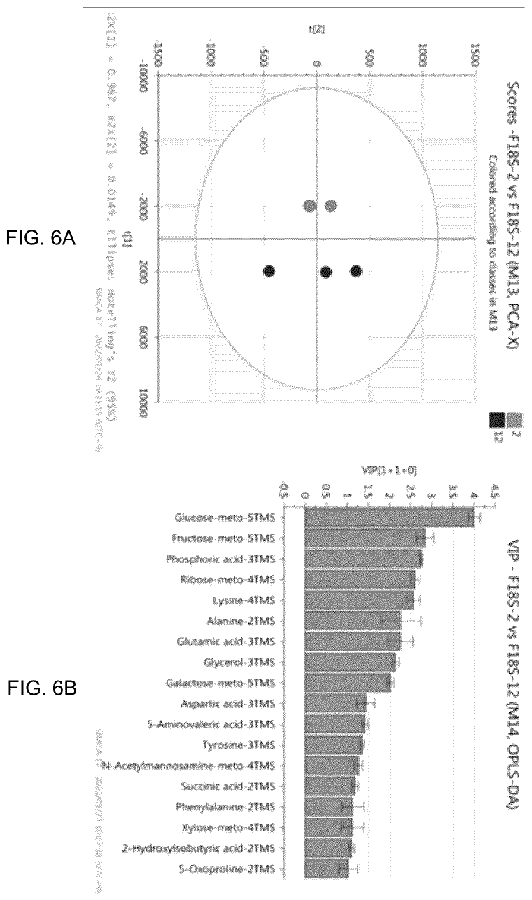

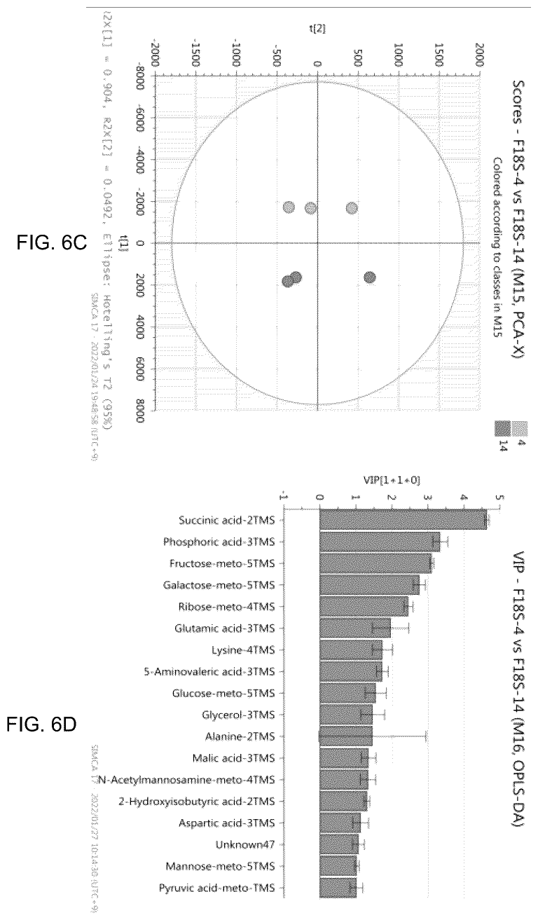

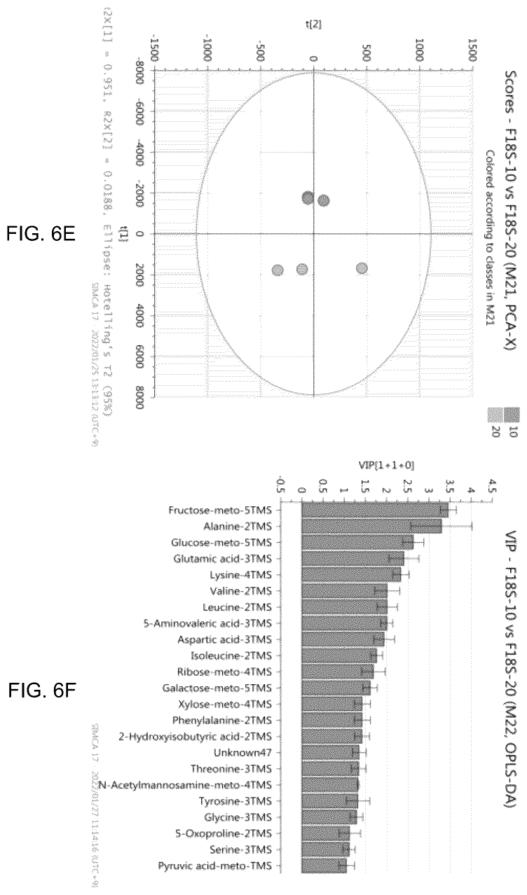

Score plots of PCA and compounds with a VIP value of 1 or higher in the OPLS-DA are shown in FIG. 6 and Table 4 shows the compounds with a VIP value of 1 or higher and their Coefficients in OPLS-DA. The results of the principal component analysis of the control group showed that the contribution of the first principal component axis (PC1) was 96.7% and that of the second principal component axis (PC2) was 1.5%. In AFO-202 group, PCA showed that PC1 and PC2 contributed 90.4% and 4.9%, respectively. In the Telmisartan group, PC1 and PC2 contributed 95.1% and 1.9%, respectively.

TABLE 4
Coefficients of Metabolite compounds with a VIP value
of 1 or higher in OPLS-DA in the different groups

| | | Coefficients | | |
|---|---|---|---|---|
| No. | Metabolites | Control | AFO-202 | Telmisartan |
| 1 | Glucose-meto-5TMS | −0.000108 | −0.000091 | −0.000139 |
| 2 | Fructose-meto-5TMS | 0.000133 | −0.000173 | −0.000209 |
| 3 | Phosphoric acid-3TMS | −0.000125 | | |
| 4 | Ribose-meto-4TMS | −0.000116 | −0.000132 | −0.000083 |
| 5 | Lysine-4TMS | −0.000116 | −0.000083 | −0.000120 |
| 6 | Alamine-2TMS | −0.000109 | −0.000003 | −0.000171 |
| 7 | Glutamic acid-3TMS | −0.000100 | −0.000101 | −0.000149 |
| 8 | Glycero-3TMS | −0.000102 | −0.000080 | |
| 9 | Galactose-meto-5TMS | −0.000092 | −0.000144 | 0.000094 |
| 10 | Aspartic acid-3TMS | −0.000063 | −0.000061 | −0.000113 |
| 11 | 5-Aminovaleric acid-3TMS | 0.000069 | −0.000103 | −0.000123 |
| 12 | Tyrosine-3TMS | −0.000064 | | −0.000043 |
| 13 | N-Acetylmannosamine-meto-4TMS | −0.000055 | −0.000068 | |
| 14 | Succinic acid-2TMS | 0.000059 | −0.000259 | |
| 15 | Phenylalanine-2TMS | −0.000049 | | −0.000087 |
| 16 | Xylose-meto-4TMS | −0.000048 | | −0.000087 |
| 17 | 2-Hydroxyisobutyric acid-2TMS | −0.000054 | −0.000072 | −0.000072 |
| 18 | 5-Oxoproline-2TMS | −0.000043 | | |
| 19 | Phosphoric acid-3TMS | | −0.000197 | |
| 20 | Malic acid-3TMS | | −0.000077 | |
| 21 | Mannose-meto-5TMS | | −0.000057 | |
| 22 | Pyruvic acid-meto-TMS | | −0.000065 | −0.000046 |
| 23 | Lactic acid-2TMS | | | |
| 24 | Valine-2TMS | | | −0.000075 |
| 25 | Leucine-2TMS | | | −0.000087 |
| 26 | Isoleucine-2TMS | | | −0.000083 |
| 27 | Threonine-3TMS | | | −0.000080 |
| 28 | Glycine-3TMS | | | −0.000058 |
| 29 | 5-Oxoproline-2TMS | | | −0.000064 |
| 30 | Serine-3TMS | | | −0.000065 |

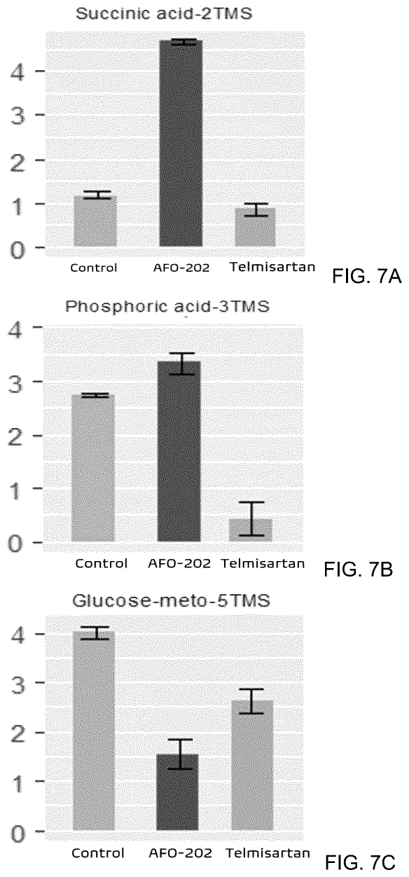

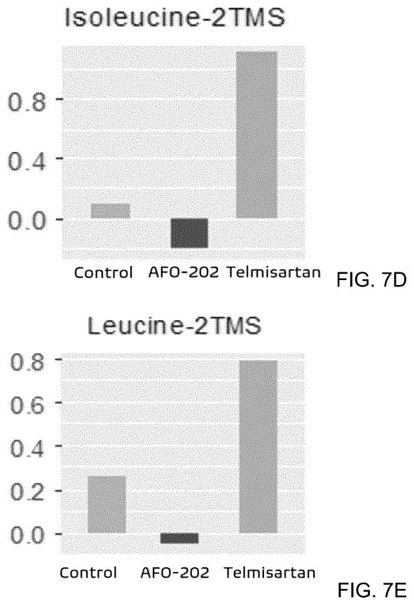

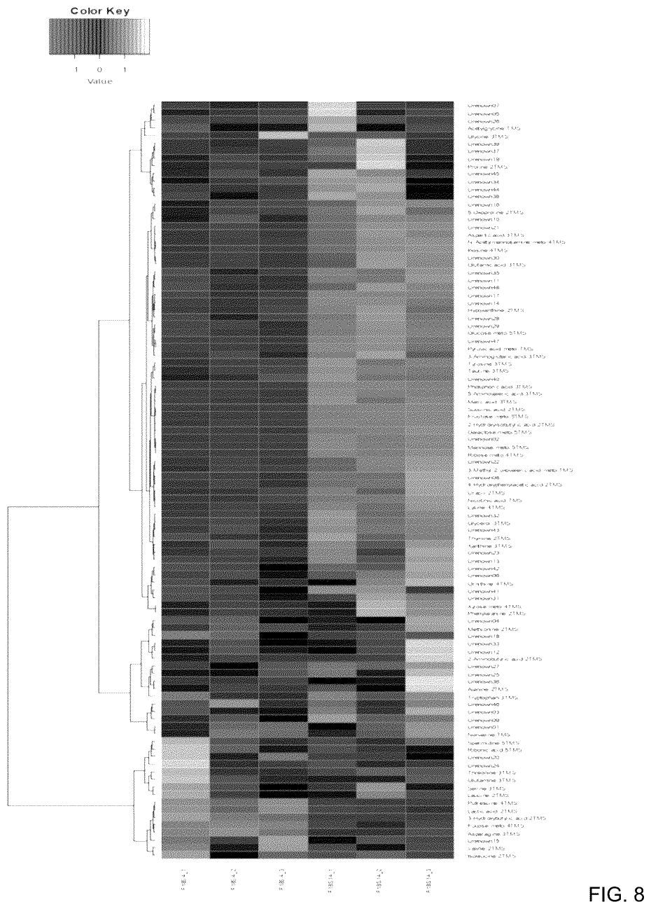

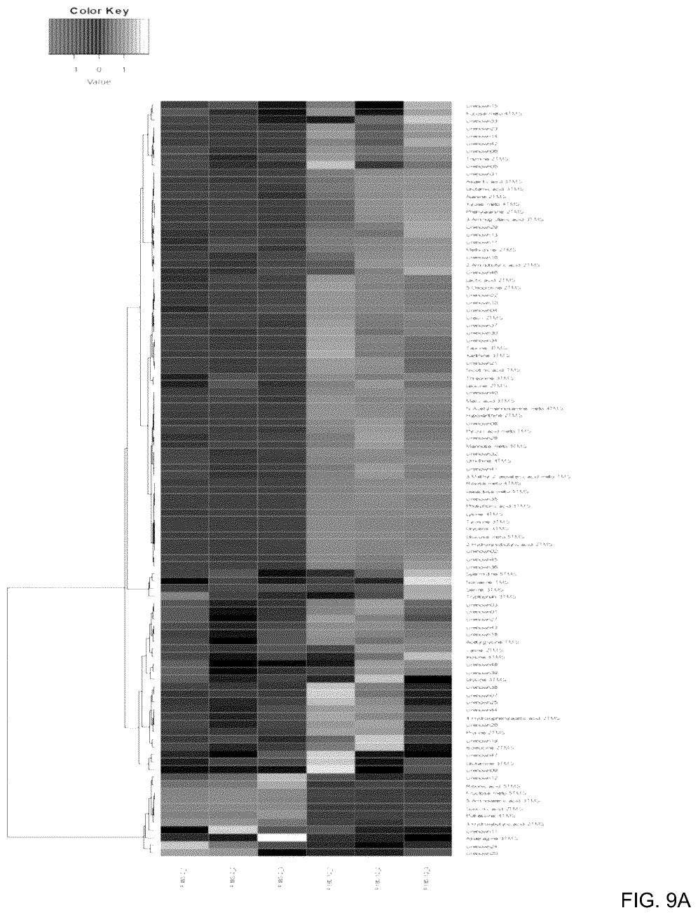

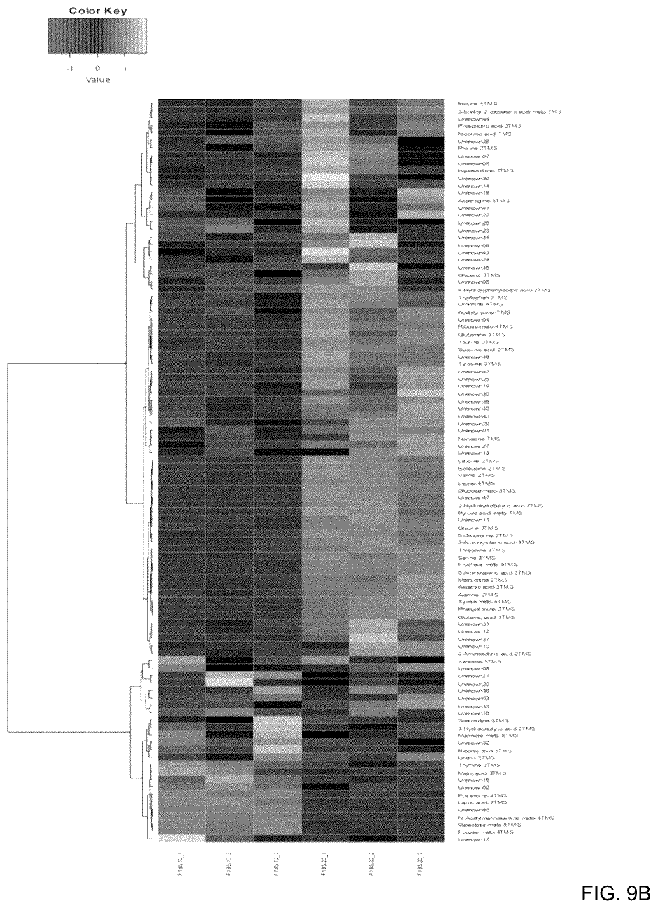

In all the groups except telmisartan, phosphoric acid shoed the highest log 2 fold change in terms of increase. Putrescine showed the highest decrease. In regard to specific compounds, increase in succinic acid was highest in AFO-202 (P-value=0.06) with statistical significance (FIG. 7A). Increase in phosphoric acid was also high in AFO-202 (FIG. 7B), though not significant (p-value=0.21). Decrease in fructose was highest in AFO-202 (FIG. 7C) (p-value=0.0007). Euclidean distance hierarchical clustering analysis demonstrating the different intensity levels of characteristic metabolites also matched the above observations (FIG. 8 and FIG. 9)

### Discussion:

This is the first study to investigate profiles of fecal gut microbiome and metabolome in a NASH model of mice with relevance to beta glucans specially so for two different beta glucans produced by different strains of same species of black yeast A. pullulans. Beta glucans are obtained from different sources and the functionality depends on the source and extraction/purification processes [D14]. The beta glucans described in the study from AFO-202 strain of the A. pullulans black yeast are unique as they are produced as an exopolysaccharide without the need for extraction/purification and hence the biological actions are superior [D15].

The AFO-202 beta glucan has been reported to have superior metabolic benefits by regularization of blood glucose levels [D5] apart from immune enhancement in immune-infectious illnesses such as COVID-19 [D11,12] and has been reported to produce positive effects on melatonin and alpha-synuclein neurotransmitters apart from improving sleep and behaviour in neurodevelopmental disorders such as ASD [D7,8]. In the NASH animal study, AFO-202 beta glucan has been able to significantly decrease inflammation-associated hepatic cell ballooning and steatosis [D10].

The N-163 beta glucan has been able to produce immune-modulatory benefits in terms of regulating dyslipidaemia evident from balance of the levels of non-esterified fatty acids [D16] and decrease in fibrosis and inflammation in NASH [D10]. The combination of AFO-202 and N-163 beta glucans has been able to decrease in pro-inflammatory markers and increase in anti-inflammatory markers in an advantageous manner in healthy human volunteers [D17], decrease the NAFLD Activity Score (NAS) in the NASH model [D10] and significantly control of immune-mediated dysregulated levels of IL-6, CRP and Ferritin in Covid-19 patients [D11,12]. In the study done on gut microbiome analysis in ASD subjects, there was efficient control of enterobacteria apart from beneficial reconstitution of the gut microbiome favourable for producing benefits in ASD by AFO-202 beta glucan [D13]. In the current study, we sought to evaluate the benefits of AFO-202 and N-163 individually and in combination in the NASH animal model.

The STAM model of NASH used in the study is a disease model, the Stelic Animal model [D10,18,19], in which mice are allowed to develop liver steatosis by injection streptozotocin solution 2 days after birth and fed with a high-fat diet. This model recapitulates most of the features of metabolic syndrome which occurs in humans wherein obesity and a high fat diet gives rise to diabetes, dyslipidaemia and liver steatosis.

Therefore, the gut microbiome profiles and fecal metabolite profiles that are present at baseline can be considered to recapitulate that which is present in metabolic syndrome [D20,21] which over time will produce pathophysiological problems in different organ systems of the body including the heart, liver, kidney apart from immune-metabolic interactions leading to a declined immune system with aging and its associated complications.

Therefore, the present study will serve as a forerunner to study the effects of the beta glucans on different aspects of metabolic syndrome associated pathologies as well as conditions associated with immune-metabolic interactions including neurological disorders in which such immune-metabolic interactions have profound implications [D20].

### Relevance of Metabolome and Microbiome to NASH:

An abundance of bacterial species, such as Proteobacteria, Enterobacteria, and Escherichia coli has been reported in humans with NAFLD[D21, 22]. In the current study, a decrease in Enterobacteria with AFO-202 was reported.

### Potentials in Neurological Illnesses:

### Neurodevelopmental and Neurodegenerative:

We have earlier reported the decrease in Enterobacteria, Escherichia coli, Akkermansia muciniphila CAG:154, Blautia spp., Coprobacillus sp., and Clostridium bolteae CAG:59, with an increase of Faecalibacterium prausnitzii and Prevotella copri after AFO-202 consumption in children with ASD [D13]. In the present study as well, the decrease in enterobacteria was highest in AFO-202 group (FIG. 5A). Succinic acid which has been reported to be lower in ASD individuals [D23] was found to increase the highest in AFO-202 group (FIGS. 7A-C). In regard to neurodegenerative diseases such as Parkinson's disease, amino acids such as isoleucine, and leucine have been found to be higher in the fecal metabolome [D24]. In the present study, decrease in these amino acids were highest in the AFO-202 group (FIGS. 7D-E).

### Other Implications:

Use of Steroids has been reported to cause increase in E-Coli, enterococcus while decrease in Bacteroides [D25]. In the current study, the control of enterobacteria with increase in Bacteroides by AFO-202 make it worthy adjuncts for medications such as steroids as well.

### Conclusion:

In summary, black yeast A. pullulans' strain AFO-202 produced beta glucans increase the gut microbial diversity, control the harmful bacteria, promote healthy ones apart from producing beneficial differences in fecal metabolites, all indicative of a healthy profile both individually and in combination in this NASH animal model. AFO-202 will serve as a worthy treatment adjunct for neurodevelopmental conditions such as ASD and neurodegenerative conditions such as PD. With further validation on the correlation between gut microbiome and fecal metabolites in specific conditions, these safety proven prebiotics have potential applications in promoting a healthy life.

### Increasing Gut Microbiota

There is increasing evidence that gut dysbiosis plays a critical role in the development and progression of several neurodevelopmental conditions such as autism spectrum disorders (ASD), neuroinflammatory conditions such as multiple sclerosis (MS) and neurodegenerative diseases such as Alzheimers (AD) and Parkinson's disease (PD). The biofilms and byproducts of the bacteria especially the Gram-negative enteric bacteria mediate the effects of the altered gut microbiome in these disease conditions. Amyloid proteins constitute the major part of the biofilms especially the Curli amyloid whose characteristics have been reported to be similar to pathological and immunomodulatory human amyloids such as Alzheimer's disease associated amyloid-β, ASD and PD associated α-synuclein [A1].

Though these reports discuss on the correlation of the levels of these amyloids, enteric bacteria and the neural diseases, there has been no simple and safe intervention with subjective and objective correlation to a clinical benefit associated by altering the gut microbiota to a beneficial advantage. We herein report the outcome of beneficial reconstitution of gut microbiota especially those of the bacteria which are associated with Alpha synuclein and Curli amyloids after consumption of Beta 1,3-1,6 Glucans in children with autism spectrum disorder in a clinical pilot study.

The emerging evidence of constant communication and interaction between the gut and the brain through the gut-brain axis has begun to unravel its significance associated with the health of the central nervous system. Any dysbiosis of the gut microbiota has been shown to influence the development and progression of neurological pathologies of developmental (autism spectrum disorder [ASD]), inflammatory (multiple sclerosis [MS]), and degenerative (Alzheimer's disease [AD] and Parkinson's disease [PD]) disorders (Kang et al., 2019). The mechanisms involve activation of the immune system; production of inflammatory cytokines and chemokines (e.g., IL-6 and TNF-α); and alteration of the gut barrier permeability, which in turn is due to the increased levels of circulating lipopolysaccharide in these neurological disorders. These mechanisms modulate the neurotrophic factors, activity of the central and peripheral nervous system, and the endocrine pathways, all of which contribute to the onset or the phenotypic expression of neuropsychiatric and neurodevelopmental disorders (Santocchi et al., 2020).

Another important player is the amyloid protein, which has self-aggregation properties. Even non-identical amyloid proteins can accelerate reciprocal amyloid aggregation in a prion-like fashion. Nearly 30 amyloidogenic proteins are encoded by humans, whereas some functional amyloids are produced by the gut microbiome (Werner et al., 2020). Of importance are cell-surface amyloid proteins called curli, which are produced by certain enterobacteria that in turn accelerate formation of α-synuclein (αSyn), which is a presynaptic neurotransmitter that is crucial in the initiation and pathogenesis of neurological disorders such as ASD, PD, AD, and MS (Sampson et al., 2020). Though these reports (Sampson et al., 2020; Werner et al., 2020; Santocchi et al., 2020) discuss the correlation of the levels of these amyloids, enteric bacteria, and neural diseases, there has been no simple and safe intervention with subjective and objective correlation to a clinical benefit that can be derived based on an associated balancing of the gut microbiota.

We herein report the outcome of beneficial reconstitution of gut microbiota, especially those of the bacteria associated with αSyn and curli amyloids after consumption of beta 1,3-1,6 glucans in children with ASD in a clinical pilot study. The beta-glucan studied (Nichi Glucan) was obtained from the AFO-202 strain of a black yeast called Aureobasidium pullulans that has beneficial advantages in metabolic disorders by alleviating glucotoxicity (Dedeepiya et al., 2012), lipotoxicity (Ganesh et al., 2014; Ikewaki et al., 2021^{a}), lipidemia-induced hepatic fibrosis (Ikewaki et al., 2021^{b}), inflammation (Ikewaki et al., 2021^{b}) apart from immune-enhancement (Ikewaki et al., 2021^{c}), and modulation in COVID-19 (Raghavan et al., 2022), which have been reported in translational and clinical studies.

The effects of this AFO-202 beta glucan in terms of behavioural (Raghavan et al., 2021a) and sleep pattern improvement (Raghavan et al., 2021^{b}), increase in levels of plasma αSyn (Raghavan et al., 2021^{a}), and serum melatonin (Raghavan et al., 2021^{b}) levels in children with ASD has been reported. We report the effects of AFO-202 beta glucan on the gut microbiome in children with ASD who participated in our pilot study.

The beta-glucan studied (Nichi Glucan) was obtained from the AFO-202 strain of a black yeast called Aureobasidium pullulans that has beneficial advantages in metabolic disorders by alleviating glucotoxicity [B5], lipotoxicity [B6, B7], lipidemia-induced hepatic fibrosis [B8], inflammation [B8] apart from immune-enhancement [B9], and modulation in COVID-19 [B10], which have been reported in translational and clinical studies. The effects of this AFO-202 beta glucan in terms of behavioural [E11] and sleep pattern improvement [B12], increase in levels of plasma αSyn [E11], and serum melatonin [B12] levels in children with ASD has been reported. We report the effects of AFO-202 beta glucan on the gut microbiome in children with ASD who participated in our pilot study.

In this randomized pilot clinical study, we have evaluated the gut microbiota of subjects with autism spectrum disorder (ASD) after consumption of Aureobasidium pullulans (black yeast) AFO-202 strain-produced beta glucan, Nichi Glucan.

### Example 1

### Methods:

The study was conducted in 18 subjects who were randomly allocated; six subjects (n=6) to the control group (Gr.1) who underwent conventional treatment comprising remedial behavioural therapies and L-carnosine 500 mg per day, and twelve subjects (n=12) Gr. 2 underwent supplementation with an Aureobasidium black yeast AFO-202 (Aureobasidium pullulans strain AFO-202 (also referred to as FO-68 [accession number: FERM BP-19327])) derived beta glucan, Nichi Glucan 0.5 g twice daily along with the conventional treatment. Stool samples from the subjects was collected at baseline and after the intervention. The samples were sequenced using Novaseq 6000 with a read length of 151 bp and taken for whole genome metagenome analysis.

Thirteen subjects (four in control (Gr.1) and nine in Nichi Glucan (Gr.2) completed the study. The sample reads were filtered for human DNA contamination. The alignment to the human genome was around 18.98%. The filtered reads were then aligned to bacterial, fungal, viral and archea genomes. The overall alignment to the bacterial genome was around 40%. However, the alignment to the viral, fungi and archea genomes was around 0.05-0.2%. De novo assembly was carried out using the pre-processed reads to obtain the scaffolds. These scaffolds were then used for gene prediction. Bacterial abundance was analysed.

### Results:

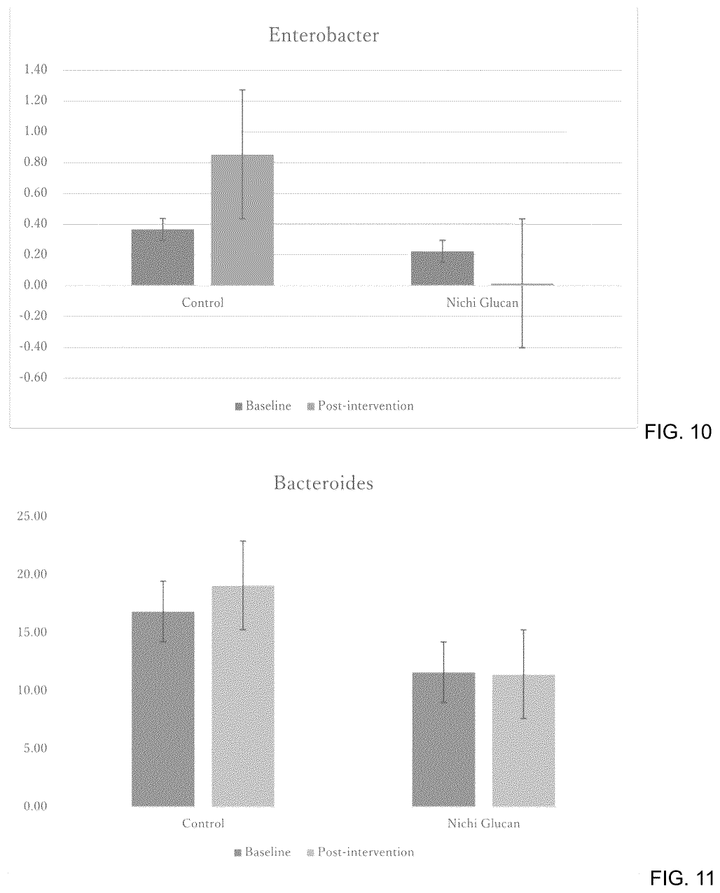

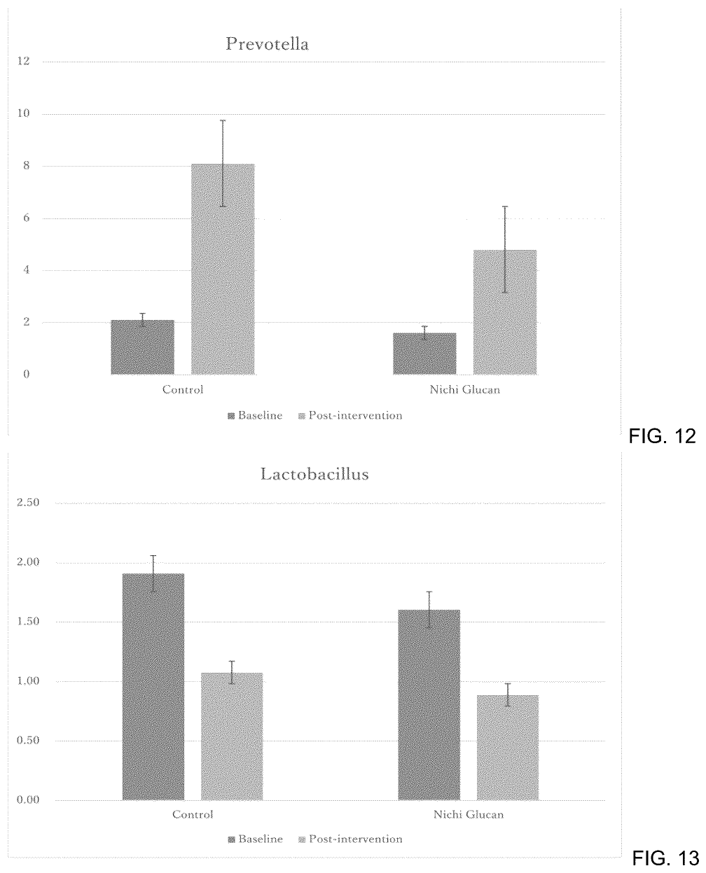

The abundance of Enterobacter was decreased almost to nil in the Nichi Glucan (Gr.2) group after intervention while it increased from 0.36% to 0.85% in the control group (FIG. 10). The abundance of Bacteroides increased from 16.84 to 19.09% in the control while it decreased from 11.60 to 11.43% in the Gr. 2 (FIG. 11) after intervention. The abundance of Prevotella increased in both Gr.1 and Gr.2 (FIG. 12). The decrease in abundance of Lactobacillus was significant in Gr.2 compared to Gr. 1 (FIG. 13).

### Conclusion:

Earlier reports have indicated a lower abundance of Prevotella, higher abundance of Lactobacillus and Bacteroides in children with autism spectrum disorders [A2]. MS patients also have been reported to have a lower abundance of Prevotella [A4]. Enterobacter produce functional amyloid proteins termed curli which promotes human amyloid α-synuclein (αSyn) pathology and the aggregation of curli and αSyn stimulates pathological and immunological processes that lead to the neurodevelopmental and neurodegenerative diseases such as ASD, MS, PD and AD [A1]. The results of this study which have produced favorable results after Nichi Glucan consumption in the gut microbiota by alleviate the pathological processes behind such neurodevelopmental and neurodegenerative diseases involving amyloid accumulations warrant larger clinical studies to recommend this as a routine food supplement in patients with neurodegenerative and neuroinflammatory conditions apart from research to explore their mechanisms and further potentials.

### Example 2

### Methods:

Eighteen subjects with ASD were randomly allocated: six subjects in the control group (Group 1): conventional treatment comprising remedial behavioural therapies and L-carnosine 500 mg per day, and 12 subjects (Group 2) underwent supplementation with Nichi Glucan 0.5 g twice daily along with the conventional treatment for 90 days.

### Results:

Whole genome metagenome (WGM) sequencing of the stool samples at baseline and after intervention, showed that among genera of relevance, the abundance of Enterobacteria was decreased almost to zero in Group 2 after intervention, whereas it increased from 0.36% to 0.85% in Group 1. The abundance of Bacteroides increased in Group 1, whereas it decreased % in Group 2. The abundance of Prevotella increased in both Group 1 and Group 2. The decrease in abundance of Lactobacillus was significant in Group 2 compared to Group 1. Among species, a decrease was seen in Escherichia coli, Akkermansia muciniphila CAG:154, Blautia spp., Coprobacillus sp., and Clostridium bolteae CAG:59, with an increase of Faecalibacterium prausnitzii and Prevotella copri, which are both beneficial.

### Conclusion:

AFO-202 beta 1,3-1,6 glucan apart from balancing the gut microbiome in children with ASD, its role in effective control of curli-producing enterobacteria that leads to α-synuclein (αSyn) misfolding and accumulation, may have a prophylactic role in Parkinson's and Alzheimer's diseases as well.

### Trial Registration:

The study was registered in India's clinical trial registry CTRI, Ref no:
CTRI/2020/10/028322. URL:
http://ctri.nic.in/Clinicaltrials/showallp.php?mid1=47623&EncHid=&userName=ken max.

### Example 3

### Methods:

This study was approved by the institutional ethics committee of the hospital in which the study took place and was registered as a clinical trial in the national clinical trial registry. The caregivers of each subject gave their informed consent for inclusion before participation in the study. The study was conducted in accordance with the Declaration of Helsinki.

### Patient Involvement:

Patients were involved in the design and conduct of this research. During the feasibility stage, priority of the research question, choice of outcome measures, and methods of recruitment were informed by discussions with patients through a focus group session and structured interviews. Once the trial has been published, participants will be informed of the results through a study newsletter suitable for a non-specialist audience.

### Study Design

The subjects enrolled in the study had received a diagnosis of ASD from a developmental pediatrician, which was verified by a psychologist using a clinical interview for a behavioural pattern that incorporated the Childhood Autism Rating Scale score.

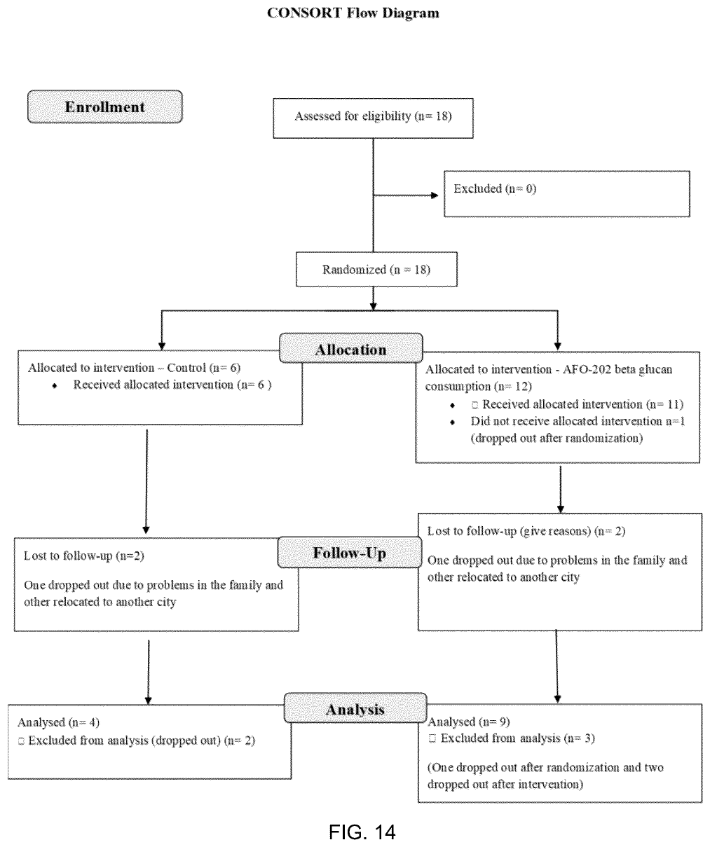

Eighteen subjects with ASD were enrolled in this prospective, open-label, pilot clinical trial comprised of two arms. The CONSORT flow diagram is presented in FIG. 14.

### Study Groups

Arm 1 or Group 1 (control group): Six subjects with ASD underwent conventional treatment comprising remedial behavioural therapies and L-carnosine 500 mg per day.

Arm 2 or Group 2 (Nichi Glucan group): 12 subjects underwent supplementation with Nichi Glucan food supplement along with conventional treatment (remedial behavioural therapies and L-carnosine 500 mg per day). Each subject consumed two sachets (0.5 g each) of Nichi Glucan daily—one sachet with a meal twice daily—for 90 days.

The inclusion and exclusion criteria along with the assessment of behavioural and sleep pattern apart from evaluation of levels of αSyn and melatonin are available in the results of the clinical trial reported earlier (Raghavan et al., 2021^{a,b})

### Faecal Sample Collection and Preparation

Faecal samples were collected at baseline and 90 days after the intervention using a sterile faecal collection kit and the samples were kept at −20° C. until they were transferred to the laboratory and processed. Samples for DNA extraction were stored at −80° C. until needed for analysis.

### DNA Extraction

Total microbial DNA was extracted from faeces of each specimen using the QIAAmp DNA Mini Kit (Qiagen) according to manufacturer's instructions. Each batch of specimens were extracted with negative buffer control (extraction control).

### Library Preparation

Whole-genome metagenome sequencing libraries were prepared. In brief, the DNA was sheared using a Covaris ultrasonicator. Sheared DNA was subjected to a sequence of enzymatic steps for repairing the ends and tailing with dA by ligation of indexed adapter sequences. These adapter-ligated fragments were then cleaned up using SPRI beads. Next, the clean fragments are indexed using limited cycle PCR to enrich the adapter-ligated molecules. Finally, the amplified products were purified and checked before sequencing.

### Metagenome Sequencing

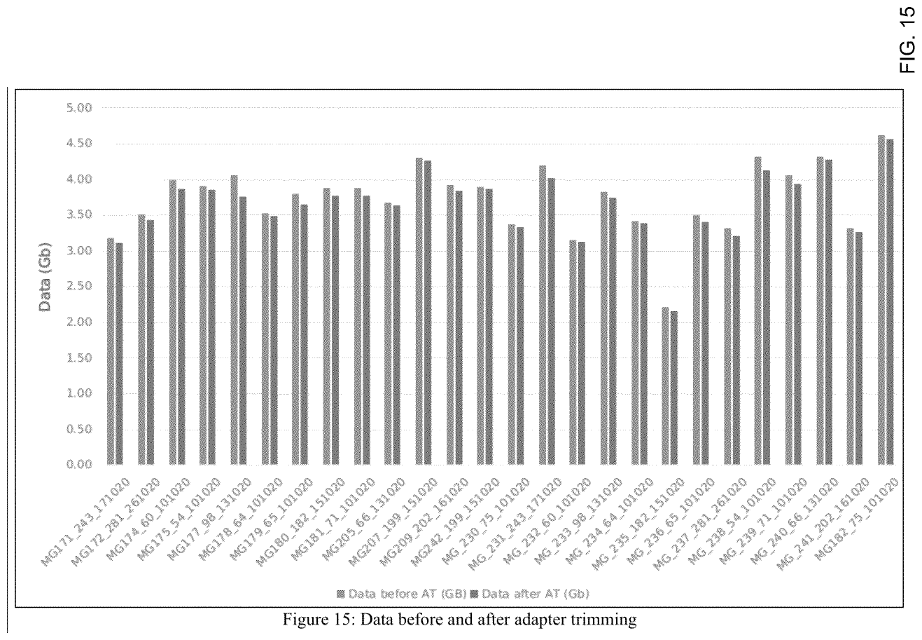

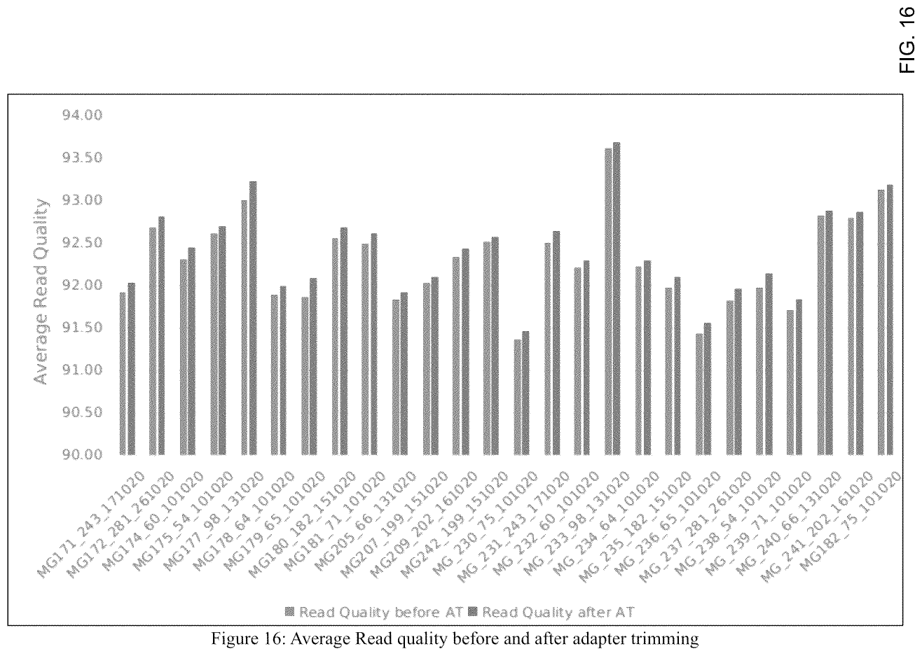

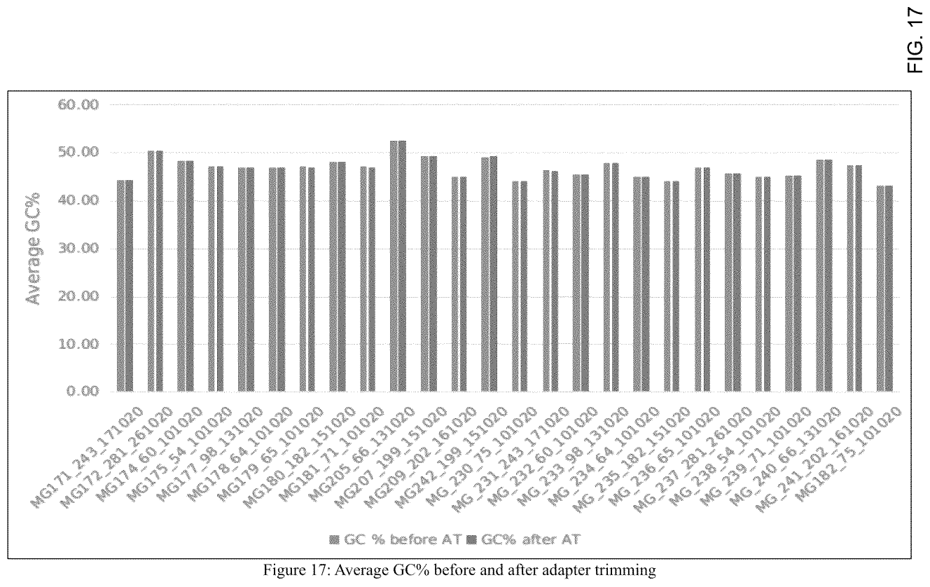

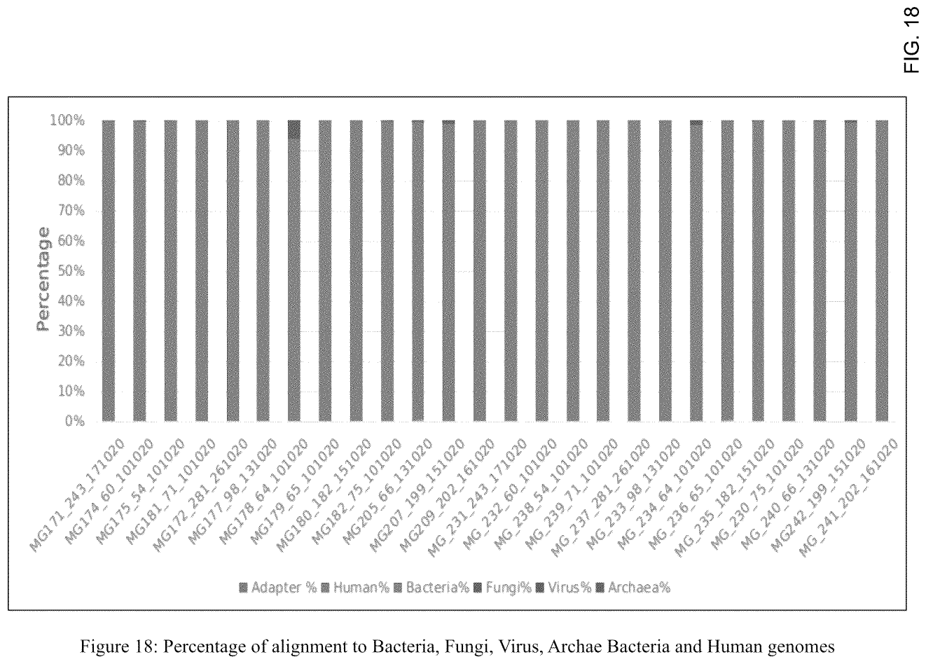

Prepared libraries were sequenced using Novaseq 6000 with a read length of 151 bp. The samples were taken for whole genome metagenome analysis. Initially, the reads were filtered for human DNA contamination (FIGS. 15-17). The alignment to the human genome was around 18.98%. The filtered reads were then aligned to bacterial, fungal, viral, and archea genomes (FIG. 18). The overall alignment to the bacterial genome was around 40%. However, the alignment to the viral, fungi, and archaea genomes was around 0.05-0.2%. De novo assembly was carried out using the preprocessed reads to obtain the scaffolds, which were then used for gene prediction. The abundances at the phylum, genus, and species level were evaluated.

### Microbiome Bioinformatics

The following bioinformatics pipeline was used to perform whole-genome-sequencing metagenomic analysis. The quality of the raw data was analysed and the adapters were trimmed. The low-processed reads were first aligned to human genome to remove unaligned reads that were then assembled using METASPADES de novo assembler for metagenomics. After assembly, the gene prediction was performed using PRODIGAL. The predicted genes were then searched against existing genes in the NCBI database using the DIAMOND MEGAN5 program. The occurrence of dominant microbial population was studied at various levels (phylum, class, order, family, and genus) based on the taxonomic abundance in the given samples. The dominance was calculated based on the amount of sequence obtained from samples, community composition, and the contig size distribution. Chimeric sequences were identified and filtered from the analysis.

### Statistical Analysis

Statistical data were analysed using Microsoft Excel statistics package analysis software. Paired t tests were also calculated using this package, and P values<0.05 were considered significant.

### Results

Eighteen patients who fulfilled all the selection criteria and none of the exclusion criteria were selected to begin the study. During enrolment, one participant in the treatment group (Group 2) dropped out before the study began. During the study, four subjects were lost to follow-up: two in Group 1 (one dropped out due to social problems in the family, and the other relocated to another city) and two in Group 2 (one dropped out due to social problems in the family, and the other relocated to another city). After excluding these four subjects, 13 subjects were included in the analysis.

The pre-processed reads were first aligned with the human genome (hg19) using BWA-MEM aligner to remove human genome contamination from the samples. The uncontaminated sequences were then taken for further alignment with known bacteria, fungi, virus, and archaea bacteria genomes using BWA MEM aligner. Around 7-12% of the reads mapped to the human genome, and the bacterial genome with 30-60% mapped reads.

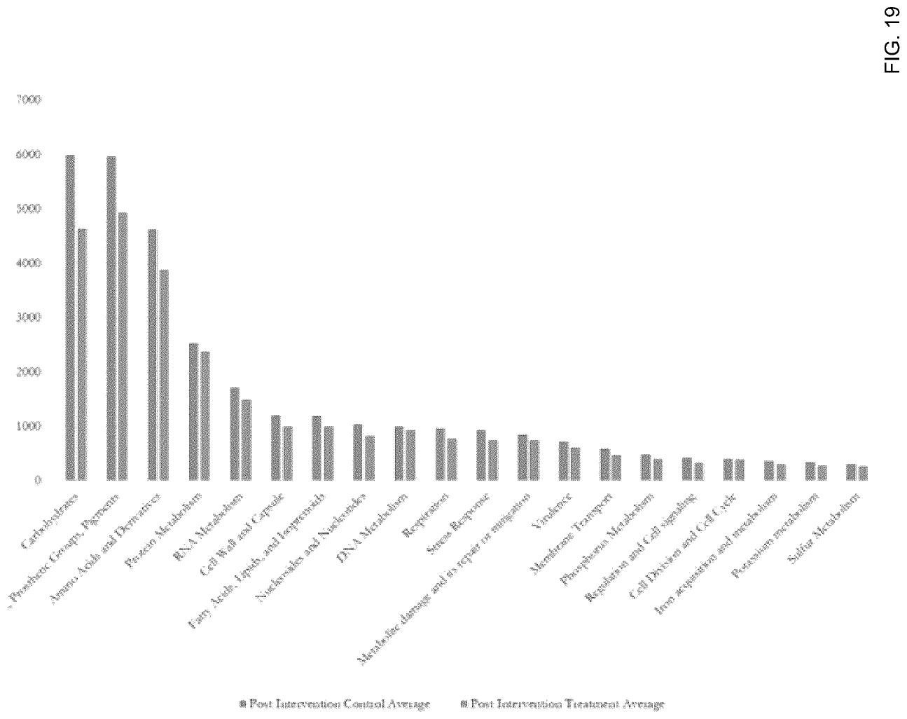

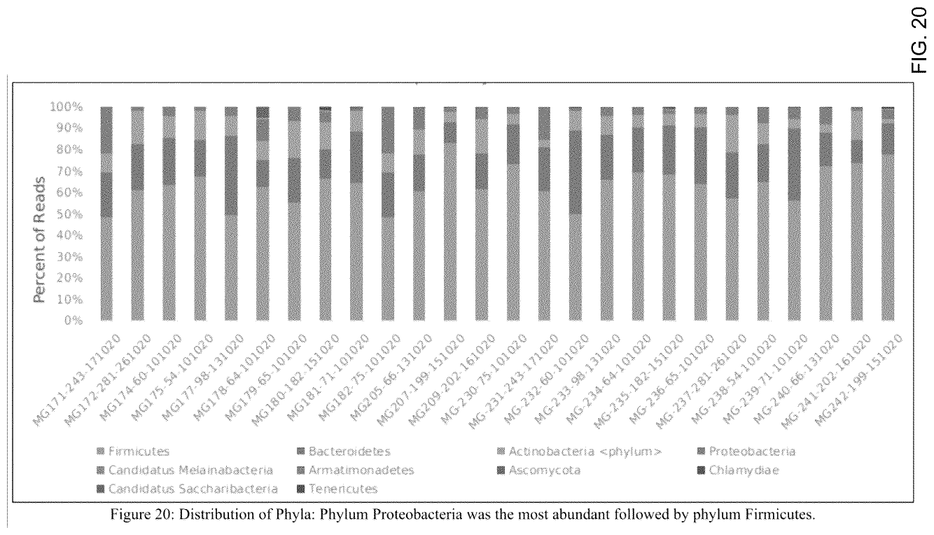

Regarding the SEED average, there was several fold decrease in all the gene annotations (metabolites and metabolic functions) in the AFO-202 Nichi Glucan treatment group including Carbohydrates, Fatty acids, lipids, virulence, metabolite damage and nitrogen metabolism (FIG. 19 and Table 5).

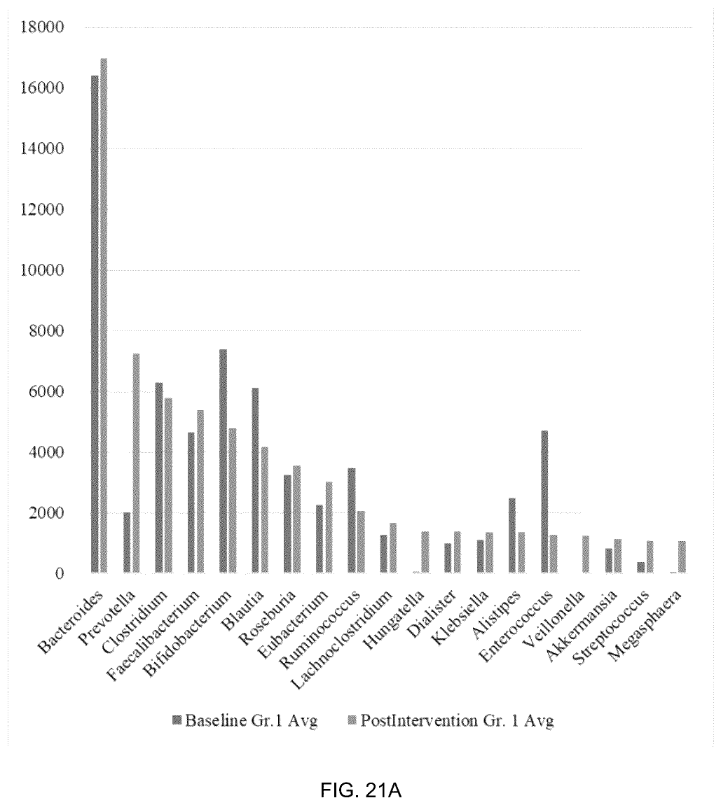

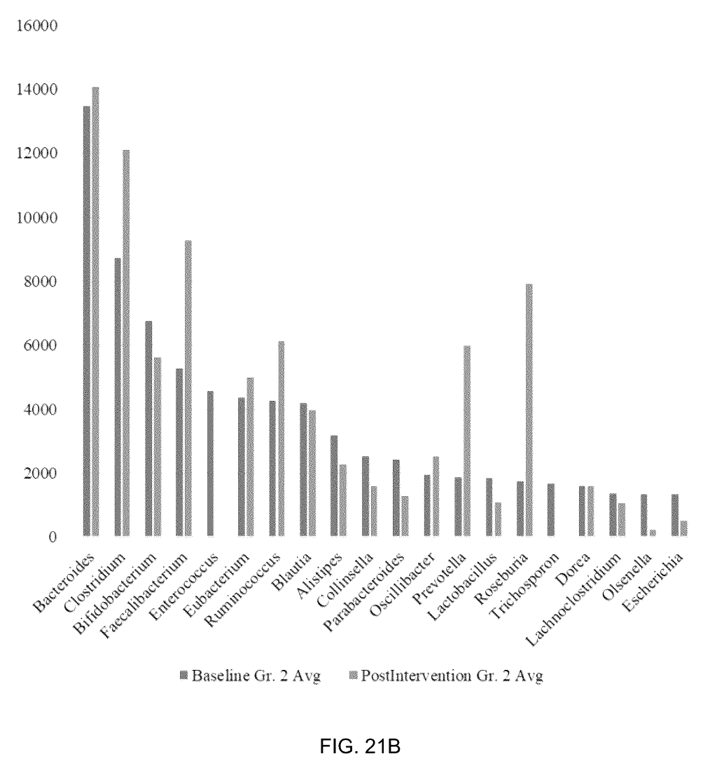

Bacterial kingdom was the most abundant organism type one. In both Group 1 (control) and Group 2 (Nichi Glucan), both before and after intervention, phylum Firmicutes was the most abundant followed by Bacteroidetes (FIG. 20). Although in Group 1 Proteobacteria was the next most abundant followed by Actinobacteria, this relationship was reversed in Group 2 (FIG. 21).

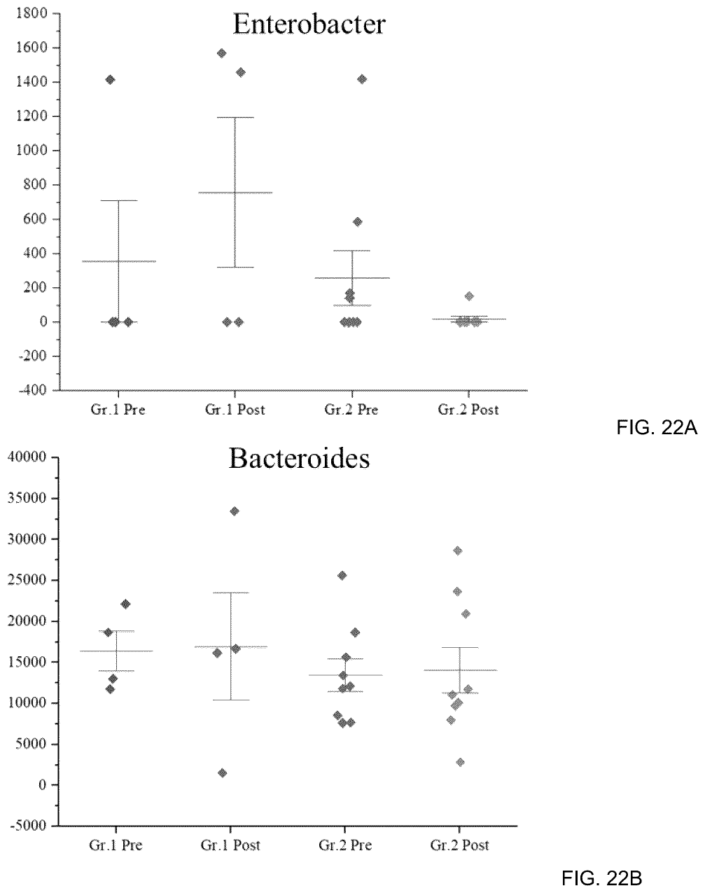

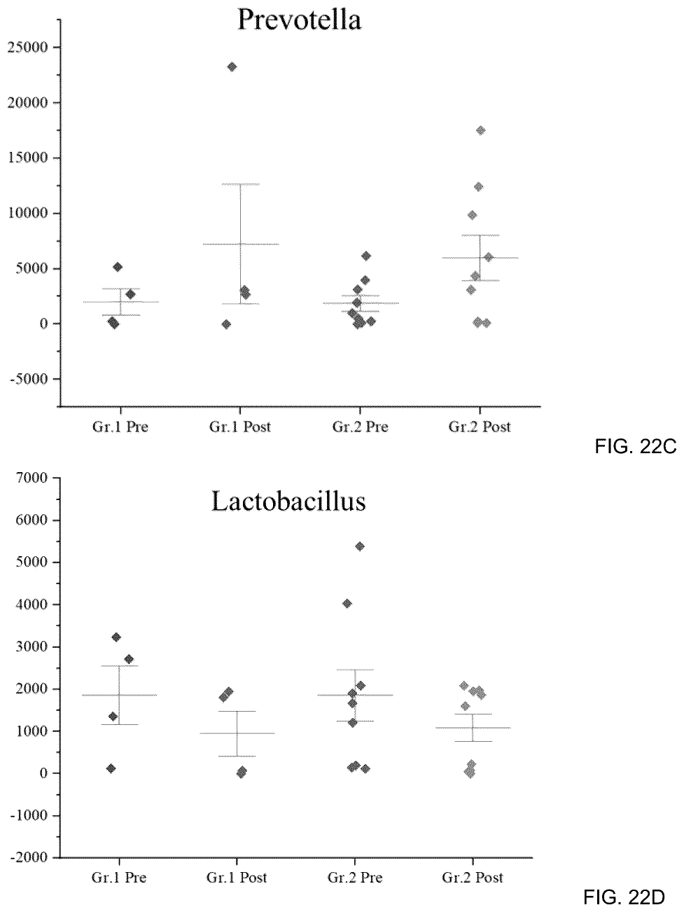

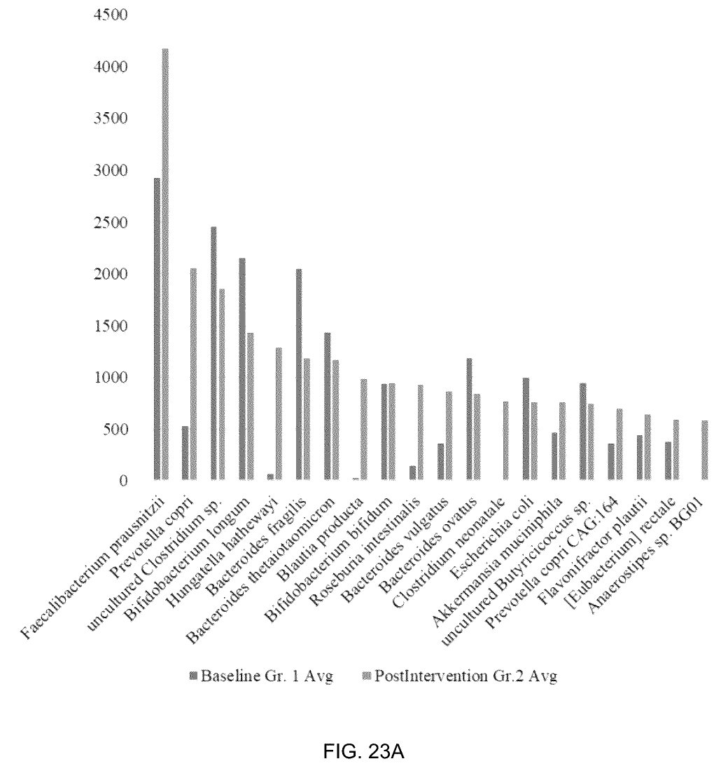

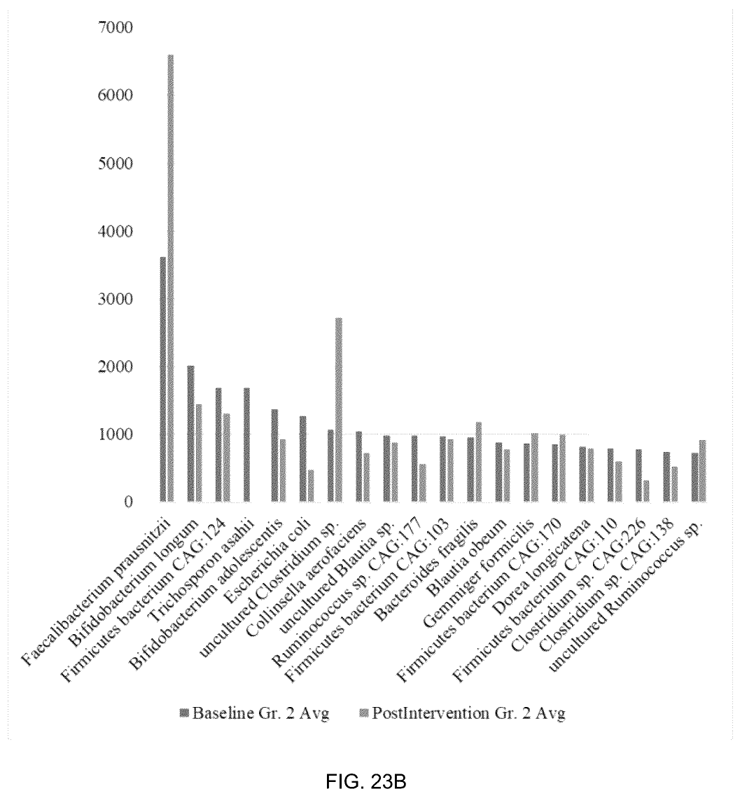

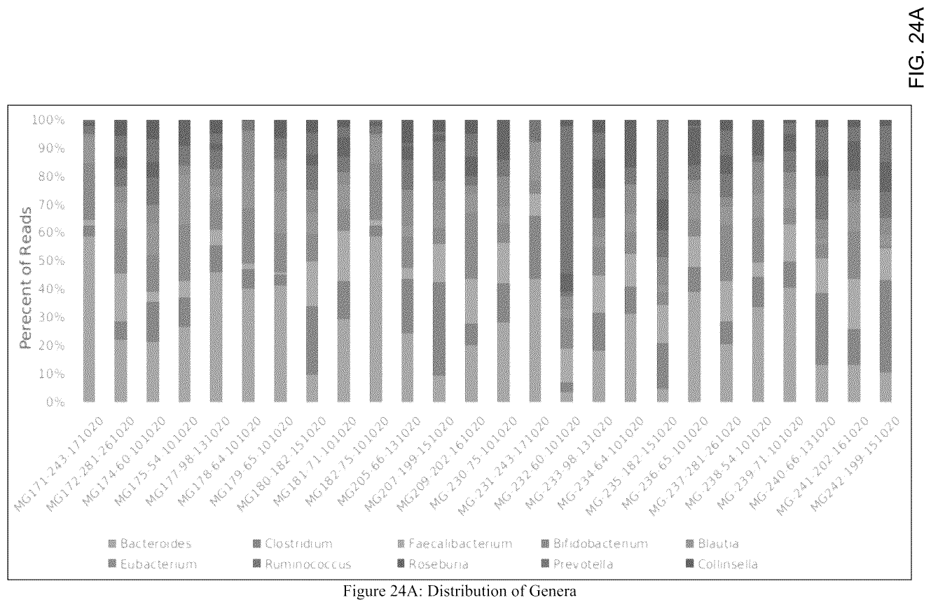

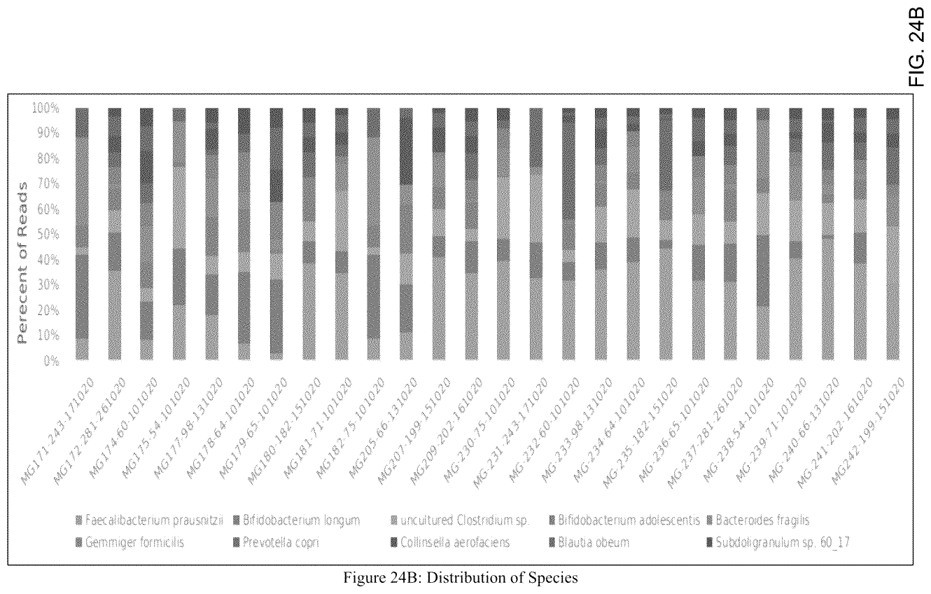

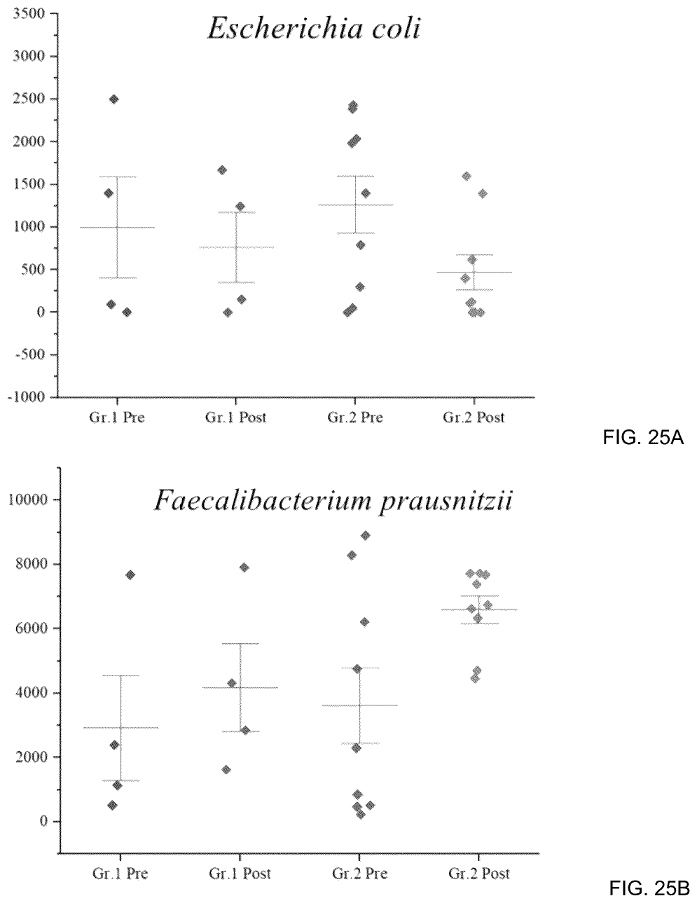

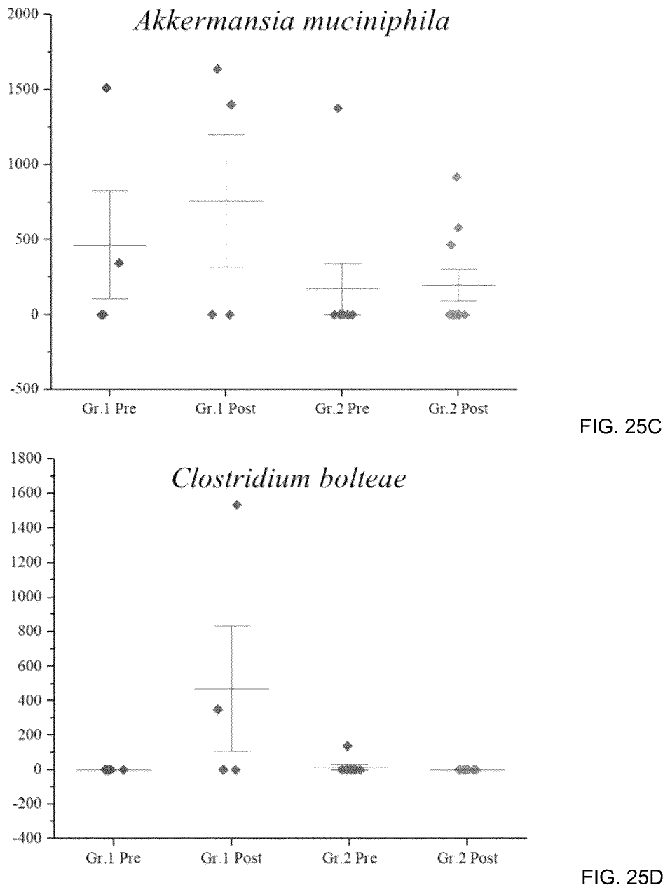

Among the genera of relevance, the abundance of Enterobacter was decreased to almost zero in Group 2 after intervention, while it increased from 0.36% to 0.85% in Group 1 (FIG. 22A). The abundance of Bacteroides increased from 16.84% to 19.09% (p-value=0.42) in Group 1, while it showed little significant difference (11.60% and 11.43%) (p value=0.46) (FIG. 22B) after intervention. The abundance of Prevotella increased in both Group 1 and Group 2 (FIG. 22C). The decrease in abundance of Lactobacillus was significant in Group 2 compared to Group 1 (FIG. 22D). Desulfovibrio decreased from 0.40% to 0.28% in Gr.2. Species abundance increased in Group 2 after intervention. Faecalibacterium prausnitzii, Bifidobacterium longum, and Firmicutes bacterium CAG:124 represented the most abundant species (FIG. 23 and FIG. 24). Escherichia coli decreased in both Group 1 and Group 2 but the difference was significant in Group 2 (p-value=0.02) (FIG. 25A). Faecalibacterium prausnitzii increased in both Group 1 and Group 2 but the difference was significant in Group 2 (p-value=0.041) (FIG. 25B). Akkermansia muciniphila CAG:154 (FIG. 25C) and Clostridium bolteae CAG:59 increased in the Group 1, whereas it decreased in Group 2 (FIG. 25D). Prevotella copri increased in both the groups. Blautia spp., Coprobacillus sp., and several Clostridium spp. decreased in both the groups.

The data of the genus and species level abundance in Gr. 1 and Gr. 2 are presented in Tables 5-9.

TABLE 5
Genus level: Mean and percentage abundance as baseline
and post-intervention in Gr. 1 (Control)

| | | | | Percentage | |
|---|---|---|---|---|---|
| | | Mean abundance | | of abundance | |
| | | | Post- | | Post- |
| S. | | | Inter- | | Inter- |
| No | Genus | Baseline | vention | Baseline | vention |
| 1 | Bacteroides | 16395 | 16956 | 16.84 | 19.09 |
| 2 | Prevotella | 2019 | 7252 | 2.07 | 8.16 |
| 3 | Clostridium | 6277 | 5792 | 6.45 | 6.52 |
| 4 | Faecalibacterium | 4646 | 5398 | 4.77 | 6.08 |
| 5 | Bifidobacterium | 7375 | 4809 | 7.58 | 5.41 |
| 6 | Blautia | 6123 | 4175 | 6.29 | 4.70 |
| 7 | Roseburia | 3257 | 3564 | 3.35 | 4.01 |
| 8 | Eubacterium | 2268 | 3016 | 2.33 | 3.40 |
| 9 | Ruminococcus | 3484 | 2066 | 3.58 | 2.33 |
| 10 | Lachnoclostridium | 1289 | 1682 | 1.32 | 1.89 |
| 11 | Hungatella | 66 | 1381 | 0.07 | 1.55 |
| 12 | Dialister | 983 | 1380 | 1.01 | 1.55 |
| 13 | Klebsiella | 1121 | 1357 | 1.15 | 1.53 |
| 14 | Alistipes | 3487 | 1352 | 2.55 | 1.52 |
| 15 | Enterococcus | 4721 | 1270 | 4.85 | 1.43 |
| 16 | Veillonella | 0 | 1244 | 0.00 | 1.40 |
| 17 | Akkermansia | 827 | 1126 | 0.85 | 1.27 |
| 18 | Streptococcus | 384 | 1093 | 0.39 | 1.23 |
| 19 | Megasphaera | 64 | 1084 | 0.07 | 1.22 |
| 20 | Anaerostipes | 393 | 1020 | 0.40 | 1.15 |
| 21 | Parabacteroides | 1686 | 971 | 1.73 | 1.09 |
| 22 | Lactobacillus | 1857 | 956 | 1.91 | 1.08 |
| 23 | Coprococcus | 897 | 915 | 0.92 | 1.03 |
| 24 | Dorea | 1352 | 862 | 1.39 | 0.97 |
| 25 | Butyricicoccus | 1339 | 815 | 1.38 | 0.94 |
| 26 | Flavonifractor | 669 | 823 | 0.69 | 0.93 |
| 27 | Escherichia | 1062 | 822 | 1.09 | 0.92 |
| 28 | Odoribacter | 1036 | 803 | 1.06 | 0.90 |
| 29 | Enterobacter | 355 | 757 | 0.36 | 0.85 |
| 30 | Subdoligranulum | 802 | 597 | 0.82 | 0.67 |
| 31 | Oscillibacter | 699 | 569 | 0.72 | 0.64 |
| 32 | Bilophila | 0 | 552 | 0.00 | 0.62 |
| 33 | Eggerthella | 829 | 548 | 0.85 | 0.62 |
| 34 | Collinsella | 1617 | 541 | 1.66 | 0.61 |
| 35 | Turicibacter | 0 | 415 | 0.00 | 0.47 |
| 36 | Mitsuokella | 0 | 409 | 0.00 | 0.46 |
| 37 | Catenibacterium | 540 | 403 | 0.55 | 0.45 |
| 38 | Fusicatenibacter | 434 | 392 | 0.45 | 0.44 |
| 39 | Romboutsia | 206 | 357 | 0.21 | 0.40 |
| 40 | Haemophilus | 88 | 346 | 0.09 | 0.39 |
| 41 | Gemmiger | 524 | 343 | 0.54 | 0.39 |
| 42 | Sutterella | 139 | 301 | 0.14 | 0.34 |
| 43 | Lachnospira | 0 | 294 | 0.00 | 0.33 |
| 44 | Mycoplasma | 292 | 251 | 0.30 | 0.28 |
| 45 | Holdemanella | 585 | 199 | 0.60 | 0.22 |
| 46 | Parasutterella | 0 | 197 | 0.00 | 0.22 |
| 47 | Weissella | 173 | 177 | 0.18 | 0.20 |
| 48 | Ruminiclostridium | 174 | 148 | 0.18 | 0.17 |
| 49 | Chlamydia | 133 | 147 | 0.14 | 0.16 |
| 50 | Dakarella | 97 | 147 | 0.10 | 0.16 |
| 51 | Lactococcus | 144 | 144 | 0.15 | 0.16 |
| 52 | Clostridioides | 107 | 126 | 0.11 | 0.14 |
| 53 | Terrisporobacter | 0 | 123 | 0.00 | 0.14 |
| 54 | Coprobacillus | 224 | 114 | 0.23 | 0.13 |
| 55 | Adlercreutzia | 405 | 101 | 0.42 | 0.11 |
| 56 | Butyrivibrio | 121 | 90 | 0.12 | 0.10 |
| 57 | Anaerotruncus | 135 | 81 | 0.14 | 0.09 |
| 58 | Bacillus | 262 | 69 | 0.27 | 0.08 |
| 59 | Agathobaculum | 54 | 67 | 0.05 | 0.08 |
| 60 | Shigella | 53 | 60 | 0.05 | 0.07 |
| 61 | Tyzzerella | 13 | 59 | 0.01 | 0.07 |
| 62 | Oribacterium | 145 | 52 | 0.15 | 0.06 |
| 63 | Intestinimonas | 91 | 50 | 0.09 | 0.06 |
| 64 | Citrobacter | 0 | 49 | 0.00 | 0.06 |
| 65 | Fusobacterium | 0 | 46 | 0.00 | 0.05 |
| 66 | Acholeplasma | 0 | 41 | 0.00 | 0.05 |
| 67 | Pseudoflavonifractor | 68 | 40 | 0.07 | 0.05 |
| 68 | Raoultella | 0 | 32 | 0.00 | 0.04 |
| 69 | Muribaculum | 0 | 31 | 0.00 | 0.03 |
| 70 | Acidiphilium | 27 | 29 | 0.03 | 0.03 |
| 71 | Paenibacillus | 110 | 28 | 0.11 | 0.03 |
| 72 | Acidaminococcus | 0 | 25 | 0.00 | 0.03 |
| 73 | Salmonella | 0 | 22 | 0.00 | 0.03 |
| 74 | Anaeromassilibacillus | 179 | 22 | 0.18 | 0.02 |
| 75 | Pantoea | 0 | 21 | 0.00 | 0.02 |
| 76 | Intestinibacter | 0 | 21 | 0.00 | 0.02 |
| 77 | Eisenbergiella | 85 | 20 | 0.09 | 0.02 |
| 78 | Prevotellamassilia | 0 | 20 | 0.00 | 0.02 |
| 79 | Anaerotignum | 0 | 17 | 0.00 | 0.02 |
| 80 | Selenomonas | 0 | 16 | 0.00 | 0.02 |
| 81 | Actinomyces | 0 | 16 | 0.00 | 0.02 |
| 82 | Paraprevotella | 26 | 10 | 0.03 | 0.01 |
| 83 | Porphyromonas | 0 | 9 | 0.00 | 0.01 |
| 84 | Barnesiella | 129 | 0 | 0.13 | 0.00 |
| 85 | Anaerococcus | 141 | 0 | 0.14 | 0.00 |
| 86 | Stenotrophomonas | 13 | 0 | 0.01 | 0.00 |
| 87 | Finegoldia | 82 | 0 | 0.08 | 0.00 |
| 88 | Pediococcus | 10 | 0 | 0.01 | 0.00 |
| 89 | Solobacterium | 27 | 0 | 0.03 | 0.00 |
| 90 | Peptoniphilus | 929 | 0 | 0.95 | 0.00 |
| 91 | Urinacoccus | 144 | 0 | 0.15 | 0.00 |
| 92 | Enterorhabdus | 46 | 0 | 0.05 | 0.00 |
| 93 | Slackia | 69 | 0 | 0.07 | 0.00 |
| 94 | Achromobacter | 59 | 0 | 0.06 | 0.00 |
| 95 | Lysinibacillus | 1130 | 0 | 1.16 | 0.00 |
| 96 | Olsenella | 192 | 0 | 0.20 | 0.00 |
| 97 | Comamonas | 131 | 0 | 0.13 | 0.00 |
| 98 | Ruthenibacterium | 63 | 0 | 0.06 | 0.00 |
| 99 | Viridibacillus | 40 | 0 | 0.04 | 0.00 |
| 100 | Rummeliibacillus | 603 | 0 | 0.62 | 0.00 |
| 101 | Gordonibacter | 735 | 0 | 0.75 | 0.00 |
| 102 | Drancourtella | 19 | 0 | 0.02 | 0.00 |
| 103 | Monoglobus | 24 | 0 | 0.02 | 0.00 |
| 104 | Lagierella | 39 | 0 | 0.04 | 0.00 |
| 105 | Peptococcus | 27 | 0 | 0.03 | 0.00 |
| 106 | Senegalimassilia | 122 | 0 | 0.13 | 0.00 |
| 107 | Libanicoccus | 25 | 0 | 0.03 | 0.00 |
| 108 | Urmitella | 24 | 0 | 0.02 | 0.00 |
| 109 | Paraclostridium | 19 | 0 | 0.02 | 0.00 |
| 110 | Acinetobacter | 1273 | 0 | 1.31 | 0.00 |
| 111 | Anaerocolumna | 9 | 0 | 0.01 | 0.00 |
| 112 | Marvinbryantia | 22 | 0 | 0.02 | 0.00 |
| 113 | Robinsoniella | 50 | 0 | 0.05 | 0.00 |
| 114 | Neglecta | 33 | 0 | 0.03 | 0.00 |
| 115 | Butyricimonas | 180 | 0 | 0.19 | 0.00 |
| 116 | unknown | 6947 | 5101 | 7.14 | 5.74 |

TABLE 6
Genus level: Mean and percentage abundance as baseline
and post-intervention in Gr. 2 (Nichi Glucan)

| | | Mean abundance | | Percentage of abundance | |
|---|---|---|---|---|---|
| S. | | | Post- | | Post- |
| No | Genus | Baseline | Intervention | Baseline | Intervention |
| 1 | Bacteroides | 13463 | 14088 | 11.60 | 11.43 |
| 2 | Clostridium | 8728 | 12105 | 7.52 | 9.83 |
| 3 | Bifidobacterium | 6771 | 5625 | 5.84 | 4.57 |
| 4 | Faecalibacterium | 5291 | 9278 | 4.56 | 7.53 |
| 5 | Enterococcus | 4583 | 40 | 3.95 | 0.03 |
| 6 | Eubacterium | 4380 | 5007 | 3.78 | 4.06 |
| 7 | Ruminococcus | 4265 | 6143 | 3.68 | 4.99 |
| 8 | Blautia | 4196 | 3973 | 3.62 | 3.22 |
| 9 | Alistipes | 3187 | 2268 | 2.75 | 1.84 |
| 10 | Collinsella | 2542 | 1590 | 2.19 | 1.29 |
| 11 | Parabacteroides | 2419 | 1307 | 2.08 | 1.06 |
| 12 | Oscillibacter | 1956 | 2542 | 1.69 | 2.06 |
| 13 | Prevotella | 1887 | 5979 | 1.63 | 4.85 |
| 14 | Lactobacillus | 1860 | 1093 | 1.60 | 0.89 |
| 15 | Roseburia | 1756 | 7932 | 1.51 | 6.44 |
| 16 | Trichosporon | 1683 | 0 | 1.45 | 0.00 |
| 17 | Dorea | 1592 | 1585 | 1.37 | 1.29 |
| 18 | Lachnoclostridium | 1360 | 1056 | 1.17 | 0.86 |
| 19 | Olsenella | 1354 | 246 | 1.17 | 0.20 |
| 20 | Escherichia | 1346 | 505 | 1.16 | 0.41 |
| 21 | Klebsiella | 1067 | 80 | 0.92 | 0.06 |
| 22 | Gemmiger | 970 | 1161 | 0.84 | 0.94 |
| 23 | Streptococcus | 925 | 521 | 0.80 | 0.42 |
| 24 | Coprococcus | 910 | 1228 | 0.78 | 1.00 |
| 25 | Subdoligranulum | 904 | 1112 | 0.78 | 0.90 |
| 26 | Eggerthella | 871 | 190 | 0.75 | 0.15 |
| 27 | Dialister | 765 | 1062 | 0.66 | 0.86 |
| 28 | Hungatella | 732 | 175 | 0.63 | 0.14 |
| 29 | Lysinibacillus | 709 | 0 | 0.61 | 0.00 |
| 30 | Acinetobacter | 677 | 0 | 0.58 | 0.00 |
| 31 | Pediococcus | 662 | 0 | 0.57 | 0.00 |
| 32 | Holdemanella | 649 | 546 | 0.56 | 0.44 |
| 33 | Butyricicoccus | 623 | 1257 | 0.54 | 1.02 |
| 34 | Catenibacterium | 620 | 470 | 0.53 | 0.38 |
| 35 | Cloacibacillus | 602 | 265 | 0.52 | 0.21 |
| 36 | Pichia | 588 | 0 | 0.51 | 0.00 |
| 37 | Odoribacter | 577 | 271 | 0.50 | 0.22 |
| 38 | Rummeliibacillus | 555 | 0 | 0.48 | 0.00 |
| 39 | Weissella | 555 | 24 | 0.48 | 0.02 |
| 40 | Flavonifractor | 545 | 729 | 0.47 | 0.59 |
| 41 | Senegalimassilia | 505 | 56 | 0.43 | 0.05 |
| 42 | Megasphaera | 464 | 1203 | 0.40 | 0.98 |
| 43 | Romboutsia | 463 | 130 | 0.40 | 0.11 |
| 44 | Desulfovibrio | 461 | 344 | 0.40 | 0.28 |
| 45 | Fusicatenibacter | 452 | 641 | 0.39 | 0.52 |
| 46 | Sutterella | 436 | 780 | 0.38 | 0.63 |
| 47 | Mycoplasma | 424 | 265 | 0.37 | 0.21 |
| 48 | Peptoniphilus | 413 | 0 | 0.36 | 0.00 |
| 49 | Ruminiclostridium | 411 | 387 | 0.35 | 0.31 |
| 50 | Anaerotruncus | 410 | 485 | 0.35 | 0.39 |
| 51 | Butyrivibrio | 404 | 315 | 0.35 | 0.26 |
| 52 | Methanobrevibacter | 395 | 368 | 0.34 | 0.30 |
| 53 | Megamonas | 392 | 501 | 0.34 | 0.41 |
| 54 | Bacillus | 326 | 150 | 0.28 | 0.12 |
| 55 | Butyricimonas | 319 | 39 | 0.28 | 0.03 |
| 56 | Anaerostipes | 318 | 485 | 0.27 | 0.39 |
| 57 | Phascolarctobacterium | 317 | 464 | 0.27 | 0.38 |
| 58 | Akkermansia | 305 | 498 | 0.26 | 0.40 |
| 59 | Gordonibacter | 304 | 0 | 0.26 | 0.00 |
| 60 | Coprobacillus | 295 | 438 | 0.25 | 0.36 |
| 61 | Coraliomargarita | 294 | 339 | 0.25 | 0.28 |
| 62 | Methanosphaera | 282 | 0 | 0.24 | 0.00 |
| 63 | Enterobacter | 257 | 17 | 0.22 | 0.01 |
| 64 | Pyramidobacter | 231 | 15 | 0.20 | 0.01 |
| 65 | Acidaminococcus | 230 | 269 | 0.20 | 0.22 |
| 66 | Paenibacillus | 229 | 184 | 0.20 | 0.15 |
| 67 | Acidiphilium | 219 | 555 | 0.19 | 0.45 |
| 68 | Adlercreutzia | 210 | 334 | 0.18 | 0.27 |
| 69 | Actinomyces | 191 | 4 | 0.16 | 0.00 |
| 70 | Succinatimonas | 190 | 383 | 0.16 | 0.31 |
| 71 | Libanicoccus | 188 | 119 | 0.16 | 0.10 |
| 72 | Angelakisella | 184 | 79 | 0.16 | 0.06 |
| 73 | Pseudoflavonifractor | 183 | 173 | 0.16 | 0.14 |
| 74 | Chlamydia | 176 | 130 | 0.15 | 0.11 |
| 75 | Anaeromassilibacillus | 175 | 135 | 0.15 | 0.11 |
| 76 | Intestinimonas | 157 | 174 | 0.14 | 0.14 |
| 77 | Duodenibacillus | 150 | 181 | 0.13 | 0.15 |
| 78 | Oribacterium | 145 | 103 | 0.33 | 0.08 |
| 79 | Clostridioides | 129 | 64 | 0.11 | 0.05 |
| 80 | Barnesiella | 118 | 84 | 0.10 | 0.07 |
| 81 | Slackia | 109 | 0 | 0.09 | 0.00 |
| 82 | Eisenbergiella | 95 | 112 | 0.08 | 0.09 |
| 83 | Acidovorax | 93 | 0 | 0.08 | 0.00 |
| 84 | Lactococcus | 92 | 0 | 0.08 | 0.00 |
| 85 | Paraprevotella | 91 | 16 | 0.08 | 0.01 |
| 86 | Shigella | 90 | 90 | 0.08 | 0.07 |
| 87 | Oxalobacter | 87 | 0 | 0.08 | 0.00 |
| 88 | Selenomonas | 84 | 231 | 0.07 | 0.19 |
| 89 | Turicibacter | 74 | 36 | 0.06 | 0.03 |
| 90 | Haemophilus | 74 | 196 | 0.06 | 0.16 |
| 91 | Pygmaiobacter | 69 | 0 | 0.06 | 0.00 |
| 92 | Urinacoccus | 64 | 0 | 0.06 | 0.00 |
| 93 | Bilophila | 63 | 613 | 0.05 | 0.50 |
| 94 | Allisonella | 62 | 54 | 0.05 | 0.04 |
| 95 | Ruthenibacterium | 51 | 69 | 0.04 | 0.06 |
| 96 | Raoultibacter | 50 | 0 | 0.04 | 0.00 |
| 97 | Comamonas | 44 | 0 | 0.04 | 0.00 |
| 98 | Neglecta | 42 | 37 | 0.04 | 0.03 |
| 99 | Atopabium | 40 | 11 | 0.03 | 0.01 |
| 100 | Christensenella | 38 | 41 | 0.03 | 0.03 |
| 101 | Massilimaliae | 37 | 32 | 0.03 | 0.03 |
| 102 | Finegoldia | 36 | 0 | 0.03 | 0.00 |
| 103 | Treponema | 36 | 48 | 0.03 | 0.04 |
| 104 | Robinsoniella | 36 | 0 | 0.03 | 0.00 |
| 105 | Enterorhabdus | 36 | 40 | 0.03 | 0.03 |
| 106 | Viridibacillus | 36 | 0 | 0.03 | 0.00 |
| 107 | Peptococcus | 35 | 0 | 0.03 | 0.00 |
| 108 | Fusobacterium | 34 | 30 | 0.03 | 0.02 |
| 109 | Terrisporobacter | 34 | 7 | 0.03 | 0.01 |
| 110 | Tyzzerella | 33 | 171 | 0.03 | 0.14 |
| 111 | Erysipelatoclostridium | 32 | 0 | 0.03 | 0.00 |
| 112 | Sporobacter | 32 | 36 | 0.03 | 0.03 |
| 113 | Mitsuokella | 31 | 432 | 0.03 | 0.35 |
| 114 | Cutaneotrichosporon | 31 | 0 | 0.03 | 0.00 |
| 115 | Marvinbryantia | 31 | 28 | 0.03 | 0.02 |
| 116 | Corallococcus | 30 | 143 | 0.03 | 0.12 |
| 117 | Anaerotignum | 28 | 18 | 0.02 | 0.01 |
| 118 | Prevotellamassilia | 24 | 158 | 0.02 | 0.13 |
| 119 | Anaerofilum | 24 | 35 | 0.02 | 0.03 |
| 120 | Synergistes | 22 | 0 | 0.02 | 0.00 |
| 121 | Alloprevotella | 22 | 22 | 0.02 | 0.02 |
| 122 | Isoptericola | 22 | 0 | 0.02 | 0.00 |
| 123 | Anaerococcus | 21 | 0 | 0.02 | 0.00 |
| 124 | Propionibacterium | 20 | 0 | 0.02 | 0.00 |
| 125 | Leuconostoc | 20 | 0 | 0.02 | 0.00 |
| 126 | Brachyspira | 19 | 95 | 0.02 | 0.08 |
| 127 | Lachnospira | 18 | 386 | 0.02 | 0.31 |
| 128 | Lagierella | 17 | 0 | 0.02 | 0.00 |
| 129 | Mogibacterium | 16 | 0 | 0.01 | 0.00 |
| 130 | Agathobaculum | 15 | 61 | 0.01 | 0.05 |
| 131 | Pseudobutyrivibrio | 15 | 35 | 0.01 | 0.03 |
| 132 | Hydrogenoanaerobacterium | 14 | 14 | 0.01 | 0.01 |
| 133 | Holdemania | 14 | 0 | 0.01 | 0.00 |
| 134 | Desulfotomaculum | 14 | 0 | 0.01 | 0.00 |
| 135 | Veillonella | 14 | 324 | 0.01 | 0.26 |
| 136 | Succinivibrio | 14 | 32 | 0.01 | 0.03 |
| 137 | Anaerocolumna | 14 | 14 | 0.01 | 0.01 |
| 138 | Mobilibacterium | 12 | 0 | 0.01 | 0.00 |
| 139 | Arabia | 12 | 0 | 0.01 | 0.00 |
| 140 | Enorma | 11 | 0 | 0.01 | 0.00 |
| 141 | Candida <Debaryomycetaceae> | 11 | 0 | 0.01 | 0.00 |
| 142 | Intestinibacter | 11 | 13 | 0.01 | 0.01 |
| 143 | Kurthia | 10 | 0 | 0.01 | 0.00 |
| 144 | Paeniclostridium | 9 | 0 | 0.01 | 0.00 |
| 145 | Paraclostridium | 8 | 0 | 0.01 | 0.00 |
| 146 | Listeria | 8 | 0 | 0.01 | 0.00 |
| 147 | Kwoniella | 8 | 0 | 0.01 | 0.00 |
| 148 | Cryptococcus | 7 | 0 | 0.01 | 0.00 |
| 149 | Fournierella | 0 | 23 | 0.00 | 0.02 |
| 150 | Sellimonas | 0 | 23 | 0.00 | 0.02 |
| 151 | Blastocystis | 0 | 34 | 0.00 | 0.03 |
| 152 | Acholeplasma | 0 | 126 | 0.00 | 0.10 |
| 153 | Azospirillum | 0 | 162 | 0.00 | 0.13 |
| 154 | Porphyromonas | 0 | 7 | 0.00 | 0.01 |
| 155 | Emergencia | 0 | 14 | 0.00 | 0.01 |
| 156 | Acetobacter | 0 | 13 | 0.00 | 0.01 |
| 157 | Dakarella | 0 | 64 | 0.00 | 0.05 |
| 158 | Parasutterella | 0 | 133 | 0.00 | 0.11 |
| 159 | Elusimicrobium | 0 | 128 | 0.00 | 0.10 |
| 160 | Drancourtella | 0 | 32 | 0.00 | 0.03 |
| 161 | Anaerovorax | 0 | 15 | 0.00 | 0.01 |
| 162 | Caldicoprobacter | 0 | 15 | 0.00 | 0.01 |
| 163 | unknown | 12707 | 16408 | 10.93 | 13.32 |

TABLE 7
Species level: Mean abundance at baseline
and post-intervention in Gr. 1 (Control)

| | | Mean abundance | |
|---|---|---|---|
| | | | Post- |
| S. | | Base- | Inter- |
| No | Species | line | vention |
| 1 | Prevotella copri | 530 | 2058 |
| 2 | uncultured Clostridium sp. | 2459 | 1860 |
| 3 | Bifidobacterium longum | 2150 | 1433 |
| 4 | Hungatella hathewayi | 65 | 1287 |
| 5 | Bacteroides fragilis | 2052 | 1189 |
| 6 | Bacteroides thetaiotaomicron | 1432 | 1172 |
| 7 | Blautia producta | 25 | 985 |
| 8 | Bifidobacterium bifidum | 941 | 946 |
| 9 | Roseboria intestinalis | 144 | 930 |
| 10 | Bacteroides vulgatus | 364 | 866 |
| 11 | Bacteroides ovatus | 1186 | 846 |
| 12 | Clostridium neonatale | 0 | 770 |
| 13 | Escherichia coli | 998 | 267 |
| 14 | Akkermansia muciniphila | 464 | 760 |
| 15 | uncultured Butyricicoccus Sp. | 948 | 751 |
| 16 | Prevotella copri CAG: 164 | 362 | 700 |
| 17 | Flavonifractor plautii | 445 | 640 |
| 18 | [Eubacterium] rectale | 380 | 594 |
| 19 | Anaerostipes sp. BG01 | 0 | 584 |
| 20 | Dialister sp. CAG: 357 | 0 | 540 |
| 21 | Lactobacillus ruminis | 628 | 536 |
| 22 | Bacteroides uniformis | 265 | 519 |
| 23 | Roseburia faecis | 625 | 507 |
| 24 | Prevotella sp. CAG: 386 | 61 | 481 |
| 25 | Eubacterium sp. CAG: 252 | 0 | 472 |
| 26 | [Clostridium] bolteae | 0 | 471 |
| 27 | [Eubacterium] eligens | 0 | 470 |
| 28 | Bifidobacterium adolescentis | 574 | 466 |
| 29 | Klebsiella pneumoniae | 397 | 461 |
| 30 | Prevotella sp. CAG: 1092 | 0 | 457 |
| 31 | Dialister succinatiphilus | 412 | 454 |
| 32 | Clostridium sp. AT4 | 12 | 450 |
| 33 | Prevotella sp. CAG: 604 | 154 | 445 |
| 34 | Firmicutes bacterium CAG: 124 | 806 | 439 |
| 35 | uncultured Blautia sp. | 1136 | 435 |
| 36 | Bacteroides xylanisolvens | 402 | 414 |
| 37 | Bilophila wadsworthia | 0 | 412 |
| 38 | Blautia obeum | 1024 | 410 |
| 39 | Prevotella sp. 885 | 48 | 406 |
| 40 | Fusicatenibacter saccharivorans | 432 | 391 |
| 41 | [Clostridium] clostridioforme | 76 | 379 |
| 42 | Megasphaera elsdenii | 0 | 376 |
| 43 | Bacteroides nordii | 56 | 369 |
| 44 | Faecalibacterium sp. CAG: 82 | 296 | 362 |
| 45 | [Ruminocossus] gnavus | 367 | 361 |
| 46 | Ruminococcus sp. CAG: 177 | 427 | 359 |
| 47 | Dorea longicatena | 402 | 358 |
| 48 | Mitsuokella multacida | 0 | 356 |
| 49 | Blautia wexlerae | 738 | 355 |
| 50 | Eubacterium sp. CAG: 251 | 0 | 351 |
| 51 | [Ruminococcus] torques | 790 | 342 |
| 52 | Prevotella multisaccharivorax | 72 | 334 |
| 53 | Odoribacter splanchnicus | 417 | 330 |
| 54 | Firmicutes bacterium CAG: 176 | 653 | 329 |
| 55 | Dialister sp. CAG: 486 | 143 | 328 |
| 56 | Gemmiger formicilis | 481 | 320 |
| 57 | Firmicutes bacterium CAG: 41 | 334 | 311 |
| 58 | Streptococcus thermophilus | 0 | 305 |
| 59 | Romboutsia timonensis | 171 | 299 |
| 60 | Subdoligranulum sp. 60_17 | 438 | 296 |
| 61 | Bacteroides oleiciplenus | 0 | 286 |
| 62 | Subdoligranulum variabile | 275 | 281 |
| 63 | Bacteroides plebeius | 0 | 280 |
| 64 | uncultured Faccalibacterium sp. | 168 | 280 |
| 65 | Roseburia hominis | 94 | 273 |
| 66 | Anaerostipes hadrus | 350 | 266 |
| 67 | Alistipes putredinis | 568 | 266 |
| 68 | butyrate-producing bacterium SS3/4 | 0 | 259 |
| 69 | Firmicutes bacterium CAG: 103 | 872 | 255 |
| 70 | Eggerthella lenta | 392 | 254 |
| 71 | Catenibacterium sp. CAG: 290 | 324 | 240 |
| 72 | Mycoplasma sp. CAG: 956 | 229 | 238 |
| 73 | Veillonella dispar | 0 | 238 |
| 74 | Oscillibacter sp. ER4 | 283 | 238 |
| 75 | Clostridium sp. CAG: 81 | 0 | 236 |
| 76 | Collinsella aerofaciens | 715 | 236 |
| 77 | Bacteroides timonenasis | 0 | 235 |
| 78 | Turicibacter sanguinis | 0 | 233 |
| 79 | Bifidobacterium sp. N5G01 | 530 | 230 |
| 80 | Bacteroides caccae | 548 | 224 |
| 81 | Clostridiales bacterium KLE1615 | 0 | 216 |
| 82 | uncultured Ruminococcus sp. | 760 | 213 |
| 83 | Bacteroides cellulosilyticus | 0 | 213 |
| 84 | Clostridium sp. CAG: 7 | 55 | 213 |
| 85 | Parabacteroides merdae | 40 | 211 |
| 86 | Clostridium sp. CAG: 389 | 291 | 208 |
| 87 | Blautia sp. CAG: 37 | 162 | 208 |
| 88 | Coprococcus eutactus | 158 | 200 |
| 89 | Veillonella atypica | 0 | 200 |
| 90 | Ruminococcus callidus | 32 | 200 |
| 91 | Holdemanella biformis | 585 | 199 |
| 92 | Lachnospiraceae bacterium TF01-11 | 0 | 199 |
| 93 | uncultured Lachnospira sp. | 0 | 190 |
| 94 | Bacteroides stercoris | 0 | 187 |
| 95 | Bifidobacterium catenulatum | 142 | 183 |
| 96 | [Eubacterium] hallii | 346 | 174 |
| 97 | Prevotella sp. CAG: 520 | 0 | 173 |
| 98 | Roseburia sp. CAG: 18 | 283 | 173 |
| 99 | Alistipes sp. HGB5 | 27 | 172 |
| 100 | Haemophilus parainfluenzae | 52 | 172 |
| 101 | Streptococcus salvarius | 71 | 169 |
| 102 | Coprococcus catus | 49 | 167 |
| 103 | Firmicutes bacterium CAG: 65 | 89 | 164 |
| 104 | Bacteroides plebeius CAG: 211 | 0 | 163 |
| 105 | Ruminococcus bromii | 158 | 163 |
| 106 | Megasphaera massiliensis | 12 | 161 |
| 107 | Enterobacter hormaechei | 47 | 158 |
| 108 | Roseburia intestinalis CAG: 13 | 0 | 153 |
| 109 | Catenibacterium mitsuokai | 198 | 149 |
| 110 | Dakarella massiliensis | 97 | 147 |
| 111 | Chlamydia trachomatis | 132 | 146 |
| 112 | Enterobacter cloacae | 70 | 145 |
| 113 | Bifidobacterium pseudocatenulatum | 251 | 145 |
| 114 | Clostridiales bacterium VE202-06 | 0 | 144 |
| 115 | Firmicutes bacterium CAG: 65_45_313 | 0 | 142 |
| 116 | [Clostridium] symbiosum | 0 | 140 |
| 117 | Veillenella parvula | 0 | 139 |
| 118 | Clostridiales bacterium 1_7_47FAA | 0 | 336 |
| 119 | Burkholderiales bacterium 1_1_47 | 0 | 135 |
| 120 | Roseburia sp. CAG: 18_43_25 | 158 | 134 |
| 121 | Bacteroides sp. HPS0048 | 64 | 132 |
| 122 | Weissella confusa | 124 | 132 |
| 123 | Clostridium sp. CAG: 122 | 0 | 131 |
| 124 | Firmicutes bacterium CAG: 424 | 146 | 131 |
| 125 | Prevotella stercorea | 0 | 127 |
| 126 | Ruminococcus sp. CAG: 254 | 445 | 127 |
| 127 | Bacteroides sp. 2_2_4 | 64 | 127 |
| 128 | Enterococcus asini | 0 | 126 |
| 129 | Blautia sp. KLE 1732 | 185 | 126 |
| 130 | Clostridioides difficile | 107 | 125 |
| 131 | Firmicutes bacterium CAG: 129_59_24 | 164 | 124 |
| 132 | [Eubacterium] siraeum | 50 | 124 |
| 133 | Clostridiales bacterium 42_27 | 184 | 120 |
| 134 | Bifidobacteriam kashiwanohense | 125 | 117 |
| 135 | Roseburia inulinivorans | 482 | 117 |
| 136 | Prevotella sp. CAG: 732 | 28 | 114 |
| 137 | Firmicutes bacterium CAG: 170 | 235 | 113 |
| 138 | Clostridium sp. CAG: 12237_41 | 0 | 113 |
| 139 | Clostridium bolteae CAG: 59 | 0 | 113 |
| 140 | Clostridium sp. ATCC BAA-442 | 67 | 113 |
| 141 | Laetobacillus ruminis CAG: 367 | 87 | 113 |
| 142 | Firmicutes bacterium CAG: 102 | 0 | 111 |
| 143 | Ruminococcus sp.5_1_39BFAA | 166 | 105 |
| 144 | Firmicutes bacterium CAG: 114 | 480 | 103 |
| 145 | Alistipes senegalensis | 628 | 102 |
| 146 | Alistipes finegoldii | 41 | 101 |
| 147 | Adlercreutzia equolifaciens | 405 | 101 |
| 148 | Sutterella sp. CAG: 351 | 132 | 100 |
| 149 | Parabacteroides distasonis | 67 | 100 |
| 150 | Parasutterella excrementihominis | 0 | 98 |
| 151 | uncultured Eubacterium sp. | 294 | 98 |
| 152 | Enterococcus avium | 292 | 97 |
| 153 | Bacteroides dorei | 235 | 97 |
| 154 | Bacteroides stercorirosoris | 0 | 97 |
| 155 | Collinsella sp. CAG: 66 | 156 | 96 |
| 156 | uncultured bacterium | 89 | 95 |
| 157 | Lactococcus garvieae | 0 | 95 |
| 158 | Prevotella sp. CAG: 474 | 21 | 95 |
| 159 | Terrisporobacter glycolicus | 0 | 94 |
| 160 | Osdillibacter sp. 57_20 | 66 | 94 |
| 161 | Veillonella sp. DORA_A_3_16_22 | 0 | 94 |
| 162 | Ruminococcus sp. CAG: 90 | 118 | 92 |
| 163 | Prevotella sp. CAG: 592 | 0 | 92 |
| 164 | Lachnospira pactinoschiza | 0 | 92 |
| 165 | Bacteroides intestinalis | 0 | 89 |
| 166 | Bacteroides caccae CAG: 21 | 231 | 88 |
| 167 | Bacteroides sp. 3_1_23 | 12 | 87 |
| 168 | Clostridiales bacterium 41_21_two_genomes | 21 | 87 |
| 169 | Faecalibacterium sp. CAG: 82-related_59_9 | 24 | 85 |
| 170 | Klebsiella michiganensis | 0 | 85 |
| 171 | Bifidobacterium sp. N4G05 | 217 | 83 |
| 172 | Proteobacteria bacterium CAG: 139 | 0 | 81 |
| 173 | Clostridiales bacterium 41_12_two_minus | 83 | 81 |
| 174 | Eubacterium sp. CAG: 248 | 0 | 80 |
| 175 | Dorea formicigenerans | 292 | 80 |
| 176 | Megasphaera sp. D1SK 18 | 0 | 79 |
| 177 | Coprobacillus sp. CAG: 235 | 197 | 79 |
| 178 | Prevotella stercorea CAG: 629 | 0 | 79 |
| 179 | Clostridiales bacterium 52_15 | 218 | 79 |
| 180 | Clostridiales bacterium 59_14 | 133 | 77 |
| 181 | Blautia sp. CAG: 37 | 163 | 77 |
| 182 | uncultured Bacteroides sp. | 0 | 76 |
| 183 | Alistipes finegoldii CAG: 68 | 0 | 75 |
| 184 | Dorea sp. CAG: 105 | 39 | 74 |
| 185 | Ruminococcus obeum CAG: 39 | 163 | 73 |
| 186 | Bacteroides cellulosilyticus CAG: 158 | 0 | 70 |
| 187 | Ruminococcus sp. CAG: 17 | 170 | 69 |
| 188 | Eubacterium sp. CAG: 38 | 0 | 69 |
| 189 | Clostridium sp. CAG: 91 | 0 | 69 |
| 190 | Klebsiella oxytoca | 0 | 68 |
| 191 | Bacteroides sp. 43_46 | 134 | 68 |
| 192 | Prevotella sp. P3-122 | 0 | 68 |
| 193 | Roseburia sp. CAG: 471 | 101 | 68 |
| 194 | Agathobaculum desmolans | 54 | 67 |
| 195 | Sutterella parvirubra | 0 | 66 |
| 196 | Eubacterium rectale CAG: 36 | 35 | 66 |
| 197 | Collinsella sp. 4_8_47FAA | 144 | 65 |
| 198 | Blantia sp. Marseille-P3201T | 68 | 65 |
| 199 | Bacteroide sp. 4_1_36 | 75 | 63 |
| 200 | Odoribacter sp. 43_10 | 61 | 62 |
| 201 | Parasutterella excrementihominis CAG: 233 | 0 | 60 |
| 202 | Bilophila sp. 4_1_30 | 0 | 60 |
| 203 | Eubacterium elugens CAG: 72 | 0 | 60 |
| 204 | Parabacteroides merdae CAG: 48 | 0 | 59 |
| 205 | Blautia sp. CAG: 37_48_57 | 87 | 59 |
| 206 | Bifidobacterium breve | 485 | 58 |
| 207 | Ruminococcus sp. CAG: 108 | 45 | 58 |
| 208 | Firmicutes bacterium CAG: 83 | 430 | 57 |
| 200 | Bacteroides sp. 41_26 | 0 | 57 |
| 210 | Anaerostipes caccae | 0 | 56 |
| 211 | uncultured Flavonifractor sp. | 65 | 56 |
| 212 | Enterococcus sp. HMSC05C03 | 31 | 56 |
| 213 | Butyricicoccus sp. BB10 | 347 | 56 |
| 214 | Alistipes putredinis CAG: 67 | 57 | 55 |
| 215 | Bacteroides sp. D20 | 18 | 54 |
| 216 | Lachnoclostridium sp. An196 | 34 | 54 |
| 217 | Dorea longicatena CAG: 42 | 29 | 53 |
| 218 | Klebsiella variicola | 0 | 53 |
| 219 | Akkermansia muciniphila CAG: 154 | 21 | 48 |
| 220 | Streptococcus pneumoniae | 36 | 48 |
| 221 | Firmicutes bacterium CAG: 129 | 73 | 48 |
| 222 | Eubacterium sp. 45_250 | 0 | 48 |
| 223 | Bacteroides sp. D22 | 51 | 47 |
| 224 | Eubacterium sp. CAG: 76 | 0 | 47 |
| 225 | Clostridiales bacterium VE202-28 | 0 | 47 |
| 226 | Oscillibacter sp. CAG: 241 | 69 | 46 |
| 227 | Blautia massiliensis | 69 | 46 |
| 228 | Blautia sp. SF-50 | 77 | 46 |
| 229 | Parabacteroides sp. D13 | 39 | 45 |
| 230 | Enterococcus faecium | 844 | 45 |
| 231 | Mitsuokella jalaludinii | 0 | 45 |
| 232 | Sutterella wadsworthensis | 0 | 44 |
| 233 | Clostridiales baterium 36_14 | 48 | 44 |
| 234 | Coprococcus sp. CAG: 131 | 66 | 44 |
| 235 | Lachnoclostridium sp. An14 | 0 | 43 |
| 236 | Lachnospiracaea bacterium 7_1_58FAA | 18 | 43 |
| 237 | Blautia sp. Marseille-P2398 | 86 | 43 |
| 238 | Eubacterium ballii CAG: 12 | 85 | 42 |
| 239 | Bacteroides sp. 1_1_30 | 49 | 42 |
| 240 | Clostridiales bacterium VE202-03 | 25 | 41 |
| 241 | Acholeplasma sp. CAG: 878 | 0 | 40 |
| 242 | Blautia sp. CAG: 52 | 175 | 40 |
| 243 | Veillonella sp. HPA0037 | 0 | 40 |
| 244 | Alistipes indistinctus | 91 | 39 |
| 245 | Ruminococcus sp. SR1/5 | 57 | 39 |
| 246 | Roseburia sp. CAG: 50 | 0 | 38 |
| 247 | Erwinia phage vB_EamM_V3 | 0 | 38 |
| 248 | Bacteroides sp. D2 | 0 | 38 |
| 249 | Eubacterium ramulus | 34 | 37 |
| 250 | Veillonella sp. oral taxon 158 | 0 | 37 |
| 251 | Clostridia bacterium UC5, 1-2H11 | 22 | 36 |
| 252 | Turicibacter sp. H121 | 0 | 36 |
| 253 | Fusobacterium sp. CAG: 815 | 0 | 36 |
| 254 | Alistipes sp. 58_9_plus | 0 | 35 |
| 255 | Prevotella sp. CAG: 873 | 0 | 34 |
| 256 | Clostridium sp. CAG: 448 | 0 | 34 |
| 257 | Clostridium sp. CAG: 492 | 0 | 34 |
| 258 | Firmicutes bacterium CAG: 822 | 0 | 34 |
| 259 | Veillonella sp. ACP1 | 0 | 34 |
| 260 | Bacteroides sp. 14(A) | 0 | 33 |
| 261 | Clostridiales bacterium NK3B98 | 0 | 33 |
| 262 | Ruminococcus sp. CAG: 108_related_41_35 | 25 | 33 |
| 263 | Bacteroides intestinalis CAG: 315 | 0 | 32 |
| 264 | Lactobacillus rogosae | 0 | 32 |
| 265 | Alistipes timonensis | 27 | 32 |
| 266 | Anaerotruncus sp. CAG: 390 | 26 | 31 |
| 267 | Prevotella sp. P4-65 | 0 | 31 |
| 268 | Blautia sp. An81 | 27 | 31 |
| 269 | Bacteroides fragilis CAG: 558 | 57 | 31 |
| 270 | Tyzzerella nexilis | 0 | 30 |
| 271 | Romboutsia ilealis | 0 | 30 |
| 272 | [Clostridium] citroniae | 0 | 29 |
| 273 | Prevotella sp. P5-108 | 0 | 29 |
| 274 | Prevotella sp. P4-76 | 0 | 29 |
| 275 | Acidiphilium sp. CAG: 727 | 27 | 29 |
| 276 | Muribaculum intestinale | 0 | 29 |
| 277 | Eubacterium sp. 41_20 | 27 | 29 |
| 278 | Shigella sonnei | 0 | 28 |
| 279 | Terrisporobacter othiniensis | 0 | 28 |
| 280 | Bifidobacterium adolescentis CAG: 119 | 0 | 28 |
| 281 | Veillonella sp. ICM51a | 0 | 28 |
| 282 | Prevotella sp. AGR2160 | 0 | 27 |
| 283 | Bacteroides sp. 3_1_13 | 11 | 27 |
| 284 | Prevotella sp. CAG: 279 | 320 | 27 |
| 285 | Intestinimonas butyriciproducens | 72 | 27 |
| 286 | Prevotella bryantii | 0 | 27 |
| 287 | Streptococcus parasanguinis | 12 | 27 |
| 288 | Clostridium sp. CAG: 43 | 0 | 27 |
| 289 | Klebsiella aerogenes | 503 | 27 |
| 290 | Ruminococcus sp. CAG: 330 | 37 | 26 |
| 291 | Clostridium sp. SS2/1 | 43 | 26 |
| 292 | uncultured Oscillibacter sp. | 53 | 26 |
| 293 | Prevotella sp. P5-64 | 0 | 26 |
| 294 | Firmicutes bacterium CAG: 341 | 281 | 25 |
| 295 | Oscillibacter sp. CAG: 241_62_21 | 32 | 25 |
| 296 | Megashaera sp. MJR8396C | 0 | 25 |
| 297 | Coprococcus sp. CAG: 131-related_45_246 | 0 | 25 |
| 298 | Ruminococcus sp. CAG: 9 | 61 | 25 |
| 299 | Prevotella sp. P5-60 | 0 | 25 |
| 300 | Bacteroides sp. CAG: 927 | 0 | 24 |
| 301 | Megashaera sp. BL7 | 0 | 24 |
| 302 | Bacteroidales bacterium 52_46 | 0 | 24 |
| 303 | Odoribacter splanchnicus CAG: 14 | 50 | 24 |
| 304 | Bacteroides sp. 43_108 | 0 | 24 |
| 305 | Citrobacter koseri | 0 | 24 |
| 306 | Clostridium sp. CAG: 221 | 0 | 23 |
| 307 | Veillonella tobetsuensis | 0 | 23 |
| 308 | Collinsella sp. TF06-26 | 144 | 23 |
| 309 | Prevotella sp. P2-180 | 0 | 23 |
| 310 | Bacteroides intestinalis CAG: 564 | 0 | 23 |
| 311 | Lachnoclostridium edouardi | 0 | 23 |
| 312 | Eggerthella sp. 1_3_56FAA | 25 | 22 |
| 313 | Klebsiella sp. MS 92-3 | 24 | 22 |
| 314 | Ruminococcus faecis | 39 | 22 |
| 315 | Bacteroides sp. 3_1_19 | 0 | 22 |
| 316 | Bacteroides stercoris CAG: 120 | 0 | 22 |
| 317 | Bacteroides sp. 1_1_14 | 170 | 21 |
| 318 | Parabacteroides johnsonii | 0 | 21 |
| 319 | Weissella cibaria | 0 | 21 |
| 320 | Prevotella sp. P4-67 | 0 | 21 |
| 321 | Intestinibacter bartlettii | 0 | 21 |
| 322 | Lachnospiraeceae bacterium 6_1_63FAA | 21 | 21 |
| 323 | Eubacterium siraeum CAG: 80 | 0 | 21 |
| 324 | Salmonella enterica | 0 | 21 |
| 325 | Prevotella sp. P4-51 | 0 | 21 |
| 326 | Clostridium botulinum | 0 | 20 |
| 327 | Clostridium nexile CAG: 348 | 0 | 20 |
| 328 | Prevotellamassilia timonensis | 0 | 20 |
| 329 | Prevotella sp. P5-125 | 0 | 20 |
| 330 | Clostridiales bacterium VE202-09 | 0 | 20 |
| 331 | Shigella flexneri | 0 | 20 |
| 332 | Eubacterium sp. CAG76_36_125 | 0 | 20 |
| 333 | Prevotella sp. P5-119 | 0 | 20 |
| 334 | Prevotella ruminicola | 0 | 19 |
| 335 | Prevotella lascolanii | 0 | 19 |
| 336 | Prevotella buccae | 0 | 19 |
| 337 | Clostridium butyricum | 0 | 19 |
| 338 | Blautia sp. An46 | 20 | 19 |
| 339 | Eggerthella sp. HGA1 | 19 | 19 |
| 340 | Coprococcus comes | 85 | 18 |
| 341 | Prevotella sp. CAG: 1185 | 0 | 18 |
| 342 | Lachnospiraeceae bacterium 5_1_63FAA | 35 | 18 |
| 343 | Bifidobacterium ruminantium | 263 | 18 |
| 344 | Bacteroides finegoldii | 0 | 18 |
| 345 | Bacteroides sp. 4_3_47FAA | 0 | 17 |
| 346 | Bacteroides sp. 3_1_40A | 0 | 17 |
| 347 | Bacteroides sp. CAG: 530 | 0 | 17 |
| 348 | Lachnospiraeceae bacterium CAG: 364 | 15 | 16 |
| 349 | Prevotella sp. CAG: 1124 | 0 | 16 |
| 350 | Bacteroides vulgatus CAG: 6 | 0 | 16 |
| 351 | Prevotella baroniae | 0 | 16 |
| 352 | Anaerotignum lactatifermentans | 0 | 16 |
| 353 | Blautia hansenii | 14 | 15 |
| 354 | Prevotella sp. CAG: 487 | 0 | 15 |
| 355 | Prevotella timonensis | 0 | 15 |
| 356 | Ruminococcus gnavus CAG: 126 | 17 | 15 |
| 357 | Megasphaera sp. NM10 | 0 | 15 |
| 358 | Prevotella buccalis | 0 | 14 |
| 359 | Prevotella sp. P5-92 | 0 | 14 |
| 360 | Streptococcus infantarius | 0 | 14 |
| 361 | Bacteroides sp. AR20 | 0 | 14 |
| 362 | Enterobacter sp. BIDMC 29 | 0 | 14 |
| 363 | Lachnospiraceae bacterium 2_1_58FAA | 13 | 14 |
| 364 | Bacteroides uniformis CAG: 3 | 18 | 14 |
| 365 | Prevotella sp. CAG: 5226 | 0 | 14 |
| 366 | Bacteroides sartorii | 0 | 14 |
| 367 | Blautia schinkii | 0 | 14 |
| 368 | [Clostridium] lavalense | 0 | 13 |
| 369 | Prevotella intermedia | 0 | 13 |
| 370 | Bacteroides mediterraneensis | 0 | 13 |
| 371 | Veillonella sp. 6_1_27 | 0 | 13 |
| 372 | Lactoccoccus lactis | 140 | 13 |
| 373 | Bacteroides faecis | 0 | 12 |
| 374 | Lachnospiraceae bacterium JC7 | 37 | 12 |
| 375 | Prevotella histicola | 0 | 11 |
| 376 | Sutterella sp. KLE1602 | 0 | 11 |
| 377 | Prevotella oralis | 0 | 11 |
| 378 | Bifidobacterium bifidum CAG: 234 | 0 | 10 |
| 379 | Prevotella sp. P4-119 | 0 | 10 |
| 380 | Prevotella paludivivens | 0 | 10 |
| 381 | Prevotella sp. tc2-28 | 0 | 10 |
| 382 | Prevotella sp. P5-126 | 0 | 10 |
| 383 | Prevotella sp. 109 | 0 | 10 |
| 384 | Prevotella brevis | 0 | 10 |
| 385 | Prevotella oris | 0 | 9 |
| 386 | Prevotella sp. DNF00663 | 0 | 9 |
| 387 | Prevotella oryzae | 0 | 9 |
| 388 | Prevotella sp. CAG: 255 | 0 | 9 |
| 389 | Prevotella sp. S7-1-8 | 0 | 9 |
| 390 | Prevotella dentalis | 0 | 9 |
| 391 | Prevotella sp. KH2C16 | 0 | 9 |
| 392 | Prevotella sp. CAG: 1058 | 0 | 9 |
| 393 | Prevotella maculosa | 0 | 9 |
| 394 | Prevotella bergensic | 0 | 9 |
| 395 | Ruminococcaceae bacterium D16 | 14 | 8 |
| 396 | Dorea formicigenerans CAG:28 | 31 | 0 |
| 397 | Rummeliibacillus stabekisii | 41 | 0 |
| 398 | Bifidobacterium pseudolongum | 30 | 0 |
| 399 | Clostridium sp. CAG: 138 | 415 | 0 |
| 400 | Blautia sp. CAG: 257 | 39 | 0 |
| 401 | Enterococcus sp. HMSC072H05 | 14 | 0 |
| 402 | Bacteroides dorei CAG: 222 | 22 | 0 |
| 403 | Bacteroides sp. CAG: 189 | 67 | 0 |
| 404 | [Desulfotomaculum] guttoideum | 17 | 0 |
| 405 | Tissierellia bacterium S5-A11 | 21 | 0 |
| 406 | Ruminococcus sp. CAG: 382 | 21 | 0 |
| 407 | Peptococcus niger | 27 | 0 |
| 408 | Oribacterium sp. C9 | 24 | 0 |
| 409 | Olsenella provencensis | 22 | 0 |
| 410 | Collinsella sp. CAG: 289 | 85 | 0 |
| 411 | Ruthenibacterium lactatiformans | 63 | 0 |
| 412 | Acinetobacter sp. NIPH 899 | 71 | 0 |
| 413 | Oribacterium sp. WCC10 | 33 | 0 |
| 414 | Clostridium sp. CAG: 609 | 259 | 0 |
| 415 | Clostridium sp. CAG: 571 | 33 | 0 |
| 416 | Butyricimonas virosa | 146 | 0 |
| 417 | Slackia piriformis | 52 | 0 |
| 418 | Firmicutes bacterium CAG: 110 | 278 | 0 |
| 419 | Achromobacter xylosoxidans | 12 | 0 |
| 420 | Lactobacillus mucosae | 321 | 0 |
| 421 | Clostridium sp. CAG: 433 | 54 | 0 |
| 422 | Clostridium sp. CAG: 226 | 155 | 0 |
| 423 | Solobacterium moorei | 27 | 0 |
| 424 | bacterium LF-3 | 22 | 0 |
| 425 | Anaerococcus prevotii | 17 | 0 |
| 426 | Olsenella sp. An188 | 24 | 0 |
| 427 | Clostridium minihomine | 16 | 0 |
| 428 | Acinetobacter sp. NIPH 2171 | 133 | 0 |
| 429 | Anaeromassilibacillus senegalensis | 19 | 0 |
| 430 | Dialister invisus | 60 | 0 |
| 431 | Firmicutes bacterium CAG: 646 | 12 | 0 |
| 432 | bacterium MS4 | 32 | 0 |
| 433 | Gordonibacter pamelaeae | 82 | 0 |
| 434 | Parabacteroides sp. HGS0025 | 18 | 0 |
| 435 | Anaeromassilibacillus sp. Marseille-P3371 | 12 | 0 |
| 436 | Peptoniphilus senegalensis | 37 | 0 |
| 437 | Clostridium sp. 7_2_43FAA | 17 | 0 |
| 438 | Peptoniphilus duerdenii | 16 | 0 |
| 439 | Comamonas testosteroni | 90 | 0 |
| 440 | Anaeromassilibacillus sp. An200 | 9 | 0 |
| 441 | Lysinibacillus sp. ZYM-1 | 44 | 0 |
| 442 | Mycoplasma sp. CAG: 472 | 53 | 0 |
| 443 | Clostridium sp. L2-50 | 37 | 0 |
| 444 | Collinsella sp. MS5 | 44 | 0 |
| 445 | Firmicutes bacterium CAG: 555 | 26 | 0 |
| 446 | Bacillus kochii | 12 | 0 |
| 447 | Faecalibacterium sp. CAG: 74_58_120 | 295 | 0 |
| 448 | Clostridium sp. CAG: 793 | 270 | 0 |
| 449 | Neglecta timonensis | 26 | 0 |
| 450 | [Clostridium] celerecrescens | 750 | 0 |
| 451 | Clostridium sp. ASB-410 | 20 | 0 |
| 452 | Enterococcus cassaliflavus | 251 | 0 |
| 453 | Eggerthella timonensis | 23 | 0 |
| 454 | Faecalibacterium sp. CAG: 74 | 439 | 0 |
| 455 | Clostridiales bacterium Marseille-P2846 | 187 | 0 |
| 456 | Anaerocococcus vaginalis | 30 | 0 |
| 457 | Anaeromassilibacillus sp. An250 | 39 | 0 |
| 458 | Acinetobacter sp. CIP 101934 | 13 | 0 |
| 459 | Firmicutes bacterium CAG: 24 | 145 | 0 |
| 460 | Firmicutes bacterium HGW-Firmicutes-16 | 22 | 0 |
| 461 | Barnesiella intestinihominis | 121 | 0 |
| 462 | Enterococcus gallinarum | 214 | 0 |
| 463 | Lysinibacillus sp. FJAT-14222 | 245 | 0 |
| 464 | Alistipes shahii | 40 | 0 |
| 465 | Peptoniphilus timonensis | 41 | 0 |
| 466 | Akkermansia sp. CAG: 344 | 85 | 0 |
| 467 | Lagierella massiliensis | 39 | 0 |
| 468 | Acinetobacter sp. LCT-H3 | 26 | 0 |
| 469 | Drancourtella massiliensis | 12 | 0 |
| 470 | Subdoligranulum sp. 4_3_54A2FAA | 88 | 0 |
| 471 | Peptoniphilus harei | 30 | 0 |
| 472 | Ruminococcus sp. CAG:9-related_41_34 | 11 | 0 |
| 473 | Ruminococcus lactaris | 22 | 0 |
| 474 | Firmicutes bacterium CAG: 24053_14 | 48 | 0 |
| 475 | Enterorhabdus caecimuris | 28 | 0 |
| 476 | Alistipes sp. Marseille-P2431 | 21 | 0 |
| 477 | Bacteroides thetaiotaomicron CAG: 40 | 42 | 0 |
| 478 | Ruminococcus flavefaciens | 30 | 0 |
| 479 | Blantis sp. Marseille-P3087 | 46 | 0 |
| 480 | Oribacterium sp. P6A1 | 25 | 0 |
| 481 | Clostridium sp. C105KSO15 | 124 | 0 |
| 482 | Achromobacter sp. Root170 | 21 | 0 |
| 483 | Clostridium sp. CAG: 264 | 101 | 0 |
| 484 | Lysinibacillus sphaerieus | 108 | 0 |
| 485 | Clostridium sp. CAG: 1024 | 39 | 0 |
| 486 | Urinacoccus sp. Marseille-P3926 | 136 | 0 |
| 487 | Enterococcus sp. FDAARGOS_375 | 30 | 0 |
| 488 | Clostridiales bacterium | 81 | 0 |
| 489 | Lysinibacillus boronitolerans | 27 | 0 |
| 490 | Collinsella bouchesdurhonensis | 32 | 0 |
| 491 | Rummeliibacillus pyenus | 562 | 0 |
| 492 | Erysipelotrichaceae bacterium NK3D112 | 33 | 0 |
| 493 | Collinsella sp. 60_9 | 26 | 0 |
| 494 | Alistipes obesi | 352 | 0 |
| 495 | Dialister invisus CAG: 218 | 290 | 0 |
| 496 | Eubacterium sp. CAG: 161 | 21 | 0 |
| 497 | Bacteroides sp. 3_1_33FAA | 22 | 0 |
| 498 | Firmicutes bacterium CAG: 110_56_8 | 45 | 0 |
| 499 | uncultured Coprococcus sp. | 83 | 0 |
| 500 | Paraclostridium bifermentans | 17 | 0 |
| 501 | Monoglobus pectinilyticus | 24 | 0 |
| 502 | Oribacterium sp. NK2B42 | 20 | 0 |
| 503 | Lactobacillus brevis | 32 | 0 |
| 504 | Senegalimassilia anaerobia | 122 | 0 |
| 505 | Acinetobacter baumannii | 228 | 0 |
| 506 | Clostidium sp. CAG: 567 | 236 | 0 |
| 507 | Coprococcus sp. ART55/1 | 22 | 0 |
| 508 | Peptoniphilus sp. HMSC075B08 | 105 | 0 |
| 509 | Enterococcus pallens | 17 | 0 |
| 510 | Finegoldia magna | 82 | 0 |
| 511 | Lysinibacillus sp. FJAT-14745 | 97 | 0 |
| 512 | Coprobacillus sp. 8_1_38FAA | 10 | 0 |
| 513 | Peptoniphilus sp. oral taxon 375 | 118 | 0 |
| 514 | uncultured crAssphage | 31 | 0 |
| 515 | Gordonibacter massiliensis | 20 | 0 |
| 516 | Olsenella sp. An290 | 19 | 0 |
| 517 | Peptoniphilus grossensis | 71 | 0 |
| 518 | Bacteroides sp. 9_1_42FAA | 31 | 0 |
| 519 | Firmicutes bacterium CAG: 176_63_11 | 30 | 0 |
| 520 | Enterococcus faecalis | 238 | 0 |
| 521 | Lactobacillus plantarum | 259 | 0 |
| 522 | Lysinibacillus fusiformis | 42 | 0 |
| 523 | Eubacterium sp. 38_16 | 21 | 0 |
| 524 | Peptoniphilus sp. HMSC062D09 | 42 | 0 |
| 525 | Alistipes sp. AG:53 | 39 | 0 |
| 526 | Bacteroidales bacterium 43_8 | 22 | 0 |
| 527 | Peptoniphilus phoceensis | 55 | 0 |
| 528 | Peptoniphilus sp. BV3AC2 | 12 | 0 |
| 529 | Clostridium sp. CAG: 302 | 267 | 0 |
| 530 | Gordonibacter urolithinfaciens | 567 | 0 |
| 531 | Acinetobacter sp. YZS-X1-1 | 115 | 0 |
| 532 | Lysinibacillus xylanilyticus | 242 | 0 |
| 533 | Acinetobacter schindleri | 91 | 0 |
| 534 | Bacteroides sp. CAG: 20 | 60 | 0 |
| 535 | Clostridium sp. CAG: 269 | 67 | 0 |
| 536 | Alistipes sp. cv1 | 20 | 0 |
| 537 | Roseburia inulinivorans CAG: 15 | 111 | 0 |
| 538 | Urmitella timonensis | 24 | 0 |
| 539 | Olsenella sp. An293 | 20 | 0 |
| 540 | Eisenbergiella tayi | 73 | 0 |
| 541 | Enterococcus saccharolyticus | 16 | 0 |
| 542 | Parabacteroides goldsteinii | 602 | 0 |
| 543 | Marvinbryantia formatexigens | 22 | 0 |
| 544 | Lysinibacillus macroides | 49 | 0 |
| 545 | Bacteroides salyersiae | 248 | 0 |
| 546 | Eubacterium sp. CAG: 146 | 52 | 0 |
| 547 | Peptoniphilus coxii | 318 | 0 |
| 548 | Libanicoccus massiliensis | 25 | 0 |
| 549 | Roseburia sp. CAG: 182 | 36 | 0 |
| 550 | Clostridium sp. CAG: 413 | 26 | 0 |
| 555 | Dorea sp. AGR2135 | 24 | 0 |
| 552 | Acinetobacter lwoffii | 12 | 0 |

TABLE 8
Species level: Mean abundance at baseline and
post-intervention in Gr. 2 (Nichi Glucan)

| | | Mean abundance | |
|---|---|---|---|
| | | | Post- |
| S. No | Species | Baseline | Intervention |
| 1 | Faecalibacterium prausnitzii | 3613 | 6596 |
| 2 | Bifidobacterium longum | 2012 | 1446 |
| 3 | Firmicutes bacterium CAG: 124 | 1684 | 1308 |
| 4 | Trichosporon asahii | 1683 | 0 |
| 5 | Bifidobacterium adolescentis | 1376 | 931 |
| 6 | Escherichia coli | 1264 | 471 |
| 7 | uncultured Clostridium sp. | 1062 | 2726 |
| 8 | Collinsella aerofaciens | 1048 | 729 |
| 9 | uncultured Blautia sp. | 981 | 879 |
| 10 | Ruminococcus sp. CAG: 177 | 977 | 565 |
| 11 | Firmicutes bacterium CAG: 103 | 972 | 925 |
| 12 | Bacteroides fragilis | 960 | 1186 |
| 13 | Blautia obeum | 877 | 777 |
| 14 | Gemmiger formicilis | 865 | 1022 |
| 15 | Firmicutes bacterium CAG: 170 | 847 | 989 |
| 16 | Dorea longicatena | 819 | 785 |
| 17 | Firmicutes bacterium CAG: 110 | 787 | 604 |
| 18 | Clostridium sp. CAG: 226 | 779 | 321 |
| 19 | Clostridium sp. CAG: 138 | 743 | 528 |
| 20 | uncultured Ruminococcus sp. | 726 | 913 |
| 21 | Bacteroides uniformis | 707 | 512 |
| 22 | Alistipes sp. CAG: 435 | 690 | 721 |
| 23 | Hungatella hathewayi | 681 | 171 |
| 24 | Holdemanella biformis | 649 | 546 |
| 25 | Oscillibacter sp. CAG: 241 | 634 | 419 |
| 26 | Firmicutes bacterium CAG: 176 | 617 | 945 |
| 27 | Firmicutes bacterium CAG: 83 | 612 | 400 |
| 28 | Subdoligranulum sp. 60_17 | 588 | 734 |
| 29 | Pichia kudriavzevii | 582 | 0 |
| 30 | Bacteroides thetaiotaomicron | 581 | 562 |
| 31 | Prevotella copri | 568 | 1458 |
| 32 | Eubacterium sp. CAG: 202 | 536 | 0 |
| 33 | Enterococcus faecium | 528 | 0 |
| 34 | Klebsiella pneumoniae | 524 | 44 |
| 35 | Rummeliibacillus pycnus | 516 | 0 |
| 36 | Senegalimassilia anaerobia | 505 | 56 |
| 37 | [Eubacterium] rectale | 499 | 1165 |
| 38 | Firmicutes bacterium CAG: 114 | 489 | 386 |
| 39 | Bifidobacterium bifidum | 476 | 418 |
| 40 | Bacteroides ovatus | 472 | 448 |
| 41 | Bacteroides vulgatus | 467 | 375 |
| 42 | Blautia wexlerae | 464 | 486 |
| 43 | Lactobacillus ruminis | 464 | 634 |
| 44 | [Ruminococcus] torques | 452 | 347 |
| 45 | Fusicatenibacter saccharivorans | 450 | 624 |
| 46 | [Eubacterium] hallii | 442 | 218 |
| 47 | Olsenella umbonata | 441 | 37 |
| 48 | Desulfovibrio piger | 434 | 303 |
| 49 | Dialister sp. CAG: 486 | 433 | 447 |
| 50 | uncultured Eubacterium sp. | 429 | 294 |
| 51 | Enterococcus avium | 425 | 0 |
| 52 | Faecalibacterium sp. CAG: 74 | 415 | 485 |
| 53 | Bacteroides caccae | 398 | 152 |
| 54 | Oscillibacter sp. CAG: 241_62_21 | 392 | 469 |
| 55 | Romboutsia timonensis | 388 | 112 |
| 56 | Eubacterium sp. CAG: 180 | 379 | 338 |
| 57 | Parabacteroides merdae | 377 | 101 |
| 58 | Pediococcus pentosaceus | 371 | 0 |
| 59 | Enterococcus faecalis | 362 | 0 |
| 60 | Clostridiales bacterium Marseille-P2846 | 358 | 355 |
| 61 | Clostridium sp. CAG: 221 | 355 | 326 |
| 62 | Butyricicoccus sp. BB10 | 334 | 177 |
| 63 | [Clostridium] celerecrescens | 333 | 0 |
| 64 | Lentisphaerae bacterium GWF2_44_16 | 332 | 307 |
| 65 | [Clostridium] bolteae | 331 | 43 |
| 66 | Clostridiales bacterium 42_27 | 329 | 463 |
| 67 | Prevotella sp. CAG: 279 | 326 | 754 |
| 68 | Bifidobacterium sp. N5G01 | 325 | 383 |
| 69 | Firmicutes bacterium CAG: 129 | 324 | 380 |
| 70 | Cloacibacillus porcorum | 322 | 248 |
| 71 | Clostridium sp. CAG: 1024 | 319 | 488 |
| 72 | Prevotella copri CAG: 164 | 318 | 771 |
| 73 | Catenibacterium mitsuokai | 312 | 274 |
| 74 | Eggerthella lenta | 309 | 60 |
| 75 | Mycoplasma sp. CAG: 956 | 306 | 117 |
| 76 | Oscillibacter sp. ER4 | 302 | 587 |
| 77 | Roseburia faecis | 301 | 662 |
| 78 | Coraliomargarita sp. CAG: 312 | 292 | 338 |
| 79 | Clostridiales bacterium 52_15 | 290 | 332 |
| 80 | Catenibacterium sp. CAG: 290 | 287 | 180 |
| 81 | Pediococcus acidilactici | 282 | 0 |
| 82 | Bacteroides dorei | 281 | 357 |
| 83 | Clostridiales bacterium 59_14 | 280 | 386 |
| 84 | Firmicutes bacterium CAG: 176_63_11 | 280 | 400 |
| 85 | Alistipes putredinis | 278 | 201 |
| 86 | Odoribacter splanchnicus | 277 | 129 |
| 87 | bacterium OL-1 | 275 | 15 |
| 88 | Parabacteroides sp. SN4 | 274 | 249 |
| 89 | [Eubacterium] eligens | 273 | 552 |
| 90 | Alistipes indistinctus | 272 | 13 |
| 91 | Alistipes obesi | 272 | 0 |
| 92 | Clostridium sp. CAG: 510 | 270 | 449 |
| 93 | Dialister sp. CAG: 357 | 270 | 360 |
| 94 | Faecalibacterium sp. CAG: 74_58_120 | 264 | 368 |
| 95 | Lactobacillus brevis | 262 | 0 |
| 96 | Bifidobacterium ruminantium | 261 | 257 |
| 97 | Anaerostipes hadrus | 260 | 422 |
| 98 | Enterococcus casseliflavus | 259 | 0 |
| 99 | Cloacibacillus sp. An23 | 257 | 0 |
| 100 | Bacteroides plebeius | 250 | 250 |
| 101 | Weissella confusa | 249 | 12 |
| 102 | Lactobacillus plantarum | 249 | 0 |
| 103 | Bacteroides sp. CAG: 545 | 246 | 379 |
| 104 | Clostridium sp. CAG: 452 | 245 | 120 |
| 105 | uncultured Faecalibacterium sp. | 243 | 437 |
| 106 | Collinsella sp. 4_8_47FAA | 239 | 166 |
| 107 | Firmicutes bacterium CAG: 555 | 237 | 281 |
| 108 | Ruminococcus sp. CAG: 724 | 236 | 367 |
| 109 | Roseburia inulinivorans | 233 | 1197 |
| 110 | Lentisphaerae bacterium GWF2_45_14 | 232 | 228 |
| 111 | Bacteroides xylanisolvens | 232 | 285 |
| 112 | uncultured Butyricicoccus sp. | 230 | 995 |
| 113 | Butyricimonas virosa | 230 | 0 |
| 114 | Bacteroides intestinalis | 230 | 135 |
| 115 | Collinsella sp. CAG: 166 | 228 | 187 |
| 116 | Ruminococcus sp. CAG: 488 | 225 | 211 |
| 117 | Coprococcus catus | 225 | 412 |
| 118 | Klebsiella aerogenes | 224 | 0 |
| 119 | Phascolarctobacterium sp. CAG: 207 | 220 | 21 |
| 120 | Acidiphilium sp. CAG: 727 | 219 | 555 |
| 121 | Parabacteroides gordonii | 217 | 0 |
| 122 | [Eubacterium] siraeum | 212 | 189 |
| 123 | Adlercreutzia equolifaciens | 210 | 334 |
| 124 | Butyrivibrio sp. CAG: 318 | 208 | 0 |
| 125 | Blautia sp. CAG: 37 | 207 | 203 |
| 126 | Clostridium sp. CAG: 448 | 203 | 192 |
| 127 | Bacteroides salyersiae | 203 | 193 |
| 128 | Coprobacillus sp. CAG: 235 | 197 | 145 |
| 129 | Clostridiales bacterium | 194 | 147 |
| 130 | Bifidobacterium pseudocatenulatum | 194 | 174 |
| 131 | Collinsella sp. TF06-26 | 189 | 155 |
| 132 | Alistipes sp. CAG: 53 | 189 | 180 |
| 133 | Clostridium sp. CAG: 571 | 189 | 14 |
| 134 | Sutterella sp. CAG: 397 | 188 | 193 |
| 135 | Libanicoccus massiliensis | 188 | 119 |
| 136 | Succinatimonas sp. CAG: 777 | 187 | 376 |
| 137 | Subdoligranulum variabile | 187 | 244 |
| 138 | Anaerotruncus sp. CAG: 390 | 185 | 275 |
| 139 | Bifidobacterium angulatum | 185 | 332 |
| 140 | Angelakisella massiliensis | 184 | 79 |
| 141 | Alistipes senegalensis | 183 | 57 |
| 142 | Megamonas funiformis | 182 | 175 |
| 143 | Ruminococcus obeum CAG: 39 | 180 | 137 |
| 144 | Acidaminococcus fermentans | 180 | 199 |
| 145 | Lactobacillus mucosae | 178 | 0 |
| 146 | Firmicutes bacterium CAG: 129_59_24 | 174 | 400 |
| 147 | Chlamydia trachomatis | 174 | 127 |
| 148 | Lentisphaerae bacterium GWF2_52_8 | 172 | 173 |
| 149 | Ruminococcus bromii | 172 | 301 |
| 150 | Eubacterium sp. CAG: 581 | 171 | 148 |
| 151 | Clostridium sp. CAG: 433 | 170 | 8 |
| 152 | Weissella cibaria | 170 | 0 |
| 153 | Methanobrevibacter smithii | 161 | 159 |
| 154 | Enterococcus gallinarum | 161 | 0 |
| 155 | Firmicutes bacterium CAG: 240 | 159 | 135 |
| 156 | Clostridium sp. CAG: 302 | 159 | 20 |
| 157 | Bacteroides plebeius CAG: 211 | 158 | 160 |
| 158 | Firmicutes bacterium CAG: 24053_14 | 158 | 158 |
| 159 | Eubacterium limosum | 157 | 0 |
| 160 | Clostridium sp. CAG: 349 | 157 | 143 |
| 161 | Clostridium sp. CAG: 43 | 156 | 162 |
| 162 | Parabacteroides sp. HGS0025 | 155 | 0 |
| 163 | Akkermansia muciniphila | 153 | 218 |
| 164 | Clostridium sp. CAG: 245 | 152 | 15 |
| 165 | Enterococcus hirae | 151 | 0 |
| 166 | Duodenibacillus massiliensis | 150 | 181 |
| 167 | Firmicutes bacterium CAG: 272 | 149 | 254 |
| 168 | Coprococcus eutactus | 148 | 100 |
| 169 | Verrucomicrobia bacterium CAG: 312_58_20 | 148 | 273 |
| 170 | Faecalibacterium sp. CAG: 82 | 147 | 398 |
| 171 | Lysinibacillus xylanilyticus | 147 | 0 |
| 172 | Firmicutes bacterium CAG: 460 | 144 | 166 |
| 173 | Peptoniphilus coxii | 141 | 0 |
| 174 | uncultured bacterium | 141 | 176 |
| 175 | Lysinibacillus sp. FJAT-14222 | 141 | 0 |
| 176 | Bifidobacterium breve | 139 | 132 |
| 177 | Clostridium sp. CAG: 245_30_32 | 138 | 9 |
| 178 | Clostridium sp. CAG: 451 | 136 | 30 |
| 179 | Sutterella wadsworthensis | 136 | 279 |
| 180 | Parabacteroides goldsteinii | 135 | 13 |
| 181 | Streptococcus mutans | 133 | 0 |
| 182 | Roseburia hominis | 132 | 630 |
| 183 | Streptococcus salivarius | 131 | 110 |
| 184 | Alistipes sp. CAG: 514 | 131 | 147 |
| 185 | Firmicutes bacterium CAG: 137 | 131 | 72 |
| 186 | Roseburia sp. CAG: 18 | 130 | 229 |
| 187 | Ruminococcus sp. 5_1_39BFAA | 129 | 152 |
| 188 | Clostridioides difficile | 127 | 64 |
| 189 | Flavonifractor plautii | 125 | 221 |
| 190 | Gordonibacter urolithinfaciens | 125 | 0 |
| 191 | Firmicutes bacterium CAG: 110_56_8 | 123 | 87 |
| 192 | Pyramidobacter sp. C12-8 | 122 | 0 |
| 193 | Ruminococcus sp. CAG: 17 | 122 | 151 |
| 194 | Bifidobacterium sp. N4G05 | 119 | 125 |
| 195 | Streptococcus thermophilus | 118 | 0 |
| 196 | Clostridium sp. CAG: 568 | 117 | 204 |
| 197 | Blautia sp. KLE 1732 | 115 | 51 |
| 198 | Parabacteroides merdae CAG: 48 | 114 | 30 |
| 199 | Dorea formicigenerans | 114 | 119 |
| 200 | Barnesiella intestinihominis | 112 | 77 |
| 201 | [Clostridium] clostridioforme | 111 | 124 |
| 202 | Intestinimonas butyriciproducens | 110 | 124 |
| 203 | Olsenella scatoligenes | 107 | 13 |
| 204 | Clostridium sp. CAG: 413 | 107 | 472 |
| 205 | uncultured Flavonifractor sp. | 105 | 138 |
| 206 | Clostridium sp. CAG: 343 | 104 | 0 |
| 207 | Alistipes shahii | 104 | 102 |
| 208 | Pyramidobacter piscolens | 103 | 0 |
| 209 | Firmicutes bacterium CAG: 238 | 103 | 75 |
| 210 | Collinsella sp. CAG: 289 | 102 | 0 |
| 211 | Bacteroides caccae CAG: 21 | 102 | 107 |
| 212 | Acinetobacter baumannii | 101 | 0 |
| 213 | Lentisphaerae bacterium GWF2_50_93 | 100 | 109 |
| 214 | Bacteroides sp. 2_2 4 | 97 | 83 |
| 215 | Eubacterium sp. CAG: 841 | 93 | 105 |
| 216 | Ruminococcus sp. CAG: 382 | 93 | 170 |
| 217 | Firmicutes bacterium CAG: 270 | 92 | 45 |
| 218 | Olsenella sp. kh2p3 | 90 | 13 |
| 219 | Phascolarctobacterium succinatutens | 90 | 420 |
| 220 | Lactobacillus fermentum | 90 | 0 |
| 221 | Mycoplasma sp. CAG: 877 | 89 | 41 |
| 222 | Bacteroides intestinalis CAG: 564 | 87 | 60 |
| 223 | Oxalobacter formigenes | 87 | 0 |
| 224 | Eisenbergiella tayi | 86 | 99 |
| 225 | Parabacteroides distasonis | 86 | 157 |
| 226 | Lentisphaerae bacterium GWF2_49_21 | 84 | 96 |
| 227 | Dorea longicatena CAG: 42 | 84 | 83 |
| 228 | Ruminococcus sp. CAG: 563 | 83 | 191 |
| 229 | Olsenella sp. KH3B4 | 83 | 16 |
| 230 | Lactobacillus ruminis CAG: 367 | 82 | 106 |
| 231 | Blautia sp. CAG: 37_48_57 | 82 | 140 |
| 232 | Alistipes sp. 56_sp_Nov_56_25 | 82 | 62 |
| 233 | Enterococcus thailandicus | 80 | 0 |
| 234 | Alistipes sp. HGB5 | 80 | 0 |
| 235 | Blautia sp. CAG: 237 | 78 | 207 |
| 236 | Bacteroides stercoris | 77 | 19 |
| 237 | Firmicutes bacterium HGW-Firmicutes-9 | 75 | 94 |
| 238 | Clostridium sp. CAG: 1193 | 75 | 125 |
| 239 | Roseburia sp. CAG: 18_43_25 | 75 | 173 |
| 240 | Firmicutes bacterium HGW-Firmicutes-16 | 74 | 73 |
| 241 | Oscillibacter sp. 57_20 | 74 | 403 |
| 242 | Olsenella provencensis | 74 | 7 |
| 243 | Olsenella sp. An188 | 74 | 0 |
| 244 | Anaerotruncus colihominis | 73 | 71 |
| 245 | Clostridium sp. CAG: 524 | 73 | 71 |
| 246 | Bacteroides coprocola CAG: 162 | 72 | 79 |
| 247 | Firmicutes bacterium CAG: 41 | 72 | 310 |
| 248 | Coprococcus comes | 71 | 125 |
| 249 | Ruminococcus sp. CAG: 90 | 71 | 9 |
| 250 | Clostridium sp. CAG: 492 | 70 | 0 |
| 251 | Pygmaiobacter massiliensis | 69 | 0 |
| 252 | Lysinibacillus sphaericus | 69 | 0 |
| 253 | Roseburia intestinalis | 69 | 1218 |
| 254 | Ruminococcus flavefaciens | 69 | 267 |
| 255 | uncultured Coprococcus sp. | 68 | 22 |
| 256 | Lactococcus lactis | 68 | 0 |
| 257 | uncultured Oscillibacter sp. | 66 | 87 |
| 258 | Gordonibacter pamelaeae | 65 | 0 |
| 259 | Bacteroides eggerthii | 64 | 0 |
| 260 | Bifidobacterium kashiwanohense | 64 | 82 |
| 261 | Clostridiales bacterium 41_12_two_minus | 63 | 195 |
| 262 | Collinsella sp. 60_9 | 63 | 17 |
| 263 | Allisonella histaminiformans | 62 | 54 |
| 264 | Megasphaera elsdenii | 61 | 262 |
| 265 | Lysinibacillus sp. FJAT-14745 | 61 | 0 |
| 266 | Ruminococcus sp. CAG: 9 | 60 | 59 |
| 267 | Urinacoccus sp. Marseille-P3926 | 60 | 0 |
| 268 | Bacteroides coprocola | 60 | 99 |
| 269 | Olsenella sp. An285 | 59 | 0 |
| 270 | Acinetobacter sp. NIPH 2171 | 59 | 0 |
| 271 | Clostridium sp. CAG: 7 | 59 | 396 |
| 272 | Bifidobacterium catenulatum | 58 | 75 |
| 273 | Olsenella sp. An290 | 58 | 0 |
| 274 | Eubacterium hallii CAG: 12 | 57 | 41 |
| 275 | Streptococcus pneumoniae | 57 | 8 |
| 276 | Collinsella sp. MSS | 56 | 0 |
| 277 | Eggerthella sp. CAG: 209 | 56 | 0 |
| 278 | Bacteroides sp. CAG: 20 | 56 | 37 |
| 279 | Clostridium sp. C105KSO15 | 55 | 0 |
| 280 | Bacteroides sp. CAG: 189 | 55 | 36 |
| 281 | Clostridium sp. CAG: 594 | 55 | 0 |
| 282 | Bacteroides sp. AR29 | 55 | 0 |
| 283 | Subdoligranulum sp. 4_3_54A2FAA | 54 | 65 |
| 284 | Ruminococcus bicirculans | 54 | 172 |
| 285 | Oscillibacter sp. 1-3 | 53 | 70 |
| 286 | Blautia sp. Marseille-P3087 | 53 | 39 |
| 287 | Turicibacter sanguinis | 52 | 26 |
| 288 | Peptoniphilus sp. oral taxon 375 | 52 | 0 |
| 289 | Olsenella sp. An293 | 52 | 0 |
| 290 | Bacteroides intestinalis CAG: 315 | 51 | 32 |
| 291 | Acinetobacter sp. YZS-X1-1 | 51 | 0 |
| 292 | Ruthenibacterium lactatiformans | 51 | 69 |
| 293 | Coprococcus eutactus CAG: 665 | 51 | 0 |
| 294 | Firmicutes bacterium CAG: 321 | 51 | 69 |
| 295 | Bacteroides sp. D20 | 50 | 32 |
| 296 | Clostridium sp. CAG: 264 | 50 | 38 |
| 297 | Blautia producta | 49 | 0 |
| 298 | Firmicutes bacterium CAG: 176_59_8 | 49 | 90 |
| 299 | Bacteroides sp. 1_1_14 | 48 | 18 |
| 300 | Erysipelotrichaceae bacterium 6_1_45 | 48 | 0 |
| 301 | Peptoniphilus sp. HMSC075B08 | 47 | 0 |
| 302 | Selenomonas bovis | 47 | 168 |
| 303 | Bacteroides sp. 4_1_36 | 45 | 27 |
| 304 | Slackia piriformis | 44 | 0 |
| 305 | Enterobacter cloacae | 44 | 0 |
| 306 | [Clostridium] citroniae | 43 | 0 |
| 307 | Ruminococcus sp. CAG: 254 | 43 | 486 |
| 308 | Alistipes finegoldii | 43 | 143 |
| 309 | Haemophilus parainfluenzae | 43 | 87 |
| 310 | Alistipes onderdonkii | 42 | 14 |
| 311 | Actinomyces sp. HPA0247 | 42 | 0 |
| 312 | Roseburia sp. CAG: 182 | 42 | 317 |
| 313 | Parabacteroides sp. merdae-related_45_40 | 41 | 10 |
| 314 | butyrate-producing bacterium SS3/4 | 41 | 248 |
| 315 | Collinsella vaginalis | 41 | 8 |
| 316 | Faecalibacterium sp. CAG: 82-related_59_9 | 41 | 99 |
| 317 | Acinetobacter schindleri | 40 | 0 |
| 318 | Bacteroides oleiciplenus | 40 | 55 |
| 319 | Megamonas rupellensis | 40 | 35 |
| 320 | Blautia sp. Marseille-P2398 | 40 | 44 |
| 321 | Clostridium sp. CAG: 81 | 40 | 238 |
| 322 | Comamonas kerstersii | 40 | 0 |
| 323 | Eggerthella sp. 1_3_56FAA | 39 | 0 |
| 324 | Enterobacter hormaechei | 39 | 0 |
| 325 | Rummeliibacillus stabekisii | 39 | 0 |
| 326 | Acinetobacter bereziniae | 39 | 0 |
| 327 | Lactobacillus pentosus | 39 | 0 |
| 328 | Eubacterium eligens CAG: 72 | 39 | 102 |
| 329 | Oscillibacter valericigenes | 38 | 58 |
| 330 | Bacteroides nordii | 38 | 0 |
| 331 | Clostridium sp. CAG: 127 | 37 | 628 |
| 332 | Eggerthella sp. HGA1 | 37 | 0 |
| 333 | [Ruminococcus] gnavus | 37 | 96 |
| 334 | Ruminococcus sp. CAG: 108 | 37 | 100 |
| 335 | Olsenella mediterranea | 37 | 0 |
| 336 | Oscillibacter sp. PC13 | 37 | 56 |
| 337 | Subdoligranulum sp. CAG: 314 | 37 | 36 |
| 338 | Oscillibacter ruminantium | 37 | 68 |
| 339 | Roseburia inulinivorans CAG: 15 | 37 | 161 |
| 340 | Finegoldia magna | 36 | 0 |
| 341 | Bacteroides sp. 3_1_23 | 36 | 42 |
| 342 | Peptococcus niger | 35 | 0 |
| 343 | Bacteroides sp. HPS0048 | 35 | 0 |
| 344 | Oribacterium sp. WCC10 | 35 | 15 |
| 345 | Firmicutes bacterium CAG: 552_39_19 | 34 | 68 |
| 346 | Paraprevotella clara CAG: 116 | 34 | 0 |
| 347 | Clostridiales bacterium 41_21_two_genomes | 34 | 338 |
| 348 | Prevotella sp. CAG: 604 | 34 | 247 |
| 349 | Intestinimonas massiliensis | 34 | 34 |
| 350 | Clostridium sp. L2-50 | 34 | 123 |
| 351 | Clostridium sp. CAG: 1000 | 34 | 0 |
| 352 | Firmicutes bacterium CAG: 24 | 33 | 9 |
| 353 | Enterococcus sp. FDAARGOS_375 | 33 | 0 |
| 354 | Prevotella sp. CAG: 891 | 33 | 34 |
| 355 | Parabacteroides sp. D13 | 33 | 60 |
| 356 | Enterococcus sp. HMSC05C03 | 33 | 0 |
| 357 | Eubacterium siraeum CAG: 80 | 33 | 39 |
| 358 | Megasphaera sp. BL7 | 33 | 74 |
| 359 | Bacteroides sp. D22 | 33 | 41 |
| 360 | Lysinibacillus macroides | 32 | 0 |
| 361 | Klebsiella sp. MS 92-3 | 32 | 0 |
| 362 | Sporobacter termitidis | 32 | 36 |
| 363 | Acinetobacter sp. NIPH 899 | 32 | 0 |
| 364 | Pseudoflavonifractor capillosus | 31 | 31 |
| 365 | Lysinibacillus fusiformis | 31 | 0 |
| 366 | Peptoniphilus grossensis | 31 | 0 |
| 367 | Firmicutes bacterium CAG: 102 | 31 | 206 |
| 368 | Ruminococcus albus | 31 | 33 |
| 369 | Cutaneotrichosporon oleaginosum | 31 | 0 |
| 370 | Firmicutes bacterium HGW-Firmicutes-21 | 31 | 0 |
| 371 | Marvinbryantia formatexigens | 31 | 28 |
| 372 | Collinsella bouchesdurhonensis | 31 | 0 |
| 373 | Acidovorax sp. 12322-1 | 31 | 0 |
| 374 | Firmicutes bacterium CAG: 321_26_22 | 31 | 51 |
| 375 | Corallococcus sp. CAG: 1435 | 30 | 143 |
| 376 | Bacteroides sp. 3_1_40A | 30 | 17 |
| 377 | Ruminococcaceae bacterium D5 | 30 | 31 |
| 378 | Bacteroidales bacterium 43_8 | 30 | 0 |
| 379 | Anaerotruncus rubiinfantis | 30 | 14 |
| 380 | Ruminococcus champanellensis | 30 | 57 |
| 381 | Clostridium sp. CAG: 349_48_7 | 30 | 25 |
| 382 | Eubacterium rectale CAG: 36 | 29 | 95 |
| 383 | Clostridium sp. CAG: 91 | 29 | 107 |
| 384 | Butyricimonas sp. An62 | 29 | 0 |
| 385 | Clostridiales bacterium GWF2_36_10 | 29 | 0 |
| 386 | Alistipes finegoldii CAG: 68 | 29 | 0 |
| 387 | Flavonifractor sp. An10 | 28 | 30 |
| 388 | Lactobacillus salivarius | 28 | 0 |
| 389 | Butyricimonas synergistica | 28 | 0 |
| 390 | Firmicutes bacterium CAG: 65 | 28 | 160 |
| 391 | Prevotella sp. CAG: 5226 | 28 | 188 |
| 392 | Romboutsia ilealis | 27 | 8 |
| 393 | Eubacterium sp. CAG: 146 | 27 | 27 |
| 394 | Clostridium sp. CAG: 609 | 27 | 19 |
| 395 | Blautia schinkii | 27 | 0 |
| 396 | Methanosphaera stadtmanae | 27 | 0 |
| 397 | Alistipes timonensis | 26 | 0 |
| 398 | Lysinibacillus sp. ZYM-1 | 26 | 0 |
| 399 | Lachnospiraceae bacterium JC7 | 26 | 17 |
| 400 | Bacteroides sp. CAG: 709 | 26 | 140 |
| 401 | Terrisporobacter glycolicus | 26 | 0 |
| 402 | Bacteroides sp. 3_1_19 | 25 | 22 |
| 403 | bacterium LF-3 | 25 | 20 |
| 404 | Bacteroides stercorirosoris | 24 | 31 |
| 405 | Peptoniphilus phoceensis | 24 | 0 |
| 406 | Oribacterium sp. P6A1 | 24 | 0 |
| 407 | Prevotellamassilia timonensis | 24 | 158 |
| 408 | Ruminococcus faecis | 24 | 10 |
| 409 | Anaerofilum sp. An201 | 24 | 35 |
| 410 | Dorea sp. 42_8 | 24 | 17 |
| 411 | Eubacterium sp. CAG: 251 | 24 | 290 |
| 412 | Clostridium sp. CAG: 389 | 23 | 45 |
| 413 | Blautia sp. SF-50 | 23 | 9 |
| 414 | Eubacterium sp. CAG: 76 | 23 | 76 |
| 415 | Clostridiales bacterium NK3B98 | 23 | 33 |
| 416 | Firmicutes bacterium ASF500 | 23 | 46 |
| 417 | Bacteroides bouchesdurhonensis | 23 | 0 |
| 418 | Eubacterium sp. 38_16 | 22 | 22 |
| 419 | Clostridium sp. HGF2 | 22 | 0 |
| 420 | Bacteroides sp. 9_1_42FAA | 22 | 16 |
| 421 | Mitsuokella jalaludinii | 22 | 280 |
| 422 | Paraprevotella clara | 22 | 0 |
| 423 | Blautia massiliensis | 22 | 7 |
| 424 | Coriobacteriaceae bacterium 68-1-3 | 22 | 0 |
| 425 | Bacteroides sp. 43_108 | 22 | 22 |
| 426 | Olsenella sp. An270 | 22 | 0 |
| 427 | Isoptericola variabilis | 22 | 0 |
| 428 | Clostridium sp. KNHs209 | 21 | 13 |
| 429 | Streptococcus parasanguinis | 21 | 61 |
| 430 | Gordonibacter massiliensis | 21 | 0 |
| 431 | Bilophila wadsworthia | 21 | 436 |
| 432 | uncultured Bacteroides sp. | 21 | 46 |
| 433 | Firmicutes bacterium CAG: 552 | 21 | 31 |
| 434 | Clostridium sp. SS2/1 | 21 | 60 |
| 435 | Parabacteroides johnsonii | 21 | 8 |
| 436 | Flavonifractor sp. An100 | 20 | 50 |
| 437 | Propionibacterium acidifaciens | 20 | 0 |
| 438 | Clostridium sp. CAG: 914 | 20 | 29 |
| 439 | Clostridium sp. 26_22 | 20 | 0 |
| 440 | Bacteroides cellulosilyticus | 20 | 82 |
| 441 | Ruminococcaceae bacterium D16 | 20 | 163 |
| 442 | Butyricicoccus pullicaecorum | 20 | 30 |
| 443 | Actinomyces sp. ICM47 | 20 | 0 |
| 444 | Olsenella sp. Marseille-P2300 | 20 | 0 |
| 445 | Collinsella tanakaei | 20 | 15 |
| 446 | Gemmiger sp. An120 | 19 | 39 |
| 447 | Pseudoflavonifractor sp. An184 | 19 | 35 |
| 448 | Peptoniphilus sp. HMSC062D09 | 19 | 0 |
| 449 | Bacteroides sp. 3_1_13 | 19 | 36 |
| 450 | Flavonifractor sp. An306 | 19 | 32 |
| 451 | Firmicutes bacterium CAG: 145 | 19 | 15 |
| 452 | Peptoniphilus timonensis | 18 | 0 |
| 453 | Eubacterium ventriosum | 18 | 70 |
| 454 | Clostridiales bacterium 43-6 | 18 | 0 |
| 455 | Cloacibacillus evryensis | 18 | 0 |
| 456 | Akkermansia muciniphila CAG: 154 | 18 | 13 |
| 457 | Oribacterium sp. C9 | 18 | 0 |
| 458 | Olsenella uli | 18 | 11 |
| 459 | Prevotella sp. CAG: 1092 | 17 | 299 |
| 460 | Slackia heliotrinireducens | 17 | 0 |
| 461 | Lagierella massiliensis | 17 | 0 |
| 462 | Firmicutes bacterium CAG: 194 | 17 | 14 |
| 463 | Lachnospiraceae bacterium 28-4 | 17 | 0 |
| 464 | Oribacterium sp. NK2B42 | 17 | 0 |
| 465 | Bacteroides eggerthii CAG: 109 | 17 | 0 |
| 466 | Eggerthella timonensis | 17 | 0 |
| 467 | Clostridium sp. CAG: 762 | 17 | 79 |
| 468 | Brachyspira sp. CAG: 484 | 17 | 91 |
| 469 | methanogenic archaeon mixed culture ISO4-G1 | 17 | 38 |
| 470 | Eggerthella sp. 51_9 | 17 | 0 |
| 471 | Ruminococcus sp. CAG: 379 | 17 | 27 |
| 472 | Bacteroides timonensis | 17 | 98 |
| 473 | Oscillibacter sp. CAG: 155 | 17 | 36 |
| 474 | Peptoniphilus senegalensis | 17 | 0 |
| 475 | Lachnospira pectinoschiza | 16 | 117 |
| 476 | Firmicutes bacterium CAG: 95 | 16 | 197 |
| 477 | Megamonas sp. Calf98-2 | 16 | 10 |
| 478 | Massilimaliae massiliensis | 16 | 14 |
| 479 | Anaeromassilibacillus sp. An200 | 16 | 17 |
| 480 | Odoribacter sp. 43_10 | 16 | 0 |
| 481 | Bacteroides mediterraneensis | 16 | 11 |
| 482 | Bacteroides dorei CAG: 222 | 16 | 7 |
| 483 | Bacteroides sp. 4_3_47FAA | 16 | 13 |
| 484 | Firmicutes bacterium CAG: 534 | 16 | 314 |
| 485 | Ruminococcaceae bacterium FB2012 | 16 | 0 |
| 486 | Clostridium disporicum | 16 | 0 |
| 487 | Neglecta timonensis | 16 | 13 |
| 488 | Bacteroides uniformis CAG: 3 | 15 | 9 |
| 489 | Clostridium bolteae CAG: 59 | 15 | 0 |
| 490 | Agathobaculum desmolans | 15 | 61 |
| 491 | Bacteroides sartorii | 15 | 5 |
| 492 | Bacteroides sp. D2 | 15 | 63 |
| 493 | Lachnoclostridium sp. An196 | 15 | 151 |
| 494 | Acidaminococcus massiliensis | 15 | 0 |
| 495 | Raoultibacter massiliensis | 15 | 0 |
| 496 | Clostridium sp. SN20 | 15 | 0 |
| 497 | Eggerthella sp. YY7918 | 15 | 0 |
| 498 | Bacteroides sp. 43_46 | 15 | 0 |
| 499 | Lachnospiraceae bacterium 7_1_58FAA | 15 | 25 |
| 500 | Eggerthellaceae bacterium AT8 | 15 | 0 |
| 501 | Alistipes sp. AL-1 | 15 | 10 |
| 502 | Ruminococcus sp. SR1/5 | 15 | 0 |
| 503 | Bacteroides fragilis CAG: 558 | 15 | 0 |
| 504 | [Clostridium] thermosuccinogenes | 14 | 0 |
| 505 | Hydrogenoanaerobacterium saccharovorans | 14 | 14 |
| 506 | Alistipes sp. 58_9_plus | 14 | 0 |
| 507 | Eubacterium sp. CAG: 86 | 14 | 92 |
| 508 | Faecalibacterium sp. CAG: 1138 | 14 | 29 |
| 509 | Actinomyces oris | 14 | 0 |
| 510 | Dorea sp. CAG: 105 | 14 | 0 |
| 511 | Eubacteriaceae bacterium CHKC1005 | 14 | 17 |
| 512 | Succinivibrio dextrinosolvens | 14 | 32 |
| 513 | Lactococcus garvieae | 14 | 0 |
| 514 | Bacteroides sp. 1_1_30 | 14 | 33 |
| 515 | [Clostridium] asparagiforme | 14 | 9 |
| 516 | Ruminococcus sp. CAG: 9-related 41_34 | 14 | 13 |
| 517 | Enterococcus sp. HMSC072H05 | 14 | 0 |
| 518 | Firmicutes bacterium CAG: 475 | 14 | 39 |
| 519 | Clostridium sp. CAG: 440 | 14 | 0 |
| 520 | Coprobacillus sp. 8_1_38FAA | 14 | 35 |
| 521 | Alloprevotella rava | 14 | 14 |
| 522 | Collinsella ihuae | 13 | 0 |
| 523 | Coprobacillus sp. CAG: 235_29_27 | 13 | 0 |
| 524 | Megasphaera sp. NM10 | 13 | 39 |
| 525 | Enterorhabdus caecimuris | 13 | 21 |
| 526 | Peptoniphilus harei | 13 | 0 |
| 527 | Prevotella sp. CAG: 755 | 13 | 13 |
| 528 | Ruminococcus sp. CAG: 579 | 13 | 0 |
| 529 | Paraprevotella xylaniphila | 13 | 0 |
| 530 | Enterococcus sp. 5B7_DIV0075 | 12 | 0 |
| 531 | Tyzzerella nexilis | 12 | 81 |
| 532 | Ruminococcus sp. CAG: 57 | 12 | 36 |
| 533 | Parabacteroides sp. Marseille-P3763 | 12 | 0 |
| 534 | Raoultibacter timonensis | 12 | 0 |
| 535 | Bacteroides thetaiotaomicron CAG: 40 | 12 | 0 |
| 536 | Clostridiales bacterium SK-Y3 | 12 | 0 |
| 537 | Eubacterium sp. 41_20 | 12 | 62 |
| 538 | Mobilibacterium timonense | 12 | 0 |
| 539 | Arabia massiliensis | 12 | 0 |
| 540 | Bacteroides vulgatus CAG: 6 | 12 | 7 |
| 541 | Lysinibacillus boronitolerans | 12 | 0 |
| 542 | Ruminococcus sp. CAG: 108-related_41_35 | 11 | 50 |
| 543 | Acinetobacter sp. LCT-H3 | 11 | 0 |
| 544 | Odoribacter splanchnicus CAG: 14 | 11 | 0 |
| 545 | Synergistes jonesii | 11 | 0 |
| 546 | Olsenella profusa | 11 | 6 |
| 547 | Synergistes sp. 3_1_syn1 | 11 | 0 |
| 548 | Bilophila sp. 4_1_30 | 11 | 78 |
| 549 | Clostridium sp. CAG: 798 | 11 | 61 |
| 550 | Bacteroides sp. 2_1_33B | 11 | 0 |
| 551 | Prevotella sp. 885 | 11 | 146 |
| 552 | Intestinibacter bartlettii | 11 | 13 |
| 553 | Enterorhabdus mucosicola | 10 | 11 |
| 554 | Bacteroides cellulosilyticus CAG: 158 | 10 | 30 |
| 555 | Enterococcus sp. 3H8_DIV0648 | 10 | 0 |
| 556 | Clostridium sp. CAG: 269 | 10 | 115 |
| 557 | Candida parapsilosis | 10 | 0 |
| 558 | Clostridium nexile CAG: 348 | 10 | 74 |
| 559 | Weissella sp. DD23 | 10 | 0 |
| 560 | Actinomyces dentalis | 9 | 0 |
| 561 | uncultured crAssphage | 9 | 9 |
| 562 | Tissierellia bacterium SS-A11 | 9 | 0 |
| 563 | Clostridium sp. ASBs410 | 9 | 0 |
| 564 | Roseburia sp. CAG: 471 | 9 | 119 |
| 565 | Paeniclostridium sordellii | 9 | 0 |
| 566 | Collinsella stercoris | 9 | 0 |
| 567 | Bacteroides coprophilus | 9 | 8 |
| 568 | Bacteroides sp. 3_1_33FAA | 9 | 17 |
| 569 | Enterococcus gilvus | 9 | 0 |
| 570 | Acidaminococcus sp. CAG: 542 | 8 | 0 |
| 571 | Alistipes sp. CAG: 29 | 8 | 30 |
| 572 | Prevotella sp. CAG: 617 | 8 | 8 |
| 573 | Leuconostoc lactis | 8 | 0 |
| 574 | Collinsella sp. An2 | 8 | 0 |
| 575 | Actinomyces sp. oral taxon 175 | 8 | 0 |
| 576 | Clostridiales bacterium VE202-21 | 8 | 0 |
| 577 | Listeria monocytogenes | 8 | 0 |
| 578 | Clostridium sp. 7_2_43FAA | 8 | 0 |
| 579 | Paraclostridium bifermentans | 8 | 0 |
| 580 | Enterococcus pallens | 8 | 0 |
| 581 | [Desulfotomaculum] guttoideum | 7 | 0 |
| 582 | Anaerococcus prevotii | 7 | 0 |
| 583 | Actinomyces viscosus | 7 | 0 |
| 584 | Bacteroides finegoldii | 7 | 33 |
| 585 | Clostridium celatum | 7 | 0 |
| 586 | Bacteroides stercoris CAG: 120 | 7 | 0 |
| 587 | Enterococcus saccharolyticus | 7 | 0 |
| 588 | Enterococcus malodoratus | 7 | 0 |
| 589 | Clostridium sp. CAG: 470 | 7 | 10 |
| 590 | Actinomyces odontolyticus | 7 | 0 |
| 591 | Peptoniphilus duerdenii | 7 | 0 |
| 592 | Actinomyces sp. ICM58 | 7 | 0 |
| 593 | Ruminococcus sp. 37_24 | 7 | 0 |
| 594 | Alistipes putredinis CAG: 67 | 7 | 9 |
| 595 | Fusobacterium mortiferum | 7 | 0 |
| 596 | Collinsella phocaeensis | 7 | 0 |
| 597 | Dialister succinatiphilus | 7 | 196 |
| 598 | Enterococcus sp. kppr-6 | 6 | 0 |
| 599 | Eubacterium callanderi | 6 | 0 |
| 600 | Sutterella wadsworthensis CAG: 135 | 6 | 18 |
| 601 | Clostridiales bacterium 36_14 | 6 | 52 |
| 602 | Prevotella lascolaii | 6 | 11 |
| 603 | Collinsella intestinalis | 6 | 0 |
| 604 | Lachnospiraceae bacterium 5_1_63FAA | 6 | 33 |
| 605 | Actinomyces sp. ICM39 | 6 | 0 |
| 606 | Acinetobacter sp. CIP 101934 | 6 | 0 |
| 607 | Acinetobacter lwoffii | 5 | 0 |
| 608 | Peptoniphilus sp. BV3AC2 | 5 | 0 |
| 609 | Bifidobacterium bifidum CAG: 234 | 5 | 0 |
| 610 | Bifidobacterium pseudocatenulatum CAG: 263 | 5 | 0 |
| 611 | Lactobacillus rhamnosus | 5 | 0 |
| 612 | Prevotella sp. CAG: 732 | 5 | 65 |
| 613 | Megamonas funiformis CAG: 377 | 5 | 0 |
| 614 | Megamonas hypermegale | 4 | 124 |
| 615 | Eubacterium sp. CAG76_36_ 125 | 4 | 30 |
| 616 | Bifidobacterium adolescentis CAG: 119 | 4 | 18 |
| 617 | Roseburia sp. CAG: 197 | 0 | 47 |
| 618 | Bacteroides sp. CAG: 98 | 0 | 41 |
| 619 | Parasutterella excrementihominis CAG: 233 | 0 | 48 |
| 620 | Clostridium sp. CAG: 122 | 0 | 98 |
| 621 | Firmicutes bacterium CAG: 313 | 0 | 124 |
| 622 | Clostridiales bacterium KLE1615 | 0 | 301 |
| 623 | Elusimicrobium sp. An273 | 0 | 124 |
| 624 | Bifidobacterium dentium | 0 | 5 |
| 625 | Clostridia bacterium UC5.1-1D1 | 0 | 8 |
| 626 | Tyzzerella sp. Marseille-P3062 | 0 | 35 |
| 627 | Acidaminococcus intestini | 0 | 7 |
| 628 | Prevotella sp. CAG: 386 | 0 | 64 |
| 629 | uncultured Lachnospira sp. | 0 | 248 |
| 630 | Clostridium sp. M62/1 | 0 | 10 |
| 631 | Sellimonas intestinalis | 0 | 23 |
| 632 | Clostridium sp. CAG: 780 | 0 | 114 |
| 633 | Clostridium sp. AT4 | 0 | 26 |
| 634 | Roseburia sp. CAG: 380 | 0 | 16 |
| 635 | Ruminococcus sp. CAG: 330 | 0 | 23 |
| 636 | Clostridium sp. CAG: 575 | 0 | 4 |
| 637 | Clostridium sp. CAG: 62 | 0 | 74 |
| 638 | Lachnospiraceae bacterium 8_1_57FAA | 0 | 11 |
| 639 | Eubacterium sp. CAG: 603 | 0 | 22 |
| 640 | Ruminococcaceae bacterium cv2 | 0 | 15 |
| 641 | Akkermansia sp. CAG: 344 | 0 | 58 |
| 642 | Clostridium sp. 44_14 | 0 | 54 |
| 643 | Clostridium sp. CAG: 628 | 0 | 107 |
| 644 | Ruminococcus sp. CAG: 624 | 0 | 32 |
| 645 | Roseburia sp. CAG: 303 | 0 | 451 |
| 646 | Lactobacillus rogosae | 0 | 33 |
| 647 | Roseburia sp. CAG: 309 | 0 | 25 |
| 648 | Acholeplasma sp. CAG: 878 | 0 | 115 |
| 649 | Shigella sonnei | 0 | 35 |
| 650 | Megasphaera elsdenii CAG: 570 | 0 | 7 |
| 651 | Fusobacterium sp. CAG: 439 | 0 | 8 |
| 652 | Bifidobacterium pseudolongum | 0 | 9 |
| 653 | Bacteroides sp. CAG: 770 | 0 | 38 |
| 654 | Bacteroides sp. 14(A) | 0 | 11 |
| 655 | Anaerovorax odorimutans | 0 | 15 |
| 656 | Bifidobacterium merycicum | 0 | 7 |
| 657 | Ruminococcus callidus | 0 | 268 |
| 658 | Eubacterium sp. CAG: 252 | 0 | 27 |
| 659 | Clostridium sp. CAG: 253 | 0 | 42 |
| 660 | Clostridium sp. ATCC BAA-442 | 0 | 20 |
| 661 | Coprobacillus sp. 28_7 | 0 | 21 |
| 662 | Clostridium sp. SCN 57-10 | 0 | 40 |
| 663 | Ruminococcus sp. CAG: 403 | 0 | 7 |
| 664 | Clostridium sp. 42_12 | 0 | 14 |
| 665 | Firmicutes bacterium CAG: 65_45_313 | 0 | 108 |
| 666 | Lachnospiraceae bacterium CAG: 25 | 0 | 8 |
| 667 | Sutterella parvirubra | 0 | 15 |
| 668 | Eubacterium sp. 45_250 | 0 | 17 |
| 669 | Firmicutes bacterium CAG: 272_52_7 | 0 | 26 |
| 670 | Clostridium sp. 29_15 | 0 | 18 |
| 671 | Veillonella dispar | 0 | 94 |
| 672 | Bacteroides sp. CAG: 530 | 0 | 145 |
| 673 | Flavonifractor sp. An82 | 0 | 15 |
| 674 | Firmicutes bacterium CAG: 449 | 0 | 81 |
| 675 | Eubacterium ramulus | 0 | 27 |
| 676 | Clostridium sp. CAG: 75 | 0 | 26 |
| 677 | Shigella flexneri | 0 | 10 |
| 678 | Coprococcus comes CAG: 19 | 0 | 9 |
| 679 | Firmicutes bacterium CAG: 341 | 0 | 52 |
| 680 | Candidatus Gastranaerophilales bacterium HUM_1 | 0 | 102 |
| 681 | Clostridium sp. CAG: 813 | 0 | 49 |
| 682 | Proteobacteria bacterium CAG: 495 | 0 | 31 |
| 683 | Coprobacillus sp. CAG: 698 | 0 | 130 |
| 684 | Prevotella stercorea | 0 | 117 |
| 685 | Gemmiger sp. An50 | 0 | 16 |
| 686 | Clostridium sp. 26_21 | 0 | 12 |
| 687 | Clostridium sp. CAG: 217 | 0 | 20 |
| 688 | Butyrivibrio crossotus | 0 | 11 |
| 689 | Coprococcus sp. CAG: 782 | 0 | 11 |
| 690 | Prevotella sp. P3-122 | 0 | 18 |
| 691 | Blautia sp. CAG: 52 | 0 | 31 |
| 692 | uncultured organism | 0 | 7 |
| 693 | Roseburia sp. 499 | 0 | 46 |
| 694 | Clostridium sp. CAG: 277 | 0 | 170 |
| 695 | Mitsuokella multacida | 0 | 126 |
| 696 | Azospirillum sp. 51_20 | 0 | 8 |
| 697 | Ruminococcus sp. DSM 100440 | 0 | 37 |
| 698 | Clostridium sp. CAG: 62_40_43 | 0 | 30 |
| 699 | Butyrivibrio crossotus CAG: 259 | 0 | 10 |
| 700 | Azospirillum sp. CAG: 239 | 0 | 82 |
| 701 | Prevotella sp. CAG: 520 | 0 | 540 |
| 702 | Lachnospiraceae bacterium TF01-11 | 0 | 111 |
| 703 | Burkholderiales bacterium 1_1_47 | 0 | 118 |
| 704 | Bacteroides massiliensis | 0 | 134 |
| 705 | Eubacterium sp. CAG: 248 | 0 | 294 |
| 706 | Clostridiales bacterium VE202-14 | 0 | 17 |
| 707 | Eubacterium sp. 36_13 | 0 | 36 |
| 708 | Veillonella atypica | 0 | 41 |
| 709 | Sutterella sp. 54_7 | 0 | 5 |
| 710 | Dakarella massiliensis | 0 | 64 |
| 711 | Clostridium ventriculi | 0 | 119 |
| 712 | Sutterella sp. CAG: 351 | 0 | 44 |
| 713 | Bacteroides acidifaciens | 0 | 9 |
| 714 | Lachnospiraceae bacterium KHCPX20 | 0 | 7 |
| 715 | Roseburia sp. CAG: 10041_57 | 0 | 38 |
| 716 | Veillonella sp. DORA_A_3_16_22 | 0 | 33 |
| 717 | Selenomonas ruminantium | 0 | 6 |
| 718 | Emergencia timonensis | 0 | 14 |
| 719 | Roseburia sp. CAG: 45 | 0 | 62 |
| 720 | Azospirillum sp. 47_25 | 0 | 8 |
| 721 | Fournierella massiliensis | 0 | 23 |
| 722 | Tenericutes bacterium HGW-Tenericutes-4 | 0 | 16 |
| 723 | Blastocystis sp. subtype 1 | 0 | 33 |
| 724 | Prevotella bryantii | 0 | 8 |
| 725 | Roseburia sp. 831b | 0 | 46 |
| 726 | Bacteroides sp. CAG: 754 | 0 | 25 |
| 727 | Roseburia sp. 40_7 | 0 | 11 |
| 728 | Roseburia sp. CAG: 100 | 0 | 70 |
| 729 | Bacteroides congonensis | 0 | 14 |
| 730 | Clostridium sp. CAG: 12237_41 | 0 | 36 |
| 731 | Lachnospiraceae bacterium 10-1 | 0 | 20 |
| 732 | Eubacterium sp. CAG: 38 | 0 | 452 |
| 733 | Azospirillum sp. CAG: 260 | 0 | 16 |
| 734 | Bacteroides ovatus CAG: 22 | 0 | 36 |
| 735 | Firmicutes bacterium CAG 194_44_15 | 0 | 7 |
| 736 | Veillonella parvula | 0 | 14 |
| 737 | Clostridium sp. CAG: 265 | 0 | 41 |
| 738 | Coprococcus sp. ART55/1 | 0 | 9 |
| 739 | Parasutterella excrementihominis | 0 | 63 |
| 740 | Roseburia intestinalis CAG: 13 | 0 | 190 |
| 741 | Roseburia sp. CAG: 50 | 0 | 13 |
| 742 | uncultured Roseburia sp. | 0 | 36 |
| 743 | Akkermansia glycaniphila | 0 | 18 |
| 744 | Mycoplasma sp. CAG: 611 | 0 | 61 |
| 745 | Eggerthella sp. CAG: 298 | 0 | 43 |
| 746 | Clostridium sp. CAG: 632 | 0 | 226 |
| 747 | Prevotella ruminicola | 0 | 7 |
| 748 | Drancourtella massiliensis | 0 | 18 |
| 749 | Lachnoclostridium sp. An14 | 0 | 19 |
| 750 | Eubacterium sp. CAG: 115 | 0 | 14 |
| 751 | Bacteroides sp. CAG: 1076 | 0 | 4 |
| 752 | Coprococcus sp. CAG: 131 | 0 | 26 |
| 753 | Veillonella sp. oral taxon 158 | 0 | 12 |
| 754 | Phascolarctobacterium sp. CAG: 266 | 0 | 14 |
| 755 | Lachnoclostridium edouardi | 0 | 11 |
| 756 | Prevotella stercorea CAG: 629 | 0 | 24 |
| 757 | Alloprevotella tannerae | 0 | 7 |
| 758 | Olsenella sp. oral taxon 807 | 0 | 8 |

TABLE 9
PostIntervention Control vs PostIntervention Treatment Seed Average Values

| | | Post | | | | Post | | |
|---|---|---|---|---|---|---|---|---|
| | Baseline | Intervention | | | Baseline | Intervention | | |
| | Control | Control | | Increase/ | Treatment | Treatment | | Increase/ |
| IDs | Average | Average | Difference | Decrease | Average | Average | Difference | Decrease |
| Carbohydrates | [symbol] | [symbol] | [symbol] | Decrease¶ | [symbol] | [symbol] | [symbol] | Decrease¶ |
| C [symbol] , Vitamins, | [symbol] | [symbol] | [symbol] | Increase | [symbol] | [symbol] | [symbol] | Decrease¶ |
| Prosthetic Groups, | | | | | | | | |
| Pigments | | | | | | | | |
| Amino Acids and | [symbol] | [symbol] | [symbol] | Decrease¶ | [symbol] | [symbol] | [symbol] | Decrease¶ |
| Derivatives | | | | | | | | |
| Protein Metabolism | [symbol] | [symbol] | [symbol] | Decrease¶ | [symbol] | [symbol] | [symbol] | Decrease¶ |
| [symbol] Metabolism | [symbol] | [symbol] | [symbol] | Increase | [symbol] | [symbol] | [symbol] | Decrease¶ |
| Cell Wall and Capsule | [symbol] | [symbol] | [symbol] | Decrease¶ | [symbol] | [symbol] | [symbol] | Decrease¶ |
| Fatty Acids, Lipids, | [symbol] | [symbol] | [symbol] | Decrease¶ | [symbol] | [symbol] | [symbol] | Decrease¶ |
| and [symbol] | | | | | | | | |
| [symbol] and | [symbol] | [symbol] | [symbol] | Decrease¶ | [symbol] | [symbol] | [symbol] | Decrease¶ |
| [symbol] | | | | | | | | |
| [symbol] Metabolism | [symbol] | [symbol] | [symbol] | Decrease¶ | [symbol] | [symbol] | [symbol] | Decrease¶ |
| Respiration | [symbol] | [symbol] | [symbol] | Decrease¶ | [symbol] | [symbol] | [symbol] | Decrease¶ |
| Stress Response | [symbol] | [symbol] | [symbol] | Decrease¶ | [symbol] | [symbol] | [symbol] | Decrease¶ |
| Metabo [symbol] damage and | [symbol] | [symbol] | [symbol] | Decrease¶ | [symbol] | [symbol] | [symbol] | Decrease¶ |
| [symbol] repair or mitigation | | | | | | | | |
| [symbol] | [symbol] | [symbol] | [symbol] | Decrease¶ | [symbol] | [symbol] | [symbol] | Decrease¶ |
| [symbol] | [symbol] | [symbol] | [symbol] | Decrease¶ | [symbol] | [symbol] | [symbol] | Decrease¶ |
| Phosphorus Metabolism | [symbol] | [symbol] | [symbol] | Decrease¶ | [symbol] | [symbol] | [symbol] | Decrease¶ |
| Regulation and | [symbol] | [symbol] | [symbol] | Decrease¶ | [symbol] | [symbol] | [symbol] | Decrease¶ |
| Cell Signaling | | | | | | | | |
| Cell Division and | [symbol] | [symbol] | [symbol] | Decrease¶ | [symbol] | [symbol] | [symbol] | Decrease¶ |
| Cell Cycle | | | | | | | | |
| [symbol] acquisition and | [symbol] | [symbol] | [symbol] | Increase | [symbol] | [symbol] | [symbol] | Decrease¶ |
| metabolism | | | | | | | | |
| Potassium metabolism | [symbol] | [symbol] | [symbol] | Decrease¶ | [symbol] | [symbol] | [symbol] | Decrease¶ |
| Sulfur Metabolism | [symbol] | [symbol] | [symbol] | Decrease¶ | [symbol] | [symbol] | [symbol] | Decrease¶ |
| Virulence, Disease | [symbol] | [symbol] | [symbol] | Decrease¶ | [symbol] | [symbol] | [symbol] | Decrease¶ |
| and Defense | | | | | | | | |
| Phages, Prophages, | [symbol] | [symbol] | [symbol] | Decrease¶ | [symbol] | [symbol] | [symbol] | Decrease¶ |
| Transposable elements, | | | | | | | | |
| Plasmids | | | | | | | | |
| Metabolism of Aromatic | [symbol] | [symbol] | [symbol] | | [symbol] | [symbol] | [symbol] | Decrease¶ |
| Compounds | | | | | | | | |
| M [symbol] and Che [symbol] | [symbol] | [symbol] | [symbol] | Increase | [symbol] | [symbol] | [symbol] | Decrease¶ |
| Thiamin | [symbol] | [symbol] | [symbol] | Increase | [symbol] | [symbol] | [symbol] | Decrease¶ |
| Nitrogen Metabolism | [symbol] | [symbol] | [symbol] | Decrease¶ | [symbol] | [symbol] | [symbol] | Decrease¶ |
| Predictions based on | [symbol] | [symbol] | [symbol] | Decrease¶ | [symbol] | [symbol] | [symbol] | Decrease¶ |
| plant-prokaryote | | | | | | | | |
| comparative analysis | | | | | | | | |
| Mito [symbol] electron | [symbol] | [symbol] | [symbol] | Decrease¶ | [symbol] | [symbol] | [symbol] | Decrease¶ |
| transport system | | | | | | | | |
| in plants | | | | | | | | |
| [symbol] and | [symbol] | [symbol] | [symbol] | Decrease¶ | [symbol] | [symbol] | [symbol] | Decrease¶ |
| [symbol] lation | | | | | | | | |
| [symbol] sugars | [symbol] | [symbol] | [symbol] | Decrease¶ | [symbol] | [symbol] | [symbol] | Decrease¶ |
| Secondary Metabolism | [symbol] | [symbol] | [symbol] | Decrease¶ | [symbol] | [symbol] | [symbol] | Decrease¶ |
| Plant Gluc [symbol] | [symbol] | [symbol] | [symbol] | Decrease¶ | [symbol] | [symbol] | [symbol] | Decrease¶ |
| Transcriptional | [symbol] | [symbol] | [symbol] | Decrease¶ | [symbol] | [symbol] | [symbol] | Decrease¶ |
| regulation | | | | | | | | |
| Phages, Prophages, | [symbol] | [symbol] | [symbol] | Decrease¶ | [symbol] | [symbol] | [symbol] | Decrease¶ |
| Transposable elements | | | | | | | | |
| Plant cell walls and | [symbol] | [symbol] | [symbol] | Decrease¶ | [symbol] | [symbol] | [symbol] | Decrease¶ |
| [symbol] surfaces | | | | | | | | |
| Central metabolism | [symbol] | [symbol] | [symbol] | Increase | [symbol] | [symbol] | [symbol] | |
| Autotrophy | [symbol] | [symbol] | [symbol] | Decrease¶ | [symbol] | [symbol] | [symbol] | Decrease¶ |
| A [symbol] Sensor and | [symbol] | [symbol] | [symbol] | Decrease¶ | [symbol] | [symbol] | [symbol] | Decrease¶ |
| transport module | | | | | | | | |
| Polyamines | [symbol] | [symbol] | [symbol] | | [symbol] | [symbol] | [symbol] | Decrease¶ |
| [symbol] Stress Response | [symbol] | [symbol] | [symbol] | | [symbol] | [symbol] | [symbol] | Decrease¶ |
| and Stationary Phase | | | | | | | | |
| Response | | | | | | | | |
| [symbol] | [symbol] | [symbol] | [symbol] | Decrease¶ | [symbol] | [symbol] | [symbol] | Decrease¶ |
| [symbol] electron | | | | | | | | |
| transport system | | | | | | | | |

[symbol]

indicates data missing or illegible when filed

### Discussion:

In this study, following earlier reports of clinical improvement in terms of sleep (Raghavan et al., 2021^{a}), behavioural pattern (Raghavan et al., 2021^{b}), plasma αSyn (Raghavan et al., 2021a) and serum melatonin increase (Raghavan et al., 2021^{b}), gut dysbiosis has been shown to have a strong correlation with the severity of symptoms in ASD (Grimaldi et al., 2018). We evaluated and compared the gut microbiota of the subjects who were supplemented with AFO-202-derived 1,3-1,6 beta glucan with those who did not take the supplement.

Several studies have reported the differences in the gut microbiota between children with ASD and neurotypical children. Reduced number of bifidobacterial and increased Clostridium spp., Desulfovibrio spp., Sutterella spp., and/or Veillonellacea was reported by Souza et al (2012). Tomova et al (2015) reported a change in Bacteroidetes/Firmicutes ratio and an increase in bifidobacterial numbers after probiotic administration.

An exclusion diet and a 6-week prebiotic intervention demonstrated lower abundance of Bifidobacterium spp. and Veillonellaceae family and higher abundance of Faecalibacterium prausnitzii and Bacteroides spp. (Grimaldi et al., 2018). Faecalibacterium, Ruminococcus, and Bifidobacterium were relatively less abundant, whereas Caloramator, Sarcina, Sutterella ceae, and Enterobacteriaceae were more abundant in children with ASD (De Angelis et al., 2013). In addition, lower abundances of the genera Prevotella, Coprococcus, and unclassified Veillonellaceae have been reported (Kang et al., 2013). Among these bacteria, increased Bacteroidaceae, Prevotellaceae, and Ruminococcaceae and decreased Prevotella copri, Faecalibacterium prausnitzii, and Haemophilus parainfluenzae have been reported (Kang et al., 2018; Oh et al., 2020).

In the present study, in line with these reports, the shift of the gut microbiome was towards a beneficial spectrum in Group 2 (Nichi Glucan) because there was a decrease in Enterobacter, Lactobacillus, Escherichia coli, Akkermansia muciniphila CAG:154, Blautia spp., Coprobacillus sp., several clostridium spp., and Clostridium bolteae CAG:59, with an increase in the abundance of Bacteroides, Prevotella, Faecalibacterium prausnitzii, and Prevotella copri. Desulfovibrio Bacteria which have been reported to be associated with PD (Murros et al., 2021) decreased in the Gr. 2.

In particular, Enterobacteria and E. coli significantly decreased in Group 2 compared to Group 1 after the intervention. Gram-negative enteric bacteria such as the Enterobacter and E. coli secrete the amyloid curli that constitutes 85% of the extracellular matrix of enteric biofilms. The curli has similarities and associations with pathological and immunomodulatory human amyloids such as amyloid-3 implicated in AD, αSyn involved in ASD and PD, and serum amyloid A associated with neuroinflammation (Miller et al., 2021). Curli causes misfolding (Al-Mazidi et al., 2021) and accumulation of the neuronal protein αSyn in the form of insoluble amyloid aggregations, leading to inflammation and neuronal dysfunction that is central to pathogenesis of Lewy-body-associated synucleinopathies, including PD and AD.

Curli-producing bacteria also increase the production and aggregation of the amyloid protein αSyn, which has been shown to propagate in a prion-like fashion from the gut to the brain via the vagus nerve and/or spinal cord, thus culminating in the neurological disorders such as ASD (Al-Mazidi et al., 2021). In this study, the significant decrease in Enterobacter and E-coli will thus be of benefit in these synculeopathies. Before the start of this study, the objective to study αSyn was to understand the effects of the beta glucan supplementation on Synaptic imbalance in presynaptic terminals, observed in ASD.

The study results showed that plasma levels of αSyn increased in Group 2 compared with Group 1 along with improvement in Childhood Autism Rating Scale score and sleep pattern which to our knowledge is the first of its kind intervention producing an observable change in the plasma synuclein levels (Al-Mazidi et al., 2021).

In a study by Ding et al., it was reported that 14 functional properties displayed differences between the ASD and healthy control groups. Four functions, including galactose metabolism, glycosyltransferase activity, glutathione metabolism, and antifolate resistance, were enriched in the ASD group. In another study by Lindefeldt et al, the relative abundance of 26 metabolic pathways was diminished after 3 months on a ketogenic diet in children with epilepsy and 3 became more relative abundant.

The group with most pathways changed was carbohydrate metabolism, showing reduction of fructooligosaccharides (FOS) and raffinose utilization, sucrose utilization, glycogen metabolism, lacto-N-biose I and galacto-N-biose metabolic pathway. The SEED average findings of the present study also reflect this beneficial outcome (FIG. 21 and Table 9)

The study of the gut microbiome has offered further new insights wherein Enterobacter increasing curli protein and αSyn deposition in the enteric nervous system having been controlled by the beta glucan food supplement, the increase in plasma αSyn levels point out to the disintegration of the amyloid deposits leading to these αSyn entering the blood stream. Indeed, natural killer (NK) cells have been shown to act as efficient scavengers of abnormal α-Syn aggregates (Earls et al., 2020), and the AFO-202 beta glucan has a proven capability to increase and activate NK cells (Ikewaki et al., 2007), which could be another probable mechanism contributing to the increased αSyn levels in the plasma, apart from positive clinical outcomes in these children with ASD.

This result highlights NK cell's potential as a promising therapeutic strategy for prophylaxis and prevention of brain disorders, and they are likely to be used for such αSyn accumulation and propagation, after relevant research on their specific pathways and variations in their capability. The NK cells have been proven to clear the amyloid deposits peripherally though not macrophages (Raghavan et al., 2021^{c}). In the central nervous system, such a role is played by the microglia (Bartels et al., 2020). βeta-glucans also rejuvenate microglia (Luna et al., 2015) that have been shown to scavenge amyloid deposits in the brain and CNS (Morato Torres et al., 2020), thus proving to be a wholesome therapeutic strategy for neurodevelopmental and neurodegenerative diseases.

Altered α-Syn protein misfolding spreading to anatomically connected regions in a prion-like manner and mediating neurodegenerative diseases such as PD (Terajima et al., 2020) and the increased risk of children with ASD in developing PD at a later stage (Swirski et al., 2014) suggests further research into these converging pathogenic pathways of neurodevelopmental and neurodegenerative diseases is needed as well as suggests that this safety-proven food supplement is a preventive strategy in subjects with ASD against PD.

Other than research into normal and abnormal α-syn being warranted, studying the implications of soluble and insoluble α-syn (Boziki et al., 2020) is important because the proportion of insoluble α-syn that was phosphorylated at Ser129 was reported to be significantly higher in brain tissue from PD patients. In addition, cell lines such as the SH-SYHY neuroblastoma (Boziki et al., 2020) will reveal the correlation of the various α-syn with the severity of symptoms and pathogenesis in these neurodegenerative diseases, apart from helping to develop novel disease-modifying strategies employing such simple nutritional supplementation.

The microbiota reconstituted in a beneficial manner in the present study with AFO-202 beta glucan must be further progressed into research to study the effects of other variants of A. pullulans beta glucan that have been shown to be anti-inflammatory. Such research could lead to mechanistic insight into the molecular pathways from the local immune responses in the gut leading to systemic inflammation and, eventually, to organ-specific autoimmunity of the CS in neuroinflammatory conditions such as MS.

### Conclusion:

Favourable reconstitution of the gut microbiota after consumption of AFO-202 beta glucan in children with ASD has been demonstrated in this study, apart from the clinical improvement already reported. The decrease in Enterobacteria demonstrates the potential of this beta-glucan supplementation for neurodevelopmental conditions such as ASD as well as neurodegenerative disorders such as PD and AD, with converging pathways of amyloid accumulations and propagation, warranting larger clinical studies and research to recommend this as a routine food supplement or an adjunct to existing therapies for prevention and management of both neurodevelopmental and neurodegenerative diseases.

### List of Abbreviations

ASD—autism spectrum disorder
WGM—Whole genome metagenome
αSyn—α-synuclein
MS—multiple sclerosis
AD—Alzheimer's disease
PD—Parkinson's disease

### Improving Behavioural Pattern and Alpha-Synuclein Levels

### Abstract:

Autism spectrum disorders (ASDs) are a wide range of disabilities in which the neurosynaptic biomarkers and mechanisms remain elusive. As there are no definite interventional modalities available to improve the behavioural pattern, remedial therapies are the only option and have varying outcomes. Based on our earlier study on the improvement of melatonin in ASD children when supplemented with a biological response modifier beta-glucan food supplement, we have evaluated the childhood autism rating scale (CARS) and alpha-synuclein levels in this randomized, parallel-group, multiple-arm clinical trial. Six subjects with ASD (n=6) Gr. 1 underwent conventional treatment comprising remedial behavioural therapies and L-Carnosine 500 mg per day, and 12 subjects (n=12) Gr. 2 underwent supplementation with the Nichi Glucan food supplement 0.5 g twice daily along with the conventional treatment.

There was a significant decrease in the CARS score in all of the children of the Nichi Glucan Gr.2 compared to the control (p-value=0.034517), by an average of 3 points in the improvement of autism's signs and symptoms, whereas the improvement was very mild or nil in Gr.1. Plasma levels of alpha-synuclein were significantly higher in Gr. 2 (Nichi Glucan) than in the control group Gr. 1 (p-value=0.091701). Improvement of the behavioural pattern CARS score and a correlating alpha-synuclein level, followed by a safe beta-glucan food supplement, warrants further research on other parameters, such as gut-microbiota evaluation, and relevant neuronal biomarkers which is likely to cast light on novel solutions.

### Trial Registration Number:

Clinical Trials Registry of India (CTRI/2020/10/028322)

### Lay Summary:

Behavioural pattern in children with Autism Spectrum Disorder has been observed to improve following consumption of Beta 1,3-1,6 Glucan food supplement. The CARS score has also shown improved, compared to the control group in this pilot clinical study along with increase of a neuronal marker Alpha-Synuclein which is usually lower in affected children compared to normal age matched controls.

### Methods:

This study was approved by the institutional ethics committee of Kenmax Medical Service Private Limited, Madurai, India and was registered as a randomized, parallel-group, and multiple-arm clinical trial in the Clinical Trials Registry of India (CTRI/2020/10/028322). The caregiver of all the subjects gave their informed consent for inclusion before participation in the study. The study was conducted in accordance with the Declaration of Helsinki.

### Study Design:

The subjects enrolled in the study had received a diagnosis of ASD by a developmental paediatrician and were verified by a psychologist using a clinical interview for a behavioural pattern that incorporated CARS.

Eighteen subjects (n=18) with ASD in total were enrolled in this prospective, open-label, pilot clinical trial comprising of two arms. The CONSORT flow diagram is presented as FIG. 14.

Arm 1 or Gr. 1: Control: Six subjects with ASD (n=6) underwent conventional treatment comprising remedial behavioural therapies and L-Carnosine 500 mg per day.

Arm 2 or Gr. 2: Treatment arm: 12 subjects (n=12) underwent supplementation with Nichi Glucan (Aureobasidium pullulans strain AFO-202 (also referred to as FO-68 [(accession number) FERM BP-19327]) derived Beta 1,3-1,6 Glucan) food supplement along with conventional treatment. Each subject consumed two sachets (0.5 g each) of Nichi Glucan daily—one sachet with a meal twice daily—for a period of 90 days.

### Inclusion Criteria:

i. Age: 3 to 18 years;
ii. Gender: Both male and female;
iii. ASD criteria as per CARS score; and,
iv. Parents/caretakers willing to provide consent for their children to actively participate in the study.

### Exclusion Criteria:

i. Subjects aged more than 18 years old;
ii. Any child with acute general illness or who has been on any antibiotic, anti-inflammatory, or antioxidant treatment in the two weeks prior to enrolment in the study;
iii. Hyperallergic to any of the investigational products; and,
iv. Subjects with long-standing infections.

### Outcome Measures:

i. Childhood autism rating scale (CARS):
The CARS was monitored at baseline and after 90 days between the Gr.1 (control) and Gr.2 (Nichi Glucan). The CARS score was calculated based on a cumulative score obtained on the CARS scale, wherein a score below 30 indicates absence of sufficient signs and symptoms indicative of autism, a score between 30 and 36 indicates mild-to-moderately severe autism, and a score from 37 to 60 is correlated with severe autism (Ramaekers et al., 2019). The psychologist who performed the assessment and the parents were blind to the participant's treatment status; hence, this is a double-blind study.
ii. Evaluation of plasma alpha-synuclein:
Human alpha-synuclein (α-syn) levels in plasma were measured in peripheral blood at baseline and after the study's completion at 90 days. The measurement was performed using the human α-synuclein (α-syn) ELISA kit (KINESISDx, USA) as per the manufacturer's instructions.

### Data Analysis:

All data were analysed using Excel software statistics package analysis software (Microsoft Office Excel(R)); Student's paired t-tests were also calculated using this package; and p-values<0.05 were considered significant.

### Results:

During enrolment, six subjects with ASD (n=6) could be enrolled in the control group (Gr. 1), whereas in the treatment group (Gr. 2), one of them dropped out before the start of the study. During the study, four subjects were lost to follow-up: two in Gr. 1 (one dropped out due to social problems in the family, and the other relocated to another city) and two in Gr. 2 (one dropped out due to social problems in the family, and the other relocated to another city). A total of 13 subjects (four in Gr. 1 and nine in Gr. 2) completed the study. One female subject was in both Gr. 1 and Gr. 2. The rest were male.

### Adverse Effects:

Only one child exhibited possible mild adverse effects related to increased bowel movements in Gr. 2 for one week after supplementation with Nichi Glucan, which settled on its own. No adverse effects were found in any of the other children.

### Score on CARS Scale:

Among the children in the control group (Gr.1), all four were in the category of severe autism, and their score at baseline ranged from 37 to 52 (mean=42.75±5.76). Among the nine children in Gr.2, two were in the mild-to-moderate category of autism (mean=33.5±2.5), whereas the remaining seven were in the category of severe autism (mean=43.71±4.80).

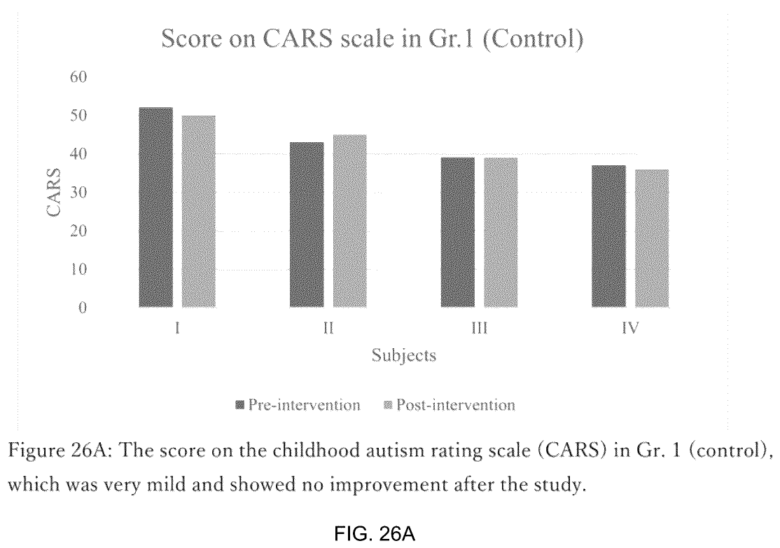

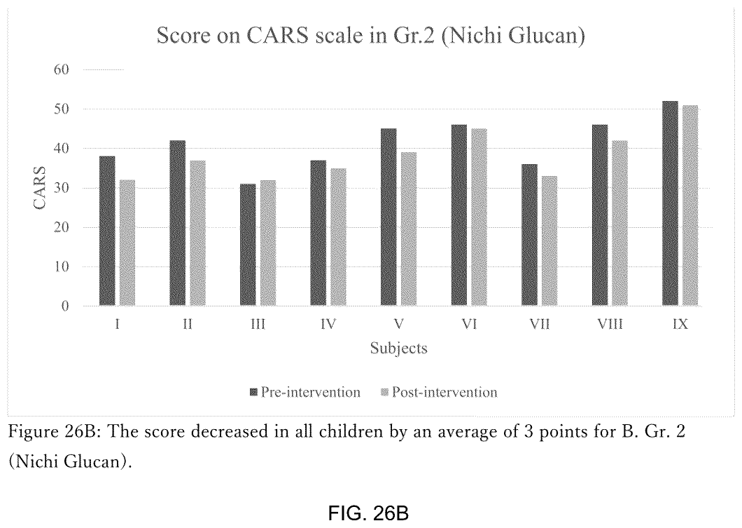

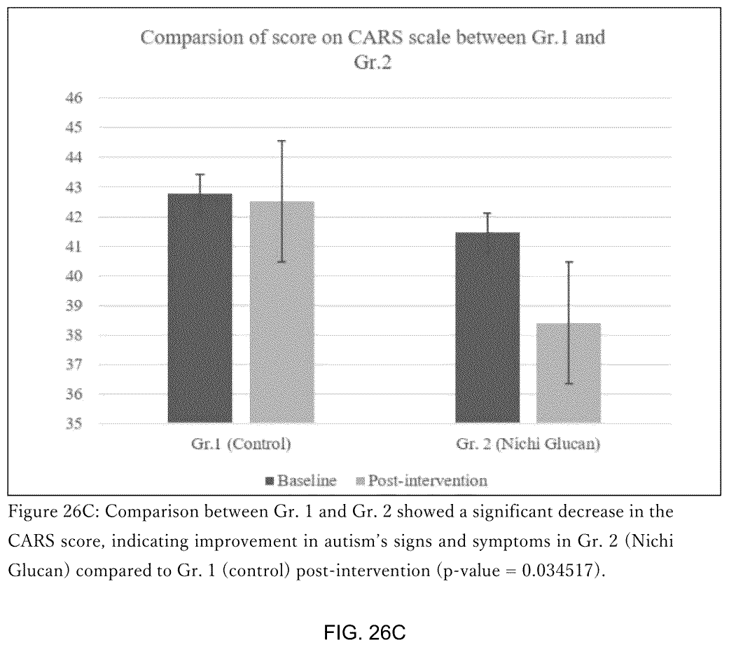

After the intervention, the mean CARS score in the four children of the control group was 42.5±5.4, while in Gr.2 (Nichi Glucan), the mean of the CARS score in the two children with mild-to-moderate autism was 32.5±0.5. In the remaining seven children, the CARS score after Nichi Glucan intervention had a mean of 40.1±5.96. Thus, there was a significant decrease in the CARS score in all of the children in the Nichi Glucan Gr.2 group compared to the control (p-value=0.034517), with an average of 3 points in the improvement of autism's signs and symptoms, whereas the improvement was very mild or nil in Gr.1 (FIG. 26).

Among the various parameters assessed on the CARS, there was visible subjective improvement in the emotional response, including reduction in irritability and anger (88%), sleep improvement (88%), speech characteristics with improvement in finger pointing and monosyllables in 77%, and improved responses to the caregiver in 77% of the children in Nichi Glucan Gr. 2, but these improvements were very mild or nil in Gr.1.

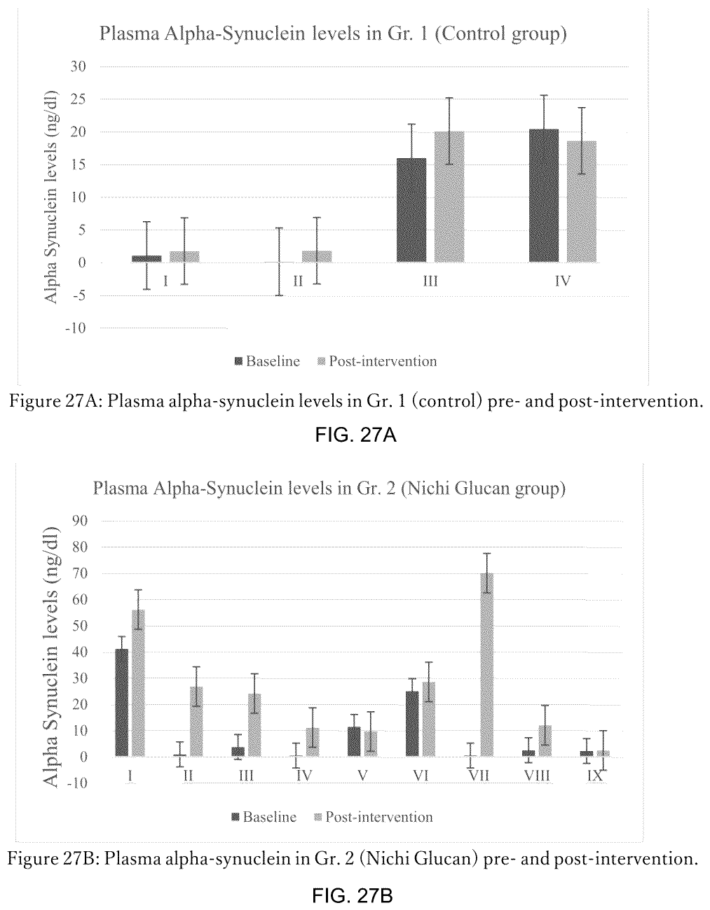

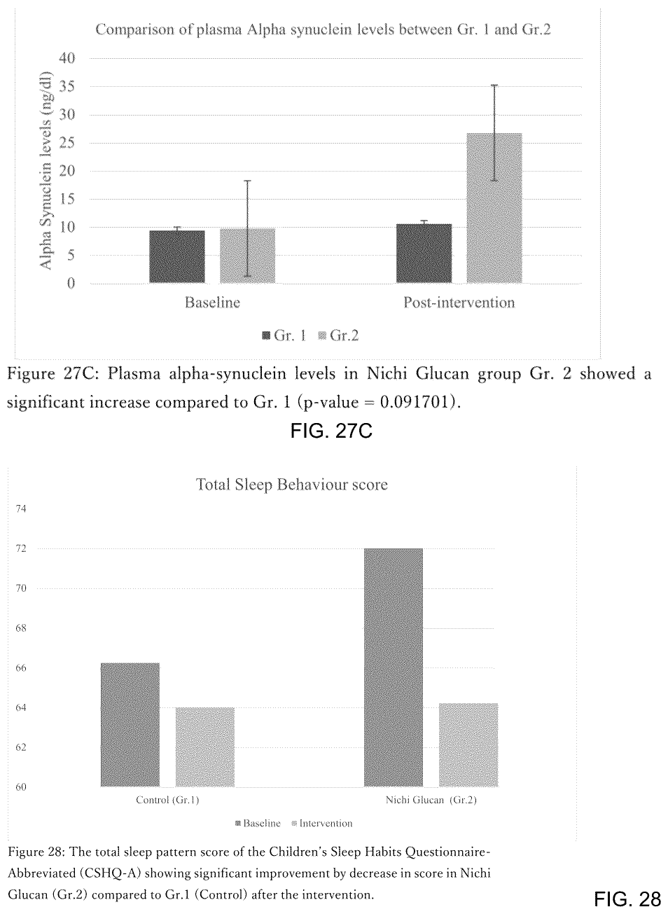

Plasma levels of alpha-synuclein ranged between 0.12 and 20.41 ng/dl (mean=9.73 ng/dl) in the control group and between 0.45 and 41.12 ng/dl (mean=9.39 ng/dl) in the treatment group at baseline. After the intervention, plasma levels of alpha-synuclein increased, with a mean increase in levels of 26.72 ng/dl in the treatment (Nichi Glucan) Gr.2 group compared to the control group Gr. 1 (mean increase=10.56 ng/dl) (p-value=0.091701) (FIG. 27).

### Discussion:

In this study of 13 subjects, the behavioural pattern evaluated by the CARS score improved in all nine subjects of Gr.2 (Nichi Glucan) (FIG. 14), especially on the emotional aspects and sleep-related parameters, and the alpha-synuclein levels increased significantly in these nine subjects compared to the control (FIG. 26). Alpha-synuclein plays a key role in the synaptic functions of neurons by regulating CADPS2 mRNA expression. There are reports that the neural overconnectivity and synapse alteration associated with the pathogenesis of ASD may actually owe their aetiology to alpha-synuclein dysregulation (Kadak et al., 2015; Sriwimol et al., 2018; Obergasteiger et al., 2014).

Further, alpha-synuclein has recently been considered one of the important biomarkers for the diagnosis of autism and ASD, wherein the levels are low compared to age-matched controls (Kadak et al., 2015; Sriwimol et al., 2018; Siddique et al. 2020). In regard to neurodegenerative diseases such as PD, the reports have been varied, with some reporting lower than normal levels and others higher. In a correlating hypothesis of the plasma alpha-synuclein level, between autism and neurodegenerative diseases, it has been proposed that alpha-synuclein aggregation in the neural synapse may lead to lower plasma levels (Sriwimol et al., 2018).

Whether the increase in alpha-synuclein levels in plasma in the ASD patients after Nichi Glucan supplementation is due to regulation/prevention of alpha-synuclein's aggregation in the neural synapse must be investigated because an earlier study on beta-glucan from yeast showed reduction in alpha-synuclein expression on the brain substantia nigra in Parkinson's rat model (Masruroh et al., 2017). However, no single mechanism, intervention, or therapy has proven its ability to regulate alpha-synuclein levels, especially in children with ASD. In our study, which is the first of its kind, the plasma alpha-synuclein levels showed significant increase after Nichi Glucan supplementation, and the levels were in line with those that were reported for children without ASD (Kadak et al., 2015; Sriwimol et al., 2018).

Studies on children with ASD have indicated there is an underlying neuroinflammatory process occurring in different regions of the brain involved in microglial activation, thus resulting in a loss of connections or underconnectivity of neurons and leading to behavioural manifestations (Shah et al., 2009). MCP-1, IL-6, IL-10, and TNF-α have been shown to be expressed in higher levels in children with autism (Shah et al., 2009). Beta-glucan has been proven to reduce the expression of inflammatory and proinflammatory markers, including II-6 and TNF-α (Ikewaki et al., 2007), apart from having a neuroprotective effect by attenuating inflammatory cytokine production through microglia (Alp et al., 2012). This mechanism of counteracting ASD inflammation by Nichi Glucan supplementation deserves further research.

In another study, beta-glucan reduced induced microglia activation and its phagocytosis of synaptic puncta and upregulation of proinflammatory cytokine (TNF-α, IL-1β, and IL-6) mRNA expression apart from promoting Tau signalling and improving cognition and brain function via the gut-brain axis (Shi et al., 2020). The mechanism by which the beta-glucan promoted behavioural improvement in the present study and correlated with the regulation of alpha-synuclein levels needs further in-depth research, not only for ASD but also for neurodegenerative diseases such as AD, PD, and so on, especially with regard to its effects on the gut-microbial ecosystem. The evolving data on the gut-brain axis and gut microbiota indicate there are two major approaches to balancing gut microbiota: probiotic and prebiotic.

Probiotic approaches, such as nutritional probiotics, faecal transplantation, and so on, involve direct administration of the beneficial microorganisms that have to colonise the gut (Peng et al., 2020). However, the gut environment must be conducive for such probiotic supplementation. This is where prebiotic approaches come in, such as Nichi Glucan, which help in regulating the gut-microbial ecosystem and preventing chronic inflammatory status Peng et al., 2020); this must be validated by future studies in terms of the effects of Nichi Glucan and gut microbiota in their relevance to ASD.

The limitation of the study is the limited number of participants, the unequal distribution of genders, and the number of participants between the groups. However, this is only a pilot study, and larger randomized, multi-centric clinical trials are warranted. Nevertheless, the study is significant as it has identified a simple nutritional supplemental intervention based on a naturally derived beta-glucan, the Nichi Glucan, which could stimulate endogenous alpha-synuclein secretion, promote better synaptic regulation, and improve the behaviour symptoms of children with autism. However, the results suggest that the benefits will be considerable when evaluated in terms of social and emotional well-being and alleviation of caregiver stress, which is extremely significant.

### Conclusion:

Patients with ASD showed improvement in behavioural symptoms and improved levels of plasma alpha-synuclein; thus, this pilot clinical study of nutritional supplementation with an AFO-202 strain of black yeast Aureobasidium pullulans produced the biological response modifier beta-glucan (Nichi Glucan). Evaluation as per the CARS score has also shown significant beneficial effects. Although further validations need to be performed, the study definitely confirms the potential of Nichi Glucan as a simple but effective food supplement to be considered as a routine in children with ASD. Further research on the mechanisms of its action in improving alpha-synuclein levels and balancing the immune system in the context of managing chronic inflammation and gut-microbiota regulation as a prebiotic is likely to improve understanding of other diseases caused by neuroinflammation such as PD and AD.

Comparison of Different Beta Glucans with AFO-202
Alpha-Synuclein Suppression Data from F26S Study

### Methods

Neuroblastoma cell line (SHSY-5Y Tet-On: SCC291) (MERCK) was suspended in 10% FCS-DMEM/F12 medium and seeded into 96-well microplates to achieve a cell count of 5×104 cells/well. After 24 hours of incubation, the cells were washed with 10% FCS-DMEM/F12 medium and new 10% FCS-DMEM/F12 medium was added.

Then, the different beta glucans (Product 1: Micelle Glucan(R) (gel or liquid type) purchased from RL-JP Co., Japan; Product. 2: Beta-glucan NEW EX (gel or liquid type) purchased from Aureo BIS, Japan; Product 3: Yeast Glucan (capsule, inside powder?) purchased from Shell Life Japan Co., Japan) were added to the medium, and the cells were cultured for 1 day. After incubation, the wells were washed three times with PBS and fixed in 0.25% glutaraldehyde-PBS for 1 hour at room temperature. After fixation, the wells were washed three times with PBS-0.05% Tween (PBS-T). After washing the wells with PBS-T, α-synuclein polyclonal antibody (proteintech Co. 10842-1-AP; x1,000 dilution) was added to the wells and incubated at room temperature for 1 h. Then, 2% BSA-PBS-T was added and left for 1 h (to block non-specific reactions).

After 1 hour, the wells were washed with PBS-T and 50 μL of biotin-labeled anti-rabbit IgG antibody (Cosmo Bio) diluted 5,000-fold was added to the wells and the reaction was carried out for 40 minutes at room temperature. After washing the wells, 50 μL of 10,000-fold diluted peroxidase-labeled streptavidin (Cosmo Bio) was added and the reaction was carried out for 20 minutes at room temperature. After washing the wells again, 50 μL of TMB (Cosmo Bio) was added to the wells, and the reaction was stopped with 0.5 M-HCl for 10 minutes.

After that, the absorbance (OD value) was measured with a microplate reader (450 nm) (Tosoh). Data were expressed as ΩOD value (sample OD value−blank OD value). The expression rate was calculated based on the control (None).

### Results:

Results are found in Table 10.

TABLE 10
Expression of α-synuclein on the neuroblastoma cell line
(SHSY-5Y Tet-On: SCC291) treated with several beta-glucans (BGs)

| | δOD: 450 nm | Expression % | Suppression % |
|---|---|---|---|
| Treatment | Mean ± SD | vs. None | vs. None |
| None | 0.776 ± 0.0211 | 100.0 | 0.0 |
| P.1: Micelle BG | 0.733 ± 0.1047 | 94.46 | 5.54 |
| 50 μg/mL | | | |
| P.2: [symbol] BG | 0.709 ± 0.0075 | 91.41 | 8.59 |
| 50 μg/mL | | | |
| P.3: [symbol] BG | 0.745 ± 0.0111 | 96.05 | 3.96 |
| 50 μg/mL | | | |
| AFO-202 | 0.630 ± 0.0644 | 81.19 | 18.81 |
| 50 μg/mL | | | |

[symbol]

indicates data missing or illegible when filed

Decrease in α-synuclein expression was highest in AFO-202 beta glucan. Since these cell lines produced α-synuclein are considered to be abnormal/misfolded and capable of aggregation, their decrease in expression is considered to be a suppression of production of abnormal α-synuclein and hence an advantage.

### Improving Sleep Pattern and Serum Melatonin

### Introduction:

Sleep problems are reported in 50 to 80% of children with Autism and Autism Spectrum disorders (ASD) [C1]. In adolescents and older children with ASD, sleep problems are higher including delayed sleep onset, shorter sleep duration and daytime sleepiness whereas in younger children bedtime resistance, sleep anxiety, parasomnias and night waking are predominant [C2]. Problems in sleep exacerbate the other features of autism such as tantrums, aggression, self-injury, inattention, hyperactivity, social interactions and repetitive behaviours, adding to the parental stress and the entire family's well-being [C1, C3].

Melatonin, a neurohormone secreted by the pineal gland which regulates circadian rhythms including sleep patterns has been shown to be released at lower levels in individuals with autism and has been shown to have a positive effect on sleep in autism by acting on relieving Anxiety, improving sensory processing, possess anti-nociceptive effects on pain, and also gastro-intestinal dysfunction or gut dysbiosis [C3]. A significant proportion of children with ASD have chronic gastrointestinal problems such as diarrhea and/or constipation, irritable bowel syndrome etc. These GIT symptoms have been related cortisol response to stress and gut dysbiosis induced chronic inflammation in ASD [C2] which in turn has associations with altered melatonin levels in autism [C2]. Thus, melatonin supplementation [C2,C4,C5] is one of the main pharmacological approaches under consideration for ASD.

Clinical studies of supplemental melatonin in ASD children have shown to improve sleep latency and quality [C2,C4,C5] in varying degrees [C3]. It is also to be noted that melatonin, though the side effects have been reported to be minimal, it has been found to be effective mostly in short term treatment of sleep disorders and the positive effects have waned during follow up (6-12 months) in specific clinical studies [C6]. Beta (β)-glucans which are naturally occurring compounds have been shown to have a wide range of biological response modifying beneficial effects in metabolism, anti-cancer as well as in reducing the stress and mental disorders by acting on the immune system related pathways [C7]. An animal study has earlier shown that melatonin levels were upregulated in the blood serum of rats in the presence of rice bran (RB) and Beta (β)-glucan present in a mushroom Sarcodon aspratus (S)'s extracts [C7,C8]. We and other research teams have earlier reported the beneficial effects of Nichi Glucan, a black yeast (Aureobasidium pullulans) AFO-202 derived 1,3-,16 beta glucan in metabolic disorders [C9,C10], cancer [C11,C12] in human clinical studies and as a suggested vaccine adjuvant for COVID-19 [C13]. Herein we undertook to study the effects of Nichi Glucan on sleep pattern and serum melatonin levels of ASD children in this pilot clinical study.

### Example 4

### Materials and Methods:

Thirteen children with ASD, four in the control group (Gr.1) and nine in the treatment group (Gr.2) age range 2.5 to 13 years were included in the study. The subjects of Gr.2 consumed 1 gram of Nichi glucan (Aureobasidium pullulans strain AFO-202 (also referred to as FO-68 [(accession number) FERM BP-19327]) derived Beta 1,3-1,6 Glucan) as food supplement along with conventional therapies while Gr. 1 underwent conventional therapies alone for a duration of 90 days. The serum melatonin levels were evaluated before and after the study along with assessment of the subjective parameters in sleep pattern by means of a questionnaire to the caregiver in both the groups.

### Results:

In the Nichi Glucan supplementation group (Gr. 2), the serum melatonin increased on an average from 238.85 ng/dl pre-intervention to 394.72 ng/dl post-intervention which was greater than the control group (Gr.1). All the children in the Nichi Glucan group (Gr.2) showed improvement in sleep pattern and quality.

### Conclusion:

Aureobasidium pullulans derived Beta 1,3-1,6 Glucan after 90-days consumption has shown visible improvement in sleep quality, pattern and serum melatonin levels in this first of its kind report in the literature which warrants a larger multicentric study for validation and in-depth research on the mechanisms to recommend this as a routine supplementation in kids with ASD to improve their quality of sleep.

### Example 5

### Materials and Methods:

This study was approved by our Institutional ethics committee of Kenmax Medical Service Private Limited, Madurai, India and registered in the Clinical trial registry of India (CTRI/2020/10/028322).

### Study Design:

The subjects enrolled in the study had clinical diagnosis of ASD by a developmental paediatrician using standard assessment verified using a clinical interview that incorporated CARS (Childhood Autism Rating Scale).

Eighteen subjects (n=18) with ASD in total were enrolled in this prospective open label pilot clinical trial comprising of two arms,

Arm 1 or Group (Gr.) 1: Control: Six subjects with ASD (n=6) underwent conventional treatment which comprised of remedial behavioural therapies and L-Carnosine 500 mg per day.

Arm 2 or Group (Gr.) 2: Treatment arm: Twelve subjects (n=12) underwent supplementation with Nichi Glucan (Aureobasidium pullulans strain AFO-202 (also referred to as FO-68 [(accession number) FERM BP-19327]) derived Beta 1,3-1,6 Glucan) food supplement along with conventional treatment. The subjects consumed 2 sachets (0.5 g each) of Nichi Glucan, one sachet with a meal twice daily for a period of 90 days.

### Inclusion Criteria:

i. Age: 3 to 18 years;
ii. 2. Gender: Both male and female;
iii. ASD criteria as per CARS (Childhood Autism Rating Scale) score; and,
iv. Parents willing to consent for their children for actively participating in the study.

### Exclusion Criteria:

i. Subjects aged more than 18 years old;
ii. Any child with acute general illness or is on any antibiotic, anti-inflammatory, or antioxidant treatment in the two weeks prior to enrolment in the study;
iii. Hyperallergic to any of the investigational products; and,
iv. Subjects with long standing infections

### Outcome Measures:

i. Sleep pattern assessment by questionnaire:
The Parent or caregiver completed a survey questionnaire, the Children's Sleep Habits Questionnaire-Abbreviated (CSHQ-A) was used in this research to assess the sleep problems which consisted of 22 questions (NICHD SECCYD-Wisconsin), with adaptations to suit the local cultural and social conditions.
ii. Evaluation of serum melatonin:
Melatonin levels in serum were measured in peripheral blood collected at daytime (and evaluation was performed using Human Melatonin ELISA Kit (BT-LAB-Bioassay Technology Laboratory kit, China)

### Data Analysis:

All data were analysed using Excel software statistics package analysis software (Microsoft Office Excel(R)); Student's paired t-tests were also calculated using this package; P-values<0.05 were considered significant.

### Results:

During enrolment, six subjects with ASD (n=6) could be enrolled in the control Gr.1 while in treatment group (Gr. 2), one of them dropped out even before start of the study. During the study, three subjects were lost to follow-up, one in Gr.1 (subject relocated to another city) and 2 in Gr. 2 (one due to social problems in the family and other relocated to another city). Totally 13 subjects (4 in Gr.1 and 9 in Gr.2) completed the study. There was one female subject in both Gr.1 and Gr. 2. The rest were male.

### Improvement in Sleep Pattern:

On the Children's Sleep Habits related Questionnaire (CSHQ), there was significant reduction in the total score especially in terms of decrease in bedtime resistance and time of onset of sleep in the Gr.2 compared to Gr.1 (Table 11). The total sleep score ranged from 66 to 67 in Gr.1 (Mean=66.25±0.5) in the Gr.1 Control group while it ranged from 62 to 75 in Gr.2 at baseline (Mean=72±5.02) in Gr.2 (Nichi Glucan) at baseline. At the end of the study the total sleep score ranged from 58 to 66 in Gr.1 (Mean=64±4) in the Gr.1 Control group while it ranged from 51 to 70 in Gr.2 (Mean=64.22±7.47) in Gr.2 (Nichi Glucan). The reduction in sleep score after intervention indicating improvement in sleep behaviour was statistically significant in Gr.2 (p value=0.009879) indicating a significant improvement in the sleep patterns of the subject in the Nichi Glucan arm while the difference in sleep score did not show any statistically significant improvement in the control arm (p value=0.153494). The total sleep score also decreased well in the Nichi Glucan group compared to the control (FIG. 28).

TABLE 11
Results of Children's Sleep Habits related Questionnaire (CSHQ), with significant
reduction in the total score indicating improvement in bedtime resistance and
time of onset of sleep in Nichi Glucan Gr. 2 compared to Control, Gr. 1

| | Gr. 1 (Mean values) | | Gr. 2 (Mean values) | |
|---|---|---|---|---|
| Parameters | Baseline | End of Study | Baseline | End of Study |
| Bedtime resistance | 28 | 25.75 | 28.5556 | 23.2222 |
| Time of Onset of Sleep | 20.25 | 20.25 | 21.8889 | 19.4444 |
| Duration of Sleep | 6 | 6 | 6.44444 | 5.55556 |
| Night waking | 6 | 6 | 6 | 6 |
| Day-time sleepiness | 6 | 6 | 9.11111 | 10 |

### Serum Melatonin Levels:

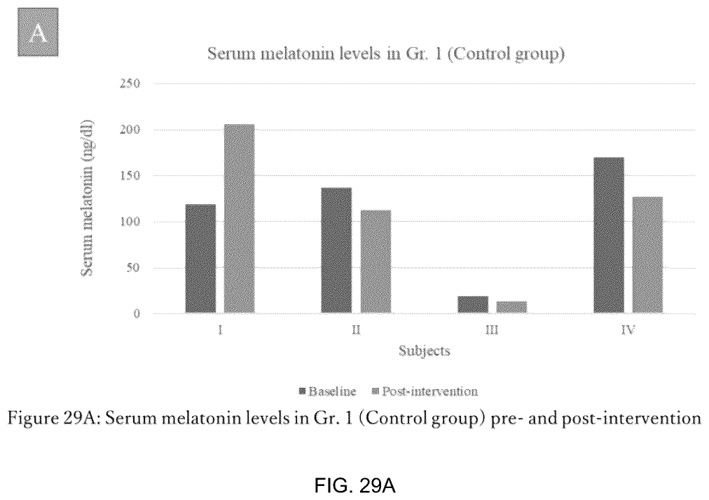

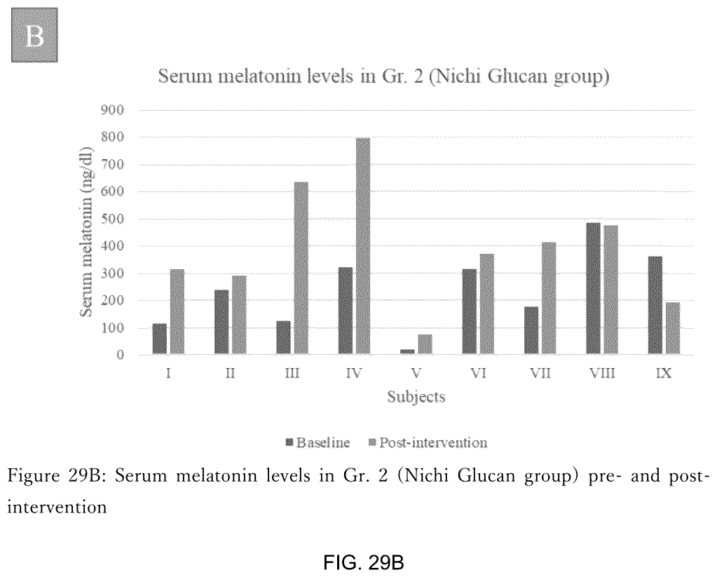

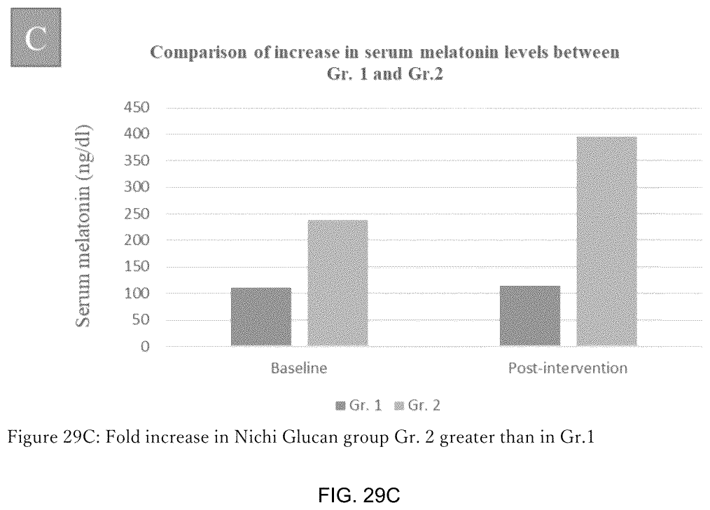

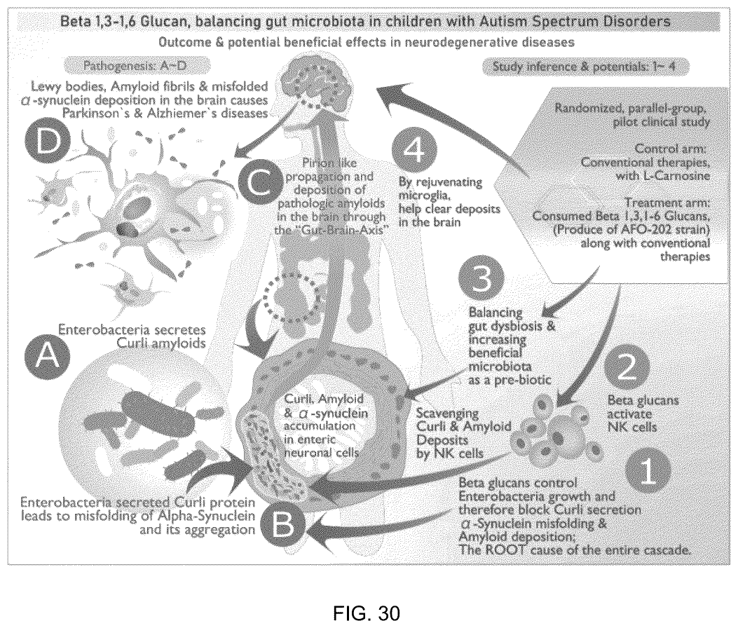

In the control group (Gr.1), the serum Melatonin increased on an average from 110.585 to only 114.11 post-intervention (FIG. 29A) while in the Nichi Glucan supplementation group (Gr.2), the serum Melatonin increased on an average from 238.85 ng/dl pre-intervention to 394.72 ng/dl post-intervention (FIG. 29B). The fold increase in Nichi Glucan group Gr. 2 was 2.29 compared to 1 in Gr.1 (FIG. 29C) though higher in Gr.2 was not statistically significant (p-value=0.065786)

### Adverse Effects:

Only one child exhibited possible mild adverse effects related to increased bowel movements in Gr. 2 for one week after supplementation with Nichi Glucan which settled on its own. There were no adverse effects in any of the other children.

### Discussion:

In this open-label clinical trial of supplementation with Nichi Glucan, we found that majority of the children in the Nichi Glucan group (Gr.2), 8 out of 9 subjects (88%) had an improvement in sleep pattern and quality of sleep observed by decrease in sleep score after Nichi Glucan supplementation. The serum melatonin increased to a greater extent in Gr. 2 compared to Gr.1. The sleep score significantly decreased in Gr. 2 compared to gr.1 (FIG. 28).

There were only minimal adverse effects. This is the first of its kind study, in which a nutritional supplement that is not a pharmacological drug has been able to improve sleep pattern with evidence in laboratory evaluation of corresponding serum melatonin and in children with ASD.

Sleep difficulties are a major problem in children with ASD with 53% having been reported to have difficulty in sleep onset (53%), 40% restless sleep, 34% night-time awakening and 32% difficulty in arousal from sleep [C14]. Lack of good sleep also affects emotional and functioning ability in turn leading to impairment in academic and social functioning and maintaining relationships in these children. Therefore, ensuring good quality sleep becomes an essential part of therapy for ASD. Among pharmacological interventions, melatonin [C2,C4,C5], trazodone, benzodiazepines, and SSRI antidepressants represent the most commonly used medications in the paediatric population [C15]. Melatonin supplementation remains the treatment of choice, given the side effects of other interventions and clinical trials having showed positive outcome of its supplementation [C15]. Nevertheless, there are reports that melatonin is more effective as short term rather than long term though those studies have mostly been in individuals without ASD [C6].

A nutritional supplement which can be simple, easy to administer and has minimal or no adverse effects will be an ideal alternative to melatonin. In the current study, Nichi Glucan which has been in consumption as a food supplement for several decades [C16] with proven benefits in metabolic disorders, cancer etc. [C9-12] has been shown to be a promising strategy based on the current study's results in terms of improvement in quality of sleep and increase in daytime serum melatonin levels.

It has been postulated that low melatonin levels in ASD children could have its etiologic origin in melatonin deficiency in mothers of these children exerting its effects during neurodevelopment in embryo [C17]. Another study has reported the clear correlation between gut microbiome profiles of children with ASD and their mothers suggesting the importance for assessing the microbiome during the early stage in mothers during pregnancy and planning of personalized treatment and prevention of ASD via microbiota modulation [C17]. Beta glucan has also been shown to reduce the underlying chronic inflammation due to gut dysbiosis and helping to modulate towards a healthy microbiome, which will be further advantageous in ASD as chronic inflammation has been shown to be associated with severity of ASD symptoms [C18], Thus, with the current study showing that beta glucans can enhance melatonin and sleep quality in children with ASD, the ability of Beta glucans to modulate gut microbiota and reverse gut dysbiosis as the possible mechanism behind the increase in levels of melatonin [C6,C19,C20], thereby improving sleep, needs further research.

This is only a pilot study and the limitation with the very less ample number is planned to be overcome by additional large-scale studies apart from studying the possible beneficial effects of Nichi Glucan on the behavioural aspects and other symptoms in patients with ASD.

### Conclusion:

Patients with ASD have shown improvement in quality of sleep and improved levels of serum melatonin, in this open label pilot clinical study of nutritional supplementation with an AFO-202 strain of black yeast Aureobasidium pullulans produced 1,3-1,6 beta Glucan (Nichi-Glucan). The efficacy of Nichi Glucan in terms of behavioural improvement and other parameters observed in this pilot study in children with ASD, when confirmed in a larger study with long-term follow-up, it is worth recommending it as a supplementary food in such children. Further in-depth evaluation of the mechanisms and their correlation with other neurological parameters is recommended, which may throw light on novel solutions and drug candidates from such findings.

### Melatonin and Gut Microbiome Correlation

### AFO-202 Study

### Methods

The study involved 18 subjects with ASD who were randomly allocated: six subjects in the control group (Group 1) underwent conventional treatment comprising remedial behavioural therapies and L-carnosine 500 mg per day, and 12 subjects (Group 2) underwent supplementation with Nichi Glucan 0.5 g twice daily along with the conventional treatment for 90 days. The subjects' stool samples were collected at baseline and after the intervention.

Whole genome metagenome (WGM) sequencing was performed.

### Results

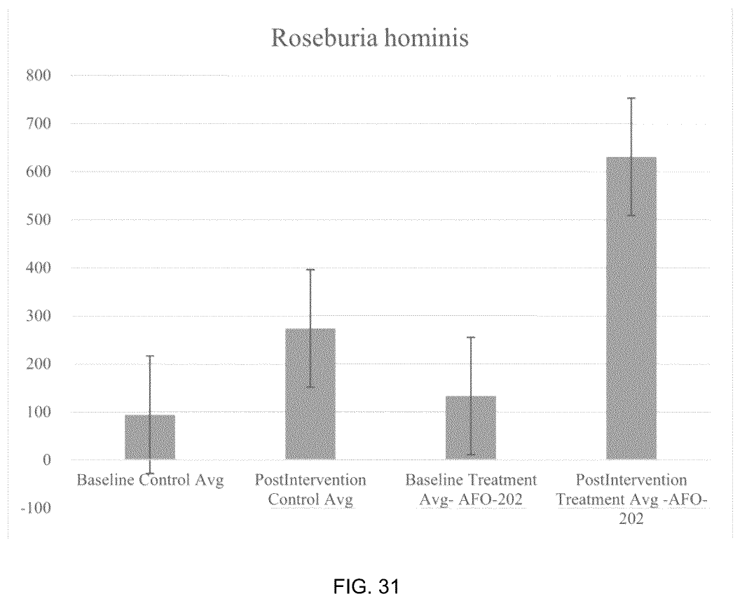

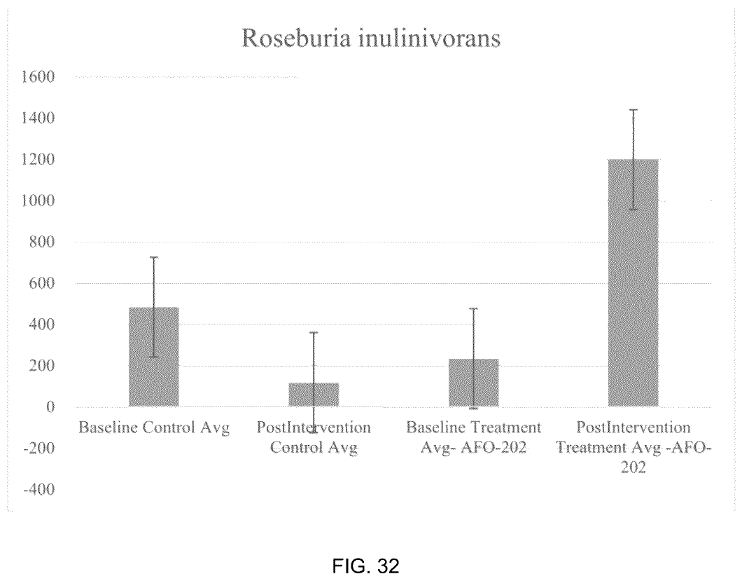

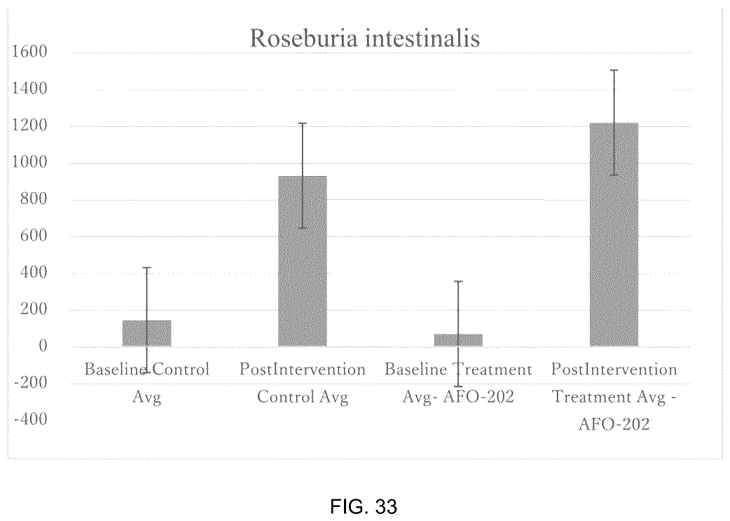

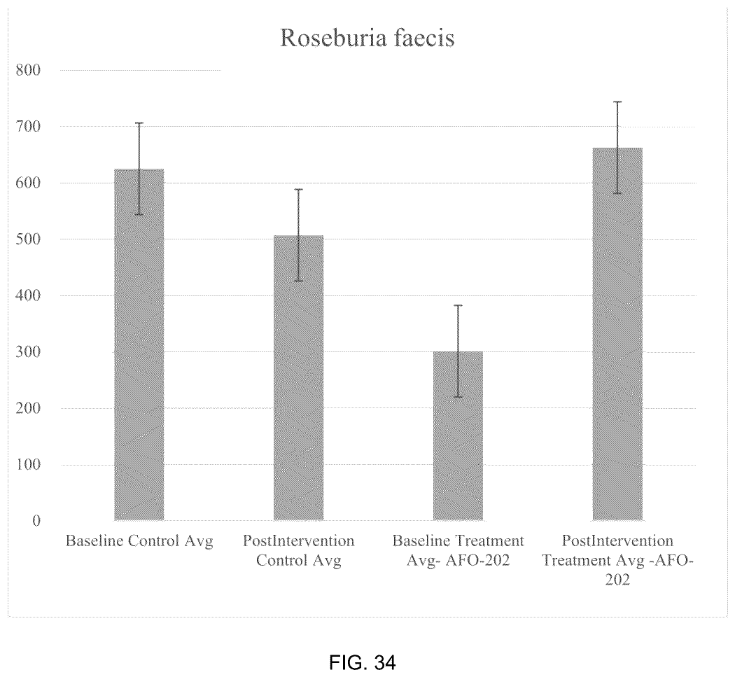

The results are shown in FIGS. 31-34.

### Inference:

All the species, R. Hominis, R. intestinalis, R. inulinivorans and R. faecis increased greatly post-intervention in AFO-202 treatment group. R. inulinivorans and R. faecis decreased in the control group. The increase in melatonin and improved sleep reported in the study (doi: 10.21203/rs.3.rs-701988/v1) can be attributed to the increase in abundance of Roseburia.

### F5s gut Microbiome in Autism and Epilepsy

### Seed Average of Gut Microbiome

### What is SEED?:

In 2004, the SEED (http://pubseed.theseed.org/) was created to provide consistent and accurate genome annotations across thousands of genomes and as a platform for discovering and developing de novo annotations. The SEED is a constantly updated integration of genomic data with a genome database, web front end, API and server scripts. It is used by many scientists for predicting gene functions and discovering new pathways

### Methods:

Eighteen subjects with ASD were enrolled in this prospective, open-label, pilot clinical trial comprised of two arms. Arm 1 or Group 1 (control group): Six subjects with ASD underwent conventional treatment comprising remedial behavioural therapies and L-carnosine 500 mg per day. Arm 2 or Group 2 (Nichi Glucan group): 12 subjects underwent supplementation with Nichi Glucan food supplement along with conventional treatment (remedial behavioural therapies and L-carnosine 500 mg per day). Each subject consumed two sachets (0.5 g each) of Nichi Glucan daily—one sachet with a meal twice daily—for 90 days.

Faecal samples were collected at baseline and 90 days after the intervention using a sterile faecal collection kit and the samples were kept at −20° C. until they were transferred to the laboratory and processed. Samples for DNA extraction were stored at −80° C. until needed for analysis.

The samples were then taken for whole genome metagenome analysis. Initially, the reads were filtered for human DNA contamination. The filtered reads were then aligned to bacterial, fungal, viral and archea genomes. De novo assembly was carried out using the pre-processed reads to obtain the scaffolds. These scaffolds were then used for gene prediction. The abundances in terms of SEED annotations were analysed.

### Results

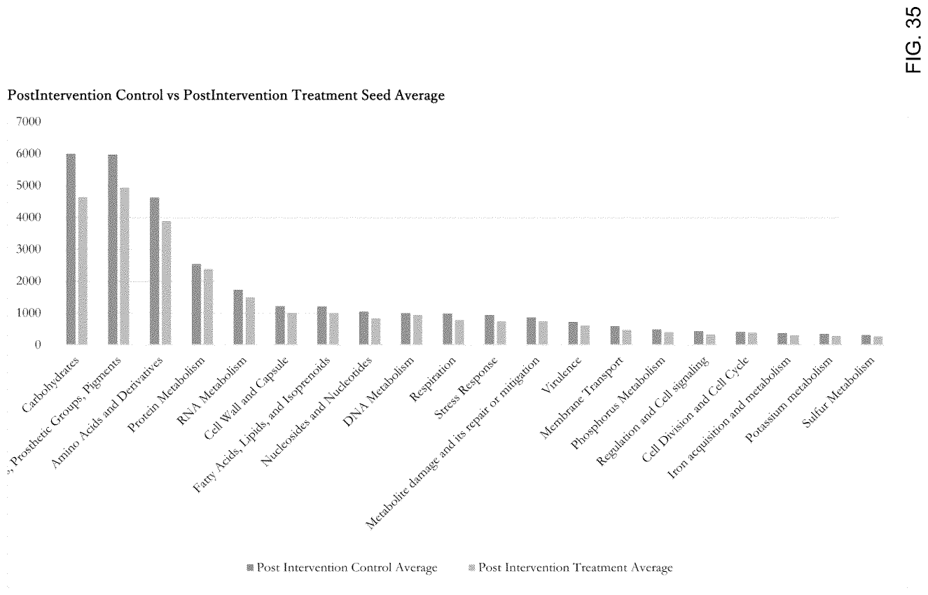

The results are shown in FIG. 35 and Table 9.

### Interpretation

There is several fold decrease in all the gene annotations (metabolites and metabolic functions) in the AFO-202 Nichi Glucan treatment group including Carbohydrates, Fatty acids, lipids, virulence, metabolite damage, nitrogen metabolism, mitochondrial electron transport system etc.

### Inference:

1. Most of the metabolic pathways and related functional genes are enriched or elevated in Autism and epilepsy;
2. A ketogenic diet which is advocated for autism and epilepsy has shown decrease in these pathways by SEED analysis in other studies; and,
3. Therefore, the several fold decrease in the SEED relative average data in AFO-202 group in the present study shows the benefits of this beta glucan in decreasing the metabolic pathways of the gut microbiota which is responsible for the positive clinical outcome reported in the study
   F5S Study—Alpha-Synuclein-Support Data from F26S Study:

### Methods

Neuroblastoma cell line (SHSY-5Y Tet-On: SCC291) (MERCK) was suspended in 10% FCS-DMEM/F12 medium and seeded into 96-well microplates to achieve a cell count of 5×104 cells/well. After 24 hours of incubation, the cells were washed with 10% FCS-DMEM/F12 medium and new 10% FCS-DMEM/F12 medium was added.

Then, β-glucan AFO-202 (50 μg/mL) and PMA (500 ng/mL) (Sigma) were added to the medium, and the cells were cultured for 1 to 3 days. After incubation, the wells were washed three times with PBS and fixed in 0.25% glutaraldehyde-PBS for 1 hour at room temperature. After fixation, the wells were washed three times with PBS-0.05% Tween (PBS-T). After washing the wells with PBS-T, 50 μL of 500-fold diluted α-synuclein polyclonal rabbit antibody (proteintech Co. 10842-1-AP) was added to the wells and incubated at room temperature for 1 h. Then, 2% BSA-PBS-T was added and left for 1 h (to block non-specific reactions).

After 1 hour, the wells were washed with PBS-T and 50 μL of biotin-labeled anti-rabbit IgG antibody (Cosmo Bio) diluted 5,000-fold was added to the wells and the reaction was carried out for 40 minutes at room temperature. After washing the wells, 50 μL of 10,000-fold diluted peroxidase-labeled streptavidin (Cosmo Bio) was added and the reaction was carried out for 20 minutes at room temperature. After washing the wells again, 50 μL of TMB (Cosmo Bio) was added to the wells, and the reaction was stopped with 0.5 M-HCl for 10 minutes.

After that, the absorbance (OD value) was measured with a microplate reader (450 nm) (Tosoh). Data were expressed as ΩOD value (sample OD value−blank OD value). The expression rate was calculated based on the control (None).

### Results

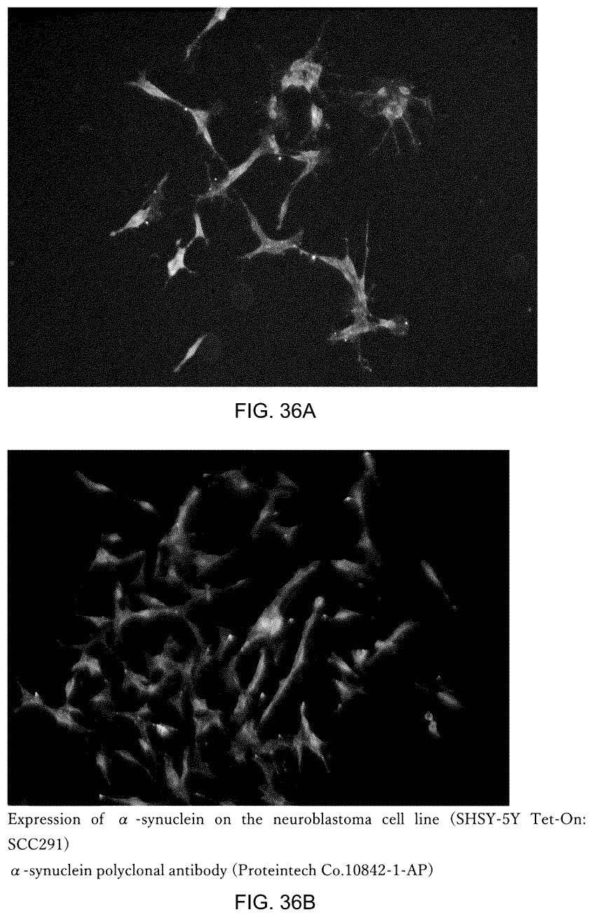

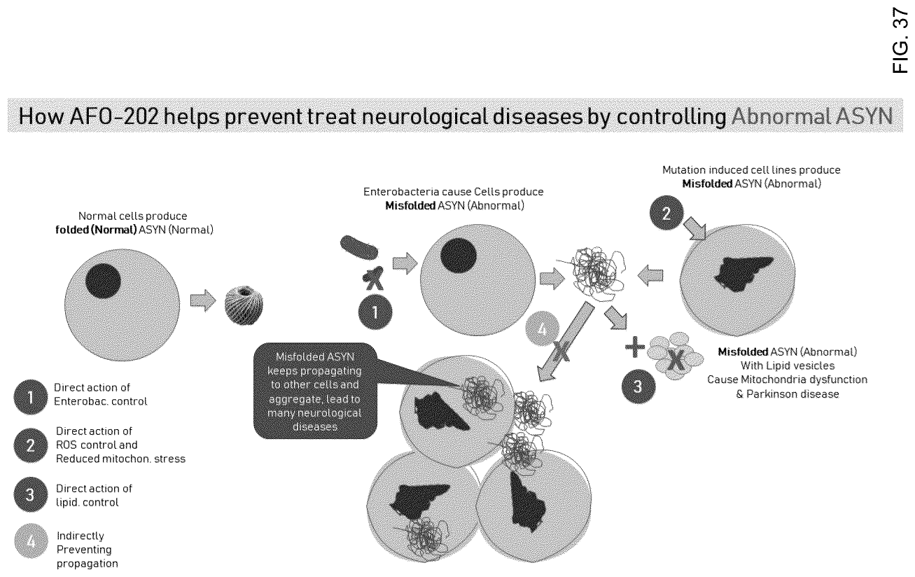

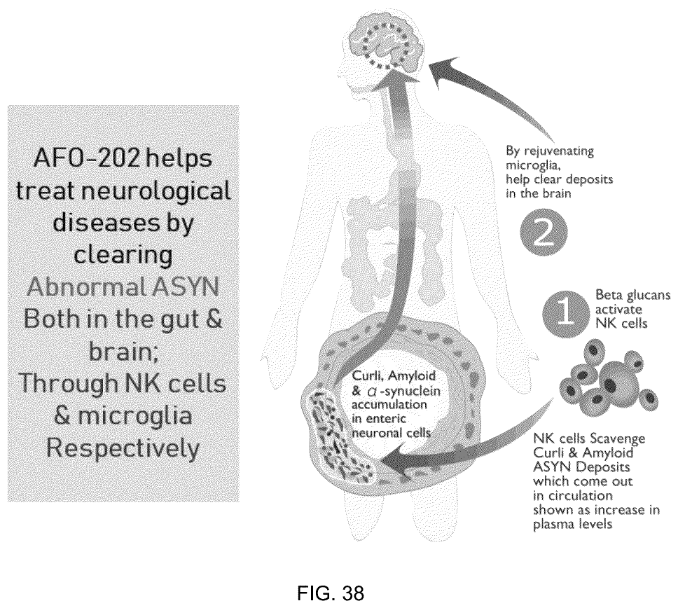

The results are shown in FIGS. 36A-B and Table 12 below.

TABLE 12
Expression of α-synuclein on the neuroblastoma cell line
(SHSY-5Y Tet-On: SCC291) stimulated beta-glucans(BGs) or PMA

| | | | | α-synuclein |
|---|---|---|---|---|
| | | | | expression (%) |
| Stimulation | Concentration | | δOD value | vs. None |
| None | | | 0.281 | 100.0 |
| BG-AF202 | 50 | μg/mL | 0.198 | 70.5 |
| PMA | 500 | ng/mL | 0.166 | 50.1 |

The neuroblastoma cell line (SHSY-5Y Tet-On: SCC291) was stimulated with BGs or PMA for 3 days. An α-synuclein polyclonal antibody (proteintech Co. 10842-1-AP) was used in this experiment (glutaraldehyde-fixed cellular ELISA).

### AFO-202 Beta Glucan Decreases α-Synuclein Expression

### Interpretation-I

Alpha synuclein expression is decreased by AFO-202 in cell lines. Since these cell lines produced ASN are abnormal/misfolded and capable of aggregation, their decrease in expression is considered to be a suppression of production of abnormal ASN and hence an advantage.

### Interpretation-II

Beta Glucans having been able to control ROS and mitochondrial Stress and hence the above suppression of abnormal αSyn could be attributed due to that mechanism.

### Interpretation-III

It is the abnormal ASN causing aggregates which can show transmission from cell to cell and prone to propagation like prions through gut brain axis and therefore AFO-202 making their production lesser at cellular level is helping to address the issue at the root cause itself.

### Interpretation-IV

Since AFO-202 can regulate dyslipidemia and because alpha-synuclein binding with oxidized lipid metabolites can lead to mitochondrial dysfunction, leading to neuronal disorders, AFO-202 (i) decreasing misfolded alpha-synuclein production and (ii) regulating lipids has an advantage.

### Interpretation-V

The increase in plasma levels of Alpha-Synuclein in the autism children (http://dx.doi.org/10.1136/bmjno-2021-000203) can be attributed to the clearing of the deposits by NK cells activated by AFO-202 beta glucan.

### Interpretation-VI

Capacity of beta glucans to activate microglia will be of advantage in clearing the aggregates in the CNS therefore attributing to the behaviour and sleep pattern improvement of AFO-202 in the autism study (http://dx.doi.org/10.1136/bmjno-2021-000203)

### Modifications and Other Embodiments

Various modifications and variations of the described glucan products, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the chemical, biological, medical, environmental, cosmetic or food arts or related fields are intended to be within the scope of the following claims.

### REFERENCES

A1. Miller A L, Bessho S, Grando K, Tükel Ç. Microbiome or Infections: Amyloid-Containing Biofilms as a Trigger for Complex Human Diseases. Front Immunol. 2021 Feb. 26; 12:638867.
A2. Kang D W, Park J G, Ilhan Z E, Wallstrom G, Labaer J, Adams J B, Krajmalnik-Brown R. Reduced incidence of Prevotella and other fermenters in intestinal microflora of autistic children. PLoS One. 2013 Jul. 3; 8(7):e68322.
A3. Liu F, Li J, Wu F, Zheng H, Peng Q, Zhou H. Altered composition and function of intestinal microbiota in autism spectrum disorders: a systematic review. Transl Psychiatry. 2019 Jan. 29; 9(1):43.
A4. Shahi S K, Freedman S N, Murra A C, Zarei K, Sompallae R, Gibson-Corley K N, Karandikar N J, Murray J A, Mangalam A K. Prevotella histicola, A Human Gut Commensal, Is as Potent as COPAXONE(R)(R) in an Animal Model of Multiple Sclerosis. Front Immunol. 2019 Mar. 22; 10:462
Al-Mazidi, S., & Al-Ayadhi, L. Y (2021). Plasma Levels of Alpha and Gamma Synucleins in Autism Spectrum Disorder: An Indicator of Severity. Medical principles and practice: international journal of the Kuwait University, Health Science Centre, 30(2), 160-167.
Bartels, T., De Schepper, S., & Hong, S. (2020). Microglia modulate neurodegeneration in Alzheimer's and Parkinson's diseases. Science (New York, N.Y.), 370(6512), 66-69.
Boziki, M. K., Kesidou, E., Theotokis, P., Mentis, A. A., Karafoulidou, E., Melnikov, M., Sviridova, A., Rogovski, V., Boyko, A., & Grigoriadis, N. (2020). Microbiome in Multiple Sclerosis; Where Are We, What We Know and Do Not Know. Brain sciences, 10(4), 234.
Ding, X., Xu, Y, Zhang, X., Zhang, L., Duan, G., Song, C., Li, Z., Yang, Y, Wang, Y, Wang, X., & Zhu, C. (2020). Gut microbiota changes in patients with autism spectrum disorders. Journal of psychiatric research, 129, 149-159.
DeAngelis, M., Piccolo, M., Vannini, L., Siragusa, S., De Giacomo, A., Serrazzanetti, D. I., Cristofori, F., Guerzoni, M. E., Gobbetti, M., & Francavilla, R. (2013). Fecal microbiota and metabolome of children with autism and pervasive developmental disorder not otherwise specified. PloS one, 8(10), e76993.
Dedeepiya, V. D., Sivaraman, G., Venkatesh, A. P., Preethy, S., & Abraham, S. J. (2012). Potential effects of nichi glucan as a food supplement for diabetes mellitus and hyperlipidemia: preliminary findings from the study on three patients from India. Case reports in medicine, 2012, 895370.
Dilsizoglu Senol, A., Samarani, M., Syan, S., Guardia, C. M., Nonaka, T., Liv, N., Latour-Lambert, P., Hasegawa, M., Klumperman, J., Bonifacino, J. S., & Zurzolo, C. (2021). α-Synuclein fibrils subvert lysosome structure and function for the propagation of protein misfolding between cells through tunneling nanotubes. PLoS biology, 19(7), e3001287.
Earls, R. H., Menees, K. B., Chung, J., Gutekunst, C. A., Lee, H. J., Hazim, M. G., Rada, B., Wood, L. B., & Lee, J. K. (2020). NK cells clear α-synuclein and the depletion of NK cells exacerbates synuclein pathology in a mouse model of α-synucleinopathy. Proceedings of the National Academy of Sciences of the United States of America, 117(3), 1762-1771.
Ganesh, J. S., Rao, Y. Y., Ravikumar, R., Jayakrishnan, G. A., Iwasaki, M., Preethy, S., & Abraham, S. J. (2014). Beneficial effects of black yeast derived 1-3, 1-6 Beta Glucan-Nichi Glucan in a dyslipidemic individual of Indian origin—a case report. Journal of dietary supplements, 11(1), 1-6.
Grimaldi, R., Gibson, G. R., Vulevic, J., Giallourou, N., Castro-Mejia, J. L., Hansen, L. H., Leigh Gibson, E., Nielsen, D. S., & Costabile, A. (2018). A prebiotic intervention study in children with autism spectrum disorders (ASDs). Microbiome, 6(1), 133.
^{a}Ikewaki N, Onaka T, Ikeue Y, Nagataki M, Kurosawa G, Dedeepiya V D, Rajmohan M, Vaddi S, Senthilkumar R, Preethy S, Abraham S J K. Beneficial effects of the AFO-202 and N-163 strains of Aureobasidium pullulans produced 1,3-1,6 beta glucans on non-esterified fatty acid levels in obese diabetic KKAy mice: A comparative study.bioRxiv 2021.07.22.453362; doi: 10.1101/2021.07.22.453362
^{b}Ikewaki N, Kurosawa G, Iwasaki M, Preethy S, Dedeepiya V D, Vaddi S, Senthilkumar R, Levy G A, Abraham S J K. Hepatoprotective effects of Aureobasidium pullulans derived Beta 1,3-1,6 biological response modifier glucans in a STAM-animal model of non-alcoholic steatohepatitis.bioRxiv 2021.07.08.451700; doi: 10.1101/2021.07.08.451700
^{c}Ikewaki N, Sonoda T, Kurosawa G, Iwasaki M, Dedeepiya V D, Senthilkumar R, Preethy S, Abraham S J K. Immune and metabolic beneficial effects of Beta 1,3-1,6 glucans produced by two novel strains of Aureobasidium pullulans in healthy middle-aged Japanese men: An exploratory study. medRxiv 2021.08.05.21261640. doi: 10.1101/2021.08.05.21261640
Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol. 2007; 51:861-73.
Kang, D. W., Adams, J. B., Coleman, D. M., Pollard, E. L., Maldonado, J., McDonough-Means, S., Caporaso, J. G., & Krajmalnik-Brown, R. (2019). Long-term benefit of Microbiota Transfer Therapy on autism symptoms and gut microbiota. Scientific reports, 9(1), 5821.
Kang, D. W., Park, J. G., Ilhan, Z. E., Wallstrom, G., Labaer, J., Adams, J. B., & Krajmalnik-Brown, R. (2013). Reduced incidence of Prevotella and other fermenters in intestinal microflora of autistic children. PloS one, 8(7), e68322.
Kang, D. W., Ilhan, Z. E., Isern, N. G., Hoyt, D. W., Howsmon, D. P., Shaffer, M., Lozupone, C. A., Hahn, J., Adams, J. B., & Krajmalnik-Brown, R. (2018). Differences in fecal microbial metabolites and microbiota of children with autism spectrum disorders. Anaerobe, 49, 121-131.
Lindefeldt, M., Eng, A., Darban, H., Bjerkner, A., Zetterström, C. K., Allander, T., Andersson, B., Borenstein, E., Dahlin, M., & Prast-Nielsen, S. (2019). The ketogenic diet influences taxonomic and functional composition of the gut microbiota in children with severe epilepsy. NPJ biofilms and microbiomes, 5(1), 5.
Luna, E., & Luk, K. C. (2015). Bent out of shape: α-Synuclein misfolding and the convergence of pathogenic pathways in Parkinson's disease. FEBS letters, 589(24 Pt A), 3749-3759.
Miller, A. L., Bessho, S., Grando, K., & Tükel, Ç. (2021). Microbiome or Infections: Amyloid-Containing Biofilms as a Trigger for Complex Human Diseases. Frontiers in immunology, 12, 638867.
Morato Torres, C. A., Wassouf, Z., Zafar, F., Sastre, D., Outeiro, T. F., & Schüle, B. (2020). The Role of Alpha-Synuclein and Other Parkinson's Genes in Neurodevelopmental and Neurodegenerative Disorders. International journal of molecular sciences, 21(16), 5724.
Murros, K. E., Huynh, V. A., Takala, T. M., & Saris, P. (2021). Desulfovibrio Bacteria Are Associated With Parkinson's Disease. Frontiers in cellular and infection microbiology, 11, 652617.
Oh, D., & Cheon, K. A. (2020). Alteration of Gut Microbiota in Autism Spectrum Disorder: An Overview. Soa—ch'ongsonyon chongsin uihak=Journal of child & adolescent psychiatry, 31(3), 131-145.
Raghavan, K., Dedeepiya, V. D., Suryaprakash, V., Rao, K. S., Ikewaki, N., Sonoda, T., Levy, G. A., Iwasaki, M., Senthilkumar, R., Preethy, S., & Abraham, S. J. (2022). Beneficial effects of novel Aureobasidium pullulans strains produced beta-1,3-1,6 glucans on interleukin-6 and D-dimer levels in COVID-19 patients; results of a randomized multiple-arm pilot clinical study. Biomedicine & pharmacotherapy=Biomedecine & pharmacotherapie, 145, 112243.
^{a}Raghavan, K., Dedeepiya, V. D., Ikewaki, N., Sonoda, T., Iwasaki., M., Preethy. S., Abraham, S. J. K. Improvement of behavioural pattern and alpha-synuclein levels in autism spectrum disorder after consumption of a beta-glucan food supplement in a randomized, parallel-group pilot clinical study. BMJ Neurology Open (In print). Preprint in MedRxiv: https://doi.org/10.1101/2021.06.28.21259619
^{b}Raghavan, K., Dedeepiya, V. D. Kandaswamy, R., Balamurugan. M., Ikewaki, N., Sonoda, T., Kurosawa, G., Iwasaki, M., Preethy, S., Abraham, S. J. K. (2021). Improvement of sleep patterns and serum melatonin levels in children with autism spectrum disorders after consumption of beta-1,3/1,6-glucan in a pilot clinical study. Research Square rs.3.rs-701988/v1; doi: 10.21203/rs.3.rs-701988/v1
^{c}Raghavan, K., Kandaswamy, R. S., Ikewaki, N., Iwasaki, M., & Abraham, S. (2021). Potentials to alleviate coagulopathy and enhance microglial function of beta (β)-glucans, making them worth a clinical study for COVID-19's neurological sequalae. Journal of the neurological sciences, 427, 117554.
Sampson, T. R., Challis, C., Jain, N., Moiseyenko, A., Ladinsky, M. S., Shastri, G. G., Thron, T., Needham, B. D., Horvath, I., Debelius, J. W., Janssen, S., Knight, R., Wittung-Stafshede, P., Gradinaru, V., Chapman, M., & Mazmanian, S. K. (2020). A gut bacterial amyloid promotes α-synuclein aggregation and motor impairment in mice. eLife, 9, e53111.
Santocchi, E., Guiducci, L., Prosperi, M., Calderoni, S., Gaggini, M., Apicella, F., Tancredi, R., Billeci, L., Mastromarino, P., Grossi, E., Gastaldelli, A., Morales, M. A., & Muratori, F. (2020). Effects of Probiotic Supplementation on Gastrointestinal, Sensory and Core Symptoms in Autism Spectrum Disorders: A Randomized Controlled Trial. Frontiers in psychiatry, 11, 550593.
Swirski, M., Miners, J. S., de Silva, R., Lashley, T., Ling, H., Holton, J., Revesz, T., & Love, S. (2014). Evaluating the relationship between amyloid-3 and α-synuclein phosphorylated at Ser129 in dementia with Lewy bodies and Parkinson's disease. Alzheimer's research & therapy, 6(5-8), 77.
Souza, N. C., Mendonca, J. N., Portari, G. V., Jordao Junior, A. A., Marchini, J. S., & Chiarello, P. G. (2012). Intestinal permeability and nutritional status in developmental disorders. Alternative therapies in health and medicine, 18(2), 19-24.
Tomova A, Husarova V, Lakatosova S, Bakos J, Vlkova B, Babinska K, Ostatnikova D. Gastrointestinal microbiota in children with autism in Slovakia. Physiol Behav. 2015; 138:179-87.
Werner, T., Horvath, I., & Wittung-Stafshede, P. (2020). Crosstalk Between Alpha-Synuclein and Other Human and Non-Human Amyloidogenic Proteins: Consequences for Amyloid Formation in Parkinson's Disease. Journal of Parkinson's disease, 10(3), 819-830.
Shen R L, Dang X Y, Dong J L, Hu X Z. Effects of oat β-glucan and barley β-glucan on fecal characteristics, intestinal microflora, and intestinal bacterial metabolites in rats. J Agric Food Chem. 2012 Nov. 14; 60(45):11301-8.
Turunen K, Tsouvelakidou E, Nomikos T, Mountzouris K C, Karamanolis D, Triantafillidis J, Kyriacou A. Impact of beta-glucan on the faecal microbiota of polypectomized patients: a pilot study. Anaerobe. 2011 December; 17(6):403-6
Zhen W, Liu Y, Shao Y, Ma Y, Wu Y, Guo F, Abbas W, Guo Y, Wang Z. Yeast β-Glucan Altered Intestinal Microbiome and Metabolome in Older Hens. Front Microbiol. 2021 Dec. 17; 12:766878.
1. Al-Mazidi, S., & Al-Ayadhi, L. Y. (2021). Plasma Levels of Alpha and Gamma Synucleins in Autism Spectrum Disorder: An Indicator of Severity. Medical principles and practice: international journal of the Kuwait University, Health Science Centre, 30(2), 160-167. https://doi.org/i0.1159/000513935
2. Alp, H., Varol, S., Celik, M. M., Altas, M., Evliyaoglu, O., Tokgoz, O., Tanriverdi, M. H., & Uzar, E. (2012). Protective effects of beta glucan and gliclazide on brain tissue and sciatic nerve of diabetic rats induced by streptozosin. Experimental diabetes research, 2012, 230342. https://doi.org/10.1155/2012/230342
3. Centers for Disease Control and Prevention. What is Autism Spectrum Disorder? Retrieved from https://www.cdc.gov/ncbddd/autism/facts.html
4. Dedeepiya, V. D., Sivaraman, G., Venkatesh, A. P., Preethy, S., & Abraham, S. J. (2012).
5. Potential effects of nichi glucan as a food supplement for diabetes mellitus and hyperlipidemia: preliminary findings from the study on three patients from India. Case reports in medicine, 2012, 895370. https://doi.org/10.1155/2012/895370
6. Ganesh, J. S., Rao, Y. Y., Ravikumar, R., Jayakrishnan, G. A., Iwasaki, M., Preethy, S., & Abraham, S. J. (2014). Beneficial effects of black yeast derived 1-3, 1-6 Beta Glucan-Nichi Glucan in a dyslipidemic individual of Indian origin—a case report. Journal of dietary supplements, 11(1), 1-6. https://doi.org/10.3109/19390211.2013.859211
7. Ikewaki, N., Fujii, N., Onaka, T., Ikewaki, S., & Inoko, H. (2007). Immunological actions of Sophy beta-glucan (beta-1,3-1,6 glucan), currently available commercially as a healthfood supplement. Microbiology and immunology, 51(9), 861-873. https://doi.org/10.1111/j.1348-0421.2007.tb03982.x
8. Ikewaki, N., Iwasaki, M., Kurosawa, G., Rao, K. S., Lakey-Beitia, J., Preethy, S., & Abraham, S. J. (2021). β-glucans: wide-spectrum immune-balancing food-supplement-based enteric (p3-WIFE) vaccine adjuvant approach to COVID-19. Human vaccines & immunotherapeutics, 1-6. Advance online publication. https://doi.org/10.1080/21645515.2021.1880210
9. Kadak, M. T., Cetin, I., Tarakcioglu, M. C., Ozer, O. F., Kacar, S., & Çimen, B. (2015). Low Serum Level α-Synuclein and Tau Protein in Autism Spectrum Disorder Compared to Controls. Neuropediatrics, 46(6), 410-415. https://doi.org/10.1055/s-0035-1565273
10. Karhu, E., Zukerman, R., Eshraghi, R. S., Mittal, J., Deth, R. C., Castejon, A. M., Trivedi, M., Mittal, R., & Eshraghi, A. A. (2020). Nutritional interventions for autism spectrum disorder. Nutrition reviews, 78(7), 515-531. https://doi.org/10.1093/nutrit/nuz092
11. Obergasteiger, J., Überbacher, C., Pramstaller, P. P., Hicks, A. A., Corti, C., & Volta, M. (2017). CADPS2 gene expression is oppositely regulated by LRRK2 and alpha-synuclein. Biochemical and biophysical research communications, 490(3), 876-881. https://doi.org/10.1016/j.bbrc.2017.06.134
12. Peng, M., Tabashsum, Z., Anderson, M., Truong, A., Houser, A. K., Padilla, J., Akmel, A., Bhatti, J., Rahaman, S. O., & Biswas, D. (2020). Effectiveness of probiotics, prebiotics, and prebiotic-like components in common functional foods. Comprehensive reviews in food science and food safety, 19(4), 1908-1933. https://doi.org/10.1111/1541-4337.12565
13. Rahayu, M., Kurniawan, S. N., Husna, M., Hermawan, H. O. (2016). The effect of beta glucan of Saccharomyces cerevisae on the decrease of alpha synuclein expression in the brain substantia nigra of parkinson's wistar strain rats (Rattus novergicus) model induced with rotenone. Malang Neurology Journal 2(1). ISSN 2442-5001. Retrieved from https://mnj.ub.ac.id/index.php/mnj/article/view/32
12. Ramaekers, V. T., Sequeira, J. M., DiDuca, M., Vrancken, G., Thomas, A., Philippe, C., Peters, M., Jadot, A., & Quadros, E. V. (2019). Improving Outcome in Infantile Autism with Folate Receptor Autoimmunity and Nutritional Derangements: A Self-Controlled Trial. Autism research and treatment, 2019, 7486431. https://doi.org/10.1155/2019/7486431
13. Shah, V. B., Williams, D. L., & Keshvara, L. (2009). beta-Glucan attenuates TLR2- and TLR4-mediated cytokine production by microglia. Neuroscience letters, 458(3), 111-115. https://doi.org/10.1016/j.neulet.2009.04.039
14. Shi, H., Yu, Y., Lin, D., Zheng, P., Zhang, P., Hu, M., Wang, Q., Pan, W., Yang, X., Hu, T., Li, Q., Tang, R., Zhou, F., Zheng, K., & Huang, X. F. (2020). β-glucan attenuates cognitive impairment via the gut-brain axis in diet-induced obese mice. Microbiome, 8(1), 143. https://doi.org/10.1186/s40168-020-00920-y
15. Siddique, A., Khan, H. F., Ali, S., Abdullah, A., Munir, H., & Ariff, M. (2020). Estimation of Alpha-Synuclein Monomer and Oligomer Levels in the Saliva of the Children With Autism Spectrum Disorder: A Possibility for an Early Diagnosis. Cureus, 12(8), e9936. https://doi.org/10.7759/cureus.9936
16. Srikantha, P., & Mohajeri, M. H. (2019). The Possible Role of the Microbiota-Gut-Brain-Axis in Autism Spectrum Disorder. International journal of molecular sciences, 20(9), 2115. https://doi.org/10.3390/ijms20092115
17. Sriwimol, W., & Limprasert, P. (2018). Significant Changes in Plasma Alpha-Synuclein and Beta-Synuclein Levels in Male Children with Autism Spectrum Disorder. BioMed research international, 2018, 4503871. https://doi.org/10.1155/2018/4503871
18. Xu, M., Mo, X., Huang, H., Chen, X., Liu, H., Peng, Z., Chen, L., Rong, S., Yang, W., Xu, S., & Liu, L. (2020). Yeast β-glucan alleviates cognitive deficit by regulating gut microbiota and metabolites in Aβ1-42-induced AD-like mice. International journal of biological macromolecules, 161, 258-270. https://doi.org/10.1016/j.ijbiomac.2020.05.180
19. Vargas, K. J., Schrod, N., Davis, T., Fernandez-Busnadiego, R., Taguchi, Y V., Laugks, U., Lucic, V., & Chandra, S. S. (2017). Synucleins Have Multiple Effects on Presynaptic Architecture. Cell reports, 18(1), 161-173. https://doi.org/10.1016/j.celrep.2016.12.023
C1. Goldman S E, Richdale A L, Clemons T, Malow B A. Parental sleep concerns in autism spectrum disorders: variations from childhood to adolescence. J Autism Dev Disord. 2012 April; 42(4):531-8.
C2. Malow B, Adkins K W, McGrew S G, Wang L, Goldman S E, Fawkes D, Burnette C. Melatonin for sleep in children with autism: a controlled trial examining dose, tolerability, and outcomes. J Autism Dev Disord. 2012 August; 42(8):1729-37
C3. Gagnon K, Godbout R. Melatonin and Comorbidities in Children with Autism Spectrum Disorder. Curr Dev Disord Rep. 2018; 5(3):197-206.
C4. Maras A, Schroder C M, Malow B A, Findling R L, Breddy J, Nir T, Shahmoon S, Zisapel N, Gringras P. Long-Term Efficacy and Safety of Pediatric Prolonged-Release Melatonin for Insomnia in Children with Autism Spectrum Disorder. J Child Adolesc Psychopharmacol. 2018 December; 28(10):699-710.
C5. Cortesi F, Giannotti F, Sebastiani T, Panunzi S, Valente D. Controlled-release melatonin, singly and combined with cognitive behavioural therapy, for persistent insomnia in children with autism spectrum disorders: a randomized placebo-controlled trial. J Sleep Res. 2012 December; 21(6):700-9. doi: 10.1111/j.1365-2869.2012.01021.x.
C6. Russcher M, Koch B C, Nagtegaal J E, van Ittersum F J, Pasker-de Jong P C, Hagen E C, van Dorp W T, Gabreëls B, Wildbergh T X, van der Westerlaken M M, Gaillard C A, Ter Wee P M. Long-term effects of melatonin on quality of life and sleep in haemodialysis patients (Melody study): a randomized controlled trial. Br J Clin Pharmacol. 2013 November; 76(5):668-79. doi: 10.1111/bcp.12093.
C7. Costa H F B, Chemical and biological characterization of an aqueous Sambucus nigra L. flower extract 2019.
C8. Dutta S D, Patel D K, Ganguly K, Lim K T. Effects of GABA/β-glucan supplements on melatonin and serotonin content extracted from natural resources. PLoS One. 2021 Mar. 5; 16(3):e0247890. doi: 10.1371/journal.pone.0247890.
C9. Dedeepiya V D, Sivaraman G, Venkatesh A P, et al. Potential effects of nichi glucan as a food supplement for diabetes mellitus and hyperlipidemia: preliminary findings from the study on three patients from India. Case Rep Med 2012 (2012):895370.
C10. Ganesh J S, Rao Y Y, Ravikumar R, Jayakrishnan G A, Iwasaki M, Preethy S, Abraham S J. Beneficial effects of black yeast derived 1-3, 1-6 Beta Glucan-Nichi Glucan in a dyslipidemic individual of Indian origin—a case report. J Diet Suppl (2014)11:1-6.
C11. Mizobuchi S, Taniwaki C, Watanabe Y, Sasaguri S. Antitumor effects of combined therapy with intraperitoneal CDDP and oral Sofy β-glucan. Abstract presented at the 63rd General Meeting of the Japanese Society of Gastroenterology, Japan (2008).
C12. Mio M. Effect of oral intake of black yeast beta-glucan on NK activity in the elderly and patients with cancer. Abstract presented at 29th Annual Meeting of the Japanese Society of Venous and Enteral Nutrition Pacifico Yokohama, Japan (2014).
C13. Katoh S, Rao K S, Suryaprakash V, Horiguchi A, Kushibiki T, Ojima K, Iwasaki M, Takeda M, Senthilkumar R, Rajmohan M, Karthick R, Preethy S, Abraham S. β-Glucans: Wide-spectrum Immune-balancing Food-supplement-based Enteric (β-WIFE) Vaccine Adjuvant Approach to COVID-19. Human Vaccines & Immunotherapeutics (2021)
C14. Parvataneni T, Srinivas S, Shah K, Patel R S. Perspective on Melatonin Use for Sleep Problems in Autism and Attention-Deficit Hyperactivity Disorder: A Systematic Review of Randomized Clinical Trials. Cureus. 2020 May 28; 12(5):e8335. doi: 10.7759/cureus.8335.
C15. Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol. 2007; 51(9):861-73. doi: 10.1111/j.1348-0421.2007.tb03982.x.
C16. Braam W, Ehrhart F, Maas APHM, Smits M G, Curfs L. Low maternal melatonin level increases autism spectrum disorder risk in children. Res Dev Disabil. 2018 November; 82:79-89. doi: 10.1016/j.ridd.2018.02.017.
C17. Li N, Yang J, Zhang J, Liang C, Wang Y, Chen B, Zhao C, Wang J, Zhang G, Zhao D, Liu Y, Zhang L, Yang J, Li G, Gai Z, Zhang L, Zhao G. Correlation of Gut Microbiome Between ASD Children and Mothers and Potential Biomarkers for Risk Assessment. Genomics Proteomics Bioinformatics. 2019 February; 17(1):26-38. doi: 10.1016/j.gpb.2019.01.002. Epub 2019 Apr. 23. PMID: 31026579; PMCID: PMC6520911.
C18. Innate Immunity and Neuroinflammation in Neuropsychiatric Conditions Including Autism Spectrum Disorders: Role of Innate Immune Memory. Harumi Jyonouchi DOI: 10.5772/intechopen.87167
C19. Cheng W Y, Lam K L, Pik-Shan Kong A, Chi-Keung Cheung P. Prebiotic supplementation (beta-glucan and inulin) attenuates circadian misalignment induced by shifted light-dark cycle in mice by modulating circadian gene expression. Food Res Int. 2020 November; 137:109437. doi: 10.1016/j.foodres.2020.109437.
C20. Shi H, Yu Y, Lin D, Zheng P, Zhang P, Hu M, Wang Q, Pan W, Yang X, Hu T, Li Q, Tang R, Zhou F, Zheng K, Huang X F. β-glucan attenuates cognitive impairment via the gut-brain axis in diet-induced obese mice. Microbiome. 2020 Oct. 2; 8(1):143. doi: 10.1186/s40168-020-00920-y.
D1. Valdes A M, Walter J, Segal E, Spector T D. Role of the gut microbiota in nutrition and health. BMJ. 2018 Jun. 13; 361:k2179.
D2. Ghaisas S, Maher J, Kanthasamy A. Gut microbiome in health and disease: Linking the microbiome-gut-brain axis and environmental factors in the pathogenesis of systemic and neurodegenerative diseases. Pharmacol Ther. 2016 February; 158:52-62.
D3. Shreiner A B, Kao J Y, Young V B. The gut microbiome in health and in disease. Curr Opin Gastroenterol. 2015 January; 31(1):69-75. doi: 10.1097/MOG.0000000000000139. PMID: 25394236; PMCID: PMC4290017.
D4. Zierer J, Jackson M A, Kastenmüller G, Mangino M, Long T, Telenti A, Mohney R P, Small K S, Bell J T, Steves C J, Valdes A M, Spector T D, Menni C. The fecal metabolome as a functional readout of the gut microbiome. Nat Genet. 2018 June; 50(6):790-795.
D5. Dedeepiya V, Sivaraman G, Venkatesh A, Preethy S, Abraham S. Potential Effects of Nichi Glucan as a Food Supplement for Diabetes Mellitus and Hyperlipidemia; Preliminary Findings from the Study on Three Patients from India. Case Reports in Medicine 2012 (2012), Article ID 895370
D6. Ganesh J S, Rao Y Y, Ravikumar R, Jayakrishnan A G, Iwasaki M, Preethy S, Abraham S. Beneficial effects of Black yeast derived 1-3, 1-6 beta glucan-Nichi Glucan in a dyslipidemic individual of Indian origin—A case report. J Diet Suppl. 2014; 11(1):1-6.
D7. Raghavan K, Dedeepiya V D, Ikewaki N, Sonoda T, Iwasaki M, Preethy S, Abraham S J K. Improvement of behavioural pattern and alpha-synuclein levels in autism spectrum disorder after consumption of a beta-glucan food supplement in a randomized, parallel-group pilot clinical study. BMJ Neurology Open (In print)
D8. Raghavan K, Dedeepiya V D, Kandaswamy R, Balamurugan M, Ikewaki N, Sonoda T, Kurosawa G, Iwasaki M, Preethy S, Abraham S J K. Improvement of sleep patterns and serum melatonin levels in children with autism spectrum disorders after consumption of beta-1,3/1,6-glucan in a pilot clinical study. Research Square rs.3.rs-701988/v1; doi: 10.21203/rs.3.rs-701988/v1
D9. Raghavan K, Dedeepiya V D, Srinivasan S, Pushkala S, Subramanian S, Ikewaki N, Iwasaki M, Senthilkumar R, Preethy S, Abraham S. Disease-modifying immune-modulatory effects of the N-163 strain of Aureobasidium pullulans-produced 1,3-1,6 Beta glucans in young boys with Duchenne muscular dystrophy: Results of an open-label, prospective, randomized, comparative clinical study. medRxiv 2021.12.13.21267706
D10. Ikewaki N, Kurosawa G, Iwasaki M, Preethy S, Dedeepiya V D, Vaddi S, Senthilkumar R, Levy G A, Abraham S J K. Hepatoprotective effects of Aureobasidium pullulans derived Beta 1,3-1,6 biological response modifier glucans in a STAM-animal model of non-alcoholic steatohepatitis.bioRxiv 2021.07.08.451700; doi: 10.1101/2021.07.08.451700
D11. Pushkala S, Seshayyan S, Theranirajan E, Sudhakar D, Raghavan K, Dedeepiya V D, Ikewaki N, Iwasaki M, Preethy S, Abraham S. Efficient control of IL-6, CRP and Ferritin in Covid-19 patients with two variants of Beta-1,3-1,6 glucans in combination, within 15 days in an open-label prospective clinical trial. medRxiv 2021.12.14.21267778; doi: 10.1101/2021.12.14.21267778
D12. Raghavan K, Dedeepiya V D, Suryaprakash V, Rao K S, Ikewaki N, Sonoda T, Levy G A, Iwasaki M, Senthilkumar R, Preethy S, Abraham S J K. Beneficial Effects of novel Aureobasidium pullulans strains produced beta-1,3-1,6 glucans on interleukin-6 and D-Dimer levels in COVID-19 patients; results of a randomized multiple-arm pilot clinical study. Biomedicine and Pharmacotherapy 2021. sciencedirect. https://doi.org/10.1016/j.biopha.2021.112243
D13. Raghavan K, Dedeepiya V D, Yamamoto N, Ikewaki N, Sonoda T, Kurosawa G, Iwasaki M, Kandaswamy R, Senthilkumar R, Preethy S, Abraham S J K. Beneficial reconstitution of gut microbiota and control of alpha-synuclein and curli-amyloids-producing enterobacteria, by beta 1,3-1,6 glucans in a clinical pilot study of autism and potentials in neurodegenerative diseases. medRxiv 2021.10.26.21265505; doi: 10.1101/2021.10.26.21265505
D14. Bashir K M I, Choi J S. Clinical and Physiological Perspectives of β-Glucans: The Past, Present, and Future. Int J Mol Sci. 2017 Sep. 5; 18(9):1906.
D15. Ikewaki N, Fujii N, Onaka T, Ikewaki S, Inoko H. Immunological actions of Sophy beta-glucan (beta-1,3-1,6 glucan), currently available commercially as a health food supplement. Microbiol Immunol. 2007; 51(9):861-73.
D16. Ikewaki N, Onaka T, Ikeue Y, Nagataki M, Kurosawa G, Dedeepiya V D, Rajmohan M, Vaddi S, Senthilkumar R, Preethy S, Abraham S J K. Beneficial effects of the AFO-202 and N-163 strains of Aureobasidium pullulans produced 1,3-1,6 beta glucans on non-esterified fatty acid levels in obese diabetic KKAy mice: A comparative study.bioRxiv 2021.07.22.453362; doi: 10.1101/2021.07.22.453362
D17. Ikewaki N, Sonoda T, Kurosawa G, Iwasaki M, Dedeepiya V D, Senthilkumar R, Preethy S, Abraham S J K. Immune and metabolic beneficial effects of Beta 1,3-1,6 glucans produced by two novel strains of Aureobasidium pullulans in healthy middle-aged Japanese men: An exploratory study. medRxiv 2021.08.05.21261640; doi: 10.1101/2021.08.05.21261640
D18. STAM Model. https://www.smccro-lab.com/service/service_disease_area/stam.html
D19. Nakashima A, Sugimoto R, Suzuki K, Shirakata Y, Hashiguchi T, Yoshida C, Nakano Y Anti-fibrotic activity of Euglena gracilis and paramylon in a mouse model of non-alcoholic steatohepatitis. Food Sci Nutr. 2018; 7(1):139-147
D20. Dantzer R. Neuroimmune Interactions: From the Brain to the Immune System and Vice Versa. Physiol Rev. 2018 Jan. 1; 98(1):477-504.
D21. KolodziejczykAA, Zheng D, Shibolet O, Elinav E. The role of the microbiome in NAFLD and NASH. EMBO Mol Med. 2019 February; 11(2):e9302. doi: 10.15252/emmm.201809302. PMID: 30591521; PMCID: PMC6365925.
D22. Chen J, Vitetta L. Gut Microbiota Metabolites in NAFLD Pathogenesis and Therapeutic Implications. Int J Mol Sci. 2020 Jul. 23; 21(15):5214. doi: 10.3390/ijms21155214. PMID: 32717871; PMCID: PMC7432372.
D23. Kang D W, Adams J B, Vargason T, Santiago M, Hahn J, Krajmalnik-Brown R. Distinct Fecal and Plasma Metabolites in Children with Autism Spectrum Disorders and Their Modulation after Microbiota Transfer Therapy. mSphere. 2020 Oct. 21; 5(5):e00314-20. doi: 10.1128/mSphere. 00314-20. PMID: 33087514; PMCID: PMC7580952.
D24. Vascellari S, Palmas V, Melis M, Pisanu S, Cusano R, Uva P, Perra D, Madau V, Sarchioto M, Oppo V, Simola N, Morelli M, Santoru M L, Atzori L, Melis M, Cossu G, Manzin A. Gut Microbiota and Metabolome Alterations Associated with Parkinson's Disease. mSystems. 2020 Sep. 15; 5(5):e00561-20. doi: 10.1128/mSystems. 00561-20. PMID: 32934117; PMCID: PMC7498685.
D25. Gibson C M, Childs-Kean L M, Naziruddin Z, Howell C K. The alteration of the gut microbiome by immunosuppressive agents used in solid organ transplantation. Transpl Infect Dis. 2021 February; 23(1):e13397.

1. A composition for improving gut microbiota, comprising a beta-glucan produced by Aureobasidium pullulans APO-202 (FERM BP-I9327).

2. The composition according to claim 1, wherein the improvement of gut microbiota comprises a decrease of Akkermansia muciniphila with an increase of beneficial bacteria including Roseburia in a gut.

3. The composition according to claim 1, wherein the composition is for prophylactic, ameliorating and/or curative treatment of autism spectrum disorders (ASD), multiple sclerosis (MS), Alzheimer's disease (AD), Parkinson's disease (PD) and/or epilepsy.

4. The composition according to claim 1, wherein the composition is for improving behavioural pattern and alpha-synuclein levels.

5. The composition according to claim 1, wherein the composition is for improving sleep pattern and serum melatonin.

6. The composition according to claim 5, wherein the improvement is in a child with autism spectrum disorder.

7. The composition according to claim 1, wherein the composition is a pharmaceutical composition.

8. The composition according to claim 1, wherein the composition is a food composition.

9. A method of improving gut microbiota in a subject, comprising administering a beta-glucan produced by Aureobasidium pullulans AFO-202 (FERM BP-19327) to the subject in need thereof.
